(19) United States
(12) Patent Application Publication
(10) Pub. No.: US 2025/0215039 A1
(43) Pub. Date: Jul. 3, 2025

(54) NUCLEOSIDES FOR TREATING CANCER

(71) Applicant: PRIMEFOUR THERAPEUTICS, INC., San Diego, CA (US)

(72) Inventors: Eckard WEBER, Los Angeles, CA (US); Michael G. CORDINGLEY, Rosemère (CA); Andrew James BURNIE, Montreal (CA); William BROWN, Montreal (CA); Chandra Mohan DARAPANENI, Montreal (CA); Marco PALADINO, Montreal (CA)

(21) Appl. No.: 18/852,024

(22) PCT Filed: Mar. 30, 2023
(86) PCT No.: PCT/US2023/016934

§ 371 Date: Sep. 27, 2024

Related U.S. Application Data
(60) Provisional application No. 63/325,487, filed on Mar. 30, 2022.

(51) Int. Cl. C07H 19/173; A61K 31/7068; A61K 31/7072; A61K 31/7076; A61K 31/708; A61K 45/06; A61P 35/00; C07H 17/02; C07H 19/067; C07H 19/073; C07H 19/167

(52) U.S. Cl. CPC C07H 19/173; A61K 31/7068; A61K 31/7072; A61K 31/7076; A61K 31/708; A61K 45/06; A61P 35/00; C07H 17/02; C07H 19/067; C07H 19/073; C07H 19/167

(57) ABSTRACT

The present disclosure provides DNA damage repair enzyme inhibitors and methods of treating or preventing a cancer in a subject by administering a DNA damage repair enzyme inhibitor to the subject.

### BACKGROUND

### Field

The present disclosure provides DNA damage repair (DDR) enzyme inhibitors and methods of treating or preventing a cancer in a subject by administering a DDR enzyme inhibitor to the subject.

### Background

The repair of DNA double-strand breaks (DSBs) is essential for the maintenance of genome stability and cell viability. DSBs can be repaired by one of three main pathways: homologous recombination (HR), non-homologous end-joining (NHEJ), and alternative NHEJ (alt-NHEJ). Microhomology-mediated end-joining (MMEJ), also referred to as polymerase theta-mediated end joining (TMEJ) due to requirement of Pol θ, is the most well characterised alt-NHEJ mechanism. (Malaby et al., Method. Enzymol. 592:103-121 (2017)). HR-mediated repair is a high-fidelity mechanism essential for accurate error-free repair, preventing cancer-predisposing genomic stability. Conversely, NHEJ and MMEJ are error-prone pathways that can leave mutational scars at the site of repair. MMEJ can function parallel to both HR and NHEJ pathways (Zatreanu et al., Nature Communications 12:3636 (2021) https://doi.org/10.1038/s41467-021-23463-8; Truong et al. PNAS 2013, 110 (19), 7720-7725); WO2021/123785).

The survival of cancer cells, unlike normal cells, is often dependent on the mis-regulation of DNA damage response pathways. For example, an increased dependency on one pathway (often mutagenic) to cope with either the inactivation of another one, or the enhanced replication stress resulting from increased proliferation. An aberrant DDR can also sensitise cancer cells to specific types of DNA damage, thus, defective DDR can be exploited to develop targeted cancer therapies. Crucially, cancer cells with impairment or inactivation of HR and NHEJ become hyper-dependent on MMEJ-mediated DNA repair. Genetic, cell biological, and biochemical data have identified Pol θ (UniProtKB—075417 (DPOLQ_HUMAN) as the key protein in MMEJ (Kent et al., Nature Structural & Molecular Biology (2015), 22(3), 230-237, Mateos-Gomez et al., Nature (2015), 518(7538), 254-257). Pol θ is multifunctional enzyme, which comprises an N-terminal helicase domain (SF2 HEL308-type) and a C-terminal low-fidelity DNA polymerase domain (A-type) (Wood & Doublie, DNA Repair (2016), 44, 22-32). Both domains have been shown to have concerted mechanistic functions in MMEJ. The helicase domain mediates the removal of RPA protein from ssDNA ends and stimulates annealing. The polymerase domain extends the ssDNA ends and fills the remaining gaps. Pol θ is encoded by the POLQ gene in mammalian genomes.

Therapeutic inactivation of Pol θ would thus disable the ability of cells to perform MMEJ and provide a targeted strategy in an array of defined tumor contexts. Pol θ has been shown to be essential for the survival of HR-defective (HRD) cells (e.g. synthetic lethal with FA/BRCA-deficiency) and is up-regulated in HRD tumor cell lines (Ceccaldi et al., Nature (2015), 518(7538), 258-262). In vivo studies also show that Pol θ is significantly over expressed in subsets of HRD ovarian, uterine and breast cancers with associated poor prognosis (Higgins et al., Oncolarget (2010), 1, 175-184, Lemee et al., PNAS (2010), 107(30), 13390-13395). Pol θ is largely repressed in normal tissues but has been shown to be upregulated in matched cancer samples thus correlating elevated expression with disease (Kawamura et al., International Journal of Cancer (2004), 109(1), 9-16). Also, its suppression or inhibition confers radio-sensitivity in tumor cells. Finally, Pol θ inhibition prevents the MMEJ-dependent functional reversion of BRCA2 mutations that underlies the emergence of cisplatin and PARPi resistance in tumors. There is a need for effective DDR enzyme inhibitors for the treatment of cancer.

### BRIEF SUMMARY

In one aspect, the present disclosure provides compounds of (i) Table 1, see below, and the pharmaceutically acceptable salts or solvates thereof, and/or tautomers thereof, and/or mono-, di-, or trisphosphates thereof; (ii) Table 2, see below, and the pharmaceutically acceptable salts or solvates thereof, and/or tautomers thereof, and/or mono-, di-, or trisphosphates thereof; (iii) Table 3, see below, and the pharmaceutically acceptable salts or solvates thereof, and/or tautomers thereof, and/or mono-, di-, or trisphosphates thereof; and (iv) Table 3A, see below, and the pharmaceutically acceptable salts or solvates thereof, and/or tautomers thereof. The compounds of Tables 1-3 and 3A, and the pharmaceutically acceptable salts or solvates thereof, and/or tautomers thereof, are collectively referred to as “Compounds of the Disclosure” or individually referred to as a “Compound of the Disclosure.”

In another aspect, the present disclosure provides a pharmaceutical composition comprising a Compound of the Disclosure and one or more pharmaceutically acceptable excipients.

In another aspect, the present disclosure provides methods of treating or preventing cancer in a subject by administering a therapeutically effective amount of a Compound of the Disclosure to a subject, e.g., a human patient, in need thereof.

In another aspect, the present disclosure provides methods of inhibiting a DDR enzyme in a subject by administering a therapeutically effective amount of a Compound of the Disclosure to a subject in need thereof.

In another aspect, the present disclosure provides methods of treating metastatic prostate cancer in a subject that has become resistant or may become resistant to androgen deprivation therapy (ADT), comprising administering to the subject an effective amount of a Compound of the Disclosure.

In another embodiment, the present disclosure provides a method, comprising administering a therapeutically effective amount of a Compound of the Disclosure to a subject in need thereof, wherein: (a) the subject has cancer; and (b) the cancer is characterized as having a biomarker, e.g., overexpression of Pol θ, Pol η, and/or Pol μ.

### BRIEF DESCRIPTION OF THE DRAWINGS

FIG. 1 is waterfall plot showing that gemcitabine is indiscriminately cytotoxic against a panel of cancer and non-cancer cell lines.

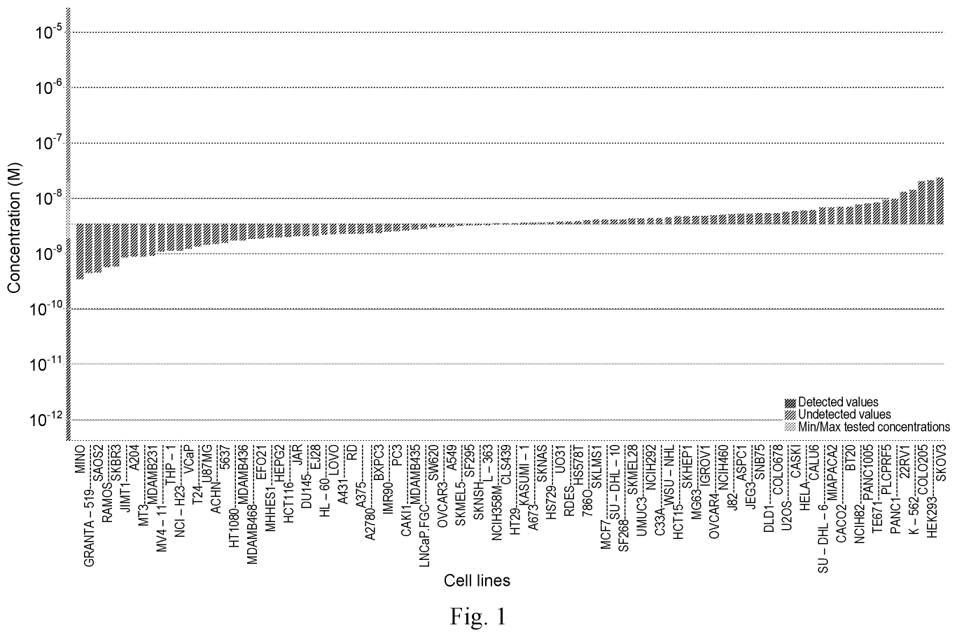

FIG. 2 is a waterall plot showing the Cpd. No. 4 is selectively cytotoxic in cancer cell lines with high DDR activity.

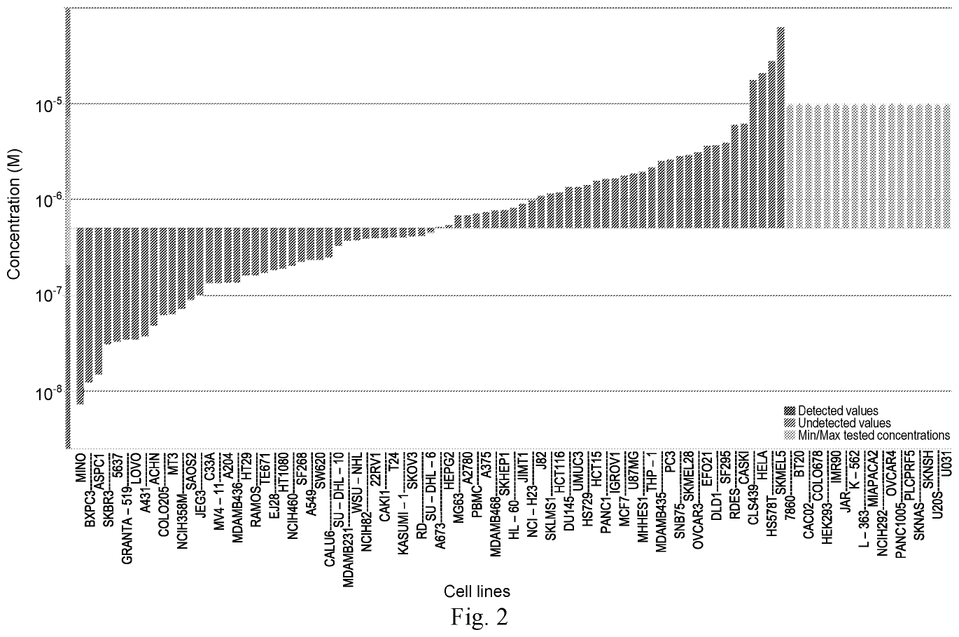

FIG. 3 is a waterall plot showing the Cpd. No. 197 is selectively cytotoxic in cancer cell lines with high DDR activity.

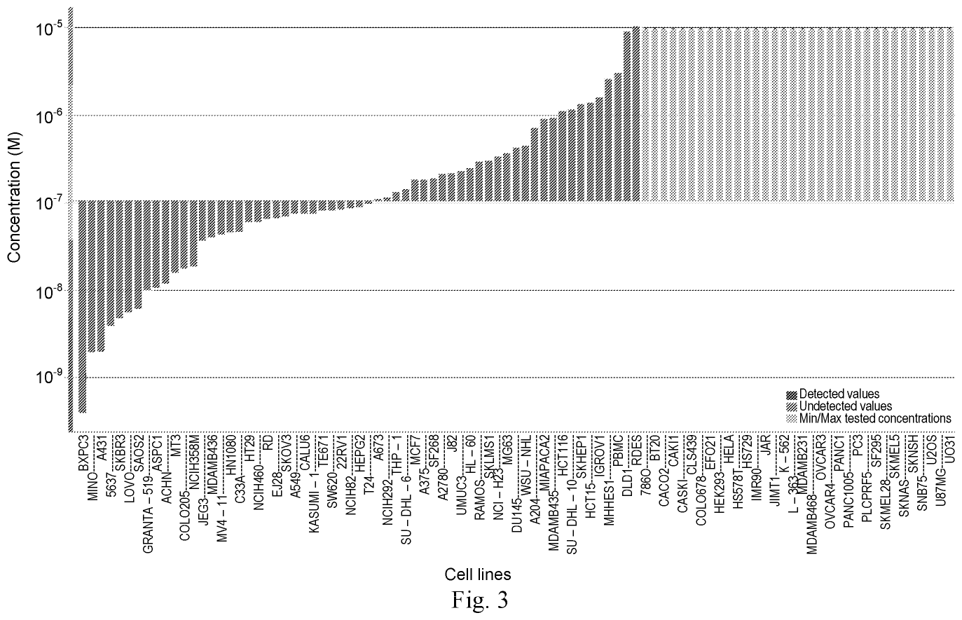

FIG. 4 is a line graph showing that cytidine is rapidly metabolized by human cytidine deaminase (CDA).

FIG. 5 is a line graph showing that gemcitabine is rapidly metabolized by human CDA.

FIG. 6 is a line graph showing that Cpd. No. 4 is more stable than cytidine and gemcitabine to metabolism by human CDA.

FIG. 7 is a line graph showing that Cpd. No. 197 is not metabolized by human CDA.

FIG. 8 is a line graph showing that Cpd. No. 6 is not metabolized by human CDA.

FIG. 9 is a line graph showing that Cpd. No. 190 is not metabolized by human CDA.

### DETAILED DESCRIPTION

### I. Compounds of the Disclosure

In one embodiment, Compounds of the Disclosure are compounds of Table 1, or a pharmaceutically acceptable salt, solvate, and/or tautomer thereof, and/or mono-, di-, or trisphosphates thereof.

TABLE 1

| Cpd. No. | Structure | Chemical Name |
|---|---|---|
| 1 | 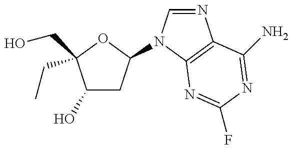 [SMILES: CC[C@]1(CO)O[C@@H](n2cnc3c(N)nc(F)nc32)C[C@@H]1O] | (2R,3S,5R)-5-(6-amino-2-fluoro-9H-purin-9- yl)-2-ethyl-2- (hydroxymethyl)tetrahydrofuran-3-ol |
| 2 | 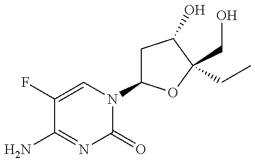 [SMILES: CC[C@]1(CO)O[C@@H](n2cc(F)c(N)nc2=O)C[C@@H]1O] | 4-amino-1-((2R,4S,5R)-5-ethyl-4-hydroxy-5- (hydroxymethyl)tetrahydrofuran-2-yl)-5- fluoropyrimidin-2(1H)-one |
| 3 | 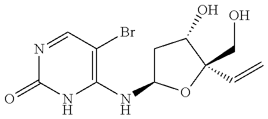 [SMILES: C=C[C@]1(CO)O[C@@H](Nc2[nH]c(=O)ncc2Br)C[C@@H]1O] | 5-bromo-6-(((2R,4S,5R)-4-hydroxy-5- (hydroxymethyl)-5-vinyltetrahydrofuran-2- yl)amino)pyrimidin-2(1H)-one |
| 4 | 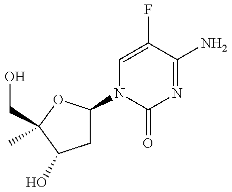 [SMILES: C[C@]1(CO)O[C@@H](n2cc(F)c(N)nc2=O)C[C@@H]1O] | 4-amino-5-fluoro-1-((2R,4S,5R)-4-hydroxy- 5-(hydroxymethyl)-5-methyltetrahydrofuran- 2-yl)pyrimidin-2(1H)-one |
| 5 | 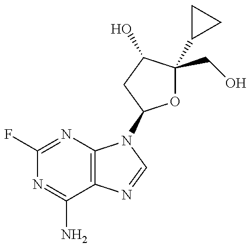 [SMILES: Nc1nc(F)nc2c1ncn2[C@H]1C[C@H](O)[C@](CO)(C2CC2)O1] | (2R,3S,5R)-5-(6-amino-2-fluoro-9H-purin-9- yl)-2-cyclopropyl-2- (hydroxymethyl)tetrahydrofuran-3-ol |
| 6 | 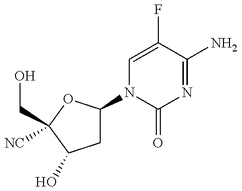 [SMILES: N#C[C@]1(CO)O[C@@H](n2cc(F)c(N)nc2=O)C[C@@H]1O] | (2R,3S,5R)-5-(4-amino-5-fluoro-2- oxopyrimidin-1(2H)-yl)-3-hydroxy-2- (hydroxymethyl)tetrahydrofuran-2- carbonitrile |
| 95 | 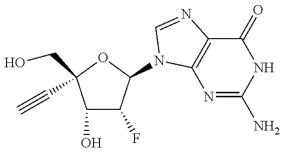 [SMILES: C#C[C@]1(CO)O[C@@H](n2cnc3c(=O)[nH]c(N)nc32)[C@H](F)[C@@H]1O] | 2-amino-9-((2R,3R,4R,5R)-5-ethynyl-3- fluoro-4-hydroxy-5- (hydroxymethyl)tetrahydrofuran-2-yl)-1,9- dihydro-6H-purin-6-one |
| 104 | 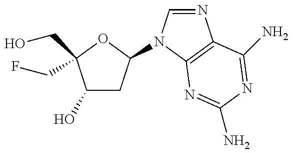 [SMILES: Nc1nc(N)c2ncn([C@H]3C[C@H](O)[C@](CO)(CF)O3)c2n1] | 2-amino-9-((2R,4S,5R)-5-(fluoromethyl)-4- hydroxy-5-(hydroxymethyl)tetrahydrofuran- 2-yl)-9H-purin-6-ol |
| 105 | 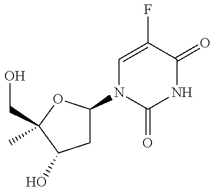 [SMILES: C[C@]1(CO)O[C@@H](n2cc(F)c(=O)[nH]c2=O)C[C@@H]1O] | 5-fluoro-1-((2R,4S,5R)-4-hydroxy-5- (hydroxymethyl)-5-methyltetrahydrofuran-2- yl)pyrimidine-2,4(1H,3H)-dione |
| 107 | 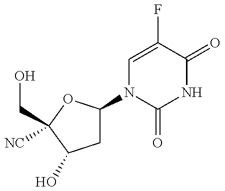 [SMILES: N#C[C@]1(CO)O[C@@H](n2cc(F)c(=O)[nH]c2=O)C[C@@H]1O] | (2R,3S,5R)-5-(5-fluoro-2,4-dioxo-3,4- dihydropyrimidin-1(2H)-yl)-3-hydroxy-2- (hydroxymethyl)tetrahydrofuran-2- carbonitrile |
| 109 | 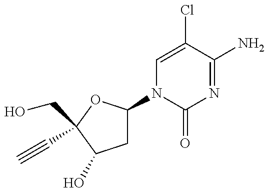 [SMILES: C#C[C@]1(CO)O[C@@H](n2cc(Cl)c(N)nc2=O)C[C@@H]1O] | 4-amino-5-chloro-1-((2R,4S,5R)-5-ethynyl- 4-hydroxy-5- (hydroxymethyl)tetrahydrofuran-2- yl)pyrimidin-2(1H)-one |
| 115 | 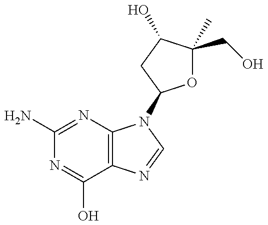 [SMILES: C[C@]1(CO)O[C@@H](n2cnc3c(O)nc(N)nc32)C[C@@H]1O] | 2-amino-9-((2R,4S,5R)-4-hydroxy-5- (hydroxymethyl)-5-methyltetrahydrofuran-2- yl)-9H-purin-6-ol |
| 112 | 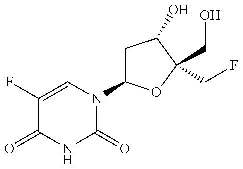 [SMILES: O=c1[nH]c(=O)n([C@H]2C[C@H](O)[C@](CO)(CF)O2)cc1F] | 5-fluoro-1-((2R,4S,5R)-5-(fluoromethyl)-4- hydroxy-5-(hydroxymethyl)tetrahydrofuran- 2-yl)pyrimidine-2,4(1H,3H)-dione |
| 126 | 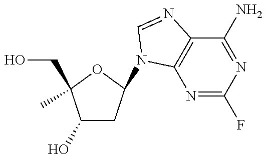 [SMILES: C[C@]1(CO)O[C@@H](n2cnc3c(N)nc(F)nc32)C[C@@H]1O] | (2R,3S,SR)-5-(6-amino-2-fluoro-9H-purin-9- yl)-2-(hydroxymethyl)-2- methyltetrahydrofuran-3-ol |
| 129 | 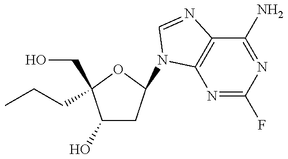 [SMILES: CCC[C@]1(CO)O[C@@H](n2cnc3c(N)nc(F)nc32)C[C@@H]1O] | (2R,3S,5R)-5-(6-amino-2-fluoro-9H-purin-9- yl)-2-(hydroxymethyl)-2- propyltetrahydrofuran-3-ol |
| 130 | 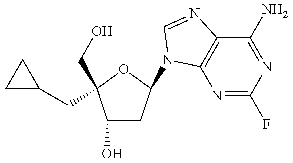 [SMILES: Nc1nc(F)nc2c1ncn2[C@H]1C[C@H](O)[C@](CO)(CC2CC2)O1] | (2R,3S,5R)-5-(6-amino-2-fluoro-9H-purin-9- yl)-2-(cyclopropylmethyl)-2- (hydroxymethyl)tetrahydrofuran-3-ol |
| 133 | 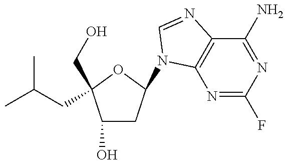 [SMILES: CC(C)C[C@]1(CO)O[C@@H](n2cnc3c(N)nc(F)nc32)C[C@@H]1O] | (2R,3S,5R)-5-(6-amino-2-fluoro-9H-purin-9- yl)-2-(hydroxymethyl)-2- isobutyltetrahydrofuran-3-ol |
| 140 | 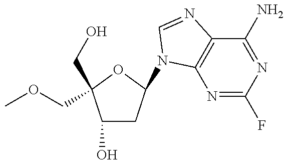 [SMILES: COC[C@]1(CO)O[C@@H](n2cnc3c(N)nc(F)nc32)C[C@@H]1O] | (2S,3S,5R)-5-(6-amino-2-fluoro-9H-purin-9- yl)-2-(hydroxymethyl)-2- (methoxymethyl)tetrahydrofuran-3-ol |
| 150 | 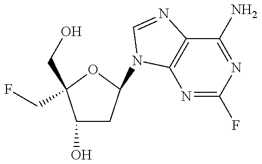 [SMILES: Nc1nc(F)nc2c1ncn2[C@H]1C[C@H](O)[C@](CO)(CF)O1] | (2R,3S,5R)-5-(6-amino-2-fluoro-9H-purin-9- yl)-2-(fluoromethyl)-2- (hydroxymethyl)tetrahydrofuran-3-ol |
| 173 | 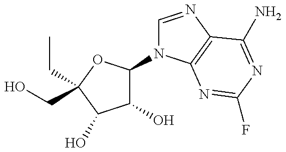 [SMILES: CC[C@]1(CO)O[C@@H](n2cnc3c(N)nc(F)nc32)[C@H](O)[C@@H]1O] | (2R,3S,4R,5R)-5-(6-amino-2-fluoro-9H- purin-9-yl)-2-ethyl-2- (hydroxymethyl)tetrahydrofuran-3,4-diol |
| 178 | 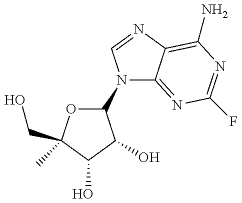 | (2R,3S,4R,5R)-5-(6-amino-2-fluoro-9H- purin-9-yl)-2-(hydroxymethyl)-2- methyltetrahydrofuran-3,4-diol |
| 190 | 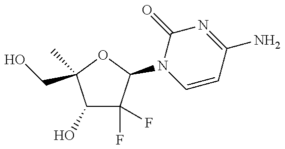 [SMILES: C[C@]1(CO)O[C@@H](n2ccc(N)nc2=O)C(F)(F)[C@@H]1O] | 4-amino-1-((2R,4R,5R)-3,3-difluoro-4- hydroxy-5-(hydroxymethyl)-5- methyltetrahydrofuran-2-yl)pyrimidin- 2(1H)-one |
| 193 | 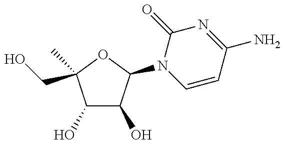 [SMILES: C[C@]1(CO)O[C@@H](n2ccc(N)nc2=O)[C@@H](O)[C@@H]1O] | 4-amino-1-((2R,3S,4S,5R)-3,4- dihydroxy-5-(hydroxymethyl)-5- methyltetrahydrofuran-2-yl)pyrimidin- 2(1H)-one |
| 195 | 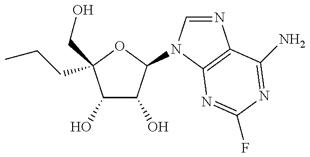 [SMILES: CCC[C@]1(CO)O[C@@H](n2cnc3c(N)nc(F)nc32)[C@H](O)[C@@H]1O] | (2R,3S,4R,5R)-5-(6-amino-2-fluoro-9H- purin-9-yl)-2-(hydroxymethyl)-2- propyltetrahydrofuran-3,4-diol |
| 197 | 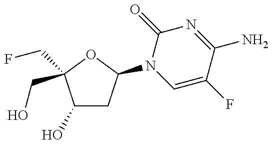 [SMILES: Nc1nc(=O)n([C@H]2C[C@H](O)[C@](CO)(CF)O2)cc1F] | 4-amino-5-fluoro-1-((2R,4S,5R)-5- (fluoromethyl)-4-hydroxy-5- (hydroxymethyl)tetrahydrofuran-2- yl)pyrimidin-2(1H)-one |
| 212 | 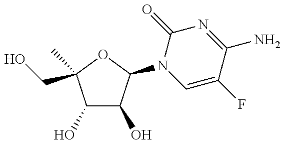 [SMILES: C[C@]1(CO)O[C@@H](n2cc(F)c(N)nc2=O)[C@@H](O)[C@@H]1O] | 4-amino-1-((2R,3S,4S,5R)-3,4- dihydroxy-5-(hydroxymethyl)-5- methyltetrahydrofuran-2-yl)-5- fluoropyrimidin-2(1H)-one |
| 223 | 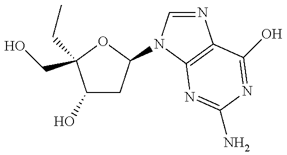 [SMILES: CC[C@]1(CO)O[C@@H](n2cnc3c(O)nc(N)nc32)C[C@@H]1O] | 2-amino-9-((2R,4S,5R)-5-ethyl-4- hydroxy-5- (hydroxymethyl)tetrahydrofuran-2-yl)- 9H-purin-6-ol |

In another embodiment, Compounds of the Disclosure are compounds of Table 2, or a pharmaceutically acceptable salt, solvate, and/or tautomer thereof, and/or mono-, di-, or trisphosphates thereof.

TABLE 2

| Cpd. | | |
|---|---|---|
| No. | Structure | Chemical Name |
| 7 | 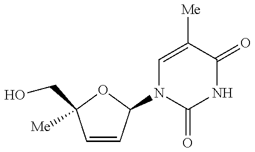 [SMILES: Cc1cn([C@H]2C=C[C@@](C)(CO)O2)c(=O)[nH]c1=O] | 1-((2R,5S)-5-(hydroxymethyl)-5-methyl- 2,5-dihydrofuran-2-yl)-5- methylpyrimidine-2,4(1H,3H)-dione |
| 8 | 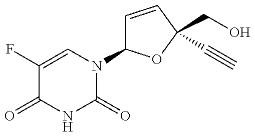 [SMILES: C#C[C@@]1(CO)C=C[C@H](n2cc(F)c(=O)[nH]c2=O)O1] | 1-((5R)-5-ethynyl-5-(hydroxymethyl)- 2,5-dihydrofuran-2-yl)-5- fluoropyrimidine-2,4(1H,3H)-dione |
| 9 | 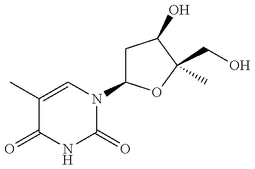 [SMILES: Cc1cn([C@H]2C[C@@H](O)[C@@](C)(CO)O2)c(=O)[nH]c1=O] | 1-((2R,4R,5R)-4-hydroxy-5- (hydroxymethyl)-5- methyltetrahydrofuran-2-yl)-5- methylpyrimidine-2,4(1H,3H)-dione |
| 10 | 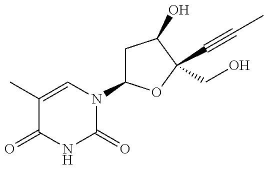 [SMILES: CC#C[C@@]1(CO)O[C@@H](n2cc(C)c(=O)[nH]c2=O)C[C@H]1O] | 1-((2R,4R,5S)-4-hydroxy-5- (hydroxymethyl)-5-(prop-1-yn-1- yl)tetrahydrofuran-2-yl)-5- methylpyrimidine-2,4(1H,3H)-dione |
| 11 | 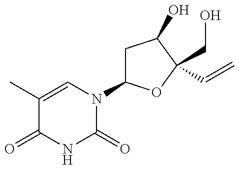 [SMILES: C=C[C@]1(CO)O[C@@H](n2cc(C)c(=O)[nH]c2=O)C[C@H]1O] | 1-((2R,4R,5R)-4-hydroxy-5- (hydroxymethyl)-5- vinyltetrahydrofuran-2-yl)-5- methylpyrimidine-2,4(1H,3H)-dione |
| 12 | 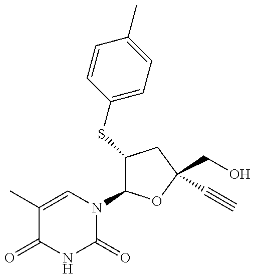 [SMILES: C#C[C@@]1(CO)C[C@@H](Sc2ccc(C)cc2)[C@H](n2cc(C)c(=O)[nH]c2=O)O1] | 1-((2R,3R,5R)-5-ethynyl-5- (hydroxymethyl)-3-(p- tolylthio)tetrahydrofuran-2-yl)-5- methylpyrimidine-2,4(1H,3H)-dione |
| 13 | 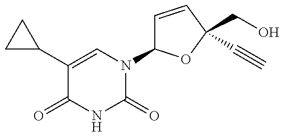 [SMILES: C#C[C@@]1(CO)C=C[C@H](n2cc(C3CC3)c(=O)[nH]c2=O)O1] | 5-cyclopropyl-1-((2R,5R)-5-ethynyl-5- (hydroxymethyl)-2,5-dihydrofuran-2- yl)pyrimidine-2,4(1H,3H)-dione |
| 14 | 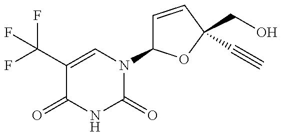 [SMILES: C#C[C@@]1(CO)C=C[C@H](n2cc(C(F)(F)F)c(=O)[nH]c2=O)O1] | 1-((2R,5R)-5-ethynyl-5- (hydroxymethyl)-2,5-dihydrofuran-2-yl)- 5-(trifluoromethyl)pyrimidine- 2,4(1H,3H)-dione |
| 15 | 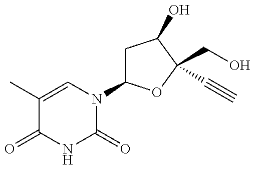 [SMILES: C#C[C@]1(CO)O[C@@H](n2cc(C)c(=O)[nH]c2=O)C[C@H]1O] | 1-((2R,4R,5R)-5-ethynyl-4-hydroxy-5- (hydroxymethyl)tetrahydrofuran-2-yl)-5- methylpyrimidine-2,4(1H,3H)-dione |
| 16 | 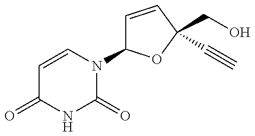 [SMILES: C#C[C@@]1(CO)C=C[C@H](n2ccc(=O)[nH]c2=O)O1] | 1-((2R,5R)-5-ethynyl-5- (hydroxymethyl)-2,5-dihydrofuran-2- yl)pyrimidine-2,4(1H,3H)-dione |
| 17 | 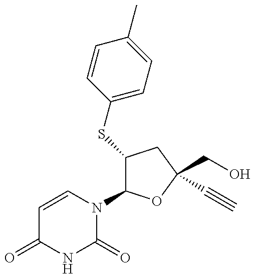 [SMILES: C#C[C@@]1(CO)C[C@@H](Sc2ccc(C)cc2)[C@H](n2ccc(=O)[nH]c2=O)O1] | 1-((2R,3R,5R)-5-ethynyl-5- (hydroxymethyl)-3-(p- tolylthio)tetrahydrofuran-2- yl)pyrimidine-2,4(1H,3H)-dione |
| 18 | 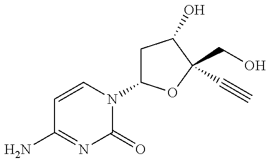 [SMILES: C#C[C@]1(CO)O[C@H](n2ccc(N)nc2=O)C[C@@H]1O] | 4-amino-1-((28,4S,5R)-5-cthynyl-4- hydroxy-5- (hydroxymethyl)tetrahydrofuran-2- yl)pyrimidin-2(1H)-one |
| 19 | 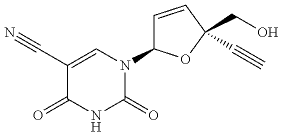 [SMILES: C#C[C@@]1(CO)C=C[C@H](n2cc(C#N)c(=O)[nH]c2=O)O1] | 1-((2R,5R)-5-ethynyl-5- (hydroxymethyl)-2,5-dihydrofuran-2-yl)- 2,4-dioxo-1,2,3,4-tetrahydropyrimidine- 5-carbonitrile |
| 20 | 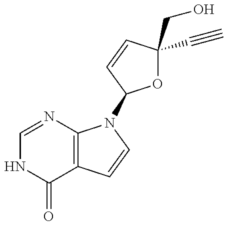 [SMILES: C#C[C@@]1(CO)C=C[C@H](n2ccc3c(=O)[nH]cnc32)O1] | 7-((2R,5R)-5-ethynyl-5- (hydroxymethyl)-2,5-dihydrofuran-2-yl)- 3,7-dihydro-4H-pyrrolo[2,3- d]pyrimidin-4-one |
| 21 | 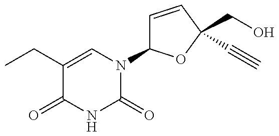 [SMILES: C#C[C@@]1(CO)C=C[C@H](n2cc(CC)c(=O)[nH]c2=O)O1] | 5-ethyl-1-((2R,5R)-5-ethynyl-5- (hydroxymethyl)-2,5-dihydrofuran-2- yl)pyrimidine-2,4(1H,3H)-dione |
| 22 | 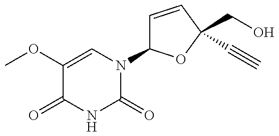 [SMILES: C#C[C@@]1(CO)C=C[C@H](n2cc(OC)c(=O)[nH]c2=O)O1] | 1-((2R,5R)-5-ethynyl-5- (hydroxymethyl)-2,5-dihydrofuran-2-yl)- 5-methoxypyrimidine-2,4(1H,3H)-dione |
| 23 | 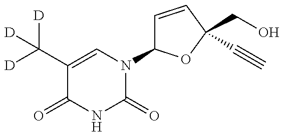 [SMILES: [2H]C([2H])([2H])c1cn([C@H]2C=C[C@@](C#C)(CO)O2)c(=O)[nH]c1=O] | 1-((2R,5R)-5-ethynyl-5- (hydroxymethyl)-2,5-dihydrofuran-2-yl)- 5-(methyl-d3)pyrimidine-2,4(1H,3H)- dione |
| 24 | 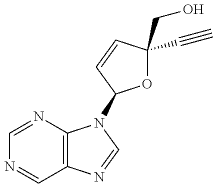 [SMILES: C#C[C@@]1(CO)C=C[C@H](n2cnc3cncnc32)O1] | ((2R,5R)-2-ethynyl-5-(9H-purin-9-yl)- 2,5-dihydrofuran-2-yl)methanol |
| 25 | 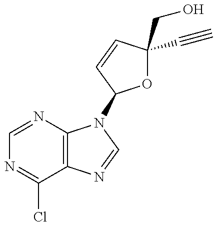 [SMILES: C#C[C@@]1(CO)C=C[C@H](n2cnc3c(Cl)ncnc32)O1] | ((2R,5R)-5-(6-chloro-9H-purin-9-yl)-2- ethynyl-2,5-dihydrofuran-2-yl)methanol |
| 26 | 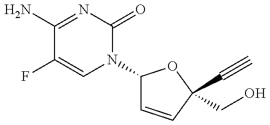 [SMILES: C#C[C@@]1(CO)C=C[C@H](n2cc(F)c(N)nc2=O)O1] | 4-amino-1-((2R,5R)-5-ethynyl-5- (hydroxymethyl)-2,5-dihydrofuran-2-yl)- 5-fluoropyrimidin-2(1H)-one |
| 27 | 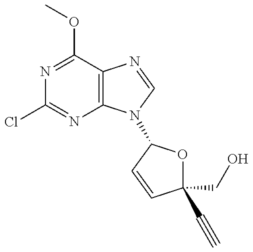 [SMILES: C#C[C@@]1(CO)C=C[C@H](n2cnc3c(OC)nc(Cl)nc32)O1] | ((2R,5R)-5-(2-chloro-6-methoxy-9H- purin-9-yl)-2-ethynyl-2,5-dihydrofuran- 2-yl)methanol |
| 28 | 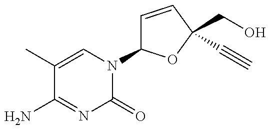 [SMILES: C#C[C@@]1(CO)C=C[C@H](n2cc(C)c(N)nc2=O)O1] | 4-amino-1-((2R,5R)-5-ethynyl-5- (hydroxymethyl)-2,5-dihydrofuran-2-yl)- 5-methylpyrimidin-2(1H)-one |
| 29 | 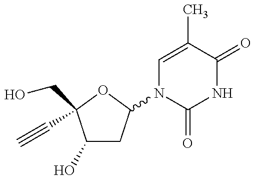 [SMILES: C#C[C@]1(CO)OC(n2cc(C)c(=O)[nH]c2=O)C[C@@H]1O] | 1-((4S,5R)-5-ethynyl-4-hydroxy-5- (hydroxymethyl)tetrahydrofuran-2-yl)-5- methylpyrimidine-2,4(1H,3H)-dione |
| 30 | 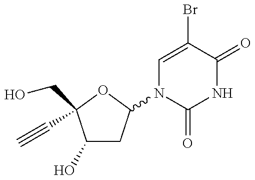 [SMILES: C#C[C@]1(CO)OC(n2cc(Br)c(=O)[nH]c2=O)C[C@@H]1O] | 5-bromo-1-((4S,5R)-5-cthynyl-4- hydroxy-5- (hydroxymethyl)tetrahydrofuran-2- yl)pyrimidine-2,4(1H,3H)-dione |
| 31 | 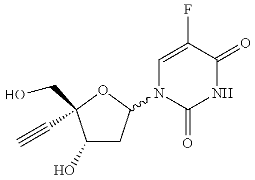 [SMILES: C#C[C@]1(CO)OC(n2cc(F)c(=O)[nH]c2=O)C[C@@H]1O] | 1-((4S,5R)-5-ethynyl-4-hydroxy-5- (hydroxymethyl)tetrahydrofuran-2-yl)-5- fluoropyrimidine-2,4(1H,3H)-dione |
| 32 | 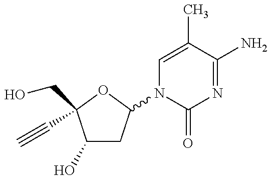 [SMILES: C#C[C@]1(CO)OC(n2cc(C)c(N)nc2=O)C[C@@H]1O] | 4-amino-1-((4S,5R)-5-ethynyl-4- hydroxy-5- (hydroxymethyl)tetrahydrofuran-2-yl)-5- methylpyrimidin-2(1H)-one |
| 33 | 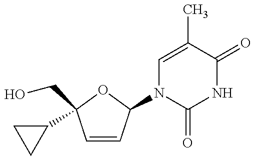 [SMILES: Cc1cn([C@H]2C=C[C@](CO)(C3CC3)O2)c(=O)[nH]c1=O] | 1-((2R,5S)-5-cyclopropyl-5- (hydroxymethyl)-2,5-dihydrofuran-2-yl)- 5-methylpyrimidine-2,4(1H,3H)-dione |
| 34 | 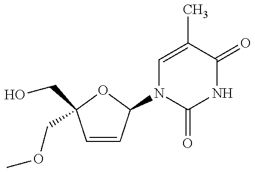 [SMILES: COC[C@@]1(CO)C=C[C@H](n2cc(C)c(=O)[nH]c2=O)O1] | 1-((2R,5S)-5-(hydroxymethyl)-5- (methoxymethyl)-2,5-dihydrofuran-2- yl)-5-methylpyrimidine-2,4(1H,3H)- dione |
| 35 | 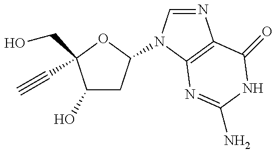 [SMILES: C#C[C@]1(CO)O[C@H](n2cnc3c(=O)[nH]c(N)nc32)C[C@@H]1O] | 2-amino-9-((2S,4S,5R)-5-ethynyl-4- hydroxy-5- (hydroxymethyl)tetrahydrofuran-2-yl)- 1,9-dihydro-6H-purin-6-one |
| 36 | 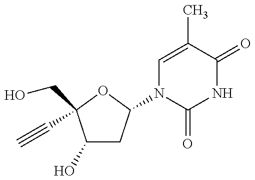 [SMILES: C#C[C@]1(CO)O[C@H](n2cc(C)c(=O)[nH]c2=O)C[C@@H]1O] | 1-((2S,4S,5R)-5-ethynyl-4-hydroxy-5- (hydroxymethyl)tetrahydrofuran-2-yl)-5- methylpyrimidine-2,4(1H,3H)-dione |
| 37 | 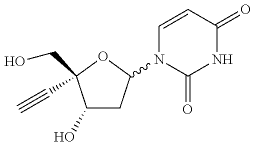 [SMILES: C#C[C@]1(CO)OC(n2ccc(=O)[nH]c2=O)C[C@@H]1O] | 1-((4S,5R)-5-ethynyl-4-hydroxy-5- (hydroxymethyl)tetrahydrofuran-2- yl)pyrimidine-2,4(1H,3H)-dione |
| 38 | 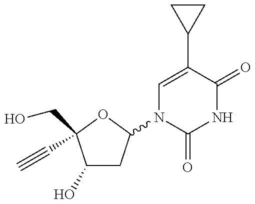 [SMILES: C#C[C@]1(CO)OC(n2cc(C3CC3)c(=O)[nH]c2=O)C[C@@H]1O] | 5-cyclopropyl-1-((4S,5R)-5-ethynyl-4- hydroxy-5- (hydroxymethyl)tetrahydrofuran-2- yl)pyrimidine-2,4(1H,3H)-dione |
| 39 | 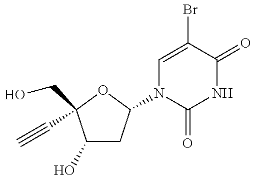 [SMILES: C#C[C@]1(CO)O[C@H](n2cc(Br)c(=O)[nH]c2=O)C[C@@H]1O] | 5-bromo-1-((2S,4S,5R)-5-ethynyl-4- hydroxy-5- (hydroxymethyl)tetrahydrofuran-2- yl)pyrimidine-2,4(1H,3H)-dione |
| 40 | 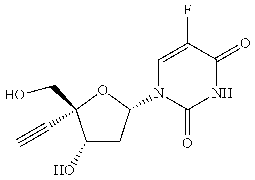 [SMILES: C#C[C@]1(CO)O[C@H](n2cc(F)c(=O)[nH]c2=O)C[C@@H]1O] | 1-((2S,4S,5R)-5-ethynyl-4-hydroxy-5- (hydroxymethyl)tetrahydrofuran-2-yl)-5- fluoropyrimidine-2,4(1H,3H)-dione |
| 41 | 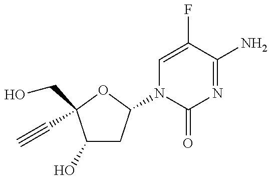 [SMILES: C#C[C@]1(CO)O[C@H](n2cc(F)c(N)nc2=O)C[C@@H]1O] | 4-amino-1-((2S,4S,5R)-5-ethynyl-4- hydroxy-5- (hydroxymethyl)tetrahydrofuran-2-yl)-5- fluoropyrimidin-2(1H)-one |
| 42 | 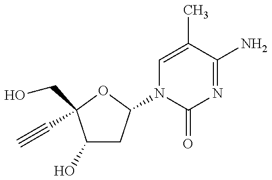 [SMILES: C#C[C@]1(CO)O[C@H](n2cc(C)c(N)nc2=O)C[C@@H]1O] | 4-amino-1-((2S,4S,5R)-5-ethynyl-4- hydroxy-5- (hydroxymethyl)tetrahydrofuran-2-yl)-5- methylpyrimidin-2(1H)-one |
| 43 | 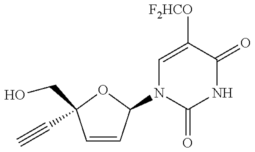 [SMILES: C#C[C@@]1(CO)C=C[C@H](n2cc(OC(F)F)c(=O)[nH]c2=O)O1] | 5-(difluoromethoxy)-1-((2R,5R)-5- ethynyl-5-(hydroxymethyl)-2,5- dihydrofuran-2-yl)pyrimidine- 2,4(1H,3H)-dione |
| 44 | 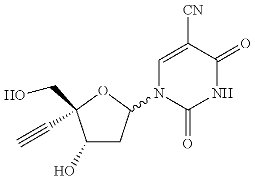 [SMILES: C#C[C@]1(CO)OC(n2cc(C#N)c(=O)[nH]c2=O)C[C@@H]1O] | 1-((4S,5R)-5-ethynyl-4-hydroxy-5- (hydroxymethyl)tetrahydrofuran-2-yl)- 2,4-dioxo-1,2,3,4-tetrahydropyrimidine- 5-carbonitrile |
| 45 | 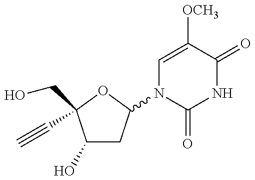 [SMILES: C#C[C@]1(CO)OC(n2cc(OC)c(=O)[nH]c2=O)C[C@@H]1O] | 1-((4S,5R)-5-ethynyl-4-hydroxy-5- (hydroxymethyl)tetrahydrofuran-2-yl)-5- methoxypyrimidine-2,4(1H,3H)-dione |
| 46 | 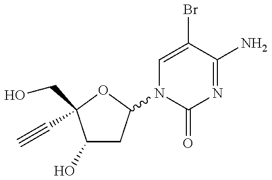 [SMILES: C#C[C@]1(CO)OC(n2cc(Br)c(N)nc2=O)C[C@@H]1O] | 4-amino-5-bromo-1-((4S,5R)-5-ethynyl- 4-hydroxy-5- (hydroxymethyl)tetrahydrofuran-2- yl)pyrimidin-2(1H)-one |
| 47 | 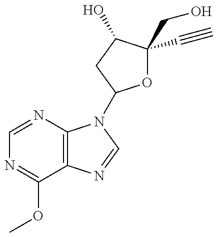 [SMILES: C#C[C@]1(CO)OC(n2cnc3c(OC)ncnc32)C[C@@H]1O] | (2R,3S)-2-cthynyl-2-(hydroxymethyl)-5- (6-methoxy-9H-purin-9- yl)tetrahydrofuran-3-ol |
| 48 | 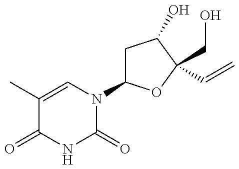 [SMILES: C=C[C@]1(CO)O[C@@H](n2cc(C)c(=O)[nH]c2=O)C[C@@H]1O] | 1-((2R,4S,5R)-4-hydroxy-5- (hydroxymethyl)-5- vinyltetrahydrofuran-2-yl)-5- methylpyrimidine-2,4(1H,3H)-dione |
| 49 | 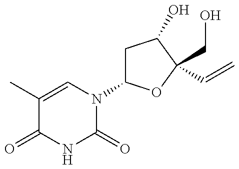 [SMILES: C=C[C@]1(CO)O[C@H](n2cc(C)c(=O)[nH]c2=O)C[C@@H]1O] | 1-((2S,4S,5R)-4-hydroxy-5- (hydroxymethyl)-5- vinyltetrahydrofuran-2-yl)-5- methylpyrimidine-2,4(1H,3H)-dione |
| 50 | 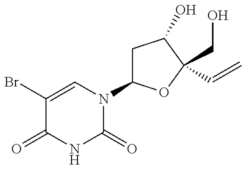 [SMILES: C=C[C@]1(CO)O[C@@H](n2cc(Br)c(=O)[nH]c2=O)C[C@@H]1O] | 5-bromo-1-((2R,4S,5R)-4-hydroxy-5- (hydroxymethyl)-5- vinyltetrahydrofuran-2-yl)pyrimidine- 2,4(1H,3H)-dione |
| 51 | 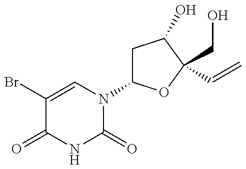 [SMILES: C=C[C@]1(CO)O[C@H](n2cc(Br)c(=O)[nH]c2=O)C[C@@H]1O] | 5-bromo-1-((2S,4S,5R)-4-hydroxy-5- (hydroxymethyl)-5- vinyltetrahydrofuran-2-yl)pyrimidine- 2,4(1H,3H)-dione |
| 51 | 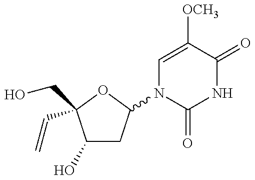 [SMILES: C=C[C@]1(CO)OC(n2cc(OC)c(=O)[nH]c2=O)C[C@@H]1O] | 1-((4S,5R)-4-hydroxy-5- (hydroxymethyl)-5- vinyltetrahydrofuran-2-yl)-5- methoxypyrimidine-2,4(1H,3H)-dione |
| 53 | 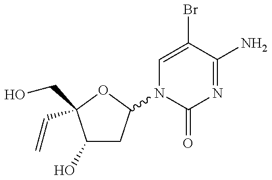 [SMILES: C=C[C@]1(CO)OC(n2cc(Br)c(N)nc2=O)C[C@@H]1O] | 4-amino-5-bromo-1-((4S,5R)-4- hydroxy-5-(hydroxymethyl)-5- vinyltetrahydrofuran-2-yl)pyrimidin- 2(1H)-one |
| 54 | 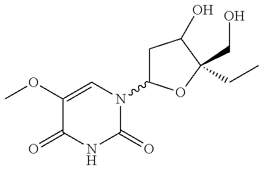 [SMILES: CC[C@]1(CO)OC(n2cc(OC)c(=O)[nH]c2=O)CC1O] | 1-((5R)-5-ethyl-4-hydroxy-5- (hydroxymethyl)tetrahydrofuran-2-yl)-5- methoxypyrimidine-2,4(1H,3H)-dione |
| 55 | 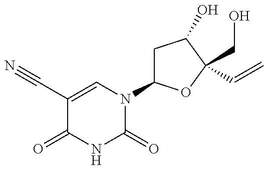 [SMILES: C=C[C@]1(CO)O[C@@H](n2cc(C#N)c(=O)[nH]c2=O)C[C@@H]1O] | 1-((2R,4S,5R)-4-hydroxy-5- (hydroxymethyl)-5- vinyltetrahydrofuran-2-yl)-2,4-dioxo- 1,2,3,4-tetrahydropyrimidine-5- carbonitrile |
| 56 | 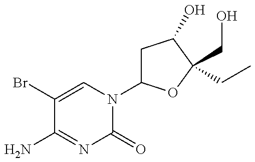 [SMILES: CC[C@]1(CO)OC(n2cc(Br)c(N)nc2=O)C[C@@H]1O] | 4-amino-5-bromo-1-((4S,5R)-5-ethyl-4- hydroxy-5- (hydroxymethyl)tetrahydrofuran-2- yl)pyrimidin-2(1H)-one |
| 57 | 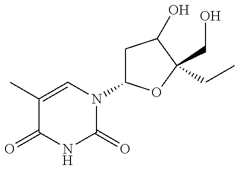 [SMILES: CC[C@]1(CO)O[C@H](n2cc(C)c(=O)[nH]c2=O)CC1O] | 1-((2S,5R)-5-ethyl-4-hydroxy-5- (hydroxymethyl)tetrahydrofuran-2-yl)-5- methylpyrimidine-2,4(1H,3H)-dione |
| 58 | 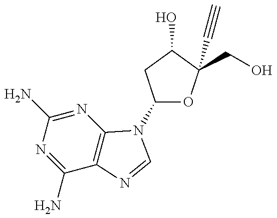 [SMILES: C#C[C@]1(CO)O[C@H](n2cnc3c(N)nc(N)nc32)C[C@@H]1O] | (2R,3S,5S)-5-(2,6-diamino-9H-purin-9- yl)-2-ethynyl-2- (hydroxymethyl)tetrahydrofuran-3-ol |
| 59 | 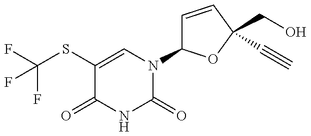 [SMILES: C#C[C@@]1(CO)C=C[C@H](n2cc(SC(F)(F)F)c(=O)[nH]c2=O)O1] | 1-((2R,5R)-5-ethynyl-5- (hydroxymethyl)-2,5-dihydrofuran-2-yl)- 5-((trifluoromethyl)thio)pyrimidine- 2,4(1H,3H)-dione |
| 60 | 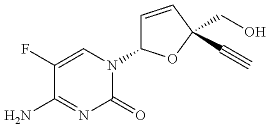 [SMILES: C#C[C@]1(CO)C=C[C@@H](n2cc(F)c(N)nc2=O)O1] | 4-amino-1-((2S,5S)-5-ethynyl-5- (hydroxymethyl)-2,5-dihydrofuran-2-yl)- 5-fluoropyrimidin-2(1H)-one |
| 61 | 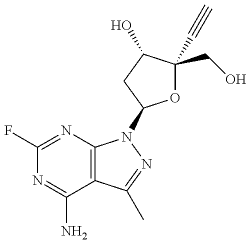 [SMILES: C#C[C@]1(CO)O[C@@H](n2nc(C)c3c(N)nc(F)nc32)C[C@@H]1O] | (2R,3S,5R)-5-(4-amino-6-fluoro-3- methyl-1H-pyrazolo[3,4-d]pyrimidin-1- yl)-2-ethynyl-2- (hydroxymethyl)tetrahydrofuran-3-ol |
| 62 | 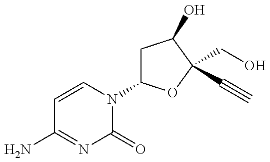 [SMILES: C#C[C@@]1(CO)O[C@H](n2ccc(N)nc2=O)C[C@H]1O] | 4-amino-1-((2S,4R,5S)-5-ethynyl-4- hydroxy-5- (hydroxymethyl)tetrahydrofuran-2- yl)pyrimidin-2(1H)-one |
| 63 | 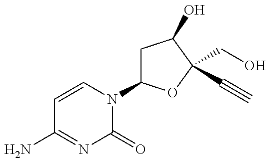 [SMILES: C#C[C@@]1(CO)O[C@@H](n2ccc(N)nc2=O)C[C@H]1O] | 4-amino-1-((2R,4R,5S)-5-ethynyl-4- hydroxy-5- (hydroxymethyl)tetrahydrofuran-2- yl)pyrimidin-2(1H)-one |
| 64 | 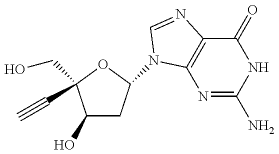 [SMILES: C#C[C@@]1(CO)O[C@H](n2cnc3c(=O)[nH]c(N)nc32)C[C@H]1O] | 2-amino-9-((2S,4R,5S)-5-ethynyl-4- hydroxy-5- (hydroxymethyl)tetrahydrofuran-2-yl)- 1,9-dihydro-6H-purin-6-one |
| 65 | 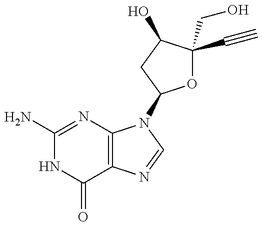 [SMILES: C#C[C@@]1(CO)O[C@@H](n2cnc3c(=O)[nH]c(N)nc32)C[C@H]1O] | 2-amino-9-((2R,4R,5S)-5-ethynyl-4- hydroxy-5- (hydroxymethyl)tetrahydrofuran-2-yl)- 1,9-dihydro-6H-purin-6-one |
| 66 | 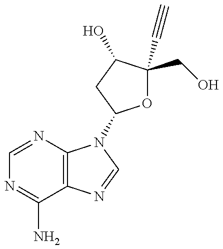 [SMILES: C#C[C@]1(CO)O[C@H](n2cnc3c(N)ncnc32)C[C@@H]1O] | (2R,3S,5S)-5-(6-amino-9H-purin-9-yl)- 2-ethynyl-2- (hydroxymethyl)tetrahydrofuran-3-ol |
| 68 | 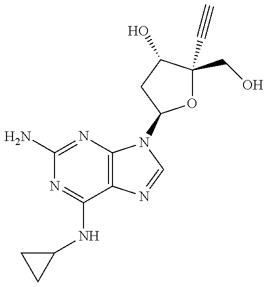 [SMILES: C#C[C@]1(CO)O[C@@H](n2cnc3c(NC4CC4)nc(N)nc32)C[C@@H]1O] | (2R,3S,5R)-S-(2-amino-6- (cyclopropylamino)-9H-purin-9-yl)-2- ethynyl-2- (hydroxymethyl)tetrahydrofuran-3-ol |
| 69 | 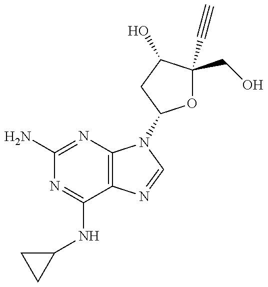 [SMILES: C#C[C@]1(CO)O[C@H](n2cnc3c(NC4CC4)nc(N)nc32)C[C@@H]1O] | (2R,3S,5S)-5-(2-amino-6- (cyclopropylamino)-9H-purin-9-yl)-2- ethynyl-2- (hydroxymethyl)tetrahydrofuran-3-ol |
| 70 | 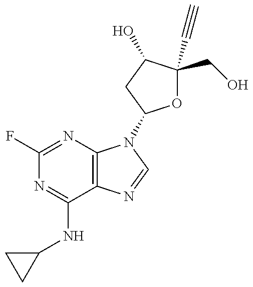 [SMILES: C#C[C@]1(CO)O[C@H](n2cnc3c(NC4CC4)nc(F)nc32)C[C@@H]1O] | (2R,3S,5S)-5-(6-(cyclopropylamino)-2- fluoro-9H-purin-9-yl)-2-ethynyl-2- (hydroxymethyl)tetrahydrofuran-3-ol |
| 71 | 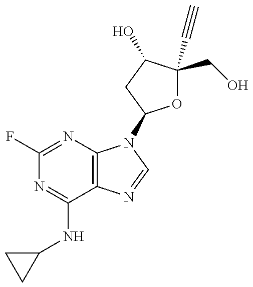 [SMILES: C#C[C@]1(CO)O[C@@H](n2cnc3c(NC4CC4)nc(F)nc32)C[C@@H]1O] | (2R,3S,5R)-5-(6-(cyclopropylamino)-2- fluoro-9H-purin-9-yl)-2-ethynyl-2- (hydroxymethyl)tetrahydrofuran-3-ol |
| 72 | 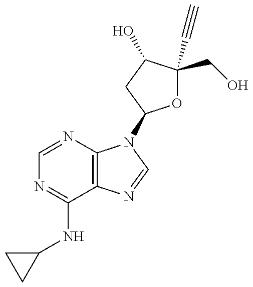 [SMILES: C#C[C@]1(CO)O[C@@H](n2cnc3c(NC4CC4)ncnc32)C[C@@H]1O] | (2R,3S,5R)-5-(6-(cyclopropylamino)- 9H-purin-9-yl)-2-ethynyl-2- (hydroxymethyl)tetrahydrofuran-3-ol |
| 73 | 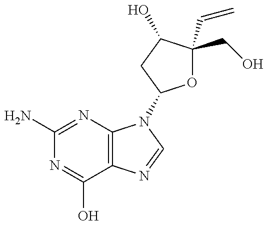 [SMILES: C=C[C@]1(CO)O[C@H](n2cnc3c(O)nc(N)nc32)C[C@@H]1O] | 2-amino-9-((2S,4S,5R)-4-hydroxy-5- (hydroxymethyl)-5- vinyltetrahydrofuran-2-yl)-9H-purin-6-ol |
| 74 | 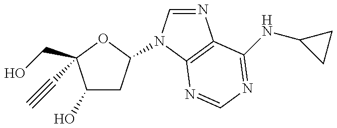 [SMILES: C#C[C@]1(CO)O[C@H](n2cnc3c(NC4CC4)ncnc32)C[C@@H]1O] | (2R,3S,5S)-5-(6-(cyclopropylamino)- 9H-purin-9-yl)-2-ethynyl-2- (hydroxymethyl)tetrahydrofuran-3-ol |
| 75 | 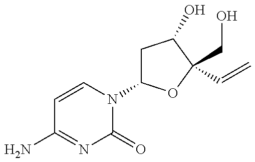 [SMILES: C=C[C@]1(CO)O[C@H](n2ccc(N)nc2=O)C[C@@H]1O] | 4-amino-1-((2S,4S,5R)-4-hydroxy-5- (hydroxymethyl)-5- vinyltetrahydrofuran-2-yl)pyrimidin- 2(1H)-one |
| 76 | 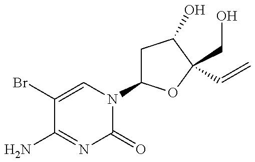 [SMILES: C=C[C@]1(CO)O[C@@H](n2cc(Br)c(N)nc2=O)C[C@@H]1O] | 4-amino-5-bromo-1-((2R,4S,5R)-4- hydroxy-5-(hydroxymethyl)-5- vinyltetrahydrofuran-2-yl)pyrimidin- 2(1H)-one |
| 77 | 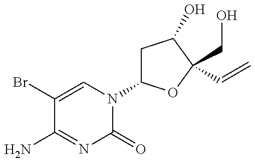 [SMILES: C=C[C@]1(CO)O[C@H](n2cc(Br)c(N)nc2=O)C[C@@H]1O] | 4-amino-5-bromo-1-((2S,4S,5R)-4- hydroxy-5-(hydroxymethyl)-5- vinyltetrahydrofuran-2-yl)pyrimidin- 2(IH)-one |
| 78 | 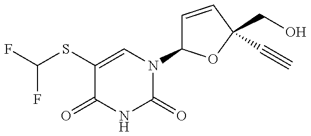 [SMILES: C#C[C@@]1(CO)C=C[C@H](n2cc(SC(F)F)c(=O)[nH]c2=O)O1] | 5-((difluoromethyl)thio)-1-((2R,5R)-5- ethynyl-5-(hydroxymethyl)-2,5- dihydrofuran-2-yl)pyrimidine- 2,4(1H,3H)-dione |
| 79 | 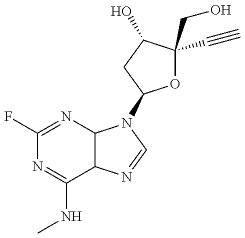 [SMILES: C#C[C@]1(CO)O[C@@H](N2C=NC3C(NC)=NC(F)=NC32)C[C@@H]1O] | (2R,3S,5R)-2-ethynyl-5-(2-fluoro-6- (methylamino)-4,5-dihydro-9H-purin-9- yl)-2-(hydroxymethyl)tetrahydrofuran-3- ol |
| 80 | 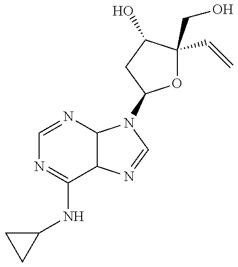 [SMILES: C=C[C@]1(CO)O[C@@H](N2C=NC3C(NC4CC4)=NC=NC32)C[C@@H]1O] | (2R,3S,5R)-5-(6-(cyclopropylamino)- 4,5-dihydro-9H-purin-9-yl)-2- (hydroxymethyl)-2- vinyltetrahydrofuran-3-ol |
| 81 | 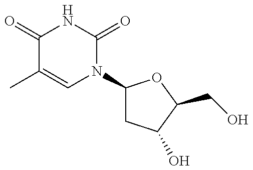 [SMILES: Cc1cn([C@@H]2C[C@@H](O)[C@H](CO)O2)c(=O)[nH]c1=O] | 1-((2S,4R,5S)-4-hydroxy-5- (hydroxymethyl)tetrahydrofuran-2-yl)-5- methylpyrimidine-2,4(1H,3H)-dione |
| 82 | 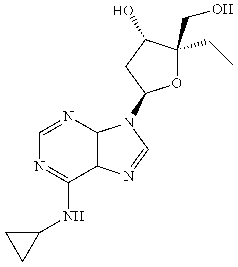 [SMILES: CC[C@]1(CO)O[C@@H](N2C=NC3C(NC4CC4)=NC=NC32)C[C@@H]1O] | (2R,3S,5R)-5-(6-(cyclopropylamino)- 4,5-dihydro-9H-purin-9-yl)-2-ethyl-2- (hydroxymethyl)tetrahydrofuran-3-ol |
| 83 | 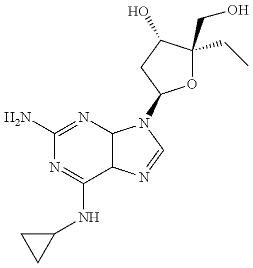 [SMILES: CC[C@]1(CO)O[C@@H](N2C=NC3C(NC4CC4)=NC(N)=NC32)C[C@@H]1O] | (2R,3S,5R)-5-(2-amino-6- (cyclopropylamino)-4,5-dihydro-9H- purin-9-yl)-2-ethyl-2- (hydroxymethyl)tetrahydrofuran-3-ol |
| 84 | 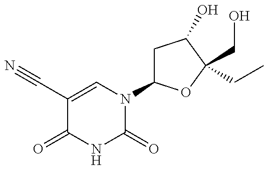 [SMILES: CC[C@]1(CO)O[C@@H](n2cc(C#N)c(=O)[nH]c2=O)C[C@@H]1O] | 1-((2R,4S,5R)-5-ethyl-4-hydroxy-5- (hydroxymethyl)tetrahydrofuran-2-yl)- 2,4-dioxo-1,2,3,4-tetrahydropyrimidine- 5-carbonitrile |
| 85 | 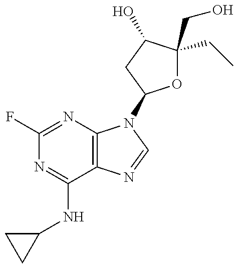 [SMILES: CC[C@]1(CO)O[C@@H](n2cnc3c(NC4CC4)nc(F)nc32)C[C@@H]1O] | (2R,3S,5R)-5-(6-(cyclopropylamino)-2- fluoro-9H-purin-9-yl)-2-ethyl-2- (hydroxymethyl)tetrahydrofuran-3-ol |
| 88 | 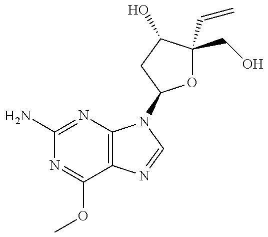 [SMILES: C=C[C@]1(CO)O[C@@H](n2cnc3c(OC)nc(N)nc32)C[C@@H]1O] | (2R,3S,5R)-5-(2-amino-6-methoxy-9H- purin-9-yl)-2-(hydroxymethyl)-2- vinyltetrahydrofuran-3-ol |
| 89 | 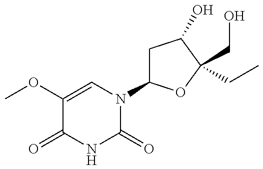 [SMILES: CC[C@]1(CO)O[C@@H](n2cc(OC)c(=O)[nH]c2=O)C[C@@H]1O] | 1-((2R,4S,5R)-5-ethyl-4-hydroxy-5- (hydroxymethyl)tetrahydrofuran-2-yl)-5- methoxypyrimidine-2,4(1H,3H)-dione |
| 90 | 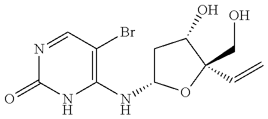 [SMILES: C=C[C@]1(CO)O[C@H](Nc2[nH]c(=O)ncc2Br)C[C@@H]1O] | 5-bromo-6-(((2S,4S,5R)-4-hydroxy-5- (hydroxymethyl)-5- vinyltetrahydrofuran-2- yl)amino)pyrimidin-2(1H)-one |
| 91 | 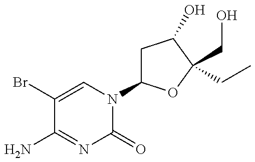 [SMILES: CC[C@]1(CO)O[C@@H](n2cc(Br)c(N)nc2=O)C[C@@H]1O] | 4-amino-5-bromo-1-((2R,4S,5R)-5- ethyl-4-hydroxy-5- (hydroxymethyl)tetrahydrofuran-2- yl)pyrimidin-2(1H)-one |
| 92 | 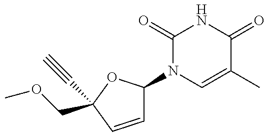 [SMILES: C#C[C@@]1(COC)C=C[C@H](n2cc(C)c(=O)[nH]c2=O)O1] | 1-((2R,5R)-5-ethynyl-5- (methoxymethyl)-2,5-dihydrofuran-2- yl)-5-methylpyrimidine-2,4(1H,3H)- dione |
| 93 | 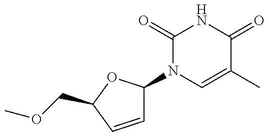 [SMILES: COC[C@@H]1C=C[C@H](n2cc(C)c(=O)[nH]c2=O)O1] | 1-((2R,5S)-5-(methoxymethyl)-2,5- dihydrofuran-2-yl)-5-methylpyrimidine- 2,4(1H,3H)-dione |
| 94 | 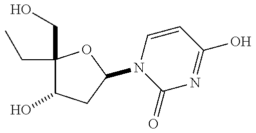 [SMILES: CC[C@]1(CO)O[C@@H](n2ccc(O)nc2=O)C[C@@H]1O] | 1-((2R,4S,5R)-5-ethyl-4-hydroxy-5- (hydroxymethyl)tetrahydrofuran-2-yl)-4- hydroxypyrimidin-2(1H)-one |
| 96 | 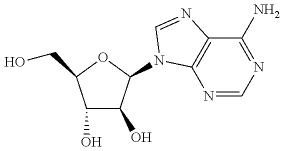 [SMILES: Nc1ncnc2c1ncn2[C@@H]1O[C@H](CO)[C@@H](O)[C@@H]1O] | (2R,3S,4S,5R)-2-(6-amino-9H-purin-9- yl)-5-(hydroxymethyl)tetrahydrofuran- 3,4-diol |
| 97 | 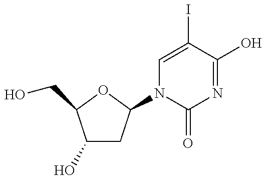 [SMILES: O=c1nc(O)c(I)cn1[C@H]1C[C@H](O)[C@@H](CO)O1] | 4-hydroxy-1-((2R,4S,5R)-4-hydroxy-5- (hydroxymethyl)tetrahydrofuran-2-yl)-5- iodopyrimidin-2(1H)-one |
| 100 | 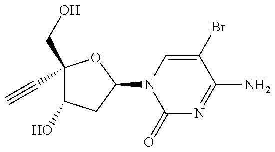 [SMILES: C#C[C@]1(CO)O[C@@H](n2cc(Br)c(N)nc2=O)C[C@@H]1O] | 4-amino-5-bromo-1-((2R,4S,5R)-5- ethynyl-4-hydroxy-5- (hydroxymethyl)tetrahydrofuran-2- yl)pyrimidin-2(1H)-one |
| 101 | 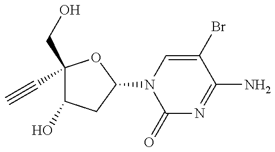 [SMILES: C#C[C@]1(CO)O[C@H](n2cc(Br)c(N)nc2=O)C[C@@H]1O] | 4-amino-5-bromo-1-((2S,4S,5R)-5- ethynyl-4-hydroxy-5- (hydroxymethyl)tetrahydrofuran-2- yl)pyrimidin-2(1H)-one |
| 110 | 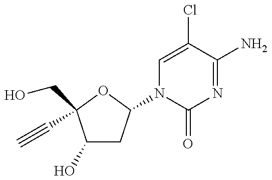 [SMILES: C#C[C@]1(CO)O[C@H](n2cc(Cl)c(N)nc2=O)C[C@@H]1O] | 4-amino-5-chloro-1-((2S,4S,5R)-5- ethynyl-4-hydroxy-5- (hydroxymethyl)tetrahydrofuran-2- yl)pyrimidin-2(1H)-one |
| 111 | 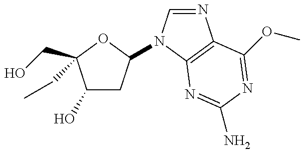 [SMILES: CC[C@]1(CO)O[C@@H](n2cnc3c(OC)nc(N)nc32)C[C@@H]1O] | (2R,3S,5R)-5-(2-amino-6-methoxy-9H- purin-9-yl)-2-ethyl-2- (hydroxymethyl)tetrahydrofuran-3-ol |
| 113 | 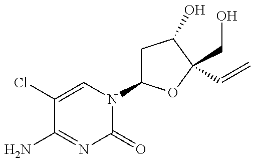 [SMILES: C=C[C@]1(CO)O[C@@H](n2cc(Cl)c(N)nc2=O)C[C@@H]1O] | 4-amino-5-chloro-1-((2R,4S,5R)-4- hydroxy-5-(hydroxymethyl)-5- vinyltetrahydrofuran-2-yl)pyrimidin- 2(1H)-one |
| 117 | 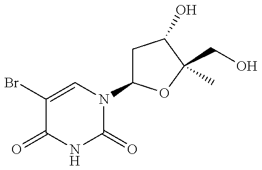 [SMILES: C[C@]1(CO)O[C@@H](n2cc(Br)c(=O)[nH]c2=O)C[C@@H]1O] | 5-bromo-1-((2R,4S,5R)-4-hydroxy-5- (hydroxymethyl)-5- methyltetrahydrofuran-2-yl)pyrimidine- 2,4(1H,3H)-dione |
| 118 | 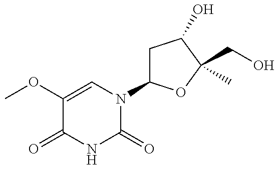 [SMILES: COc1cn([C@H]2C[C@H](O)[C@@](C)(CO)O2)c(=O)[nH]c1=O] | 1-((2R,4S,5R)-4-hydroxy-5- (hydroxymethyl)-5- methyltetrahydrofuran-2-yl)-5- methoxypyrimidine-2,4(1H,3H)-dione |
| 119 | 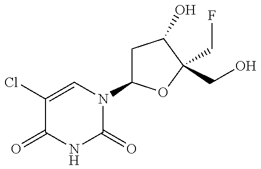 [SMILES: O=c1[nH]c(=O)n([C@H]2C[C@H](O)[C@](CO)(CF)O2)cc1Cl] | 5-chloro-1-((2R,4S,5R)-5- (fluoromethyl)-4-hydroxy-5- (hydroxymethyl)tetrahydrofuran-2- yl)pyrimidine-2,4(1H,3H)-dione |
| 120 | 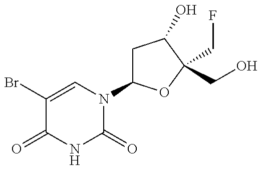 [SMILES: O=c1[nH]c(=O)n([C@H]2C[C@H](O)[C@](CO)(CF)O2)cc1Br] | 5-bromo-1-((2R,4S,5R)-5- (fluoromethyl)-4-hydroxy-5- (hydroxymethyl)tetrahydrofuran-2- yl)pyrimidine-2,4(1H,3H)-dione |
| 121 | 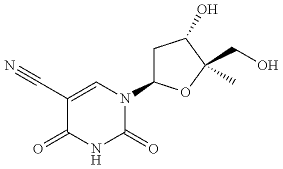 [SMILES: C[C@]1(CO)O[C@@H](n2cc(C#N)c(=O)[nH]c2=O)C[C@@H]1O] | 1-((2R,4S,5R)-4-hydroxy-5- (hydroxymethyl)-5- methyltetrahydrofuran-2-yl)-2,4-dioxo- 1,2,3,4-tetrahydropyrimidine-5- carbonitrile |
| 122 | 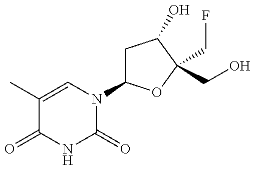 [SMILES: Cc1cn([C@H]2C[C@H](O)[C@](CO)(CF)O2)c(=O)[nH]c1=O] | 1-((2R,4S,5R)-5-(fluoromethyl)-4- hydroxy-5- (hydroxymethyl)tetrahydrofuran-2-yl)-5- methylpyrimidine-2,4(1H,3H)-dione |
| 128 | 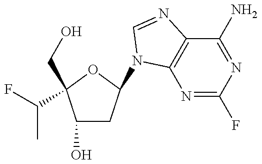 [SMILES: CC(F)[C@]1(CO)O[C@@H](n2cnc3c(N)nc(F)nc32)C[C@@H]1O] | (2R,3S,5R)-5-(6-amino-2-fluoro-9H- purin-9-yl)-2-((R)-1-fluoroethyl)-2- (hydroxymethyl)tetrahydrofuran-3-ol |
| 131 | 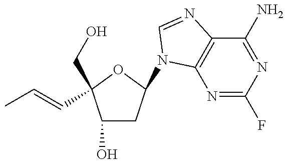 [SMILES: C/C=C/[C@]1(CO)O[C@@H](n2cnc3c(N)nc(F)nc32)C[C@@H]1O] | (2R,3S,5R)-5-(6-amino-2-fluoro-9H- purin-9-yl)-2-(hydroxymethyl)-2-((E)- prop-1-en-1-yl)tetrahydrofuran-3-ol |
| 132 | 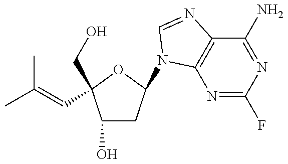 [SMILES: CC(C)=C[C@]1(CO)O[C@@H](n2cnc3c(N)nc(F)nc32)C[C@@H]1O] | (2R,3S,5R)-5-(6-amino-2-fluoro-9H- purin-9-yl)-2-(hydroxymethyl)-2-(2- methylprop-1-en-1-yl)tetrahydrofuran-3- ol |
| 134 | 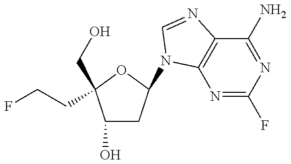 [SMILES: Nc1nc(F)nc2c1ncn2[C@H]1C[C@H](O)[C@](CO)(CCF)O1] | (2R,3S,5R)-5-(6-amino-2-fluoro-9H- purin-9-yl)-2-(2-fluoroethyl)-2- (hydroxymethyl)tetrahydrofuran-3-ol |
| 135 | 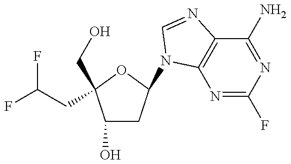 [SMILES: Nc1nc(F)nc2c1ncn2[C@H]1C[C@H](O)[C@](CO)(CC(F)F)O1] | (2R,3S,5R)-5-(6-amino-2-fluoro-9H- purin-9-yl)-2-(2,2-difluoroethyl)-2- (hydroxymethyl)tetrahydrofuran-3-ol |
| 136 | 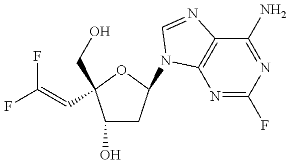 [SMILES: Nc1nc(F)nc2c1ncn2[C@H]1C[C@H](O)[C@@](C=C(F)F)(CO)O1] | (2R,3S,5R)-5-(6-amino-2-fluoro-9H- purin-9-yl)-2-(2,2-difluorovinyl)-2- (hydroxymethyl)tetrahydrofuran-3-ol |
| 137 | 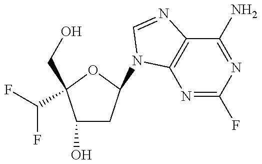 [SMILES: Nc1nc(F)nc2c1ncn2[C@H]1C[C@H](O)[C@](CO)(C(F)F)O1] | (2R,3S,5R)-5-(6-amino-2-fluoro-9H- purin-9-yl)-2-(difluoromethyl)-2- (hydroxymethyl)tetrahydrofuran-3-ol |
| 138 | 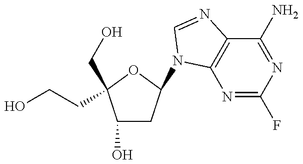 [SMILES: Nc1nc(F)nc2c1ncn2[C@H]1C[C@H](O)[C@](CO)(CCO)O1] | (2R,3S,5R)-5-(6-amino-2-fluoro-9H- purin-9-yl)-2-(2-hydroxyethyl)-2- (hydroxymethyl)tetrahydrofuran-3-ol |
| 139 | 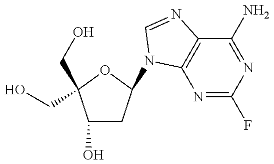 | ((3S,5R)-5-(6-amino-2-fluoro-9H-purin- 9-yl)-3-hydroxytetrahydrofuran-2,2- diyl)dimethanol |
| 141 | 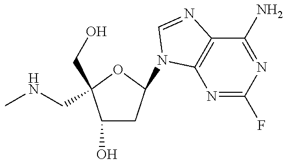 [SMILES: CNC[C@]1(CO)O[C@@H](n2cnc3c(N)nc(F)nc32)C[C@@H]1O] | (2R,3S,5R)-5-(6-amino-2-fluoro-9H- purin-9-yl)-2-(hydroxymethyl)-2- ((mcthylamino)methyl)tetrahydrofuran- 3-ol |
| 142 | 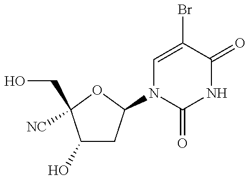 [SMILES: N#C[C@]1(CO)O[C@@H](n2cc(Br)c(=O)[nH]c2=O)C[C@@H]1O] | (2R,3S,5R)-5-(5-bromo-2,4-dioxo-3,4- dihydropyrimidin-1(2H)-yl)-3-hydroxy- 2-(hydroxymethyl)tetrahydrofuran-2- carbonitrile |
| 143 | 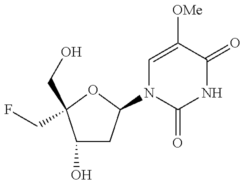 [SMILES: COc1cn([C@H]2C[C@H](O)[C@](CO)(CF)O2)c(=O)[nH]c1=O] | 1-((2R,4S,5R)-5-(fluoromethyl)-4- hydroxy-5- (hydroxymethyl)tetrahydrofuran-2-yl)-5- methoxypyrimidine-2,4(1H,3H)-dione |
| 144 | 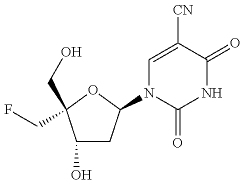 [SMILES: N#Cc1cn([C@H]2C[C@H](O)[C@](CO)(CF)O2)c(=O)[nH]c1=O] | 1-((2R,4S,5R)-5-(fluoromethyl)-4- hydroxy-5- (hydroxymethyl)tetrahydrofuran-2-yl)- 2,4-dioxo-1,2,3,4-tetrahydropyrimidine- 5-carbonitrile |
| 145 | 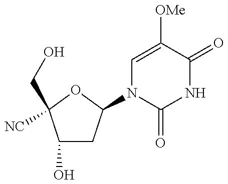 [SMILES: COc1cn([C@H]2C[C@H](O)[C@@](C#N)(CO)O2)c(=O)[nH]c1=O] | (2R,3S,5R)-3-hydroxy-2- (hydroxymethyl)-5-(5-methoxy-2,4- dioxo-3,4-dihydropyrimidin-1(2H)- yl)tetrahydrofuran-2-carbonitrile |
| 147 | 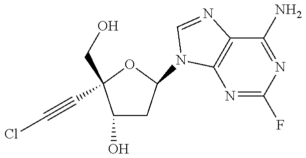 [SMILES: Nc1nc(F)nc2c1ncn2[C@H]1C[C@H](O)[C@@](C#CCl)(CO)O1] | (2R,3S,5R)-5-(6-amino-2-fluoro-9H- purin-9-yl)-2-(chloroethynyl)-2- (hydroxymethyl)tetrahydrofuran-3-ol |
| 148 | 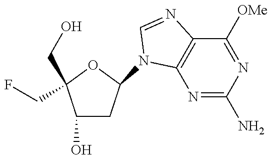 [SMILES: COc1nc(N)nc2c1ncn2[C@H]1C[C@H](O)[C@](CO)(CF)O1] | (2R,3S,5R)-5-(2-amino-6-methoxy-9H- purin-9-yl)-2-(fluoromethyl)-2- (hydroxymethyl)tetrahydrofuran-3-ol |
| 149 | 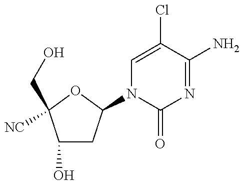 [SMILES: N#C[C@]1(CO)O[C@@H](n2cc(Cl)c(N)nc2=O)C[C@@H]1O] | (2R,3S,5R)-5-(4-amino-5-chloro-2- oxopyrimidin-1(2H)-yl)-3-hydroxy-2- (hydroxymethyl)tetrahydrofuran-2- carbonitrile |
| 151 | 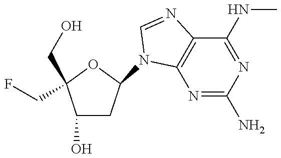 [SMILES: CNc1nc(N)nc2c1ncn2[C@H]1C[C@H](O)[C@](CO)(CF)O1] | (2R,3S,5R)-5-(2-amino-6- (methylamino)-9H-purin-9-yl)-2- (fluoromethyl)-2- (hydroxymethyl)tetrahydrofuran-3-ol |
| 153 | 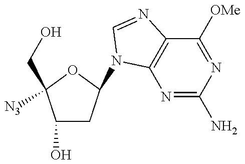 [SMILES: COc1nc(N)nc2c1ncn2[C@H]1C[C@H](O)[C@](CO)(N=[N+]=[N-])O1] | (2R,3S,5R)-5-(2-amino-6-methoxy-9H- purin-9-yl)-2-azido-2- (hydroxymethyl)tetrahydrofuran-3-ol |
| 154 | 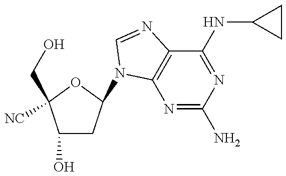 [SMILES: N#C[C@]1(CO)O[C@@H](n2cnc3c(NC4CC4)nc(N)nc32)C[C@@H]1O] | (2R,3S,SR)-5-(2-amino-6- (cyclopropylamino)-9H-purin-9-yl)-3- hydroxy-2- (hydroxymethyl)tetrahydrofuran-2- carbonitrile |
| 157 | 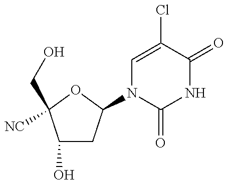 [SMILES: N#C[C@]1(CO)O[C@@H](n2cc(Cl)c(=O)[nH]c2=O)C[C@@H]1O] | (2R,3S,5R)-5-(5-chloro-2,4-dioxo-3,4- dihydropyrimidin-1(2H)-yl)-3-hydroxy- 2-(hydroxymethyl)tetrahydrofuran-2- carbonitrile |
| 158 | 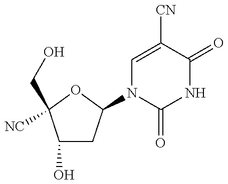 [SMILES: N#Cc1cn([C@H]2C[C@H](O)[C@@](C#N)(CO)O2)c(=O)[nH]c1=O] | 1-((2R,4S,5R)-5-cyano-4-hydroxy-5- (hydroxymethyl)tetrahydrofuran-2-yl)- 2,4-dioxo-1,2,3,4-tetrahydropyrimidine- 5-carbonitrile |
| 159 | 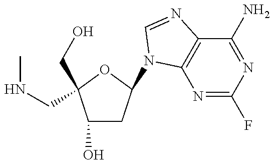 [SMILES: CNC[C@]1(CO)O[C@@H](n2cnc3c(N)nc(F)nc32)C[C@@H]1O] | (2R,3S,5R)-5-(6-amino-2-fluoro-9H- purin-9-yl)-2-(hydroxymethyl)-2- ((methylamino)methyl)tetrahydrofuran- 3-ol |
| 160 | 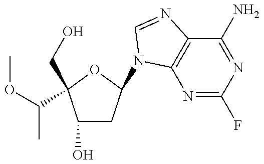 [SMILES: COC(C)[C@]1(CO)O[C@@H](n2cnc3c(N)nc(F)nc32)C[C@@H]1O] | (2R,3S,5R)-5-(6-amino-2-fluoro-9H- purin-9-yl)-2-(hydroxymethyl)-2-((R)-1- methoxyethyl)tetrahydrofuran-3-ol |
| 161 | 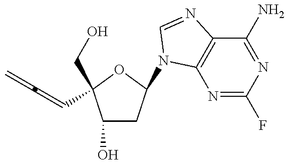 | (2R,3S,5R)-5-(6-amino-2-fluoro-9H- purin-9-yl)-2-(hydroxymethyl)-2-(propa- 1,2-dien-1-yl)tetrahydrofuran-3-ol |
| 162 | 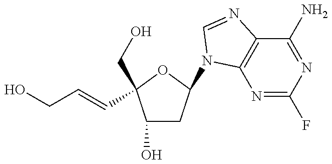 [SMILES: Nc1nc(F)nc2c1ncn2[C@H]1C[C@H](O)[C@@](/C=C/CO)(CO)O1] | (2R,3S,5R)-5-(6-amino-2-fluoro-9H- purin-9-yl)-2-(hydroxymethyl)-2-((E)-3- hydroxyprop-1-en-1-yl)tetrahydrofuran- 3-ol |
| 163 | 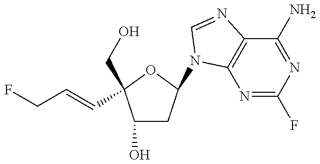 [SMILES: Nc1nc(F)nc2c1ncn2[C@H]1C[C@H](O)[C@@](/C=C/CF)(CO)O1] | (2R,3S,5R)-5-(6-amino-2-fluoro-9H- purin-9-yl)-2-((E)-3-fluoroprop-1-en-1- yl)-2-(hydroxymethyl)tetrahydrofuran-3- ol |
| 164 | 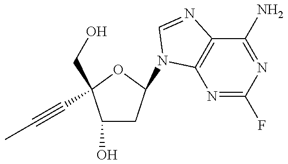 [SMILES: CC#C[C@]1(CO)O[C@@H](n2cnc3c(N)nc(F)nc32)C[C@@H]1O] | (2R,3S,5R)-5-(6-amino-2-fluoro-9H- purin-9-yl)-2-(hydroxymethyl)-2-(prop- 1-yn-1-yl)tetrahydrofuran-3-ol |
| 165 | 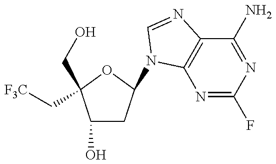 [SMILES: Nc1nc(F)nc2c1ncn2[C@H]1C[C@H](O)[C@](CO)(CC(F)(F)F)O1] | (2R,3S,5R)-5-(6-amino-2-fluoro-9H- purin-9-yl)-2-(hydroxymethyl)-2-(2,2,2- trifluoroethyl)tetrahydrofuran-3-ol |
| 166 | 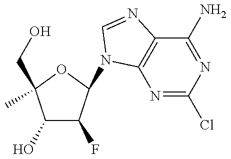 [SMILES: C[C@]1(CO)O[C@@H](n2cnc3c(N)nc(Cl)nc32)[C@@H](F)[C@@H]1O] | (2R,3R,4S,5R)-5-(6-amino-2-chloro-9H- purin-9-yl)-4-fluoro-2-(hydroxymethyl)- 2-methyltetrahydrofuran-3-ol |
| 167 | 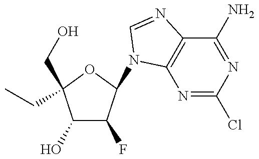 [SMILES: CC[C@]1(CO)O[C@@H](n2cnc3c(N)nc(Cl)nc32)[C@@H](F)[C@@H]1O] | (2R,3R,4S,5R)-5-(6-amino-2-chloro-9H- purin-9-yl)-2-ethyl-4-fluoro-2- (hydroxymethyl)tetrahydrofuran-3-ol |
| 176 | 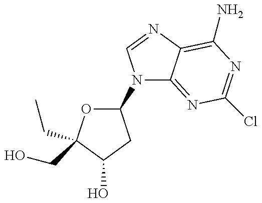 [SMILES: CC[C@]1(CO)O[C@@H](n2cnc3c(N)nc(Cl)nc32)C[C@@H]1O] | (2R,3S,5R)-5-(6-amino-2-chloro-9H- purin-9-yl)-2-ethyl-2- (hydroxymethyl)tetrahydrofuran-3-ol |
| 177 | 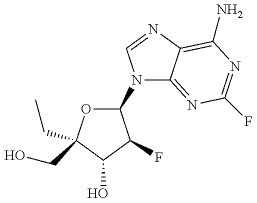 [SMILES: CC[C@]1(CO)O[C@@H](n2cnc3c(N)nc(F)nc32)[C@@H](F)[C@@H]1O] | (2R,3R,4S,5R)-5-(6-amino-2-fluoro-9H- purin-9-yl)-2-ethyl-4-fluoro-2- (hydroxymethyl)tetrahydrofuran-3-ol |
| 179 | 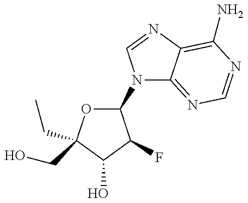 [SMILES: CC[C@]1(CO)O[C@@H](n2cnc3c(N)ncnc32)[C@@H](F)[C@@H]1O] | (2R,3R,4S,5R)-5-(6-amino-9H-purin-9- yl)-2-ethyl-4-fluoro-2- (hydroxymethyl)tetrahydrofuran-3-ol |
| 180 | 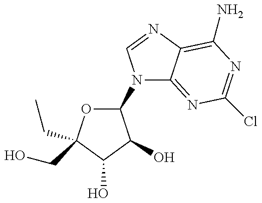 [SMILES: CC[C@]1(CO)O[C@@H](n2cnc3c(N)nc(Cl)nc32)[C@@H](O)[C@@H]1O] | (2R,3S,4S,SR)-5-(6-amino-2-chloro-9H- purin-9-yl)-2-ethyl-2- (hydroxymethyl)tetrahydrofuran-3,4-diol |
| 181 | 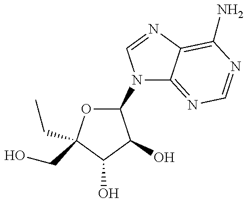 [SMILES: CC[C@]1(CO)O[C@@H](n2cnc3c(N)ncnc32)[C@@H](O)[C@@H]1O] | (2R,3S,4S,5R)-5-(6-amino-9H-purin-9- yl)-2-ethyl-2- (hydroxymethyl)tetrahydrofuran-3,4-diol |
| 182 | 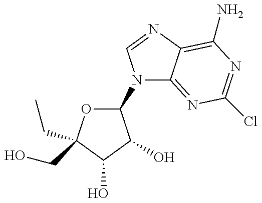 [SMILES: CC[C@]1(CO)O[C@@H](n2cnc3c(N)nc(Cl)nc32)[C@H](O)[C@@H]1O] | (2R,3S,4R,5R)-5-(6-amino-2-chloro-9H- purin-9-yl)-2-ethyl-2- (hydroxymethyl)tetrahydrofuran-3,4-diol |
| 183 | 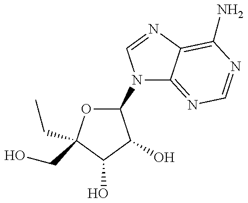 [SMILES: CC[C@]1(CO)O[C@@H](n2cnc3c(N)ncnc32)[C@H](O)[C@@H]1O] | (2R,3S,4R,5R)-5-(6-amino-9H-purin-9- yl)-2-ethyl-2- (hydroxymethyl)tetrahydrofuran-3,4-diol |
| 186 | 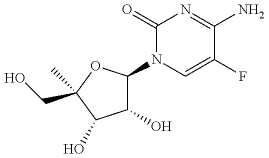 [SMILES: C[C@]1(CO)O[C@@H](n2cc(F)c(N)nc2=O)[C@H](O)[C@@H]1O] | 4-amino-1-((2R,3R,4S,5R)-3,4- dihydroxy-5-(hydroxymethyl)-5- methyltetrahydrofuran-2-yl)-5- fluoropyrimidin-2(1H)-one |
| 188 | 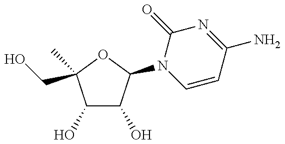 [SMILES: C[C@]1(CO)O[C@@H](n2ccc(N)nc2=O)[C@H](O)[C@@H]1O] | 4-amino-1-((2R,3R,4S,5R)-3,4- dihydroxy-5-(hydroxymethyl)-5- methyltetrahydrofuran-2- yl)pyrimidin-2(1H)-one |
| 189 | 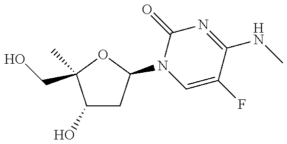 [SMILES: CNc1nc(=O)n([C@H]2C[C@H](O)[C@@](C)(CO)O2)cc1F] | 5-fluoro-1-((2R,4S,5R)-4-hydroxy-5- (hydroxymethyl)-5- methyltetrahydrofuran-2-yl)-4- (methylamino)pyrimidin-2(1H)-one |
| 191 | 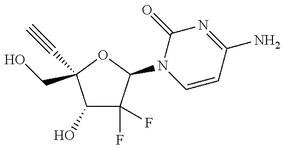 [SMILES: C#C[C@]1(CO)O[C@@H](n2ccc(N)nc2=O)C(F)(F)[C@@H]1O] | 4-amino-1-((2R,4R,5R)-5-ethynyl- 3,3-difluoro-4-hydroxy-5- (hydroxymethyl)tetrahydrofuran-2- yl)pyrimidin-2(1H)-one |
| 192 | 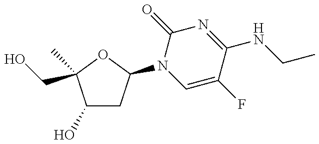 [SMILES: CCNc1nc(=O)n([C@H]2C[C@H](O)[C@@](C)(CO)O2)cc1F] | 4-(ethylamino)-5-fluoro-1- ((2R,4S,5R)-4-hydroxy-5- (hydroxymethyl)-5- methyltetrahydrofuran-2- yl)pyrimidin-2(1H)-one |
| 194 | 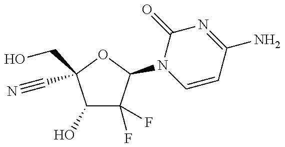 [SMILES: N#C[C@]1(CO)O[C@@H](n2ccc(N)nc2=O)C(F)(F)[C@@H]1O] | (2R,3R,5R)-5-(4-amino-2- oxopyrimidin-1(2H)-yl)-4,4-difluoro- 3-hydroxy-2- (hydroxymethyl)tetrahydrofuran-2- carbonitrile |
| 196 | 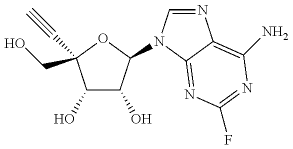 [SMILES: C#C[C@]1(CO)O[C@@H](n2cnc3c(N)nc(F)nc32)[C@H](O)[C@@H]1O] | (2R,3S,4R,5R)-5-(6-amino-2-fluoro- 9H-purin-9-yl)-2-ethynyl-2- (hydroxymethyl)tetrahydrofuran-3,4- diol |
| 199 | 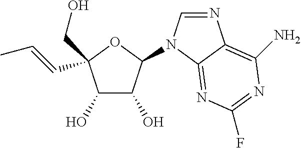 [SMILES: C/C=C/[C@]1(CO)O[C@@H](n2cnc3c(N)nc(F)nc32)[C@H](O)[C@@H]1O] | (2R,3S,4R,5R)-5-(6-amino-2-fluoro- 9H-purin-9-yl)-2-(hydroxymethyl)-2- ((E)-prop-1-en-1-yl)tetrahydrofuran- 3,4-diol |
| 200 | 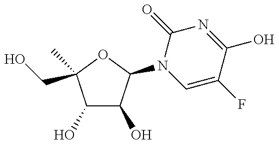 [SMILES: C[C@]1(CO)O[C@@H](n2cc(F)c(O)nc2=O)[C@@H](O)[C@@H]1O] | 1-((2R,3S,4S,5R)-3,4-dihydroxy-5- (hydroxymethyl)-5- methyltetrahydrofuran-2-yl)-5- fluoro-4-hydroxypyrimidin-2(1H)- one |
| 201 | 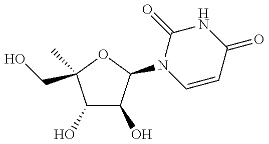 [SMILES: C[C@]1(CO)O[C@@H](n2ccc(=O)[nH]c2=O)[C@@H](O)[C@@H]1O] | 1-((2R,3S,4S,5R)-3,4-dihydroxy-5- (hydroxymethyl)-5- methyltetrahydrofuran-2- yl)pyrimidine-2,4(1H,3H)-dione |
| 204 | 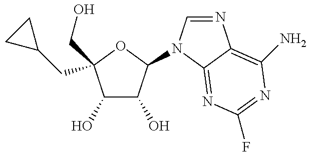 [SMILES: Nc1nc(F)nc2c1ncn2[C@@H]1O[C@@](CO)(CC2CC2)[C@@H](O)[C@H]1O] | (2R,3S,4R,5R)-5-(6-amino-2-fluoro- 9H-purin-9-yl)-2- (cyclopropylmethyl)-2- (hydroxymethyl)tetrahydrofuran-3,4- diol |
| 205 | 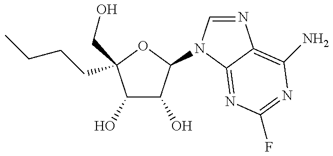 [SMILES: CCCC[C@]1(CO)O[C@@H](n2cnc3c(N)nc(F)nc32)[C@H](O)[C@@H]1O] | (2R,3S,4R,5R)-5-(6-amino-2-fluoro- 9H-purin-9-yl)-2-butyl-2- (hydroxymethyl)tetrahydrofuran-3,4- diol |
| 206 | 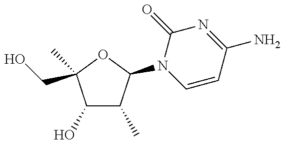 [SMILES: C[C@H]1[C@H](n2ccc(N)nc2=O)O[C@](C)(CO)[C@H]1O] | 4-amino-1-((2R,3R,4S,5R)-4- hydroxy-5-(hydroxymethyl)-3,5- dimethyltetrahydrofuran-2- yl)pyrimidin-2(1H)-one |
| 207 | 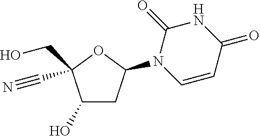 [SMILES: N#C[C@]1(CO)O[C@@H](n2ccc(=O)[nH]c2=O)C[C@@H]1O] | (2R,3S,5R)-5-(2,4-dioxo-3,4- dihydropyrimidin-1(2H)-yl)-3- hydroxy-2- (hydroxymethyl)tetrahydrofuran-2- carbonitrile |
| 209 | 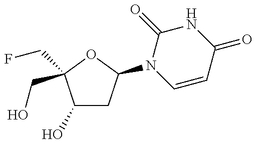 [SMILES: O=c1ccn([C@H]2C[C@H](O)[C@](CO)(CF)O2)c(=O)[nH]1] | 1-((2R,4S,5R)-5-(fluoromethyl)-4- hydroxy-5- (hydroxymethyl)tetrahydrofuran-2- yl)pyrimidine-2,4(1H,3H)-dione |
| 211 | 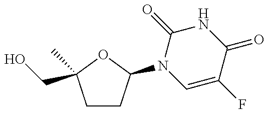 [SMILES: C[C@@]1(CO)CC[C@H](n2cc(F)c(=O)[nH]c2=O)O1] | 5-fluoro-1-((2R,5S)-5- (hydroxymethyl)-5- methyltetrahydrofuran-2- yl)pyrimidine-2,4(1H,3H)-dione |
| 216 | 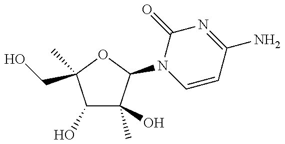 [SMILES: C[C@@]1(O)[C@H](n2ccc(N)nc2=O)O[C@](C)(CO)[C@H]1O] | 4-amino-1-((2R,3S,4S,5R)-3,4- dihydroxy-5-(hydroxymethyl)-3,5- dimethyltetrahydrofuran-2- yl)pyrimidin-2(1H)-one |
| 217 | 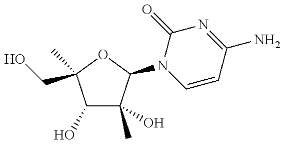 [SMILES: C[C@]1(O)[C@H](n2ccc(N)nc2=O)O[C@](C)(CO)[C@H]1O] | 4-amino-1-((2R,3R,4S,5R)-3,4- dihydroxy-5-(hydroxymethyl)-3,5- dimethyltetrahydrofuran-2- yl)pyrimidin-2(1H)-one |
| 218 | 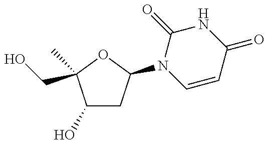 [SMILES: C[C@]1(CO)O[C@@H](n2ccc(=O)[nH]c2=O)C[C@@H]1O] | 1-((2R,4S,SR)-4-hydroxy-5- (hydroxymethyl)-5- methyltetrahydrofuran-2- yl)pyrimidine-2,4(1H,3H)-dione |
| 220 | 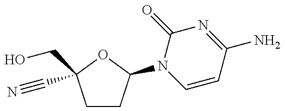 [SMILES: N#C[C@@]1(CO)CC[C@H](n2ccc(N)nc2=O)O1] | (2R,5R)-5-(4-amino-2-oxopyrimidin- 1(2H)-yl)-2- (hydroxymethyl)tetrahydrofuran-2- carbonitrile |
| 221 | 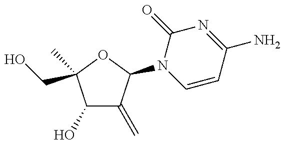 [SMILES: C=C1[C@H](n2ccc(N)nc2=O)O[C@](C)(CO)[C@H]1O] | 4-amino-1-((2R,4S,5R)-4-hydroxy-5- (hydroxymethyl)-5-methyl-3- methylenetetrahydrofuran-2- yl)pyrimidin-2(1H)-one |
| 222 | 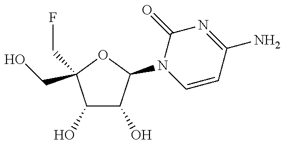 [SMILES: Nc1ccn([C@@H]2O[C@@](CO)(CF)[C@@H](O)[C@H]2O)c(=O)n1] | 4-amino-1-((2R,3R,4S,5R)-5- (fluoromethyl)-3,4-dihydroxy-5- (hydroxymethyl)tetrahydrofuran-2- yl)pyrimidin-2(1H)-one |
| 224 | 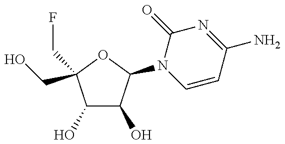 [SMILES: Nc1ccn([C@@H]2O[C@@](CO)(CF)[C@@H](O)[C@@H]2O)c(=O)n1] | 4-amino-1-((2R,3S,4S,5R)-5- (fluoromethyl)-3,4-dihydroxy-5- (hydroxymethyl)tetrahydrofuran-2- yl)pyrimidin-2(1H)-one |
| 225 | 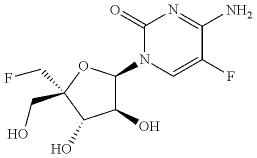 [SMILES: Nc1nc(=O)n([C@@H]2O[C@@](CO)(CF)[C@@H](O)[C@@H]2O)cc1F] | 4-amino-5-fluoro-1-((2R,3S,4S,5R)- 5-(fluoromethyl)-3,4-dihydroxy-5- (hydroxymethyl)tetrahydrofuran-2- yl)pyrimidin-2(1H)-one |
| 226 | 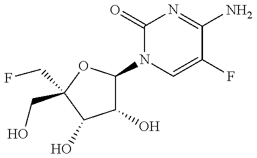 [SMILES: Nc1nc(=O)n([C@@H]2O[C@@](CO)(CF)[C@@H](O)[C@H]2O)cc1F] | 4-amino-5-fluoro-1-((2R,3R,4S,5R)- 5-(fluoromethyl)-3,4-dihydroxy-5- (hydroxymethyl)tetrahydrofuran-2- yl)pyrimidin-2(1H)-one |
| 227 | 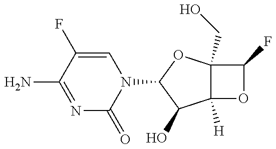 [SMILES: Nc1nc(=O)n([C@@H]2O[C@@]3(CO)[C@@H](F)O[C@H]3[C@H]2O)cc1F] | 4-amino-5-fluoro-1- ((1R,3R,4R,5S,7R)-7-fluoro-4- hydroxy-1-(hydroxymethyl)-2,6- dioxabicyclo[3.2.0]heptan-3- yl)pyrimidin-2(1H)-one |
| 228 | 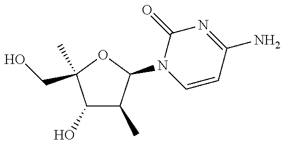 [SMILES: C[C@@H]1[C@H](n2ccc(N)nc2=O)O[C@](C)(CO)[C@H]1O] | 4-amino-1-((2R,3S,4S,5R)-4- hydroxy-5-(hydroxymethyl)-3,5- dimethyltetrahydrofuran-2- yl)pyrimidin-2(1H)-one |
| 229 | 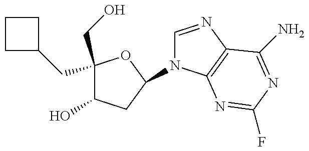 [SMILES: Nc1nc(F)nc2c1ncn2[C@H]1C[C@H](O)[C@](CO)(CC2CCC2)O1] | (2R,3S,5R)-5-(6-amino-2-fluoro-9H- purin-9-yl)-2-(cyclobutylmethyl)-2- (hydroxymethyl)tetrahydrofuran-3-ol |
| 230 | 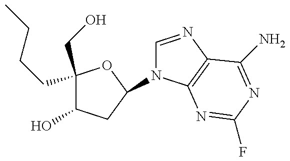 [SMILES: CCCC[C@]1(CO)O[C@@H](n2cnc3c(N)nc(F)nc32)C[C@@H]1O] | (2R,3S,SR)-5-(6-amino-2-fluoro-9H- purin-9-yl)-2-butyl-2- (hydroxymethyl)tetrahydrofuran-3-ol |
| 231 | 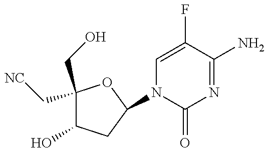 [SMILES: N#CC[C@]1(CO)O[C@@H](n2cc(F)c(N)nc2=O)C[C@@H]1O] | 2-((2R,3S,5R)-5-(4-amino-5-fluoro- 2-oxopyrimidin-1(2H)-yl)-3- hydroxy-2- (hydroxymethyl)tetrahydrofuran-2- yl)acetonitrile |
| 232 | 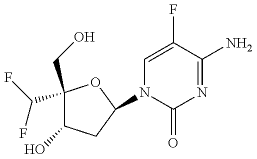 [SMILES: Nc1nc(=O)n([C@H]2C[C@H](O)[C@](CO)(C(F)F)O2)cc1F] | 4-amino-1-((2R,4S,5R)-5- (difluoromethyl)-4-hydroxy-5- (hydroxymethyl)tetrahydrofuran-2- yl)-5-fluoropyrimidin-2(1H)-one |
| 233 | 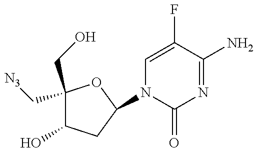 [SMILES: [N-]=[N+]=NC[C@]1(CO)O[C@@H](n2cc(F)c(N)nc2=O)C[C@@H]1O] | 4-amino-1-((2R,4S,5R)-5- (azidomethyl)-4-hydroxy-5- (hydroxymethyl)tetrahydrofuran-2- yl)-5-fluoropyrimidin-2(1H)-one |
| 234 | 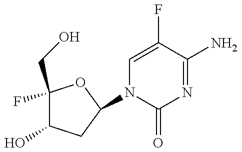 [SMILES: Nc1nc(=O)n([C@H]2C[C@H](O)[C@@](F)(CO)O2)cc1F] | 4-amino-5-fluoro-1-((2R,4S,5S)-5- fluoro-4-hydroxy-5- (hydroxymethyl)tetrahydrofuran-2- yl)pyrimidin-2(1H)-one |
| 234 | 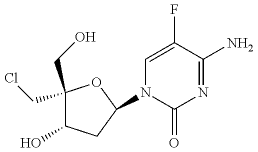 [SMILES: Nc1nc(=O)n([C@H]2C[C@H](O)[C@](CO)(CCl)O2)cc1F] | 4-amino-1-((2R,4S,5R)-5- (chloromethyl)-4-hydroxy-5- (hydroxymethyl)tetrahydrofuran-2- yl)-5-fluoropyrimidin-2(1H)-one |
| 236 | 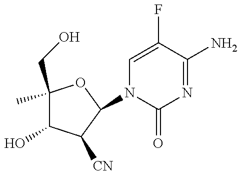 [SMILES: C[C@]1(CO)O[C@@H](n2cc(F)c(N)nc2=O)[C@@H](C#N)[C@@H]1O] | (2R,3S,4S,5R)-2-(4-amino-5-fluoro- 2-oxopyrimidin-1(2H)-yl)-4- hydroxy-5-(hydroxymethyl)-5- methyltetrahydrofuran-3-carbonitrile |
| 237 | 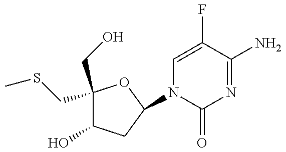 [SMILES: CSC[C@]1(CO)O[C@@H](n2cc(F)c(N)nc2=O)C[C@@H]1O] | 4-amino-5-fluoro-1-((2R,4S,SR)-4- hydroxy-5-(hydroxymethyl)-5- ((methylthio)methyl)tetrahydrofuran- 2-yl)pyrimidin-2(1H)-one |
| 238 | 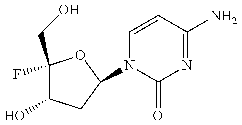 [SMILES: Nc1ccn([C@H]2C[C@H](O)[C@@](F)(CO)O2)c(=O)n1] | 4-amino-1-((2R,4S,5S)-5-fluoro-4- hydroxy-5- (hydroxymethyl)tetrahydrofuran-2- yl)pyrimidin-2(1H)-one |
| 239 | 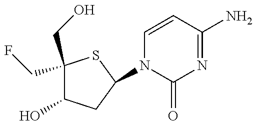 [SMILES: Nc1ccn([C@H]2C[C@H](O)[C@](CO)(CF)S2)c(=O)n1] | 4-amino-1-((2R,4S,5R)-5- (fluoromethyl)-4-hydroxy-5- (hydroxymethyl)tetrahydrothiophen- 2-yl)pyrimidin-2(1H)-one |
| 240 | 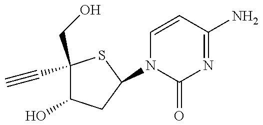 [SMILES: C#C[C@]1(CO)S[C@@H](n2ccc(N)nc2=O)C[C@@H]1O] | 4-amino-1-((2R,4S,5R)-5-ethynyl-4- hydroxy-5- (hydroxymethyl)tetrahydrothiophen- 2-yl)pyrimidin-2(1H)-one |
| 241 | 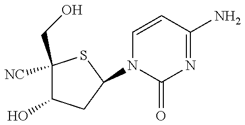 [SMILES: N#C[C@]1(CO)S[C@@H](n2ccc(N)nc2=O)C[C@@H]1O] | (2R,3S,5R)-5-(4-amino-2- oxopyrimidin-1(2H)-yl)-3-hydroxy- 2- (hydroxymethyl)tetrahydrothiophene- 2-carbonitrile |
| 242 | 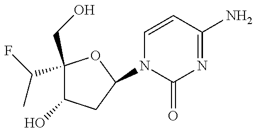 [SMILES: CC(F)[C@]1(CO)O[C@@H](n2ccc(N)nc2=O)C[C@@H]1O] | 4-amino-1-((2R,4S,5R)-5-(1- fluoroethyl)-4-hydroxy-5- (hydroxymethyl)tetrahydrofuran-2- yl)pyrimidin-2(1H)-one |
| 243 | 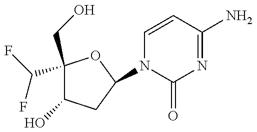 [SMILES: Nc1ccn([C@H]2C[C@H](O)[C@](CO)(C(F)F)O2)c(=O)n1] | 4-amino-1-((2R,4S,5R)-5- (difluoromethyl)-4-hydroxy-5- (hydroxymethyl)tetrahydrofuran-2- yl)pyrimidin-2(1H)-one |
| 244 | 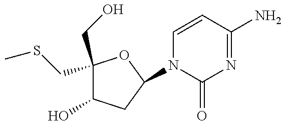 [SMILES: CSC[C@]1(CO)O[C@@H](n2ccc(N)nc2=O)C[C@@H]1O] | 4-amino-1-((2R,4S,5R)-4-hydroxy-5- (hydroxymethyl)-5- ((methylthio)methyl)tetrahydrofuran- 2-yl)pyrimidin-2(1H)-one |
| 245 | 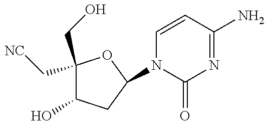 [SMILES: N#CC[C@]1(CO)O[C@@H](n2ccc(N)nc2=O)C[C@@H]1O] | 2-((2R,3S,5R)-5-(4-amino-2- oxopyrimidin-1(2H)-yl)-3-hydroxy- 2-(hydroxymethyl)tetrahydrofuran-2- yl)acetonitrile |
| 246 | 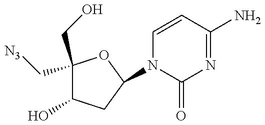 [SMILES: [N-]=[N+]=NC[C@]1(CO)O[C@@H](n2ccc(N)nc2=O)C[C@@H]1O] | 4-amino-1-((2R,4S,5R)-5- (azidomethyl)-4-hydroxy-5- (hydroxymethyl)tetrahydrofuran-2- yl)pyrimidin-2(1H)-one |
| 247 | 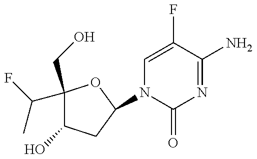 [SMILES: CC(F)[C@]1(CO)O[C@@H](n2cc(F)c(N)nc2=O)C[C@@H]1O] | 4-amino-5-fluoro-1-((2R,4S,5R)-5- (1-fluoroetbyl)-4-hydroxy-5- (hydroxymethyl)tetrahydrofuran-2- yl)pyrimidin-2(1H)-one |
| 248 | 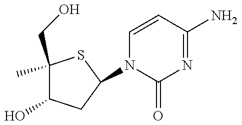 [SMILES: C[C@]1(CO)S[C@@H](n2ccc(N)nc2=O)C[C@@H]1O] | 4-amino-1-((2R,4S,5R)-4-hydroxy-5- (hydroxymethyl)-5- methyltetrahydrothiophen-2- yl)pyrimidin-2(1H)-one |
| 249 | 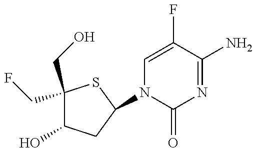 [SMILES: Nc1nc(=O)n([C@H]2C[C@H](O)[C@](CO)(CF)S2)cc1F] | 4-amino-5-fluoro-1-((2R,4S,5R)-5- (fluoromethyl)-4-hydroxy-5- (hydroxymethyl)tetrahydrothiophen- 2-yl)pyrimidin-2(1H)-one |
| 250 | 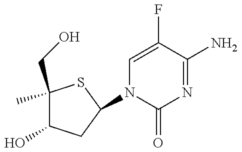 [SMILES: C[C@]1(CO)S[C@@H](n2cc(F)c(N)nc2=O)C[C@@H]1O] | 4-amino-5-fluoro-1-((2R,4S,5R)-4- hydroxy-5-(hydroxymethyl)-5- methyltetrahydrothiophen-2- yl)pyrimidin-2(1H)-one |
| 251 | 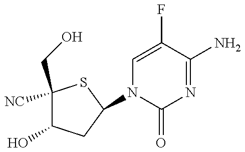 [SMILES: N#C[C@]1(CO)S[C@@H](n2cc(F)c(N)nc2=O)C[C@@H]1O] | (2R,3S,5R)-5-(4-amino-5-fluoro-2- oxopyrimidin-1(2H)-yl)-3-hydroxy- 2- (hydroxymethyl)tetrahydrothiophene- 2-carbonitrile |
| 252 | 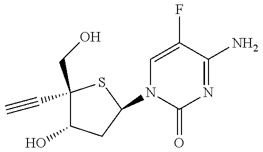 [SMILES: C#C[C@]1(CO)S[C@@H](n2cc(F)c(N)nc2=O)C[C@@H]1O] | 4-amino-1-((2R,4S,5R)-5-ethynyl-4- hydroxy-5- (hydroxymethyl)tetrahydrothiophen- 2-yl)-5-fluoropyrimidin-2(1H)-one |
| 253 | 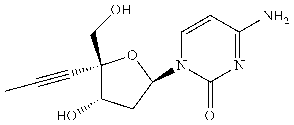 [SMILES: CC#C[C@]1(CO)O[C@@H](n2ccc(N)nc2=O)C[C@@H]1O] | 4-amino-1-((2R,4S,5R)-4-hydroxy-5- (hydroxymethyl)-5-(prop-1-yn-1- yl)tetrahydrofuran-2-yl)pyrimidin- 2(1H)-one |
| 254 | 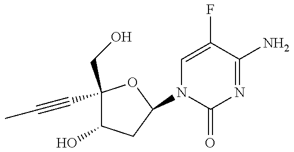 [SMILES: CC#C[C@]1(CO)O[C@@H](n2cc(F)c(N)nc2=O)C[C@@H]1O] | 4-amino-5-fluoro-1-((2R,4S,5R)-4- hydroxy-5-(hydroxymethyl)-5-(prop- 1-yn-1-yl)tetrahydrofuran-2- yl)pyrimidin-2(1H)-one |
| 255 | 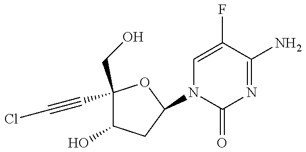 [SMILES: Nc1nc(=O)n([C@H]2C[C@H](O)[C@@](C#CCl)(CO)O2)cc1F] | 4-amino-1-((2R,4S,5R)-5- (chloroethynyl)-4-hydroxy-5- (hydroxymethyl)tetrahydrofuran-2- yl)-5-fluoropyrimidin-2(1H)-one |
| 256 | 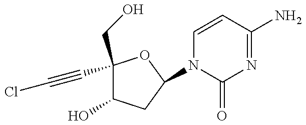 [SMILES: Nc1ccn([C@H]2C[C@H](O)[C@@](C#CCl)(CO)O2)c(=O)n1] | 4-amino-1-((2R,4S,5R)-5- (chloroethynyl)-4-hydroxy-5- (hydroxymethyl)tetrahydrofuran-2- yl)pyrimidin-2(1H)-one |

In another embodiment, Compounds of the Disclosure are compounds of Table 3, or a pharmaceutically acceptable salt, solvate, and/or tautomer thereof, and/or mono-, di-, or trisphosphates thereof.

TABLE 3

| Cpd. No. | Structure | Chemical Name |
|---|---|---|
| 169 | 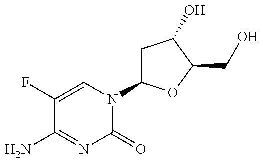 [SMILES: Nc1nc(=O)n([C@H]2C[C@H](O)[C@@H](CO)O2)cc1F] | 4-amino-5-fluoro-1-((2R,4S,5R)-4- hydroxy-5- (hydroxymethyl)tetrahydrofuran-2- yl)pyrimidin-2(1H)-one (5-fluoro-2′-deoxycytidine) |
| 170 | 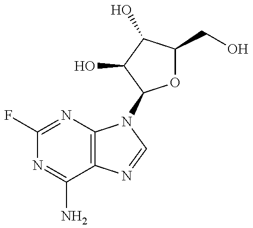 [SMILES: Nc1nc(F)nc2c1ncn2[C@@H]1O[C@H](CO)[C@@H](O)[C@@H]1O] | (2R,3S,4S,5R)-2-(6-amino-2-fluoro- 9H-purin-9-yl)-5- (hydroxymethyl)tetrahydrofuran-3,4- diol (2-fluoroadenine 9-beta-D- arabinofuranoside) |
| 171 | 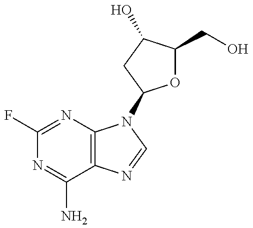 [SMILES: Nc1nc(F)nc2c1ncn2[C@H]1C[C@H](O)[C@@H](CO)O1] | (2R,3S,5R)-5-(6-amino-2-fluoro-9H- purin-9-yl)-2- (hydroxymethyl)tetrahydrofuran-3-ol (2-fluoro-2′-deoxyadenosine) |
| 172 | 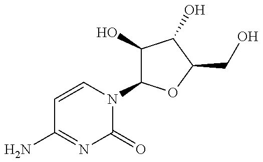 [SMILES: Nc1ccn([C@@H]2O[C@H](CO)[C@@H](O)[C@@H]2O)c(=O)n1] | 4-amino-1-((2R,3S,4S,5R)-3,4- dihydroxy-5- (hydroxymethyl)tetrahydrofuran-2- yl)pyrimidin-2(1H)-one (cytosine beta-D-arabinofuranoside) |
| 123 | 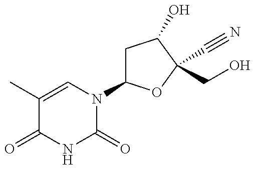 [SMILES: Cc1cn([C@H]2C[C@H](O)[C@@](C#N)(CO)O2)c(=O)[nH]c1=O] | (2R,3S,5R)-3-hydroxy-2- (hydroxymethyl)-5-(5-methyl-2,4-dioxo- 3,4-dihydropyrimidin-1(2H)- yl)tetrahydrofuran-2-carbonitrile |
| 184 | 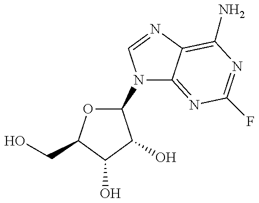 [SMILES: Nc1nc(F)nc2c1ncn2[C@@H]1O[C@H](CO)[C@@H](O)[C@H]1O] | (2R,3R,4S,5R)-2-(6-amino-2-fluoro-9H- purin-9-yl)-5- (hydroxymethyl)tetrahydrofuran-3,4-diol (2-Fluoroadenosine) |
| 185 | 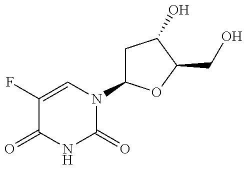 [SMILES: O=c1[nH]c(=O)n([C@H]2C[C@H](O)[C@@H](CO)O2)cc1F] | 5-fluoro-1-((2R,4S,5R)-4-hydroxy-5- (hydroxymethyl)tetrahydrofuran-2- yl)pyrimidine-2,4(1H,3H)-dione (5-Fluoro-2′-deoxyuridine) |
| 198 | 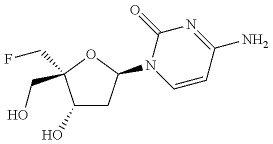 [SMILES: Nc1ccn([C@H]2C[C@H](O)[C@](CO)(CF)O2)c(=O)n1] | 4-amino-1-((2R,4S,5R)-5- (fluoromethyl)-4-hydroxy-5- (hydroxymethyl)tetrahydrofuran-2- yl)pyrimidin-2(1H)-one |
| 210 | 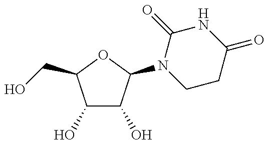 [SMILES: O=C1CCN([C@@H]2O[C@H](CO)[C@@H](O)[C@H]2O)C(=O)N1] | 1-((2R,3R,4S,5R)-3,4-dihydroxy-5- (hydroxymethyl)tetrahydrofuran-2- yl)dihydropyrimidine-2,4(1H,3H)- dione (tetrahydrouridine) |
| 219 | 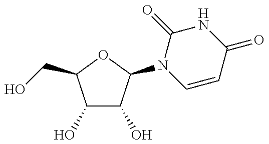 [SMILES: O=c1ccn([C@@H]2O[C@H](CO)[C@@H](O)[C@H]2O)c(=O)[nH]1] | 1-((2R,3S,4S,5R)-3,4-dihydroxy-5- (hydroxymethyl)tetrahydrofuran-2- yl)pyrimidine-2,4(1H,3H)-dione (uracil 1-beta-d-arabinofuranoside) |

Tables 1-3 and 3A, see below, provide chemical structures and the associated chemical names generated by Chemdraw® Professional version 20.1.1.125. In the event of any ambiguity between their chemical structure and chemical name, Compounds of the Disclosure are defined by their structure.

In another embodiment, a Compound of the Disclosure is a monophosphate of a compound of any one of Tables 1-3, or a pharmaceutically acceptable salt thereof. See Schemes 1A and 1B.

In another embodiment, a Compound of the Disclosure is a diphosphate of a compound of any one of Tables 1-3, or a pharmaceutically acceptable salt thereof. See Schemes 1A and 1B.

In another embodiment, a Compound of the Disclosure is a triphosphate of a compound of any one of Tables 1-3, or a pharmaceutically acceptable salt thereof. See Schemes 1A and 1B. In another embodiment, Compounds of the Disclosure are compounds of Table 3A, or a pharmaceutically acceptable salt thereof.

TABLE 3A

| Cpd. No. | Structure | Chemical Name |
|---|---|---|
| 202 | 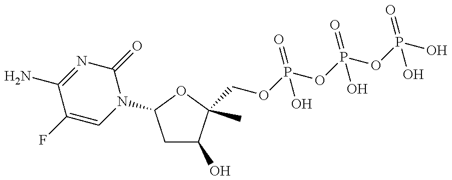 [SMILES: C[C@]1(COP(=O)(O)OP(=O)(O)OP(=O)(O)O)O[C@@H](n2cc(F)c(N)nc2=O)C[C@@H]1O] | ((2R,3S,5R)-5-(4-amino-5- fluoro-2-oxopyrimidin- 1(2H)-yl)-3-hydroxy-2- methyltetrahydrofuran-2- yl)methyl tetrahydrogen triphosphate |
| 203 | 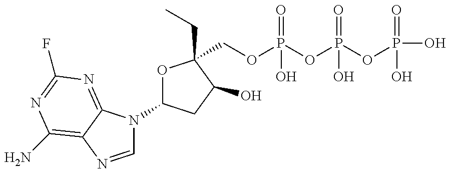 [SMILES: CC[C@]1(COP(=O)(O)OP(=O)(O)OP(=O)(O)O)O[C@@H](n2cnc3c(N)nc(F)nc32)C[C@@H]1O] | ((2R,3S,5R)-5-(6-amino-2- fluoro-9H-purin-9-yl)-2- ethyl-3- hydroxytetrahydrofuran-2- yl)methyl tetrahydrogen triphosphate |
| 208 | 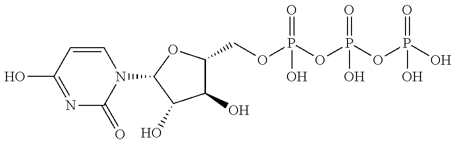 [SMILES: O=c1nc(O)ccn1[C@@H]1O[C@H](COP(=O)(O)OP(=O)(O)OP(=O)(O)O)[C@@H](O)[C@@H]1O] | ara-cytidine 5′ triphosphate |
| 213 | 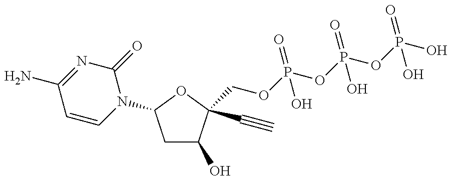 [SMILES: C#C[C@]1(COP(=O)(O)OP(=O)(O)OP(=O)(O)O)O[C@@H](n2ccc(N)nc2=O)C[C@@H]1O] | ((2R,3S,5R)-5-(4-amino-2- oxopyrimidin-1(2H)-yl)-2- ethynyl-3- hydroxytetrahydrofuran-2- yl)methyl tetrahydrogen triphosphate |
| 214 | 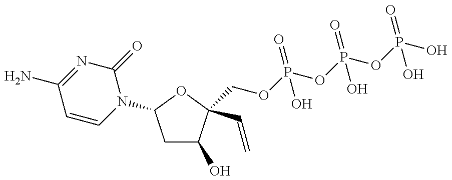 [SMILES: C=C[C@]1(COP(=O)(O)OP(=O)(O)OP(=O)(O)O)O[C@@H](n2ccc(N)nc2=O)C[C@@H]1O] | ((2R,3S,5R)-5-(4-amino-2- oxopyrimidin-1(2H)-yl)-3- hydroxy-2- vinyltetrahydrofuran-2- yl)methyl tetrahydrogen triphosphate |
| 215 | 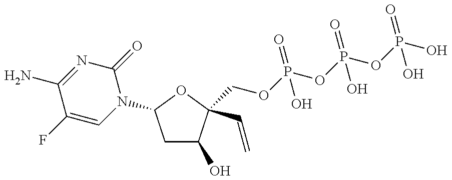 [SMILES: C=C[C@]1(COP(=O)(O)OP(=O)(O)OP(=O)(O)O)O[C@@H](n2cc(F)c(N)nc2=O)C[C@@H]1O] | ((2R,3S,5R)-5-(4-amino-5- fluoro-2-oxopyrimidin- 1(2H)-yl)-3-hydroxy-2- vinyltetrahydrofuran-2- yl)methyl tetrahydrogen triphosphate |

In another embodiment, the disclosure provides a pharmaceutical composition comprising a Compound of the Disclosure, or a pharmaceutically acceptable salt or solvate thereof, or a tautomer thereof, and one or more pharmaceutically acceptable excipients.

### II. Therapeutic Methods and Uses

Compounds of the Disclosure may be phosphorylated in a cell of a subject by the addition of one, two, or three phosphate groups to form the corresponding mono-, di-, or triphosphates as shown in Scheme A for Cpd. No. 4. Without wishing to be bound by any particular theory, these phosphorylated Compounds of the Disclosure inhibit DDR enzymes following admiration to a subject. As such, these compounds can be used, for example, to treat or prevent cancers wherein DDR enzymes play a causative role. Mono-, di-, or triphosphates may exist as pharmaceutically acceptable salts as shown in Scheme 1B for Cpd. No. 4.

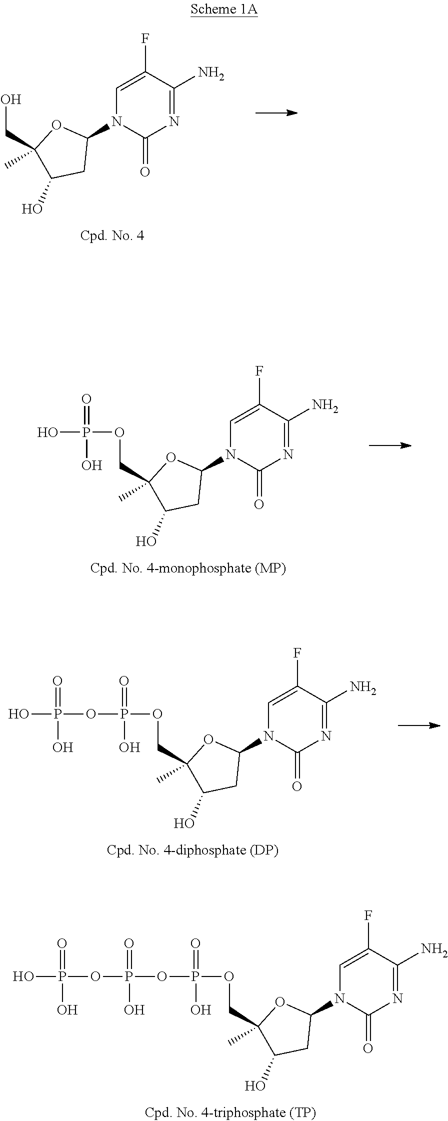 [SMILES: C[C@]1(CO)O[C@@H](n2cc(F)c(N)nc2=O)C[C@@H]1O ; C[C@]1(COP(=O)(O)O)O[C@@H](n2cc(F)c(N)nc2=O)C[C@@H]1O ; C[C@]1(COP(=O)(O)OP(=O)(O)O)O[C@@H](n2cc(F)c(N)nc2=O)C[C@@H]1O ; C[C@]1(COP(=O)(O)OP(=O)(O)OP(=O)(O)O)O[C@@H](n2cc(F)c(N)nc2=O)C[C@@H]1O]

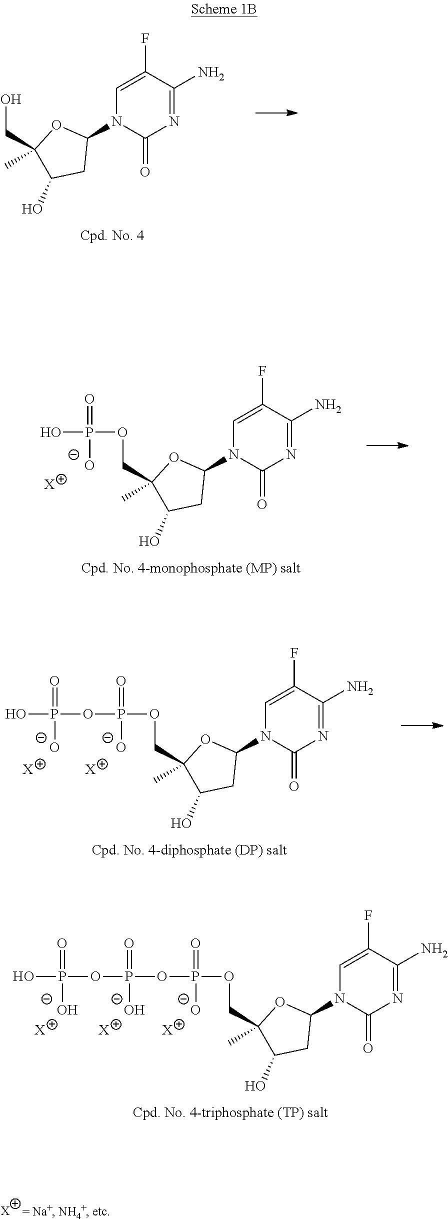

Again, without wishing to be bound by any particular theory, cancer cells often acquire mutations in DDR genes, making them dependent on remaining DNA repair pathways. Dependence on TMEJ, for example, is characterized by an increased Pol θ expression and is associated with poor patient prognosis. Inhibition of Pol θ in Pol θ-dependent cancers leads to synthetic lethality. This is well described for malignancies deficient in homologous recombination, e.g., due to mutations in BRCA1 or BRCA2. See, e.g., Schrempt et al., Trends in Cancer 7:98-111 (2021) https://doi.org/10.1016/J.trecan.2020.09.007.

In one embodiment, a Compound of the Disclosure is synthetic lethal in cancers with HR or NHEJ deficiency.

A Compound of the Disclosure, or pharmaceutical composition thereof, can be administered to a subject in need thereof, e.g., a subject already suffering from cancer; a subject suspected of having cancer; or a subject at risk of acquiring cancer. When a Compound of the Disclosure is administered to a subject at risk of acquiring cancer, the intention is to try to avoid the cancer in the subject, e.g., by preventing or reducing the expression of a DDR enzyme, e.g., by preventing or reducing Pol θ expression.

In one embodiment, the disclosure provides a method of treating or preventing a cancer in a subject in need thereof, the method comprising administering a therapeutically effective amount of a Compound of the Disclosure, or pharmaceutical composition thereof, to the subject.

In another embodiment, the disclosure provides a method of treating cancer in a subject in need thereof, the method comprising administering a therapeutically effective amount of a Compound of the Disclosure, or pharmaceutical composition thereof, to the subject.

In another embodiment, the disclosure provides a method of preventing cancer in a subject in need thereof, the method comprising administering a therapeutically effective amount of a Compound of the Disclosure, or pharmaceutical composition thereof, to the subject.

In another embodiment, the disclosure provides a Compound of the Disclosure, or pharmaceutical composition thereof, for use in treating or preventing cancer in a subject.

In another embodiment, the disclosure provides a Compound of the Disclosure, or pharmaceutical composition thereof, for use in treating cancer.

In another embodiment, the disclosure provides a Compound of the Disclosure, or pharmaceutical composition thereof, for use in preventing cancer.

In another embodiment, the disclosure provides the use of a Compound of the Disclosure, or pharmaceutical composition thereof, in the manufacture of a medicament for treating or preventing cancer in a subject.

In another embodiment, the disclosure provides the use of a Compound of the Disclosure, or pharmaceutical composition thereof, in the manufacture of a medicament for treating a cancer in a subject.

In another embodiment, the disclosure provides the use of a Compound of the Disclosure, or pharmaceutical composition thereof, in the manufacture of a medicament for preventing cancer in a subject. In some embodiments, the Compound of the Disclosure is a compound of Table I, or pharmaceutical composition thereof.

In another embodiment, the subject is (a) not infected with the HIV virus, (b) not suspected of being infected with the HIV virus, (c) not being treated for the HIV virus, and/or (d) not being treated to prevent the HIV virus.

In another embodiment, the disclosure provides a method of inhibiting one or more DDR enzymes in a subject in need thereof, the method comprising administering to the subject a therapeutically effective amount of a Compound of the Disclosure.

In another embodiment, the disclosure provides a method of inhibiting Pol θ in a subject in need thereof, the method comprising administering to the subject a therapeutically effective amount of a Compound of the Disclosure.

In another embodiment, the disclosure provides a method of inhibiting Pol η in a subject in need thereof, the method comprising administering to the subject a therapeutically effective amount of a Compound of the Disclosure.

In another embodiment, the disclosure provides a method of inhibiting Pol μ in a subject in need thereof, the method comprising administering to the subject a therapeutically effective amount of a Compound of the Disclosure.

In another embodiment, the disclosure provides a method for treating cancer in subject in need thereof comprising administering a therapeutically effective amount of a Compound of the Disclosure, wherein the cells of the cancer are suspected of or exhibit deficiency of a DDR enzyme. In some embodiments, the deficiency is a reduction in the activity of a DDR enzyme. In some embodiments, the deficiency is an absence of activity of a DDR enzyme. The deficiency of the DDR enzyme may be caused by any means that results in the deficiency of the DDR enzyme including, but not limited to, genetic variations of the gene encoding the DDR enzyme including mutations, e.g. point mutations, substitutions, deletions, single nucleotide polymorphisms (SNPs), haplotypes, chromosome abnormalities, Copy Number Variation (CNV), epigenetics, DNA inversions, reduction in expression and/or mis-localisation.

In another embodiment, the disclosure provides a method for treating cancer in subject in need thereof comprising administering a therapeutically effective amount of a Compound of the Disclosure, wherein the cells of the cancer are suspected of or exhibit amplification of a DDR enzyme. Without wishing to be bound by any particular theory, DDR gene amplification, e.g., overexpression, can lead to chemotherapy resistance and poor overall survival by augmenting DDR. See, e.g., Wu et al., Theranostics 10:3939-3951 (2020).

In one embodiment, the DDR enzyme is encoded by at least one homologous recombination (HR) gene.

In another embodiment, the at least one HR gene is ATM, ATR, BRCA1, BRCA2, BARD1, RAD51C, RAD50, CHEK1, CHEK2, FANCA, FANCB, FANCC, FANCD2, FANCE, FANCF, FANCG, FANCI, FANCL, FANCM, PALB2 (FANCN), FANCP (BYBD12), ERCC4 (FANCQ), FPEN, CDK12, MRE11, NBS1, NBN, CLASPIN, BLM, WRN, SMARCA2, SMARCA4 IJGi1, RPA1, BRIP1 and/or PTEN.

In another embodiment, the cancer is a HR deficient cancer.

In another embodiment, the DDR enzyme is encoded by at least one non-homologous end-joining deficiency (NHEJD) gene.

In another embodiment, the at least one NHEJD gene is: LIG4, NHEJI, POLL, POLM, PRKDC, XRCC4, XRCC6, RCC6, and/or DCLRE1C.

In another embodiment, the cancer is a NHEJD-deficient cancer.

The therapeutic methods and uses provided herein comprise administering a Compound of the Disclosure to a subject having cancer in an amount which is effective to achieve its intended purpose. While individual needs vary, determination of optimal ranges of effective amounts of each component is within the skill of the art. Typically, the Compound of the Disclosure is administered in an amount from about 0.01 mg/kg to about 500 mg/kg, about 0.05 mg/kg to about 100 mg/kg, about 0.05 mg/kg to about 50 mg/kg, or about 0.05 mg/kg to about 10 mg/kg. In one embodiment, the Compound of the Disclosure is administered once a day. In another embodiment, the Compound of the Disclosure is administered twice a day. In one embodiment, the Compound of the Disclosure is administered three times a day. In one embodiment, the Compound of the Disclosure is administered four times a day. These dosages are exemplary, but there can be individual instances in which higher or lower dosages are merited, and such are within the scope of this disclosure. In practice, the physician determines the actual dosing regimen that is most suitable for an individual subject, which can vary with the age, weight, and response of the particular subject.

A unit dose may comprise from about 0.01 mg to about 1000 mg, e.g., about 1 mg to about 500 mg, e.g., about 1 mg to about 250 mg, e.g., about 1 mg to about 100 mg of the Compound of the Disclosure. For example, the unit oral dose of the Compound of the Disclosure may comprise, for example, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, or 100 mg. The unit dose may be administered one or more times daily, e.g., as one or more tablets or capsules. The unit dose may also be administered by any suitable route, e.g., orally, by IV, inhalation or subcutaneously to the subject. In practice, the physician determines the actual dosing regimen that is most suitable for an individual subject, which can vary with the age, weight, and response of the particular subject.

In one embodiment, the Compound of the Disclosure is administered to a subject in an amount from about 0.1 mg to about 1000 mg once a day, twice a day, three times a day, or four times a day. In another embodiment, the Compound of the Disclosure is administered to a subject in an amount from about 1 mg to about 100 mg per day.

In one embodiment, the Compound of the Disclosure is administered to the subject in a single dose. In another embodiment, the Compound of the Disclosure is administered to the subject in two divided doses. In another embodiment, the Compound of the Disclosure is administered to the subject in three divided doses. In another embodiment, the Compound of the Disclosure is administered to the subject in four divided doses.

The Compound of the Disclosure can be administered to a subject in the form of a raw chemical or as part of a pharmaceutical composition containing the Compound of the Disclosure combined with a suitable pharmaceutically acceptable carrier. Such a carrier can be selected from pharmaceutically acceptable excipients, vehicles, and auxiliaries. The term “pharmaceutically acceptable carrier,” “pharmaceutically acceptable vehicle,” or “pharmaceutically acceptable vehicle” encompasses any of the standard pharmaceutical carriers, solvents, surfactants, or vehicles. Suitable pharmaceutically acceptable vehicles include aqueous vehicles and nonaqueous vehicles. Standard pharmaceutical carriers and their formulations are described in Remington's Pharmaceutical Sciences, Mack Publishing Co., Easton, PA, 19th ed. 1995.

A pharmaceutical composition comprising the Compound of the Disclosure can contain from about 0.01 to 99 percent by weight, e.g., from about 0.25 to 75 percent by weight, of the Compound of the Disclosure, e.g., about 1%, about 5%, about 10%, about 15%, about 20%, about 25%, about 30%, about 35%, about 40%, about 45%, about 50%, about 55%, about 60%, about 65%, about 70%, or about 75% by weight of the Compound of the Disclosure.

The Compound of the Disclosure, or pharmaceutical composition comprising the Compound of the Disclosure, can be administered by any suitable route, for example by oral, buccal, inhalation, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration to a subject. Dosage forms depend on the route administration. Dosage forms include, but are not limited to, tablets, dragees, slow release lozenges, capsules, liquid solutions, liquid suspensions, oral/nasal spray, transdermal patch, thin dissolvable film, ointments, sustained or controlled release implants, mouth rinses and mouth washes, gels, hair rinses, hair gels, and shampoos, and suppositories, as well as suitable solutions for administration by intravenous infusion, and suitable suspensions for administration subcutaneous injection, and suitable powders for reconstitution. Parenteral administration can be accomplished using a needle and syringe or using other technique known in the art. In one embodiment, the Compound of the Disclosure is administered orally to the subject. In one embodiment, the Compound of the Disclosure is administered subcutaneously to the subject. In one embodiment, the Compound of the Disclosure is administered intravenously to the subject.

The Compound of the Disclosure and pharmaceutical compositions comprising the Compound of the Disclosure may be administered to any subject which may experience the beneficial effects of, for example, inhibiting one or more DDR enzymes.

The pharmaceutical preparations provided herein are manufactured by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries can be suitable flow-regulating agents and lubricants. Suitable auxiliaries include, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of a Compound of the Disclosure may be administered to a subject. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension including, for example, sodium carboxymethyl cellulose, sorbitol, and/or dextran. Optionally, the suspension may also contain stabilizers and other additives.

Therapeutically effective amounts of a Compound of the Disclosure formulated in accordance with standard pharmaceutical practices are administered to a subject in need thereof. Whether such a treatment is indicated depends on the individual case and is subject to medical assessment (diagnosis) that takes into consideration signs, symptoms, and/or malfunctions that are present, the risks of developing particular signs, symptoms and/or malfunctions, and other factors.

Pharmaceutical compositions include those wherein a Compound of the Disclosure is administered in an effective amount to achieve its intended purpose. The exact formulation, route of administration, and dosage is determined by an individual physician in view of the diagnosed condition or disease. Dosage amount and interval can be adjusted individually to provide levels of the Compound of the Disclosure that is sufficient to maintain therapeutic effects.

Toxicity and therapeutic efficacy of the Compound of the Disclosure can be determined by standard pharmaceutical procedures in cell cultures or experimental animals, e.g., for determining the maximum tolerated dose (MTD) of a compound, which defines as the highest dose that causes no toxicity in a subject. The dose ratio between the maximum tolerated dose and therapeutic effects is the therapeutic index. The dosage can vary within this range depending upon the dosage form employed, and the route of administration utilized. Determination of a therapeutically effective amount is well within the capability of those skilled in the art, especially in light of the detailed disclosure provided herein.

A therapeutically effective amount of the Compound of the Disclosure required for use in therapy varies with the nature of the cancer being treated, the length of time that activity is desired, and the age and the condition of the subject, and ultimately is determined by the attendant physician. For example, dosage amounts and intervals can be adjusted individually to provide plasma levels of a Compound of the Disclosure that are sufficient to maintain the desired therapeutic effects. The desired dose conveniently can be administered in a single dose, or as multiple doses administered at appropriate intervals, for example as one, two, three, four or more subdoses per day.

The therapeutic methods and uses of the present disclosure can be accomplished by administering a Compound of the Disclosure as a neat compound or as a pharmaceutical composition.

Administration of a pharmaceutical composition, or a neat Compound of the Disclosure can be performed before, during, or after the clinical diagnosis of the cancer. Typically, the pharmaceutical compositions are sterile, and contain no toxic, carcinogenic, or mutagenic compounds that would cause an adverse reaction when administered.

The present disclosure encompasses the preparation and use of salts of a Compound of the Disclosure. As used herein, a “pharmaceutically acceptable salt” refers to salts or zwitterionic forms of a Compound of the Disclosure. Salts of a Compound of the Disclosure can be prepared during the final isolation and purification of the compound or separately by reacting the compound with a suitable acid. The pharmaceutically acceptable salts of a Compound of the Disclosure can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Non-limiting examples of salts of a Compound of the Disclosure include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, para-toluenesulfonate, undecanoate, lactate, citrate, tartrate, gluconate, methanesulfonate, ethanedisulfonate, benzene sulfonate, and p-toluenesulfonate salts.

The present disclosure encompasses the preparation and use of solvates of a Compound of the Disclosure. Solvates typically do not significantly alter the physiological activity or toxicity of the compounds, and as such may function as pharmacological equivalents. The term “solvate” as used herein is a combination, physical association and/or solvation of a compound with a solvent molecule such as, e.g. a disolvate, monosolvate or hemisolvate, where the ratio of solvent molecule to compound is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. A Compound of the Disclosure can be present as solvated forms with a pharmaceutically acceptable solvent, such as water, methanol, and ethanol. It is intended that the disclosure includes both solvated and unsolvated forms of a RTI. One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by E.C. van Tonder et al., AAPS Pharm. Sci. Tech., 5(1): Article 12 (2004), and A. L. Bingham et al., Chem. Commun. 603-604 (2001). A typical, non-limiting, process of preparing a solvate would involve dissolving a RTI in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvate in a crystal of the solvate.

The Compound of the Disclosure is typically are administered in admixture with a pharmaceutical carrier to give a pharmaceutical composition selected with regard to the intended route of administration and standard pharmaceutical practice. Pharmaceutical compositions for use in accordance with the present disclosure are formulated in a conventional manner using one or more physiologically acceptable carriers comprising excipients and/or auxiliaries that facilitate processing of the Compound of the Disclosure.

These pharmaceutical compositions can be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of a Compound of the Disclosure is administered orally, the composition typically is in the form of a tablet, capsule, powder, solution, or elixir. When administered in tablet form, the composition additionally can contain a solid carrier, such as a gelatin or an adjuvant. The tablet, capsule, and powder contain about 0.01% to about 95%, and preferably from about 1% to about 50%, of a RTI, or a pharmaceutically acceptable salt or solvate thereof, or a tautomer thereof. When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.1% to about 90%, and preferably about 1% to about 50%, by weight, of a Compound of the Disclosure.

When a therapeutically effective amount of a Compound of the Disclosure is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, an isotonic vehicle.

A Compound of the Disclosure can be readily combined with pharmaceutically acceptable carriers well-known in the art. Standard pharmaceutical carriers are described in Remington's Pharmaceutical Sciences, Mack Publishing Co., Easton, PA, 19th ed. 1995. Such carriers enable the active agents to be formulated as tablets, pills, dragees, capsules, liquids, gels, syrups, slurries, suspensions and the like, for oral ingestion by a subject to be treated. Pharmaceutical preparations for oral use can be obtained by adding a Compound of the Disclosure to a solid excipient, optionally grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

A Compound of the Disclosure can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the Compound of the Disclosure in water-soluble form. Additionally, suspensions of a Compound of the Disclosure can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

In particular, a Compound of the Disclosure can be administered orally in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. A Compound of the Disclosure also can be injected parenterally, for example, intravenously, intramuscularly, subcutaneously, or intracoronarily. For parenteral administration, a Compound of the Disclosure typically used in the form of a sterile aqueous solution which can contain other substances, for example, salts or monosaccharides, such as mannitol or glucose, to make the solution isotonic with blood.

### III. Cancer

In some embodiments, Compounds of the Disclosure, or pharmaceutical compositions thereof, are administered to a subject to treat or prevent cancer. In some embodiments the cancer is characterized as overexpressing one or more DDR enzymes, e.g., a DDR enzyme listed in Table C.

Examples of treatable and preventable cancers include, but are not limited to, any one or more of the cancers of Table 4.

TABLE 4

| | | | |
|---|---|---|---|
| adrenal cancer | acinic cell carcinoma | acoustic neuroma | acral lentigious melanoma |
| acrospiroma | acute eosinophilic leukemia | acute erythroid leukemia | acute lymphoblastic leukemia |
| acute megakaryoblastic leukemia | acute monocytic leukemia | acute promyelocytic leukemia | adenocarcinoma |
| adenoid cystic carcinoma | adenoma | adenomatoid odontogenic tumor | adenosquamous carcinoma |
| adipose tissue neoplasm | adrenocortical carcinoma | adult T-cell leukemia/lymphoma | aggressive NK-cell leukemia |
| AIDS-related lymphoma | alveolar rhabdomyosarcoma | alveolar soft part sarcoma | ameloblastic fibroma |
| anaplastic large cell lymphoma | anaplastic thyroid cancer | angioimmunoblastic | angiomyolipoma |
| | | T-cell lymphoma | |
| angiosarcoma | astrocytoma | atypical teratoid rhabdoid tumor | B-cell chronic |
| | | | lymphocytic leukemia |
| B-cell prolymphocytic leukemia | B-cell lymphoma | basal cell carcinoma | biliary tract cancer |
| bladder cancer | blastoma | bone cancer | Brenner tumor |
| Brown tumor | Burkitt's lymphoma | breast cancer | brain cancer |
| carcinoma | carcinoma in situ | carcinosarcoma | cartilage tumor |
| cementoma | myeloid sarcoma | chondroma | chordoma |
| choriocarcinoma | choroid plexus papilloma | clear-cell sarcoma of the kidney | craniopharyngioma |
| cutaneous T-cell lymphoma | cervical cancer | colorectal cancer | Degos disease |
| desmoplastic small | diffuse large B-cell lymphoma | dysembryoplastic | dysgerminoma |
| round cell tumor | | neuroepithelial tumor | |
| embryonal carcinoma | endocrine gland neoplasm | endodermal sinus tumor | enteropathy-associated |
| | | | T-cell lymphoma |
| esophageal cancer | fetus in fetu | fibroma | fibrosarcoma |
| follicular lymphoma | follicular thyroid cancer | ganglioneuroma | gastrointestinal cancer |
| germ cell tumor | gestational choriocarcinoma | giant cell fibroblastoma | giant cell tumor of the bone |
| glial tumor | glioblastoma multiforme | glioma | gliomatosis cerebri |
| glucagonoma | gonadoblastoma | granulosa cell tumor | gynandroblastoma |
| gallbladder cancer | gastric cancer | hairy cell leukemia | hemangioblastoma |
| head and neck cancer | hemangiopericytoma | hematological cancer | hepatoblastoma |
| hepatosplenic T-cell lymphoma | Hodgkin's lymphoma | non-Hodgkin's lymphoma | invasive lobular carcinoma |
| intestinal cancer | kidney cancer | laryngeal cancer | lentigo maligna |
| lethal midline carcinoma | leukemia | leydig cell tumor | liposarcoma |
| lung cancer | lymphangioma | lymphangiosarcoma | lymphoepithelioma |
| lymphoma | acute lymphocytic leukemia | acute myelogeous leukemia | chronic lymphocytic leukemia |
| liver cancer | small cell lung cancer | non-small cell lung cancer | MALT lymphoma |
| malignant fibrous histiocytoma | malignant peripheral nerve | malignant triton tumor | mantle cell lymphoma |
| | sheath tumor | | |
| marginal zone B-cell lymphoma | mast cell leukemia | mediastinal germ cell tumor | medullary carcinoma of the breast |
| medullary thyroid cancer | medulloblastoma | melanoma | meningioma |
| merkel cell cancer | mesothelioma | metastatic urothelial carcinoma | mixed Mullerian tumor |
| mucinous tumor | multiple myeloma | muscle tissue neoplasm | mycosis fungoides |
| myxoid liposarcoma | myxoma | myxosarcoma | nasopharyngeal carcinoma |
| neurinoma | neuroblastoma | neurofibroma | neuroma |
| nodular melanoma | ocular cancer | oligoastrocytoma | oligodendroglioma |
| oncocytoma | optic nerve sheath meningioma | optic nerve tumor | oral cancer |
| osteosarcoma | ovarian cancer | Pancoast tumor | papillary thyroid cancer |
| paraganglioma | pinealoblastoma | pineocytoma | pituicytoma |
| pituitary adenoma | pituitary tumor | plasmacytoma | polyembryoma |
| precursor T-lymphoblastic | primary central nervous | primary effusion lymphoma | preimary peritoneal cancer |
| lymphoma | system lymphoma | | |
| prostate cancer | pancreatic cancer | pharyngeal cancer | pseudomyxoma periotonei |
| renal cell carcinoma | renal medullary carcinoma | retinoblastoma | rhabdomyoma |
| rhabdomyosarcoma | Richter's transformation | rectal cancer | sarcoma |
| Schwannomatosis | seminoma | Sertoli cell tumor | sex cord-gonadal stromal tumor |
| signet ring cell carcinoma | skin cancer | small blue round cell tumors | small cell carcinoma |
| soft tissue sarcoma | somatostatinoma | soot wart | spinal tumor |
| splenic marginal zone lymphoma | squamous cell carcinoma | synovial sarcoma | Sezary's disease |
| small intestine cancer | squamous carcinoma | stomach cancer | T-cell lymphoma |
| testicular cancer | thecoma | thyroid cancer | transitional cell carcinoma |
| throat cancer | urachal cancer | urogenital cancer | urothelial carcinoma |
| uveal melanoma | uterine cancer | verrucous carcinoma | visual pathway glioma |
| vulvar cancer | vaginal cancer | Waldenstrom's macroglobulinemia | Warthin's tumor |
| Wilms' tumor | colon cancer | pancreatic ductal cancer | |

In another embodiment, the cancer is a solid tumor.

In another embodiment, the cancer a hematological cancer. Exemplary hematological cancers include, but are not limited to, the cancers listed in Table 5.

TABLE 5

| | |
|---|---|
| acute lymphocytic leukemia (ALL) | acute eosinophilic leukemia |
| acute myeloid leukemia (AML) | acute erythroid leukemia |
| chronic lymphocytic leukemia (CLL) | acute lymphoblastic leukemia |
| small lymphocytic lymphoma (SLL) | acute megakaryoblastic leukemia |
| multiple myeloma (MM) | acute monocytic leukemia |
| Hodgkins lymphoma (HL) | acute promyelocytic leukemia |
| non-Hodgkin's lymphoma (NHL) | acute myelogeous leukemia |
| mantle cell lymphoma (MCL) | B-cell prolymphocytic leukemia |
| marginal zone B-cell lymphoma | B-cell lymphoma |
| splenic marginal zone lymphoma | MALT lymphoma |
| follicular lymphoma (FL) | precursor T-lymphoblastic lymphoma |
| Waldenstrom's macroglobulinemia (WM) | T-cell lymphoma |
| diffuse large B-cell lymphoma (DLBCL) | mast cell leukemia |
| marginal zone lymphoma (MZL) | adult T cell leukemia/lymphoma |
| hairy cell leukemia (HCL) | aggressive NK-cell leukemia |
| Burkitt's lymphoma (BL) | angioimmunoblastic T-cell lymphoma |
| Richter's transformation | |

In another embodiment, the hematological cancer is acute lymphocytic leukemia, chronic lymphocytic leukemia (including B-cell chronic lymphocytic leukemia), or acute myeloid leukemia.

In another embodiment, the cancer is a leukemia, for example, a acute monocytic leukemia, acute myelogenous leukemia, chronic myelogenous leukemia, chronic lymphocytic leukemia, or mixed lineage leukemia (MLL). In another embodiment the cancer is NUT-midline carcinoma. In another embodiment the cancer is multiple myeloma. In another embodiment the cancer is a lung cancer such as small cell lung cancer (SCLC). In another embodiment the cancer is a neuroblastoma. In another embodiment the cancer is Burkitt's lymphoma. In another embodiment the cancer is cervical cancer. In another embodiment the cancer is esophageal cancer. In another embodiment the cancer is ovarian cancer. In another embodiment the cancer is colorectal cancer. In another embodiment, the cancer is prostate cancer. In another embodiment, the cancer is breast cancer.

In another embodiment, the cancer is acute monocytic leukemia, acute myelogenous leukemia, chronic myelogenous leukemia, chronic lymphocytic leukemia mixed lineage leukemia, NUT-midline carcinoma, multiple myeloma, small cell lung cancer, non-small cell lung cancer, neuroblastoma, Burkitt's lymphoma, cervical cancer, esophageal cancer, ovarian cancer, colorectal cancer, prostate cancer, breast cancer, bladder cancer, ovary cancer, glioma, sarcoma, esophageal squamous cell carcinoma, or papillary thyroid carcinoma.

In another embodiment, the cancer is breast, colon, lung, pancreatic ductal, prostate, ovarian, or head and neck cancer.

In another embodiment, the cancer is colon, rectum, lung, stomach, breast, ovary, or head and neck cancer.

In another embodiment, the cancer is breast cancer. In another embodiment, the cancer is colon cancer. In another embodiment, the cancer is lung cancer, e.g., small cell lung cancer or non-small cell lung cancer. In another embodiment, the cancer is pancreatic ductal cancer. In another embodiment, the cancer is prostate cancer. In another embodiment, the cancer is ovarian cancer. In another embodiment, the cancer is head and neck cancer.

In another embodiment, the cells of the cancer overexpress one or more DDR enzymes.

In another embodiment, the cells of the cancer overexpress one or more polymerases/enzymes encoded by the genes of Table A.

TABLE A

| Gene Name | Accession Number |
|---|---|
| POLA1 | NM_001330360 |
| POLB | NM_002690 |
| POLD1 | NM_002691 |
| POLD2 | NM_001127218 |
| POLD3 | NM_006591 |
| POLD4 | NM_021173 |
| POLE (POLE1) | NM_006231 |
| POLE2 | NM_002692 |
| POLE3 | NM_017443 |
| POLE4 | NM_019896 |
| REV3L (POLZ) | NM_002912 |
| MAD2L2 (REV7) | NM_006341 |
| REV1 (REV1L) | NM_016316 |
| POLG | NM_002693 |
| POLH | NM_006502 |
| POLI (RAD30B) | NM_007195 |
| POLQ | NM_199420 |
| POLK (DINB1) | NM_016218 |
| POLL | NM_013274 |
| POLM | NM_013284 |
| POLN (POL4P) | NM_181808 |
| PRIMPOL | NM_152683 |
| DNTT | NM_004088 |

In another embodiment, the cells of the cancer overexpress one or more DDR enzymes of Table C.

TABLE C

| Polymerase/enzyme | Gene | Repair Pathway |
|---|---|---|
| Pol β (beta) | POLB | BER, NER |
| Pol δ (delta) | POLD1 | TLS, HR |
| (replication and repair) | | |
| Pol λ (lambda) | POLL | TLS, NHEJ, |
| Pol θ (theta) | POLQ | Alt-NHEJ, TLS |
| Pol ζ (zeta) | REV3L | TLS |
| Pol η (eta) | POLH | TLS, HR |
| Pol ι (iota) | POLI | TLS |
| Pol κ (kappa) | POLK | TLS, HR |
| Pol ν (nu) | POLN | TLS, HR |
| Pol Prim | PRIM | TLS |
| (Polymerase-Primase) | | |
| Rev1 (term. | REV1 | TLS |
| Deoxy. Cyd Trans) | | |
| Pol μ (mu) | POLM | TLS, NHEJ, |
| TdT (Terminal | DNTT | NHEJ |
| Deoxy. NT Trans) | | |
| Pol γ (gamma) | POLG | mtDNA repl./repair |

In another embodiment, the cancer overexpresses Pol θ.

In another embodiment, the cancer overexpresses Pol η.

In another embodiment, the cancer overexpresses Pol μ.

In another embodiment, the cancer overexpresses TdT.

In another embodiment, the cancer is breast cancer, and the cells of the cancer exhibit deficiency or loss of function of BRCA1 and/or BRCA2 genes.

In another embodiment, the cancer is prostate cancer, and the cells of the cancer exhibit deficiency or loss of function of BRCA1 and or BRCA2 genes.

In another embodiment, the cancer is ovarian cancer, and the cells of the cancer exhibit deficiency or loss of function of BRCA1 and/or BRCA2 genes.

In another embodiment, the cancer is pancreatic cancer, and the cells of the cancer exhibit deficiency or loss of function of BRCA1 and/or BRCA2 genes.

### IV. Combination Therapy with Optional Therapeutic Agents

In one embodiment, a therapeutically effective amount of a Compound of the Disclosure is administered to a subject having cancer as a single agent.

In another embodiment, a therapeutically effective amount of a Compound of the Disclosure is administered to a subject having cancer in combination with a therapeutically effective amount of an optional therapeutic agent. In some embodiments, the optional therapeutic agent comprises one or more anticancer agents and/or therapies.

In another embodiment, a Compound of the Disclosure is administered to a subject having cancer in combination with one optional therapeutic agent. In another embodiment, a Compound of the Disclosure is administered to a subject having cancer in combination with two optional therapeutic agents. In another embodiment, a Compound of the Disclosure is administered to a subject having cancer in combination with three optional therapeutic agents.

The Compound of the Disclosure and the optional therapeutic agent can be administered in combination under one or more of the following conditions: at different periodicities, at different durations, at different concentrations, by different administration routes, etc.

In one embodiment, the Compound of the Disclosure and the optional therapeutic agent are administered in combination to a subject as part of a single pharmaceutical composition.

In another embodiment, the Compound of the Disclosure and the optional therapeutic agent are administered in combination to a subject separately, e.g., as two or more separate pharmaceutical compositions. For example, two or more separate pharmaceutical compositions—one comprising the Compound of the Disclosure and one or more comprising one or more optional therapeutic agents—are administered to a subject. The separate pharmaceutical compositions can be administered to the subject, for example, at different periodicities, at different durations, or by the same or different administration routes, e.g., the Compound of the Disclosure can be administered orally and the optionally therapeutic agent can be administered intravenously.

In another embodiment, the Compound of the Disclosure is administered to the subject prior to the one or more optional therapeutic agents, e.g., 0.5, 1, 2, 3, 4, 5, 10, 12, or 18 hours, 1, 2, 3, 4, 5, or 6 days, or 1, 2, 3, or 4 weeks prior to the administration of the one or more optional therapeutic agents.

In another embodiment, the Compound of the Disclosure is administered to the subject after the one or more optional therapeutic agents, e.g., 0.5, 1, 2, 3, 4, 5, 10, 12, or 18 hours, 1, 2, 3, 4, 5, or 6 days, or 1, 2, 3, or 4 weeks after the administration of the one or more optional therapeutic agents.

In another embodiment, the Compound of the Disclosure and the one or more optional therapeutic agents are administered concurrently.

In another embodiment, the optional therapeutic agent comprises a poly ADP ribose polymerase (PARP) inhibitor, an ATM inhibitor, a wee1 inhibitor, a CHK inhibitor, and/or an ATR inhibitor.

In another embodiment, the cancer cells of the subject are resistant to PARP inhibition.

In another embodiment, the optional therapeutic agent comprises a PARP inhibitor. In another embodiment, the PARP inhibitor is olaparib, rucaparib, niraparib or talazoparib.

In another embodiment, the ATM inhibitor is ATM inhibitors AZD0156 MS3541.

In another embodiment, the ATR inhibitor is AZD6738, M4344, or M6620.

In another embodiment, the wee1 inhibitor is AZD1775.

In another embodiment, the disclosure provides methods for the treatment of breast cancer, wherein the optional therapeutic agent comprises Soltamox® (tamoxifen), Arimidex® (anastrozole), Femara® (letrozole), Aromasin® (exemestane), Herceptin® (trastuzumab), Abraxane® (paclitaxel), Cytoxan® (cyclophosphamide), Taxol® (paclitaxel), Afinitor® (everolimus), Taxotere® (docetaxel), Xeloda® (capecitabine), Trexall® (methotrexate), Faslodex (fulvestrant), Adriamycin® (doxorubicin), Perjeta® (pertuzumab), Gemzar (gemcitabine), Tykerb® (lapatinib), Adrucil® (fluorouracil), Ibrance® (palbociclib), Verzenio® (abemaciclib), Fareston® (toremifene), Halaven® (eribulin), Menest, Kadcyla® (ado-trastuzumab emtransine), Androxy® (fluoxymesterone), Avastin® (bevacizumab), esterified estrogens, Herzuma® (trastuzumab), Ixempra® (ixabepilone), Kanjinti® (trastuzumab), Kisqali® (ribociclib), Ogivri® (trastuzumab), Ontruzant® (trastuzumab), Tepadina® (thiotepa), Trazimera® (trastuzumab), Velban® (vinblastine), Piqray® (alpelisib), Tecentriq® (atezolizumab), Enhertu® (fam-trastuzumab deruxtecan), Herceptin, Hylecta™ (hyaluronidase/trastuzumab), Infugem® (gemcitabine), Kisqali® Femara® Co-Pack (ribociclib and letrozole), Talzenna® (talazoparib), Trodelvy® (sacituzumab) or Tukysa™ (tukatinib).

In another embodiment, the disclosure provides methods for the treatment of colon cancer, wherein the the optional therapeutic agent comprises Xeloda® (capecitabine), Eloxatin® (oxaliplatin), fluorouracil, Avastin® (bevacizumab), leucovorin, Camptosarg (irinotecan), Stivarga® (regorafenib), Erbitux® (cetuximab), Vectibix® (panitumumab), Lonsurf® (tipiracil/trifluridine), Zaltrap® (ziv-aflibercept), Betaseron® (interferon beta-ib), Fusilev® (levoleucovorin), Wellcocorin® (methotrexate), Keytruda® (pembrolizumab), Mvasi® (bevacizumab-awwb), Cyramza® (ramucirumab), Yervoy® (ipilmumab), Opdivo® (nivolumab), Braftovi® (encorafenib), Khapzory® (levoleucovorin) or Zirabev® (bevacizumab-bvzr).

In another embodiment, the disclosure provides methods for the treatment lung cancer, wherein the optional therapeutic agent comprises Etopophos® (etoposide), Hycamtin® (topotecan), VePesid® (etoposide), Toposar® (etoposide), Opdivo® (nivolumab), Keytruda® (pembrolizumab), Tecentriq® (atezolizumab), Imfinizi® (durvalumab), methotrexate, cyclophosphamide, Carboplatin, Cisplatin, docetaxel, Gemcitabine, Irinotecan, Paclitaxel, Pemetrexed, Vinblastine, or Vinorelbine.

In another embodiment, the disclosure provides methods for the treatment of pancreatic ductal cancer, wherein the optional therapeutic agent comprises Gemzar® (Gemcitabine), fluorouracil, Afmnitor® (everolimus), Tarceva® (erlotinib), Abraxane® (paclitaxel), capecitabine, Sutent® (sunitinib), pancreatin, methotrexate, Zanosar® (streptozocin), Mutamycin® (mitomycin), Onivyde® (irinotecan), bevacizumab, cetuximab, Infugem® (gemcitabine) or Lynparza® (olaparib).

In some embodiments, the method is for the treatment of head and neck cancer, wherein the optional therapeutic agent comprises Erbituz® (cetuximab), Taxotere® (docetaxel), Trexall® (methotrexate), Keytruda® (pembrolizumab) or Opdivo® (nivolumab).

In another embodiment, the disclosure provides methods for the treatment of prostate cancer, wherein the optional therapeutic agent comprises Suprefact® (buserelin), Firmagon® (degarelix), Zoladex® (goserelin), Vantas® (histrelin), Eligard® (leuprolide), Orgovyx® (relugolix), Trelstar® (triptorelin), Casodex® (bicalutamide), Eulexin® (flutamide), Nilandron® (nilutamide), Zytiga® (biraterone acetate), Erleada® (apalutamide), or Xtandi® (enzalutamide).

In another embodiment, the optional therapeutic agent comprises a STING agonist.

In another embodiment, the optional therapeutic agent is an epigenetic drug. As used herein, the term “epigenetic drug” refers to a therapeutic agent that targets an epigenetic regulator. Examples of epigenetic regulators include the histone lysine methyltransferases, histone arginine methyl transferases, histone demethylases, histone deacetylases, histone acetylases, and DNA methyltransferases. Histone deacetylase inhibitors include, but are not limited to, vorinostat.

Non-limiting examples of therapies and anticancer agents that can be used in combination with a Compound of the Disclosure include surgery, radiotherapy (e.g., gamma-radiation, neutron beam radiotherapy, electron beam radiotherapy, proton therapy, brachytherapy, and systemic radioactive isotopes), endocrine therapy, a biologic response modifier (e.g., an interferon, an interleukin, tumor necrosis factor (TNF), hyperthermia and cryotherapy, an agent to attenuate any adverse effect (e.g., an antiemetic), and any other approved chemotherapeutic drug.

Examples of antiproliferative compounds include, but are not limited to, an aromatase inhibitor; an anti-estrogen; an anti-androgen; a gonadorelin agonist; a topoisomerase I inhibitor; a topoisomerase II inhibitor; a microtubule active agent; an alkylating agent; a retinoid, a carontenoid, or a tocopherol; a cyclooxygenase inhibitor; an MMP inhibitor; an mTOR inhibitor; an antimetabolite; a platin compound; a methionine aminopeptidase inhibitor; a bisphosphonate; an antiproliferative antibody; a heparanase inhibitor; an inhibitor of Ras oncogenic isoforms; a telomerase inhibitor; a proteasome inhibitor; a compound used in the treatment of hematologic malignancies; a Flt-3 inhibitor; an Hsp90 inhibitor; a kinesin spindle protein inhibitor; a MEK inhibitor; an antitumor antibiotic; a nitrosourea; a compound targeting/decreasing protein or lipid kinase activity, a compound targeting/decreasing protein or lipid phosphatase activity, or any further anti-angiogenic compound.

Nonlimiting exemplary aromatase inhibitors include, but are not limited to, steroids, such as atamestane, exemestane, and formestane, and non-steroids, such as aminoglutethimide, roglethimide, pyridoglutethimide, trilostane, testolactone, ketokonazole, vorozole, fadrozole, anastrozole, and letrozole.

Nonlimiting anti-estrogens include, but are not limited to, tamoxifen, fulvestrant, raloxifene, and raloxifene hydrochloride. Anti-androgens include, but are not limited to, bicalutamide. Gonadorelin agonists include, but are not limited to, abarelix, goserelin, and goserelin acetate.

Exemplary topoisomerase I inhibitors include, but are not limited to, topotecan, gimatecan, irinotecan, camptothecin and its analogues, 9-nitrocamptothecin, and the macromolecular camptothecin conjugate PNU-166148. Topoisomerase II inhibitors include, but are not limited to, anthracyclines, such as doxorubicin, daunorubicin, epirubicin, idarubicin, and nemorubicin; anthraquinones, such as mitoxantrone and losoxantrone; and podophillotoxines, such as etoposide and teniposide.

Microtubule active agents include microtubule stabilizing, microtubule destabilizing compounds, and microtubulin polymerization inhibitors including, but not limited to, taxanes, such as paclitaxel and docetaxel; vinca alkaloids, such as vinblastine, vinblastine sulfate, vincristine, and vincristine sulfate, and vinorelbine; discodermolides; cochicine and epothilones and derivatives thereof.

Exemplary nonlimiting alkylating agents include cyclophosphamide, ifosfamide, melphalan, and nitrosoureas, such as carmustine and lomustine.

Exemplary nonlimiting cyclooxygenase inhibitors include Cox-2 inhibitors, 5-alkyl substituted 2-arylaminophenylacetic acid and derivatives, such as celecoxib, rofecoxib, etoricoxib, valdecoxib, or a 5-alkyl-2-arylaminophenylacetic acid, such as lumiracoxib.

Exemplary nonlimiting matrix metalloproteinase inhibitors (“MMP inhibitors”) include collagen peptidomimetic and nonpeptidomimetic inhibitors, tetracycline derivatives, batimastat, marimastat, prinomastat, metastat, BMS-279251, BAY 12-9566, TAA211, MMI270B, and AAJ996.

Exemplary nonlimiting mTOR inhibitors include compounds that inhibit the mammalian target of rapamycin (mTOR) and possess antiproliferative activity such as sirolimus, everolimus, CCI-779, and ABT578.

Exemplary nonlimiting antimetabolites include 5-fluorouracil (5-FU), capecitabine, gemcitabine, DNA demethylating compounds, such as 5-azacytidine and decitabine, methotrexate and edatrexate, and folic acid antagonists, such as pemetrexed.

Exemplary nonlimiting platin compounds include carboplatin, cis-platin, cisplatinum, and oxaliplatin.

Exemplary nonlimiting methionine aminopeptidase inhibitors include bengamide or a derivative thereof and PPI-2458.

Exemplary nonlimiting bisphosphonates include etridonic acid, clodronic acid, tiludronic acid, pamidronic acid, alendronic acid, ibandronic acid, risedronic acid, and zoledronic acid.

Exemplary nonlimiting antiproliferative antibodies include trastuzumab, trastuzumab-DMI, cetuximab, bevacizumab, rituximab, PR064553, and 2C4. The term “antibody” is meant to include intact monoclonal antibodies, polyclonal antibodies, multispecific antibodies formed from at least two intact antibodies, and antibody fragments, so long as they exhibit the desired biological activity.

Exemplary nonlimiting heparanase inhibitors include compounds that target, decrease, or inhibit heparin sulfate degradation, such as PI-88 and OGT2115.

The term “an inhibitor of Ras oncogenic isoforms,” such as H-Ras, K-Ras, or N-Ras, as used herein refers to a compound which targets, decreases, or inhibits the oncogenic activity of Ras, for example, a farnesyl transferase inhibitor, such as L-744832, DK8G557, tipifarnib, and lonafarnib.

Exemplary nonlimiting telomerase inhibitors include compounds that target, decrease, or inhibit the activity of telomerase, such as compounds that inhibit the telomerase receptor, such as telomestatin.

Exemplary nonlimiting proteasome inhibitors include compounds that target, decrease, or inhibit the activity of the proteasome including, but not limited to, bortezomid.

The phrase “compounds used in the treatment of hematologic malignancies” as used herein includes FMS-like tyrosine kinase inhibitors, which are compounds targeting, decreasing or inhibiting the activity of FMS-like tyrosine kinase receptors (Flt-3R); interferon, I-β-D-arabinofuransylcytosine (ara-c), and bisulfan; and ALK inhibitors, which are compounds which target, decrease, or inhibit anaplastic lymphoma kinase.

Exemplary nonlimiting Flt-3 inhibitors include PKC412, midostaurin, a staurosporine derivative, SUI 1248, and MLN518.

Exemplary nonlimiting HSP90 inhibitors include compounds targeting, decreasing, or inhibiting the intrinsic ATPase activity of HSP90; or degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins, or antibodies that inhibit the ATPase activity of HSP90, such as 17-allylamino, 17-demethoxygeldanamycin (17AAG), a geldanamycin derivative; other geldanamycin related compounds; radicicol and HDAC inhibitors.

The phrase “a compound targeting/decreasing a protein or lipid kinase activity; or a protein or lipid phosphatase activity; or any further anti-angiogenic compound” as used herein includes a protein tyrosine kinase and/or serine and/or threonine kinase inhibitor or lipid kinase inhibitor, such as a) a compound targeting, decreasing, or inhibiting the activity of the platelet-derived growth factor-receptors (PDGFR), such as a compound that targets, decreases, or inhibits the activity of PDGFR, such as an N-phenyl-2-pyrimidine-amine derivatives, such as imatinib, SUIO1, SU6668, and GFB-111; b) a compound targeting, decreasing, or inhibiting the activity of the fibroblast growth factor-receptors (FGFR); c) a compound targeting, decreasing, or inhibiting the activity of the insulin-like growth factor receptor I (IGF-IR), such as a compound that targets, decreases, or inhibits the activity of IGF-IR; d) a compound targeting, decreasing, or inhibiting the activity of the Trk receptor tyrosine kinase family, or ephrin B4 inhibitors; e) a compound targeting, decreasing, or inhibiting the activity of the Axl receptor tyrosine kinase family; f) a compound targeting, decreasing, or inhibiting the activity of the Ret receptor tyrosine kinase; g) a compound targeting, decreasing, or inhibiting the activity of the Kit/SCFR receptor tyrosine kinase, such as imatinib; h) a compound targeting, decreasing, or inhibiting the activity of the c-Kit receptor tyrosine kinases, such as imatinib; i) a compound targeting, decreasing, or inhibiting the activity of members of the c-Abl family, their gene-fusion products (e.g. Bcr-Ab kinase) and mutants, such as an N-phenyl-2-pyrimidine-amine derivative, such as imatinib or nilotinib; PD180970; AG957; NSC 680410; PD173955; or dasatinib; j) a compound targeting, decreasing, or inhibiting the activity of members of the protein kinase C (PKC) and Raf family of serine/threonine kinases, members of the MEK, SRC, JAK, FAK, PDK1, PKB/Akt, and Ras/MAPK family members, and/or members of the cyclin-dependent kinase family (CDK), such as a staurosporine derivative disclosed in U.S. Pat. No. 5,093,330, such as midostaurin; examples of further compounds include UCN-01, safingol, BAY 43-9006, bryostatin 1, perifosine; ilmofosine; RO 318220 and RO 320432; GO 6976; Isis 3521; LY333531/LY379196; a isochinoline compound; a farnesyl transferase inhibitor; PD184352 or QAN697, or AT7519; k) a compound targeting, decreasing or inhibiting the activity of a protein-tyrosine kinase, such as imatinib mesylate or a tyrphostin, such as Tyrphostin A23/RG-50810; AG 99, Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); l) a compound targeting, decreasing, or inhibiting the activity of the epidermal growth factor family of receptor tyrosine kinases (EGFR, ErbB2, ErbB3, ErbB4 as homo- or heterodimers) and their mutants, such as CP 358774, ZD 1839, ZM 105180; trastuzumab, cetuximab, gefitinib, erlotinib, OS1-774, C1-1033, EKB-569, GW-2016, antibodies E1, E2.4, E2.5, E6.2, E6.4, E2.11, E6.3 and E7.6.3, and 7H-pyrrolo-[2,3-d]pyrimidine derivatives; and m) a compound targeting, decreasing, or inhibiting the activity of the c-Met receptor.

Exemplary compounds that target, decrease, or inhibit the activity of a protein or lipid phosphatase include inhibitors of phosphatase 1, phosphatase 2A, or CDC25, such as okadaic acid or a derivative thereof.

Further anti-angiogenic compounds include compounds having another mechanism for their activity unrelated to protein or lipid kinase inhibition, e.g., thalidomide and TNP-470.

Additional, nonlimiting, exemplary chemotherapeutic compounds, one or more of which may be used in combination with islatravir, include: daunorubicin, adriamycin, Ara-C, VP-16, teniposide, mitoxantrone, idarubicin, carboplatinum, PKC412, 6-mercaptopurine (6-MP), fludarabine phosphate, octreotide, SOM230, FTY720, 6-thioguanine, cladribine, 6-mercaptopurine, pentostatin, hydroxyurea, 2-hydroxy-1H-isoindole-1,3-dione derivatives, 1-(4-chloroanilino)-4-(4-pyridylmethyl)phthalazine or a pharmaceutically acceptable salt thereof, 1-(4-chloroanilino)-4-(4-pyridylmethyl)phthalazine succinate, angiostatin, endostatin, anthranilic acid amides, ZD4190, ZD6474, SU5416, SU6668, bevacizumab, rhuMAb, rhuFab, macugon; FLT-4 inhibitors, FLT-3 inhibitors, VEGFR-2 IgGI antibody, RPI 4610, bevacizumab, porfimer sodium, anecortave, triamcinolone, hydrocortisone, 11-a-epihydrocotisol, cortex olone, 17a-hydroxyprogesterone, corticosterone, desoxycorticosterone, testosterone, estrone, dexamethasone, fluocinolone, a plant alkaloid, a hormonal compound and/or antagonist, a biological response modifier, such as a lymphokine or interferon, an antisense oligonucleotide or oligonucleotide derivative, shRNA, and siRNA.

In another embodiment, the optional therapeutic agent is an immune checkpoint inhibitor. Examples of immune checkpoint inhibitors include PD-1 inhibitors, PD-L1 inhibitors, CTLA-4 inhibitors, LAG3 inhibitors, TIM3 inhibitors, cd47 inhibitors, and B7-H1 inhibitors. Thus, in one embodiment, islatravir is administered in combination with an immune checkpoint inhibitor is selected from the group consisting of a PD-1 inhibitor, a PD-L1 inhibitor, a CTLA-4 inhibitor, a LAG3 inhibitor, a TIM3 inhibitor, and a cd47 inhibitor.

In another embodiment, the immune checkpoint inhibitor is a programmed cell death (PD-1) inhibitor. PD-1 is a T-cell coinhibitory receptor that plays a pivotal role in the ability of tumor cells to evade the host's immune system. Blockage of interactions between PD-1 and PD-L1, a ligand of PD-1, enhances immune function and mediates antitumor activity. Examples of PD-1 inhibitors include antibodies that specifically bind to PD-1. Particular anti-PD-1 antibodies include, but are not limited to nivolumab, pembrolizumab, STI-A1014, and pidilzumab. For a general discussion of the availability, methods of production, mechanism of action, and clinical studies of anti-PD-1 antibodies, see U.S. 2013/0309250, U.S. Pat. Nos. 6,808,710, 7,595,048, 8,008,449, 8,728,474, 8,779,105, 8,952,136, 8,900,587, 9,073,994, 9,084,776, and Naido et al., British Journal of Cancer 111:2214-19 (2014).

In another embodiment, the immune checkpoint inhibitor is a PD-L1 (also known as B7-H1 or CD274) inhibitor. Examples of PD-L1 inhibitors include antibodies that specifically bind to PD-L1. Particular anti-PD-L1 antibodies include, but are not limited to, avelumab, atezolizumab, durvalumab, and BMS-936559. For a general discussion of the availability, methods of production, mechanism of action, and clinical studies, see U.S. Pat. No. 8,217,149, U.S. 2014/0341917, U.S. 2013/0071403, WO 2015036499, and Naido et al., British Journal of Cancer 111:2214-19 (2014).

In another embodiment, the immune checkpoint inhibitor is a CTLA-4 inhibitor. CTLA-4, also known as cytotoxic T-lymphocyte antigen 4, is a protein receptor that downregulates the immune system. CTLA-4 is characterized as a “brake” that binds costimulatory molecules on antigen-presenting cells, which prevents interaction with CD28 on T cells and also generates an overtly inhibitory signal that constrains T cell activation. Examples of CTLA-4 inhibitors include antibodies that specifically bind to CTLA-4. Particular anti-CTLA-4 antibodies include, but are not limited to, ipilimumab and tremelimumab. For a general discussion of the availability, methods of production, mechanism of action, and clinical studies, see U.S. Pat. Nos. 6,984,720, 6,207,156, and Naido et al., British Journal of Cancer 111:2214-19 (2014).

In another embodiment, the immune checkpoint inhibitor is a LAG3 inhibitor. LAG3, Lymphocyte Activation Gene 3, is a negative co-simulatory receptor that modulates T cell homeostatis, proliferation, and activation. In addition, LAG3 has been reported to participate in regulatory T cells (Tregs) suppressive function. A large proportion of LAG3 molecules are retained in the cell close to the microtubule-organizing center, and only induced following antigen specific T cell activation. U.S. 2014/0286935. Examples of LAG3 inhibitors include antibodies that specifically bind to LAG3. Particular anti-LAG3 antibodies include, but are not limited to, GSK2831781. For a general discussion of the availability, methods of production, mechanism of action, and studies, see, U.S. 2011/0150892, U.S. 2014/0093511, U.S. 20150259420, and Huang et al., Immunity 21:503-13 (2004).

In another embodiment, the immune checkpoint inhibitor is a TIM3 inhibitor. TIM3, T-cell immunoglobulin and mucin domain 3, is an immune checkpoint receptor that functions to limit the duration and magnitude of T_{H}1 and T_{C}1 T-cell responses. The TIM3 pathway is considered a target for anticancer immunotherapy due to its expression on dysfunctional CD8⁺ T cells and Tregs, which are two reported immune cell populations that constitute immunosuppression in tumor tissue. Anderson, Cancer Immunology Research 2:393-98 (2014). Examples of TIM3 inhibitors include antibodies that specifically bind to TIM3. For a general discussion of the availability, methods of production, mechanism of action, and studies of TIM3 inhibitors, see U.S. 20150225457, U.S. 20130022623, U.S. Pat. No. 8,522,156, Ngiow et al., Cancer Res 71: 6567-71 (2011), Ngiow, et al., Cancer Res 71:3540-51 (2011), and Anderson, Cancer Immunology Res 2:393-98 (2014).

In another embodiment, the immune checkpoint inhibitor is a cd47 inhibitor. See Unanue, E. R., PNAS 110:10886-87 (2013).

The term “antibody” is meant to include intact monoclonal antibodies, polyclonal antibodies, multispecific antibodies formed from at least two intact antibodies, and antibody fragments, so long as they exhibit the desired biological activity. In another embodiment, “antibody” is meant to include soluble receptors that do not possess the Fc portion of the antibody. In one embodiment, the antibodies are humanized monoclonal antibodies and fragments thereof made by means of recombinant genetic engineering.

Another class of immune checkpoint inhibitors include polypeptides that bind to and block PD-1 receptors on T-cells without triggering inhibitor signal transduction. Such peptides include B7-DC polypeptides, B7-H1 polypeptides, B7-1 polypeptides and B7-2 polypeptides, and soluble fragments thereof, as disclosed in U.S. Pat. No. 8,114,845.

Another class of immune checkpoint inhibitors include compounds with peptide moieties that inhibit PD-1 signaling. Examples of such compounds are disclosed in U.S. Pat. No. 8,907,053.

Another class of immune checkpoint inhibitors include inhibitors of certain metabolic enzymes, such as indoleamine 2,3 dioxygenase (IDO), which is expressed by infiltrating myeloid cells and tumor cells. The IDO enzyme inhibits immune responses by depleting amino acids that are necessary for anabolic functions in T cells or through the synthesis of particular natural ligands for cytosolic receptors that are able to alter lymphocyte functions. Pardoll, Nature Reviews. Cancer 12:252-64 (2012); Löb, Cancer Immunol Immunother 58:153-57 (2009). Particular IDO blocking agents include, but are not limited to levo-1-methyl typtophan (L-1MT) and 1-methyl-tryptophan (IMT). Qian et al., Cancer Res 69:5498-504 (2009); and Lob et al., Cancer Immunol Immunother 58:153-7 (2009).

In one embodiment, the immune checkpoint inhibitor is nivolumab, pembrolizumab, pidilizumab, STI-A1110, avelumab, atezolizumab, durvalumab, STI-A1014, ipilimumab, tremelimumab, GSK2831781, BMS-936559 or MED14736.

In one embodiment, the optional therapeutic agent comprises one of the anti-cancer drugs or anti-cancer drug combinations listed in Table 6.

TABLE 6

| | | | |
|---|---|---|---|
| Abemaciclib | Abiraterone | Abraxane (Paclitaxel | ABVD |
| | Acetate | Albumin-stabilized | |
| | | Nanoparticle | |
| | | Formulation) | |
| ABVE | ABVE-PC | AC | Acalabrutinib |
| AC-T | Actemra | Adcetris (Brentuximab | ADE |
| | (Tocilizumab) | Vedotin) | |
| Ado-Trastuzumab | Adriamycin | Afatinib Dimaleate | Afinitor |
| Emtansine | (Doxorubicin | | (Everolimus) |
| | Hydrochloride) | | |
| Akynzeo | Aldara | Aldesleukin | Alecensa |
| (Netupitant and | (Imiquimod) | | (Alectinib) |
| Palonosetron | | | |
| Hydrochloride) | | | |
| Alectinib | Alemtuzumab | Alimta (Pemetrexed | Aliqopa |
| | | Disodium) | (Copanlisib |
| | | | Hydrochloride) |
| Alkeran for | Alkeran Tablets | Aloxi (Palonosetron | Alunbrig |
| Injection | (Melphalan) | Hydrochloride) | (Brigatinib) |
| (Melphalan | | | |
| Hydrochloride) | | | |
| Ameluz | Amifostine | Aminolevulinic Acid | Anastrozole |
| (Aminolevulinic | | | |
| Acid) | | | |
| Apalutamide | Aprepitant | Aranesp (Darbepoetin | Aredia |
| | | Alfa) | (Pamidronate |
| | | | Disodium) |
| Arimidex | Aromasin | Arranon (Nelarabine) | Arsenic Trioxide |
| (Anastrozole) | (Exemestane) | | |
| Arzerra | Asparaginase | Atezolizumab | Avastin |
| (Ofatumumab) | Erwinia | | (Bevacizumab) |
| | chrysanthemi | | |
| Avelumab | Axicabtagene | Axitinib | Azacitidine |
| | Ciloleucel | | |
| Azedra | Bavencio | BEACOPP | Beleodaq |
| (lobenguane I 131) | (Avelumab) | | (Belinostat) |
| Belinostat | Bendamustine | Bendeka (Bendamustine | BEP |
| | Hydrochloride | Hydrochloride) | |
| Besponsa | Bevacizumab | Bexarotene | Bicalutamide |
| (Inotuzumab | | | |
| Ozogamicin) | | | |
| BiCNU | Binimetinib | Bleomycin | Blinatumomab |
| (Carmustine) | | | |
| Blincyto | Bortezomib | Bosulif (Bosutinib) | Bosutinib |
| (Blinatumomab) | | | |
| Braftovi | Brentuximab | Brigatinib | BuMel |
| (Encorafenib) | Vedotin | | |
| Busulfan | Busulfex | Cabazitaxel | Cabometyx |
| | (Busulfan) | | (Cabozantinib-S- |
| | | | Malate) |
| Cabozantinib-S- | CAF | Calquence | Campath |
| Malate | | (Acalabrutinib) | (Alemtuzumab) |
| Camptosar | Capecitabine | CAPOX | Carac |
| (Irinotecan | | | (Fluorouracil-- |
| Hydrochloride) | | | Topical) |
| Carboplatin | CARBOPLATIN- | Carfilzomib | Carmustine |
| | TAXOL | | |
| Carmustine | Casodex | CEM | Cemiplimab-rwlc |
| Implant | (Bicalutamide) | | |
| Ceritinib | Cerubidine | Cervarix (Recombinant | Cetuximab |
| | (Daunorubicin | HPV Bivalent Vaccine) | |
| | Hydrochloride) | | |
| CEV | Chlorambucil | CHLORAMBUCIL- | CHOP |
| | | PREDNISONE | |
| Cisplatin | Cladribine | Clofarabine | Clolar |
| | | | (Clofarabine) |
| CMF | Cobimetinib | Cometriq (Cabozantinib- | Copanlisib |
| | | S-Malate) | Hydrochloride |
| COPDAC | Copiktra | COPP | COPP-ABV |
| | (Duvelisib) | | |
| Cosmegen | Cotellic | Crizotinib | CVP |
| (Dactinomycin) | (Cobimetinib) | | |
| Cyclophosphamide | Cyramza | Cytarabine | Cytarabine |
| | (Ramucirumab) | | Liposome |
| Cytosar-U | Dabrafenib | Dacarbazine | Dacogen |
| (Cytarabine) | | | (Decitabine) |
| Dacomitinib | Dactinomycin | Daratumumab | Darbepoetin Alfa |
| Darzalex | Dasatinib | Daunorubicin | Daunorubicin |
| (Daratumumab) | | Hydrochloride | Hydrochloride |
| | | | and Cytarabine |
| | | | Liposome |
| Decitabine | Defibrotide | Defitelio (Defibrotide | Degarelix |
| | Sodium | Sodium) | |
| Denileukin | Denosumab | DepoCyt (Cytarabine | Dexamethasone |
| Diftitox | | Liposome) | |
| Dexrazoxane | Dinutuximab | Docetaxel | Doxil |
| Hydrochloride | | | (Doxorubicin |
| | | | Hydrochloride |
| | | | Liposome) |
| Doxorubicin | Doxorubicin | Dox-SL (Doxorubicin | Durvalumab |
| Hydrochloride | Hydrochloride | Hydrochloride | |
| | Liposome | Liposome) | |
| Duvelisib | Efudex | Eligard (Leuprolide | Elitek |
| | (Fluorouracil-- | Acetate) | (Rasburicase) |
| | Topical) | | |
| Ellence | Elotuzumab | Eloxatin (Oxaliplatin) | Eltrombopag |
| (Epirubicin | | | Olamine |
| Hydrochloride) | | | |
| Emend | Empliciti | Enasidenib Mesylate | Encorafenib |
| (Aprepitant) | (Elotuzumab) | | |
| Enzalutamide | Epirubicin | EPOCH | Epoetin Alfa |
| | Hydrochloride | | |
| Epogen (Epoetin | Erbitux | Eribulin Mesylate | Erivedge |
| Alfa) | (Cetuximab) | | (Vismodegib) |
| Erleada | Erlotinib | Erwinaze (Asparaginase | Ethyol |
| (Apalutamide) | Hydrochloride | Erwinia chrysanthemi) | (Amifostine) |
| Etopophos | Etoposide | Etoposide Phosphate | Evacet |
| (Etoposide | | | (Doxorubicin |
| Phosphate) | | | Hydrochloride |
| | | | Liposome) |
| Everolimus | Evista (Raloxifene | Evomela (Melphalan | Exemestane |
| | Hydrochloride) | Hydrochloride) | |
| 5-FU (Fluorouracil | 5-FU | Fareston (Toremifene) | Farydak |
| Injection) | (Fluorouracil-- | | (Panobinostat |
| | Topical) | | lactate) |
| Faslodex | FEC | Femara (Letrozole) | Filgrastim |
| (Fulvestrant) | | | |
| Firmagon | Fludarabine | Fluoroplex (Fluorouracil- | Fluorouracil |
| (Degarelix) | Phosphate | -Topical) | Injection |
| Fluorouracil-- | Flutamide | FOLFIRI | FOLFIRI- |
| Topical | | | BEVACIZUMAB |
| FOLFIRI- | FOLFIRINOX | FOLFOX | Folotyn |
| CETUXIMAB | | | (Pralatrexate) |
| Fostamatinib | FU-LV | Fulvestrant | Fusilev |
| Disodium | | | (Leucovorin |
| | | | Calcium) |
| Gardasil | Gardasil 9 | Gazyva (Obinutuzumab) | Gefitinib |
| (Recombinant | (Recombinant | | |
| HPV Quadrivalent | HPV Nonavalent | | |
| Vaccine) | Vaccine) | | |
| Gemcitabine | GEMCITABINE- | GEMCITABINE- | Gemtuzumab |
| Hydrochloride | CISPLATIN | OXALIPLATIN | Ozogamicin |
| Gemzar | Gilotrif (Afatinib | Gleevec (Imatinib | Gliadel Wafer |
| (Gemcitabine | Dimaleate) | Mesylate) | (Carmustine |
| Hydrochloride) | | | Implant) |
| Glucarpidase | Goserelin Acetate | Granisetron | Granisetron |
| | | | Hydrochloride |
| Granix | Halaven (Eribulin | Hemangeol (Propranolol | Herceptin |
| (Filgrastim) | Mesylate) | Hydrochloride) | (Trastuzumab) |
| HPV Bivalent | HPV Nonavalent | HPV Quadrivalent | Hycamtin |
| Vaccine, | Vaccine, | Vaccine, Recombinant | (Topotecan |
| Recombinant | Recombinant | | Hydrochloride) |
| Hydrea | Hydroxyurea | Hyper-CVAD | Ibrance |
| (Hydroxyurea) | | | (Palbociclib) |
| Ibritumomab | Ibrutinib | ICE | Iclusig (Ponatinib |
| Tiuxetan | | | Hydrochloride) |
| Idarubicin | Idelalisib | Idhifa (Enasidenib | Ifex (Ifosfamide) |
| Hydrochloride | | Mesylate) | |
| Ifosfamide | IL-2 | Imatinib Mesylate | Imbruvica |
| | (Aldesleukin) | | (Ibrutinib) |
| Imfinzi | Imiquimod | Imlygic (Talimogene | Inlyta (Axitinib) |
| (Durvalumab) | | Laherparepvec) | |
| Inotuzumab | Interferon Alfa- | Interleukin-2 | Intron A |
| Ozogamicin | 2b, Recombinant | (Aldesleukin) | (Recombinant |
| | | | Interferon Alfa- |
| | | | 2b) |
| Iobenguane I 131 | Ipilimumab | Iressa (Gefitinib) | Irinotecan |
| | | | Hydrochloride |
| Irinotecan | Istodax | Ivosidenib | Ixabepilone |
| Hydrochloride | (Romidepsin) | | |
| Liposome | | | |
| Ixazomib Citrate | Ixempra | Jakafi (Ruxolitinib | JEB |
| | (Ixabepilone) | Phosphate) | |
| Jevtana | Kadcyla (Ado- | Kepivance (Palifermin) | Keytruda |
| (Cabazitaxel) | Trastuzumab | | (Pembrolizumab) |
| | Emtansine) | | |
| Kisqali | Kymriah | Kyprolis (Carfilzomib) | Lanreotide |
| (Ribociclib) | (Tisagenlecleucel) | | Acetate |
| Lapatinib | Larotrectinib | Lartruvo (Olaratumab) | Lenalidomide |
| Ditosylate | Sulfate | | |
| Lenvatinib | Lenvima | Letrozole | Leucovorin |
| Mesylate | (Lenvatinib | | Calcium |
| | Mesylate) | | |
| Leukeran | Leuprolide | Levulan | Libtayo |
| (Chlorambucil) | Acetate | Kerastik (Aminolevulinic | (Cemiplimab- |
| | | Acid) | rwlc) |
| LipoDox | Lomustine | Lonsurf (Trifluridine and | Lorbrena |
| (Doxorubicin | | Tipiracil Hydrochloride) | (Lorlatinib) |
| Hydrochloride | | | |
| Liposome) | | | |
| Lorlatinib | Lumoxiti | Lupron (Leuprolide | Lupron Depot |
| | (Moxetumomab | Acetate) | (Leuprolide |
| | Pasudotox-tdfk) | | Acetate) |
| Lutathera | Lutetium (Lu 177- | Lynparza (Olaparib) | Marqibo |
| (Lutetium Lu 177- | Dotatate) | | (Vincristine |
| Dotatate) | | | Sulfate |
| | | | Liposome) |
| Matulane | Mechlorethamine | Megestrol Acetate | Mekinist |
| (Procarbazine | Hydrochloride | | (Trametinib) |
| Hydrochloride) | | | |
| Mektovi | Melphalan | Melphalan | Mercaptopurine |
| (Binimetinib) | | Hydrochloride | |
| Mesna | Mesnex (Mesna) | Methotrexate | Methylnaltrexone |
| | | | Bromide |
| Midostaurin | Mitomycin C | Mitoxantrone | Mogamulizumab- |
| | | Hydrochloride | kpkc |
| Moxetumomab | Mozobil | Mustargen | MVAC |
| Pasudotox-tdfk | (Plerixafor) | (Mechlorethamine | |
| | | Hydrochloride) | |
| Myleran | Mylotarg | Nanoparticle Paclitaxel | Navelbine |
| (Busulfan) | (Gemtuzumab | (Paclitaxel Albumin- | (Vinorelbine |
| | Ozogamicin) | stabilized Nanoparticle | Tartrate) |
| | | Formulation) | |
| Necitumumab | Nelarabine | Neratinib Maleate | Nerlynx |
| | | | (Neratinib |
| | | | Maleate) |
| Netupitant and | Neulasta | Neupogen (Filgrastim) | Nexavar |
| Palonosetron | (Pegfilgrastim) | | (Sorafenib |
| Hydrochloride | | | Tosylate) |
| Nilandron | Nilotinib | Nilutamide | Ninlaro |
| (Nilutamide) | | | (Ixazomib |
| | | | Citrate) |
| Niraparib Tosylate | Nivolumab | Nplate (Romiplostim) | Obinutuzumab |
| Monohydrate | | | |
| Odomzo | OEPA | Ofatumumab | OFF |
| (Sonidegib) | | | |
| Olaparib | Olaratumab | Omacetaxine | Oncaspar |
| | | Mepesuccinate | (Pegaspargase) |
| Ondansetron | Onivyde | Ontak (Denileukin | Opdivo |
| Hydrochloride | (Irinotecan | Diftitox) | (Nivolumab) |
| | Hydrochloride | | |
| | Liposome) | | |
| OPPA | Osimertinib | Oxaliplatin | Paclitaxel |
| Paclitaxel | PAD | Palbociclib | Palifermin |
| Albumin-stabilized | | | |
| Nanoparticle | | | |
| Formulation | | | |
| Palonosetron | Palonosetron | Pamidronate Disodium | Panitumumab |
| Hydrochloride | Hydrochloride | | |
| | and Netupitant | | |
| Panobinostat | Pazopanib | PCV | PEB |
| Lactate | Hydrochloride | | |
| Pegaspargase | Pegfilgrastim | Peginterferon Alfa-2b | PEG-Intron |
| | | | (Peginterferon |
| | | | Alfa-2b) |
| Pembrolizumab | Pemetrexed | Perjeta (Pertuzumab) | Pertuzumab |
| | Disodium | | |
| Plerixafor | Pomalidomide | Pomalyst | Ponatinib |
| | | (Pomalidomide) | Hydrochloride |
| Portrazza | Poteligeo | Pralatrexate | Prednisone |
| (Necitumumab) | (Mogamulizumab- | | |
| | kpkc) | | |
| Procarbazine | Procrit (Epoetin | Proleukin (Aldesleukin) | Prolia |
| Hydrochloride | Alfa) | | (Denosumab) |
| Promacta | Propranolol | Provenge (Sipuleucel-T) | Purinethol |
| (Eltrombopag | Hydrochloride | | (Mercaptopurine) |
| Olamine) | | | |
| Purixan | Radium 223 | Raloxifene | Ramucirumab |
| (Mercaptopurine) | Dichloride | Hydrochloride | |
| Rasburicase | R-CHOP | R-CVP | Recombinant |
| | | | Human |
| | | | Papillomavirus |
| | | | (HPV) Bivalent |
| | | | Vaccine |
| Recombinant | Recombinant | Recombinant Interferon | Regorafenib |
| Human | Human | Alfa-2b | |
| Papillomavirus | Papillomavirus | | |
| (HPV) Nonavalent | (HPV) | | |
| Vaccine | Quadrivalent | | |
| | Vaccine | | |
| Relistor | R-EPOCH | Retacrit (Epoetin Alfa) | Revlimid |
| (Methylnaltrexone | | | (Lenalidomide) |
| Bromide) | | | |
| Rheumatrex | Ribociclib | R-ICE | Rituxan |
| (Methotrexate) | | | (Rituximab) |
| Rituxan Hycela | Rituximab | Rituximab and | Rolapitant |
| (Rituximab and | | Hyaluronidase Human | Hydrochloride |
| Hyaluronidase | | | |
| Human) | | | |
| Romidepsin | Romiplostim | Rubidomycin | Rubraca |
| | | (Daunorubicin | (Rucaparib |
| | | Hydrochloride) | Camsylate) |
| Rucaparib | Ruxolitinib | Rydapt (Midostaurin) | Sancuso |
| Camsylate | Phosphate | | (Granisetron) |
| Sclerosol | Siltuximab | Sipuleucel-T | Somatuline Depot |
| Intrapleural | | | (Lanreotide |
| Aerosol (Talc) | | | Acetate) |
| Sonidegib | Sorafenib | Sprycel (Dasatinib) | STANFORD V |
| | Tosylate | | |
| Sterile Talc | Steritalc (Talc) | Stivarga (Regorafenib) | Sunitinib Malate |
| Powder (Talc) | | | |
| Sustol | Sutent (Sunitinib | Sylatron (Peginterferon | Sylvant |
| (Granisetron) | Malate) | Alfa-2b) | (Siltuximab) |
| Synribo | Tabloid | TAC | Tafinlar |
| (Omacetaxine | (Thioguanine) | | (Dabrafenib) |
| Mepesuccinate) | | | |
| Tagrisso | Talc | Talimogene | Tamoxifen |
| (Osimertinib) | | Laherparepvec | Citrate |
| Tarabine PFS | Tarceva (Erlotinib | Targretin (Bexarotene) | Tasigna |
| (Cytarabine) | Hydrochloride) | | (Nilotinib) |
| Tavalisse | Taxol (Paclitaxel) | Taxotere (Docetaxel) | Tecentriq |
| (Fostamatinib | | | (Atezolizumab) |
| Disodium) | | | |
| Temodar | Temozolomide | Temsirolimus | Thalidomide |
| (Temozolomide) | | | |
| Thalomid | Thioguanine | Thiotepa | Tibsovo |
| (Thalidomide) | | | (Ivosidenib) |
| Tisagenlecleucel | Tocilizumab | Tolak (Fluorouracil-- | Topotecan |
| | | Topical) | Hydrochloride |
| Toremifene | Torisel | Totect (Dexrazoxane | TPF |
| | (Temsirolimus) | Hydrochloride) | |
| Trabectedin | Trametinib | Trastuzumab | Treanda |
| | | | (Bendamustine |
| | | | Hydrochloride) |
| Trexall | Trifluridine and | Trisenox (Arsenic | Tykerb (Lapatinib |
| (Methotrexate) | Tipiracil | Trioxide) | Ditosylate) |
| | Hydrochloride | | |
| Unituxin | Uridine Triacetate | VAC | Valrubicin |
| (Dinutuximab) | | | |
| Valstar | Vandetanib | VAMP | Varubi |
| (Valrubicin) | | | (Rolapitant |
| | | | Hydrochloride) |
| Vectibix | VeIP | Velcade (Bortezomib) | Vemurafenib |
| (Panitumumab) | | | |
| Venclexta | Venetoclax | Verzenio (Abemaciclib) | Vidaza |
| (Venetoclax) | | | (Azacitidine) |
| Vinblastine Sulfate | Vincristine | Vincristine Sulfate | Vinorelbine |
| | Sulfate | Liposome | Tartrate |
| VIP | Vismodegib | Vistogard (Uridine | Vitrakvi |
| | | Triacetate) | (Larotrectinib |
| | | | Sulfate) |
| Vizimpro | Voraxaze | Vorinostat | Votrient |
| (Dacomitinib) | (Glucarpidase) | | (Pazopanib |
| | | | Hydrochloride) |
| Vyxeos | Xalkori | Xeloda (Capecitabine) | XELIRI |
| (Daunorubicin | (Crizotinib) | | |
| Hydrochloride and | | | |
| Cytarabine | | | |
| Liposome) | | | |
| XELOX | Xgeva | Xofigo (Radium 223 | Xtandi |
| | (Denosumab) | Dichloride) | (Enzalutamide) |
| Yervoy | Yescarta | Yondelis (Trabectedin) | Zaltrap (Ziv- |
| (Ipilimumab) | (Axicabtagene | | Aflibercept) |
| | Ciloleucel) | | |
| Zarxio (Filgrastim) | Zejula (Niraparib | Zelboraf (Vemurafenib) | Zevalin |
| | Tosylate | | (Ibritumomab |
| | Monohydrate) | | Tiuxetan) |
| Zinecard | Ziv-Aflibercept | Zofran (Ondansetron | Zoladex |
| (Dexrazoxane | | Hydrochloride) | (Goserelin |
| Hydrochloride) | | | Acetate) |
| Zoledronic Acid | Zolinza | Zometa (Zoledronic | Zydelig |
| | (Vorinostat) | Acid) | (Idelalisib) |
| Zykadia | Zytiga | | |
| (Ceritinib) | (Abiraterone | | |
| | Acetate) | | |

For a more detailed description of anticancer agents and other optional therapeutic agents, those skilled in the art are referred to any number of instructive manuals including, but not limited to, the Physician's Desk Reference and to Goodman and Gilman's “Pharmaceutical Basis of Therapeutics” tenth edition, Eds. Hardman et al., 2002.

In another embodiment, the methods of treating cancer provided herein comprise administering a Compound of the Disclosure to a subject in combination with radiation therapy. The methods provided herein are not limited by the types, amounts, or delivery and administration systems used to deliver the therapeutic dose of radiation to a patient. For example, the patient may receive photon radiotherapy, particle beam radiation therapy, other types of radiotherapies, and combinations thereof. In some embodiments, the radiation is delivered to the patient using a linear accelerator. In still other embodiments, the radiation is delivered using a gamma knife.

The source of radiation can be external or internal to the patient. External radiation therapy is most common and involves directing a beam of high-energy radiation to a tumor site through the skin using, for instance, a linear accelerator. While the beam of radiation is localized to the tumor site, it is nearly impossible to avoid exposure of normal, healthy tissue. However, external radiation is usually well tolerated by patients. Internal radiation therapy involves implanting a radiation-emitting source, such as beads, wires, pellets, capsules, particles, and the like, inside the body at or near the tumor site including the use of delivery systems that specifically target cancer cells (e.g., using particles attached to cancer cell binding ligands). Such implants can be removed following treatment, or left in the body inactive. Types of internal radiation therapy include, but are not limited to, brachytherapy, interstitial irradiation, intracavity irradiation, radioimmunotherapy, and the like.

The subject may optionally receive radiosensitizers (e.g., metronidazole, misonidazole, intra-arterial Budr, intravenous iododeoxyuridine (IudR), nitroimidazole, 5-substituted-4-nitroimidazoles, 2H-isoindolediones, [[(2-bromoethyl)-amino]methyl]-nitro-1H-imidazole-1-ethanol, nitroaniline derivatives, DNA-affinic hypoxia selective cytotoxins, halogenated DNA ligand, 1,2,4 benzotriazine oxides, 2-nitroimidazole derivatives, fluorine-containing nitroazole derivatives, benzamide, nicotinamide, acridine-intercalator, 5-thiotretrazole derivative, 3-nitro-1,2,4-triazole, 4,5-dinitroimidazole derivative, hydroxylated texaphrins, cisplatin, mitomycin, tiripazamine, nitrosourea, mercaptopurine, methotrexate, fluorouracil, bleomycin, vincristine, carboplatin, epirubicin, doxorubicin, cyclophosphamide, vindesine, etoposide, paclitaxel, heat (hyperthermia), and the like), radioprotectors (e.g., cysteamine, aminoalkyl dihydrogen phosphorothioates, amifostine (WR 2721), IL-1, IL-6, and the like). Radiosensitizers enhance the killing of tumor cells. Radioprotectors protect healthy tissue from the harmful effects of radiation.

Any type of radiation can be administered to a subject, so long as the dose of radiation is tolerated by the patient without unacceptable negative side-effects. Suitable types of radiotherapy include, for example, ionizing (electromagnetic) radiotherapy (e.g., X-rays or gamma rays) or particle beam radiation therapy (e.g., high linear energy radiation). Ionizing radiation is defined as radiation comprising particles or photons that have sufficient energy to produce ionization, i.e., gain or loss of electrons (as described in, for example, U.S. Pat. No. 5,770,581 incorporated herein by reference in its entirety). The effects of radiation can be at least partially controlled by the clinician. In one embodiment, the dose of radiation is fractionated for maximal target cell exposure and reduced toxicity.

In one embodiment, the total dose of radiation administered to a subject is about 0.01 Gray (Gy) to about 100 Gy. In another embodiment, about 10 Gy to about 65 Gy (e.g., about 15 Gy, 20 Gy, 25 Gy, 30 Gy, 35 Gy, 40 Gy, 45 Gy, 50 Gy, 55 Gy, or 60 Gy) are administered over the course of treatment. While in some embodiments a complete dose of radiation can be administered over the course of one day, the total dose is ideally fractionated and administered over several days. Desirably, radiotherapy is administered over the course of at least about 3 days, e.g., at least 5, 7, 10, 14, 17, 21, 25, 28, 32, 35, 38, 42, 46, 52, or 56 days (about 1-8 weeks). Accordingly, a daily dose of radiation will comprise approximately 1-5 Gy (e.g., about 1 Gy, 1.5 Gy, 1.8 Gy, 2 Gy, 2.5 Gy, 2.8 Gy, 3 Gy, 3.2 Gy, 3.5 Gy, 3.8 Gy, 4 Gy, 4.2 Gy, or 4.5 Gy), or 1-2 Gy (e.g., 1.5-2 Gy). The daily dose of radiation should be sufficient to induce destruction of the targeted cells. If stretched over a period, in one embodiment, radiation is not administered every day, thereby allowing the animal to rest and the effects of the therapy to be realized. For example, radiation desirably is administered on 5 consecutive days, and not administered on 2 days, for each week of treatment, thereby allowing 2 days of rest per week. However, radiation can be administered 1 day/week, 2 days/week, 3 days/week, 4 days/week, 5 days/week, 6 days/week, or all 7 days/week, depending on the animal's responsiveness and any potential side effects. Radiation therapy can be initiated at any time in the therapeutic period. In one embodiment, radiation is initiated in week 1 or week 2, and is administered for the remaining duration of the therapeutic period. For example, radiation is administered in weeks 1-6 or in weeks 2-6 of a therapeutic period comprising 6 weeks for treating, for instance, a solid tumor. Alternatively, radiation is administered in weeks 1-5 or weeks 2-5 of a therapeutic period comprising 5 weeks. These exemplary radiotherapy administration schedules are not intended, however, to limit the methods provided herein.

### V. Kits

In another embodiment, the present disclosure provides kits comprising a Compound of the Disclosure, or a pharmaceutical composition thereof, and instructions for administering the compound or composition to a subject having cancer.

In another embodiment, the present disclosure provides kits comprising a Compound of the Disclosure, or a pharmaceutical composition thereof, packaged in a manner that facilitates their use to practice methods of the present disclosure.

In another embodiment, the kit includes a Compound of the Disclosure, or a pharmaceutical composition thereof, packaged in a container, such as a sealed bottle or vessel, with a label affixed to the container or included in the kit that describes use of the compound or composition to practice the method of the disclosure. In one embodiment, the compound or composition is packaged in a unit dosage form. The kit may include a single dose or multiple doses of a Compound of the Disclosure, or a pharmaceutical composition thereof.

In another embodiment, the kit includes an optional therapeutic agent.

In another embodiment, the present disclosure provides kits for carrying out the methods described herein, the kit comprising (i) a Compound of the Disclosure; and (ii) and instructions for administering the Compound of the Disclosure to a patient having cancer. In some embodiments, the kit further comprises at an optional therapeutic agent for the treatment of cancer. In some embodiments, the kit further comprises instructions for administering the Compound of the Disclosure together with at least one second therapeutic agent for the treatment of cancer.

### VI. Biomarkers

The term “biomarker” as used herein refers to any biological compound, such as a gene, a protein, a fragment of a protein, a peptide, a polypeptide, a nucleic acid, etc., or chromosome abnormality, such as a chromosome translocation, that can be detected and/or quantified in a subject in vivo or in a biological sample obtained from a subject. A biomarker can be the entire intact molecule, or it can be a portion or fragment thereof. In one embodiment, the expression level of the biomarker is measured. The expression level of the biomarker can be measured, for example, by detecting the protein or RNA, e.g., mRNA, level of the biomarker. In some embodiments, portions or fragments of biomarkers can be detected or measured, for example, by an antibody or other specific binding agent. In some embodiments, a measurable aspect of the biomarker is associated with a given state of the subject, such as the subject's age. For biomarkers that are detected at the protein or RNA level, such measurable aspects may include, for example, the presence, absence, or concentration, i.e., expression level, of the biomarker in the subject, or biological sample obtained from the subject. For biomarkers that are detected at the nucleic acid level, such measurable aspects may include, for example, allelic versions of the biomarker or type, rate, and/or degree of mutation of the biomarker, also referred to herein as mutation status.

For biomarkers that are detected based on expression level of protein or RNA, expression level measured between different phenotypic statuses can be considered different, for example, if the mean or median expression level of the biomarker in the different groups is calculated to be statistically significant. Common tests for statistical significance include, among others, t-test, ANOVA, Kruskal-Wallis, Wilcoxon, Mann-Whitney, Significance Analysis of Microarrays, odds ratio, etc. Biomarkers, alone or in combination, provide measures of relative likelihood that a subject belongs to one phenotypic status or another. Therefore, they are useful, inter alia, as markers for disease and as indicators that particular therapeutic treatment regimens will likely result in beneficial patient outcomes. The term “overexpression” indicates that the expression level of the biomarker in the subject having a disease, condition, or disorder is amplified, e.g., above the mean or median expression level of the biomarker in, e.g., a normal undiseased subject.

Biomarkers include, but are not limited to, DDR enzymes, e.g., Pol η, Pol μ, or Pol θ, or genes, e.g., POLH, POLM, or POLQ. In one embodiment, the measurable aspect of the biomarker is its expression status. In another embodiment, the measurable aspect of the biomarker is elevated levels, e.g., overexpression, of the biomarker. In one embodiment, the measurable aspect of the biomarker is its mutation status.

In one embodiment, the biomarker is Pol θ expression which is differentially present in a subject of one phenotypic status, e.g., a subject having cancer, as compared with another phenotypic status, e.g., a normal undiseased subject or a subject having a cancer without overexpression of Pol θ. In one embodiment, the biomarker is overexpression of Pol θ, e.g., in cancer cells. In one embodiment, the biomarker is overexpression of POLQ, e.g., in cancer cells.

In another embodiment, the biomarker is Pol 9 expression which is differentially present in a subject of one phenotypic status, as compared with another phenotypic status. In one embodiment, the biomarker is overexpression of Pol η, e.g., in cancer cells. In one embodiment, the biomarker is overexpression of POLH, e.g., in cancer cells.

In another embodiment, the biomarker is Pol μ expression which is differentially present in a subject of one phenotypic status as compared with another phenotypic status. In one embodiment, the biomarker is overexpression of Pol μ, e.g., in cancer cells. In one embodiment, the biomarker is overexpression of POLM, e.g., in cancer cells.

In one embodiment, the biomarker is BRCA1 expression which is differentially present in a subject of one phenotypic status, e.g., a subject having cancer as compared with another phenotypic status, e.g., a normal undiseased subject or a subject having a cancer without overexpression of BRCA1. In one embodiment, the biomarker is overexpression of BRCA1 in cancer cells.

In one embodiment, the biomarker is BRCA2 expression which is differentially present in a subject of one phenotypic status, e.g., a subject having cancer as compared with another phenotypic status, e.g., a normal undiseased subject or a subject having a cancer without overexpression of BRCA2. In one embodiment, the biomarker is overexpression of BRCA1 in cancer cells.

In one embodiment, the biomarker is Pol θ, Pol η, or Pol μ expression which is differentially present in a subject of one phenotypic status, e.g., a subject after administration of a Compound of the Disclosure, as compared with another phenotypic status, e.g., a normal undiseased subject or a subject before administration of a Compound of the Disclosure. In some embodiments, the biomarker is decreased expression of Pol θ, Pol η, or Pol μ caused by administration of a Compound of the Disclosure to a subject.

Biomarker standards can be predetermined, determined concurrently, or determined after a biological sample is obtained from the subject. Biomarker standards for use with the methods described herein can, for example, include data from samples from subjects without cancer and/or data from samples from subjects with a cancer. Comparisons can be made to establish predetermined threshold biomarker standards for different classes of subjects, e.g., diseased vs. non-diseased subjects. The standards can be run in the same assay or can be known standards from a previous assay.

A biomarker is differentially present between different phenotypic status groups if the mean or median expression or mutation levels of the biomarker is calculated to be different, i.e., higher or lower, between the groups. Thus, biomarkers provide an indication that a subject, e.g., a subject having cancer, belongs to one phenotypic status or another.

In addition to individual biological compounds, e.g., Pol θ, the term “biomarker” as used herein is meant to include groups, sets, or arrays of multiple biological compounds. The term “biomarker” may comprise one, two, three, four, five, six, seven, eight, nine, ten, fifteen, twenty, twenty five, thirty, or more, biological compounds. In embodiment, the biomarker comprises one, two, or three biological compounds.

The determination of the expression level or mutation status of a biomarker in a subject can be performed using any of the many methods known in the art. Any method known in the art for quantitating specific proteins and/or detecting biomarker expression, e.g., Pol θ, Pol η, or Pol μ expression, and/or or the expression or mutation levels of any other biomarker(s) in a patient or a biological sample may be used in the methods of the disclosure. Examples include, but are not limited to, PCR (polymerase chain reaction), or RT-PCR, flow cytometry, Northern blot, Western blot, immunoassays, e.g., ELISA (enzyme linked immunosorbent assay), RIA (radioimmunoassay), Simoa™, gene chip analysis of RNA expression, immunohistochemistry, immunofluorescence, or mass-spectroscopy. See, e.g., Slagle et al. Cancer 83:1401 (1998). Certain embodiments of the disclosure include methods wherein biomarker RNA expression (transcription) is determined. Other embodiments of the disclosure include methods wherein protein expression in the biological sample is determined. See, e.g., Harlow et al., Antibodies: A Laboratory Manual, Cold Spring Harbor Laboratory, Cold Spring Harbor, NY, (1988); Ausubel et al., Current Protocols in Molecular Biology, John Wiley & Sons, New York 3rd Edition, (1995); Kamel and Al-Amodi, Genomics Proteomics Bioinformatics 15:220-235 (2017). For northern blot or RT-PCR analysis, RNA is isolated from tissue sample using RNAse free techniques. Such techniques are commonly known in the art.

In one embodiment of the disclosure, a biological sample is obtained from the subject and the biological sample is assayed for determination of a biomarker expression or mutation status. In some embodiments, the biological sample is blood from the subject. In some embodiments, the biological sample is the cancer tissue or cells of the patient.

In another embodiment of the disclosure, Northern blot analysis of biomarker transcription in a tumor cell sample is performed. Northern analysis is a standard method for detection and/or quantitation of mRNA levels in a sample. Initially, RNA is isolated from a sample to be assayed using Northern blot analysis. In the analysis, the RNA samples are first separated by size via electrophoresis in an agarose gel under denaturing conditions. The RNA is then transferred to a membrane, crosslinked and hybridized with a labeled probe. Typically, Northern hybridization involves polymerizing radiolabeled or nonisotopically labeled DNA, in vitro, or generation of oligonucleotides as hybridization probes. Typically, the membrane holding the RNA sample is prehybridized or blocked prior to probe hybridization to prevent the probe from coating the membrane and, thus, to reduce non-specific background signal. After hybridization, typically, unhybridized probe is removed by washing in several changes of buffer. Stringency of the wash and hybridization conditions can be designed, selected and implemented by any practitioner of ordinary skill in the art. Detection is accomplished using detectably labeled probes and a suitable detection method. Radiolabeled and non-radiolabled probes and their use are well known in the art. The presence and or relative levels of expression of the biomarker being assayed can be quantified using, for example, densitometry.

In another embodiment, biomarker expression and/or mutation status is determined using RT-PCR. RT-PCR allows detection of the progress of a PCR amplification of a target gene in real time. Design of the primers and probes required to detect expression and/or mutation status of a biomarker of the disclosure is within the skill of a practitioner of ordinary skill in the art. RT-PCR can be used, for example, to determine the level of RNA encoding a biomarker of the disclosure in a tissue sample. In an embodiment of the disclosure, RNA from the biological sample is isolated, under RNAse free conditions, than converted to DNA by treatment with reverse transcriptase. Methods for reverse transcriptase conversion of RNA to DNA are well known in the art. A description of PCR is provided in the following references: Mullis et al., Cold Spring Harbor Symp. Quant. Biol. 51:263 (1986); EP 50,424; EP 84,796; EP 258,017; EP 237,362; EP 201,184; U.S. Pat. Nos. 4,683,202; 4,582,788; 4,683,194.

RT-PCR probes depend on the 5′-3′ nuclease activity of the DNA polymerase used for PCR to hydrolyze an oligonucleotide that is hybridized to the target amplicon (biomarker gene). RT-PCR probes are oligonucleotides that have a fluorescent reporter dye attached to the 5′ end and a quencher moiety coupled to the 3′ end (or vice versa). These probes are designed to hybridize to an internal region of a PCR product. In the unhybridized state, the proximity of the fluor and the quench molecules prevents the detection of fluorescent signal from the probe. During PCR amplification, when the polymerase replicates a template on which an RT-PCR probe is bound, the 5′-3′ nuclease activity of the polymerase cleaves the probe. This decouples the fluorescent and quenching dyes and FRET no longer occurs. Thus, fluorescence increases in each cycle, in a manner proportional to the amount of probe cleavage. Fluorescence signal emitted from the reaction can be measured or followed over time using equipment which is commercially available using routine and conventional techniques.

In another embodiment of the disclosure, expression of proteins encoded by biomarkers are detected by western blot analysis. A western blot (also known as an immunoblot) is a method for protein detection in a given sample of tissue homogenate or extract. It uses gel electrophoresis to separate denatured proteins by mass. The proteins are then transferred out of the gel and onto a membrane (e.g., nitrocellulose or polyvinylidene fluoride (PVDF)), where they are detected using a primary antibody that specifically bind to the protein. The bound antibody can then detected by a secondary antibody that is conjugated with a detectable label (e.g., biotin, horseradish peroxidase or alkaline phosphatase). Detection of the secondary label signal indicates the presence of the protein.

In another embodiment of the disclosure, the expression of a protein encoded by a biomarker is detected by enzyme-linked immunosorbent assay (ELISA). In one embodiment of the disclosure, “sandwich ELISA” comprises coating a plate with a capture antibody; adding sample wherein any antigen present binds to the capture antibody; adding a detecting antibody which also binds the antigen; adding an enzyme-linked secondary antibody which binds to detecting antibody; and adding substrate which is converted by an enzyme on the secondary antibody to a detectable form. Detection of the signal from the secondary antibody indicates presence of the biomarker antigen protein.

In one embodiment, present disclosure provides methods of treating a subject having cancer, the method comprising: (a) determining whether a biomarker, e.g., overexpression of Pol θ, Pol η, or Pol μ, is present or absent in a biological sample taken from the subject; and (b) administering Compound of the Disclosure to the subject if the biomarker is present in the biological sample.

In another embodiment, the present disclosure provides a method of identifying whether a subject having cancer as a candidate for treatment with a Compound of the Disclosure, the method comprising: (a) determining whether a biomarker, e.g., overexpression of Pol θ, Pol η, or Pol μ, is present or absent in a biological sample taken from the subject; and (b) identifying the subject as being a candidate for treatment if the biomarker is present; or (c) identifying the subject as not being a candidate for treatment if the biomarker is absent.

In another embodiment, the present disclosure provides a method of predicting treatment outcome in a subject having cancer, the method comprising determining whether a biomarker, e.g., overexpression of Pol θ, Pol η, or Pol μ, is present or absent in a biological sample taken from the subject, wherein (a) the presence of the biomarker in the biological sample indicates that administering a Compound of the Disclosure to the subject will likely cause a favorable therapeutic response; and (b) the absence of the biomarker in the biological sample indicates that administering a Compound of the Disclosure to the subject will likely cause an unfavorable therapeutic response.

In another embodiment, the present disclosure provides a method, comprising administering a therapeutically effective amount of a Compound of the Disclosure to a subject in need thereof, wherein: (a) the subject has cancer; and (b) the cancer is characterized as having a biomarker, e.g., overexpression of one or more DNA damage repair enzymes, e.g., overexpression of Pol θ, Pol η, or Pol μ.

In another embodiment, the present disclosure provides a method of identifying whether a patient having metastatic prostate cancer is a candidate for treatment with a Compound of the Disclosure during or after receiving ADT, the method comprising:
(a) determining whether an increased expression of LINE-1, BRCA1, BRCA2, Pol θ, Pol η, or Pol μ, is present or absent in a biological sample taken from the subject during or following ADT; and
(b) identifying the subject as being a candidate for treatment if an increased expression of LINE-1, BRCA1, BRCA2, Pol θ, Pol η, or Pol μ is present; or
(c) identifying the subject as not being a candidate for treatment if an increased expression of LINE-1, BRCA1, BRCA2, Pol θ, Pol η, or Pol μ is absent.

In another embodiment, the present disclosure provides a method, comprising administering a therapeutically effective amount a Compound of the Disclosure to a subject in need thereof, wherein:
(a) the subject has metastatic prostate cancer;
(b) the subject has undergone or is undergoing ADT; and
(c) the metastatic prostate cancer is characterized as having an increased expression of LINE-1, BRCA1, BRCA2, Pol θ, Pol η, or Pol μ.

In another embodiment, the present disclosure provides a method of treating a subject having cancer, the method comprising:
(a) determining whether an overexpression of one or more DNA damage repair enzymes, e.g., Pol θ, Pol η, and/or Pol μ, is present or absent in a biological sample taken from the subject; and
(b) administering a therapeutically effective amount a Compound of the Disclosure to the subject if an overexpression of one or more DNA damage repair enzymes is present in the biological sample.

In another embodiment, the present disclosure provides a method of identifying whether a subject having cancer as a candidate for treatment with a Compound of the Disclosure, the method comprising:
(a) determining whether an overexpression of one or more DNA damage repair enzymes, e.g., Pol θ, Pol η, and/or Pol μ, is present or absent in a biological sample taken from the subject; and
(b) identifying the subject as being a candidate for treatment if an overexpression of one or more DNA damage repair enzymes is present; or
(c) identifying the subject as not being a candidate for treatment if overexpression of one or more DNA damage repair enzymes is absent.

In another embodiment, the present disclosure provides a method of predicting treatment outcome in a subject having cancer, the method comprising determining whether an overexpression of one or more DNA damage repair enzymes, e.g., Pol θ, Pol η, and/or Pol μ, is present or absent in a biological sample taken from the subject, wherein:
(a) the presence of an overexpression of one or more DNA damage repair enzymes in the biological sample indicates that administering a Compound of the Disclosure to the subject will likely cause a favorable therapeutic response; and
(b) the absence of an overexpression of one or more DNA damage repair enzymes in the biological sample indicates that administering a Compound of the Disclosure to the subject will likely cause an unfavorable therapeutic response.

In another embodiment, the present disclosure provides a Compound of the Disclosure for use in treating a subject having cancer, wherein:
(a) the expression level of a biomarker, e.g., one or more DNA damage repair enzymes, e.g., Pol θ, Pol η, and/or Pol μ, in a biological sample taken from the subject is determined; and
(b) a therapeutically effective amount the Compound of the Disclosure is to be administered to the subject if there is an overexpression the of one or more biomarkers in the biological sample.

In another embodiment, the present disclosure provides the use of a Compound of the Disclosure in the manufacture of a medicament for treating a subject having cancer, wherein:
(a) the expression level of a biomarker, e.g., one or more DNA damage repair enzymes, e.g., Pol θ, Pol η, and/or Pol μ, is determined in a biological sample taken from the subject; and
(b) a therapeutically effective amount the Compound of the Disclosure is to be administered to the subject if there is an overexpression of the one or more biomarkers in the biological sample.

### VII. Definitions

The term “DNA damage repair enzyme inhibitor” or “DDR enzyme inhibitor” as used herein refers to a compound that inhibits the activity of one or more human DNA damage repair enzymes with a half-maximal inhibitory concentration (IC₅₀) of 100 μM or less. In some embodiments, the DDR enzyme is any one or more of the enzymes listed in Table C. In one embodiment, a DNA repair enzyme inhibitor inhibits human DNA repair enzyme activity an IC₅₀ of 10 μM or less. In another embodiment, a DNA repair enzyme inhibitor inhibits human DNA repair enzyme activity with an IC₅₀ of 1 μM or less. In another embodiment, a DNA repair enzyme inhibitor inhibits human DNA repair enzyme activity with an IC₅₀ of 500 nM or less. In another embodiment, a DNA repair enzyme inhibitor inhibits human DNA repair enzyme activity with an IC₅₀ of 100 nM or less. In another embodiment, a DNA repair enzyme inhibitor inhibits human DNA repair enzyme activity with an IC₅₀ of 50 nM or less. In another embodiment, a DNA repair enzyme inhibitor inhibits human DNA repair enzyme activity with an IC₅₀ of 10 nM or less. Representative DNA repair enzymes are encoded by genes provided in Table B. In some embodiments, phosphorylated Compounds of the Disclosure, see Scheme 1A, are DDR enzyme inhibitors. In some embodiments, the triphosphates of Compounds of the Disclosure are DDR enzyme inhibitors.

TABLE B

| | | | |
|---|---|---|---|
| | | Chromosome | Accession |
| Gene Name* | Activity | location | number |

Base excision repair (BER) (DNA glycosylases: major altered base released)

| | | | |
|---|---|---|---|
| UNG | U | 12q24.11 | NM_080911 |
| SMUG1 | U | 12q13.13 | NM_014311 |
| MBD4 | U or T opposite G at CpG sequences | 3q21.3 | NM_003925 |
| TDG | U, T or ethenoC opposite G | 12q23.3 | NM_003211 |
| OGG1 | 8-oxoG opposite C | 3p25.3 | NM_016821 |
| MUTYH (MYH) | A opposite 8-oxoG | 1p34.1 | NM_012222 |
| NTHL1 (NTH1) | Ring-saturated or fragmented pyrimidines | 16p13.3 | NM_002528 |
| MPG | 3-meA, ethenoA, hypoxanthine | 16p13.3 | NM_002434 |
| NEIL1 | Removes thymine glycol | 15q24.2 | NM_024608 |
| NEIL2 | Removes oxidative products of pyrimidines | 8p23.1 | NM_145043 |
| NEIL3 | Removes oxidative products of pyrimidines | 4q34 | NM_018248 |

Other BER and strand break joining factors

| | | | |
|---|---|---|---|
| APEX1 (APE1) | AP endonuclease | 14q11.2 | NM_001641 |
| APEX2 | AP endonuclease | Xp11.21 | NM_014481 |
| LIG3 | DNA Ligase III | 17q12 | NM_013975 |
| XRCC1 | LIG3 accessory factor | 19q13.31 | NM_006297 |
| PNKP | Converts some DNA breaks to ligatable ends | 19q13.33 | NM_007254 |
| APLF | Accessory factor for DNA end-joining | 2p13.3 | NM_173545 |
| HMCES | Reacts with AP sites | 3q21.3 | NM_020187 |

Poly(ADP-ribose) polymerase (PARP) enzymes that bind to DNA

| | | | |
|---|---|---|---|
| PARP1 (ADPRT) | Protects strand interruptions | 1g42.12 | NM_001618 |
| PARP2 (ADPRTL2) | PARP-like enzyme | 14q11.2 | NM_005484 |
| PARP3 (ADPRTL3) | PARP-like enzyme | 3p21.1 | NM_001003931 |
| PARG | Poly(ADP-ribose) glycohydrolase | 10q11.23 | NM_003631 |
| PARPBP | Binds PARP and modulates recombination | 12q23.2 | NM_001319988 |

Direct reversal of damage

| | | | |
|---|---|---|---|
| MGMT | O⁶-meG alkyltransferase | 10q26.3 | NM_002412 |
| ALKBH2 (ABH2) | 1-meA dioxygenase | 12q24.11 | NM_001001655 |
| ALKBH3 (DEPC1) | 1-meA dioxygenase | 11p11.2 | NM_139178 |

Repair of DNA-protein crosslinks

| | | | |
|---|---|---|---|
| TDP1 | Removes 3′-tyrosylphosphate and 3′- | 14q32.11 | NM_018319 |
| | phosphoglycolate from DNA | | |
| TDP2 (TTRAP) | 5′- and 3′-tyrosyl DNA phosphodiesterase | 6p22.3 | NM_016614 |
| SPRTN (Spartan) | Reads ubiquitylation | 1q42.12-q43 | NM_032018 |

Mismatch excision repair (MMR)

| | | | |
|---|---|---|---|
| MSH2 | Mismatch (MSH2-MSH6) and loop (MSH2-MSH3) | 2p21 | NM_000251 |
| MSH3 | recognition | 5q14.1 | NM_002439 |
| MSH6 | | 2p16.3 | NM_000179 |
| MLH1 | MutL homologs, forming heterodimer | 3p22.3 | NM_000249 |
| PMS2 | | 7p22.1 | NM_000535 |
| MSH4 | MutS homologs specialized for meiosis | 1p31.1 | NM_002440 |
| MSH5 | | 6p21.33 | NM_002441 |
| MLH3 | MutL homologs of unknown function | 14q24.3 | NM_014381 |
| PMS1 | | 2q32.2 | NM_000534 |
| PMS2P3 (PMS2L3) | | 7q11.23 | NM_005395 |
| HFM1 | Helicase in meiotic-crossover formation | 1p22.2 | NM_001017975 |

Nucleotide excision repair (NER) (XP = xeroderma pigmentosum)

| | | | |
|---|---|---|---|
| XPC | Binds DNA distortions | 3p25.1 | NM_004628 |
| RAD23B | | 9q31.2 | NM_002874 |
| CETN2 | | Xq28 | NM_004344 |
| RAD23A | Substitutes for RAD23B | 19p13.13 | NM_005053 |
| XPA | Binds damaged DNA in preincision complex | 9q22.33 | NM_000380 |
| DDB1 | Complex defective in XP group E | 11q12.2 | NM_001923 |
| DDB2 (XPE) | | 11p11.2 | NM_000107 |
| RPA1 | Binds DNA in preincision complex | 17p13.3 | NM_002945 |
| RPA2 | | 1p35.3 | NM_002946 |
| RPA3 | | 7p21.3 | NM_002947 |
| TFIIH | Catalyzes unwinding in preincision complex | | |
| ERCC3 (XPB) | 3′ to 5′ DNA helicase | 2q14.3 | NM_000122 |
| ERCC2 (XPD) | 5′ to 3′ DNA helicase | 19q13.32 | NM_000400 |
| GTF2H1 | Core TFIIH subunit p62 | 11p15.1 | NM_005316 |
| GTF2H2 | Core TFIIH subunit p44 | 5q13.2 | NM_001515 |
| GTF2H3 | Core TFIIH subunit p34 | 12q24.31 | NM_001516 |
| GTF2H4 | Core TFIIH subunit p52 | 6p21.33 | NM_001517 |
| GTF2H5 (TTDA) | Core TFIIH subunit p8 | 6p25.3 | NM_207118 |
| GTF2E2 | TFIIE subunit, mutated in TTD | 8p12 | NM_002095 |
| CDK7 | Kinase subunits of TFIIH | 5q13.2 | NM_001799 |
| CCNH | | 5q14.3 | NM_001239 |
| MNAT1 | | 14q23.1 | NM_002431 |
| ERCC5 (XPG) | 3′ incision | 13q33.1 | NM_000123 |
| ERCC1 | 5′ incision DNA binding subunit | 19q13.32 | NM_001983 |
| ERCC4 (XPF) | 5′ incision catalytic subunit | 16p13.12 | NM_005236 |
| LIG1 | DNA ligase | 19q13.32 | NM_000234 |

NER-related

| | | | |
|---|---|---|---|
| ERCC8 (CSA) | Cockayne syndrome and UV-Sensitive Syndrome; | 5q12.1 | NM_000082 |
| ERCC6 (CSB) | Needed for transcription-coupled NER | 10q11.23 | NM_000124 |
| UVSSA (KIAA1530) | | 4p16.3 | NM_020894 |
| XAB2 (HCNP) | Pre-mRNA splicing; Associates with | 19p13.2 | NM_020196 |
| | PRPF19, CSA, CSB | | |
| MMS19 | Iron-sulfur cluster loading and transport | 10q24.1 | NM_022362 |

Homologous recombination

| | | | |
|---|---|---|---|
| RAD51 | Homologous pairing | 15q15.1 | NM_002875 |
| RAD51B | Rad51 homolog | 14q24.1 | NM_002877 |
| RAD51D | Rad51 homolog | 17q12 | NM_002878 |
| HELQ (HEL308) | DNA helicase in RAD51 paralog complex | 4q21.23 | NM_133636 |
| SWI5 | Accessory factor for loading RAD51 | 9q34.11 | NM_001040011 |
| SWSAP1 | Shu subunits, RAD51 recruitment | 19p13.2 | NM_175871 |
| ZSWIM7 (SWS1) | | 17p12 | NM_001042697 |
| SPIDR | | 8q11.21 | NM_001080394 |
| PDS5B | | 13q13.1 | NM_015032 |
| DMC1 | Rad51 homolog, meiosis | 22q13.1 | NM_007068 |
| XRCC2 | DNA break and crosslink repair | 7q36.1 | NM_005431 |
| XRCC3 | | 14q32.33 | NM_005432 |
| RAD52 | Accessory factors for recombination | 12p13.33 | NM_134424 |
| RAD54L | | 1p34.1 | NM_003579 |
| RAD54B | | 8q22.1 | NM_012415 |
| BRCA1 | Accessory factor for transcription and | 17q21.31 | NM_007294 |
| | recombination, E3 Ubiquitin ligase | | |
| BARD1 | BRCA1-associated | 2q35 | NM_000465 |
| ABRAXAS1 | In BRCA1 A complex | 4q21.23 | NM_139076 |
| PAXIP1 (PTIP) | MDC1 paralog in 53BP1 pathway | 7q36.2 | NM_007349 |
| SMC5 | Recruit cohesion during HR | 9q21.12 | NM_015110 |
| SMC6 | SMC5, SMC6 | 2p24.2 | NM_001142286 |
| SHLD1 | Suppressing end-resection | 20p12.3 | NM_001303477 |
| SHLD2 (FAM35A) | | 10q23.2 | NM_001330112 |
| SHLD3 | | 5q12.3 | NM_001365341 |
| SEM1 (SHEM1) | BRCA2 associated | 7q21.3 | NM_006304 |
| (DSS1) | | | |
| RAD50 | ATPase in complex with MRE11A, NBS1 | 5q23.3 | NM_005732 |
| MRE11A | 3′ exonuclease, defective in ATLD | 11q21 | NM_005590 |
| | (ataxia-telangiectasia-like disorder) | | |
| NBN (NBS1) | Mutated in Nijmegen breakage syndrome | 8q21.3 | NM_002485 |
| RBBP8 (CtIP) | Promotes DNA end resection | 18q11.2 | NM_002894 |
| MUS81 | Subunits of structure-specific DNA nuclease | 11q13.1 | NM_025128 |
| EME1 (MMS4L) | | 17q21.33 | NM_152463 |
| EME2 | | 16p13.3 | NM_001257370 |
| SLX1A (GIYD1) | Subunit of SLX1-SLX4 structure-specific nuclease, | 16p11.2 | NM_001014999 |
| SLX1B (GIYD2) | two identical tandem genes in the human genome | 16p11.2 | NM_024044 |
| GEN1 | Nuclease cleaving Holliday junctions | 2p24.2 | NM_182625 |

Fanconi anemia (Tolerance and repair of DNA crosslinks and other adducts in DNA)

| | | | |
|---|---|---|---|
| FANCA | FANCA | 16q24.3 | NM_000135 |
| FANCB | FANCB | Xp22.31 | NM_152633 |
| FANCC | FANCC | 9q22.32 | NM_000136 |
| BRCA2 (FANCD1) | Cooperation with RADS1, essential function | 13q13.1 | NM_000059 |
| FANCD2 | Target for monoubiquitination | 3p25.3 | NM_033084 |
| FANCE | FANCE | 6p21.31 | NM_021922 |
| FANCF | FANCF | 11p14.3 | NM_022725 |
| FANCG (XRCC9) | FANCG | 9p13.3 | NM_004629 |
| FANCI (KIAA1794) | target for monoubiquitination | 15q26.1 | NM_018193 |
| BRIP1 (FANCJ) | DNA helicase, BRCA1-interacting | 17q23 | NM_032043 |
| FANCL | FANCL | 2p16.1 | NM_018062 |
| FANCM | helicase/translocase | 14q21.3 | NM_020937 |
| PALB2 (FANCN) | Co-localizes with BRCA2 (FANCD1) | 16p12.1 | NM_024675 |
| RAD51C (FANCO) | Rad51 homolog , FANCO | 17q23.2 | NM_002876 |
| SLX4(FANCP) | nuclease subunit/scaffold SLX4, FANCP | 16p13.3 | NM_032444 |
| FAAP20 (C1orf86) | FANCA - associated | 1p36.33 | NM_182533 |
| FAAP24 (C19orf40) | FAAP24 | 19q13.11 | NM_152266 |
| FAAP100 | Part of FA core complex | 17q25.3 | NM_025161 |
| UBE2T (FANCT) | E2 ligase for FANCL | 1q32.1 | NM_014176 |

Non-homologous end-joining

| | | | |
|---|---|---|---|
| XRCC6 (Ku70) | DNA end binding subunit | 22q13.2 | NM_001469 |
| XRCC5 (Ku80) | DNA end binding subunit | 2q35 | NM_021141 |
| PRKDC | DNA-dependent protein kinase catalytic subunit | 8q11.21 | NM_006904 |
| LIG4 | Ligase | 13q33.3 | NM_002312 |
| XRCC4 | Ligase accessory factor | 5q14.2 | NM_003401 |
| DCLRE1C (Artemis) | Nuclease | 10p13 | NM_022487 |
| NHEJ1 (XLF, | End-joining factor | 2q35 | NM_024782 |
| Cernumnos) | | | |

Modulation of nucleotide pools

| | | | |
|---|---|---|---|
| NUDT1 (MTH1) | 8-oxoGTPase | 7p22.3 | NM_002452 |
| DUT | dUTPase | 15q21.1 | NM_001948 |
| RRM2B (p53R2) | p53-inducible ribonucleotide reductase | 8q22.3 | NM_015713 |
| | small subunit 2 homolog | | |
| PARK7 (DJ-1) | Guanine glycation repair | 1p36.23 | NM_007262 |
| DNPH1 | Hydrolase for 5-hydroxymethyl deoxyuridine | 6p21.1 | NM_006443 |
| NUDT15 (MTH2) | Hydrolysis of thiopurines? | 13q14.2 | NM_018283 |
| NUDT18 (MTH3) | Hydrolysis of 8-hydroxypurine diphosphates | 8p21.3 | NM_024815 |

DNA polymerases

| | | | |
|---|---|---|---|
| POLA1 | DNA synthesis at resected ends | Xp22.1-p21.3 | NM_001330360 |
| POLB | BER in nuclear DNA | 8p11.21 | NM_002690 |
| POLD1 | Pol delta subunits | 19q13.33 | NM_002691 |
| POLD2 | POLD1 (catalytic subunit), POLD2 | 7p13 | NM_001127218 |
| | (Also subunit of pol zeta), | | |
| POLD3 | POLD3 (Also subunit of pol zeta), | 11q13.4 | NM_006591 |
| POLD4 | POLD4 (auxiliary subunit) | 11q13.2 | NM_021173 |
| POLE (POLE1) | Pol epsilon subunits | 12q24.33 | NM_006231 |
| POLE2 | | 14q21.3 | NM_002692 |
| POLE3 | | 9g32 | NM_017443 |
| POLE4 | | 2p12 | NM_019896 |
| REV3L (POLZ) | DNA pol zeta catalytic subunit, essential function | 6q21 | NM_002912 |
| MAD2L2 (REV7) | DNA pol zeta and shieldin subunit | 1p36.22 | NM_006341 |
| REV1 (REV1L) | dCMP transferase | 2g11.2 | NM_016316 |
| POLG | Mitochondrial DNA repair and replication | 15q26.1 | NM_002693 |
| POLH | xeroderma pigmentosum (XP) variant | 6p21.1 | NM_006502 |
| POLI (RAD30B) | Lesion bypass | 18q21.2 | NM_007195 |
| POLQ | TMEJ (alt-EJ) | 3q13.33 | NM_199420 |
| POLK (DINB1) | Lesion bypass and NER | 5q13.3 | NM_016218 |
| POLL | Gap-filling during non-homologous end-joining | 10q24.32 | NM_013274 |
| POLM | Gap filling during non-homologous end-joining | 7p13 | NM_013284 |
| POLN (POL4P) | Lesion bypass/recombination? | 4p16.3 | NM_181808 |
| PRIMPOL | Primase and DNA-directed polymerase | 4q35.1 | NM_152683 |
| DNTT | Terminal transferase | 10q24.1 | NM_004088 |

Editing and processing nucleases

| | | | |
|---|---|---|---|
| FEN1 (DNase IV) | 5′ nuclease | 11q12.2 | NM_004111 |
| FAN1 (MTMR15) | 5′ nuclease interacting with FANCD2 | 15q13.2 | NM_014967 |
| TREX1 | 3′ exonuclease | 3p21.31 | NM_033629 |
| TREX2 | 3′ exonuclease | Xq28 | NM_080701 |
| EXO1 (HEX1) | S′exonuclease | 1q43 | NM_003686 |
| APTX (aprataxin) | Processing of DNA single-strand interruptions | 9p21.1 | NM_175073 |
| SPO11 | endonuclease | 20q13.32 | NM_012444 |
| ENDOV | incision 3′ of hypoxanthine and uracil in DNA and | 17q25.3 | NM_173627 |
| | inosine in RNA | | |
| DNA2 | Seckel Syndrome 8 | 10q21.3 | NM_001080449 |
| DCLRE1A (SNM1A) | 5′-3′ exonuclease, DNA crosslink repair | 10q25.3 | NM_014881 |
| DCLRE1B (SNM1B) | 5′-3′ exonuclease, APOLLO | 1p13.2 | NM_022836 |
| EXO5 | exonuclease | 1p34.2 | NM_001346946 |

Ubiquitination and modification

| | | | |
|---|---|---|---|
| UBE2A (RAD6A) | Ubiquitin-conjugating enzyme | Xq24-q25 | NM_003336 |
| UBE2B (RAD6B) | Ubiquitin-conjugating enzyme | 5q31.1 | NM_003337 |
| RAD18 | E3 ubiquitin ligase | 3p25.3 | NM_020165 |
| SHPRH | E3 ubiquitin ligase, SWI/SNF related, homolog | 6q24.3 | NM_001042683 |
| | of S. cerevisiae Rad5 | | |
| HLTF (SMARCA3) | E3 ubiquitin ligase, SWI/SNF related, homolog | 3q25.1-q26.1 | NM_003071 |
| | of S. cerevisiae Rad5 | | |
| RNF168 | E3 ubiquitin ligase for DSB repair; | 3q29 | NM_152617 |
| | ATM-like and RIDDLE syndrome | | |
| RNF8 | E3 ubiquitin ligase for DSB repair | 6p21.2 | NM_003958 |
| RNF4 | E3 ubiquitin ligase | 4p16.3 | NM_001185009 |
| UBE2V2 (MMS2) | Ubiquitin-conjugating complex | 8q11.21 | NM_003350 |
| UBE2N (UBC13) | | 12q22 | NM_003348 |
| USP1 | Ubiquitin-specific protease for FANCD2, PCNA | 1p31.3 | NM_003368 |
| WDR48 | Necessary for USP1 activity | 3p22.2 | NM_020839 |
| HERC2 | Control of several DNA repair factors | 15q13.1 | NM_004667 |

Chromatin Structure and Modification

| | | | |
|---|---|---|---|
| H2AX (H2AFX) | Histone, phosphorylated after DNA damage | 11q23.3 | NM_002105 |
| CHAF1A (CAF1) | Chromatin assembly factor | 19p13.3 | NM_005483 |
| SETMAR (METNASE) | DNA damage-associated histone | 3p26 | NM_006515 |
| | methylase and nuclease | | |
| ATRX | Chromatin remodeling, transcription factor | Xq21.1 | NM_000489 |

Genes defective in diseases associated with sensitivity to DNA damaging agents

| | | | |
|---|---|---|---|
| BLM | Bloom syndrome helicase | 15q26.1 | NM_000057 |
| RMI1 | In BLM-TOP3A complex | 9g21.32 | NM_001358291 |
| TOP3A | Topoisomerase IIIa | 17p11.2 | NM_004618 |
| WRN | Werner syndrome helicase/3′ - exonuclease | 8p12 | NM_000553 |
| RECQL4 | Rothmund-Thompson syndrome | 8q24.3 | NM_004260 |
| ATM | ataxia telangiectasia | 11q22.3 | NM_000051 |
| MPLKIP (TTDN1) | non-photosensitive form of trichothiodystrophy | 7p14 | NM_138701 |

Other identified genes with known or suspected DNA repair function

| | | | |
|---|---|---|---|
| RPA4 | Similar to RPA2 | Xp21.33 | NM_013347 |
| PRPF19 (PSO4) | Pre-mRNA splicing; DNA crosslink repair; | 11q12.2 | NM_014502 |
| | binding to SETMAR | | |
| RECQL (RECQ1) | DNA helicase | 12p12.1 | NM_002907 |
| RECQL5 | DNA helicase | 17q25.1 | NM_001003715 |
| RDM1 (RAD52B) | Similar to RAD52 | 17q12 | NM_145654 |
| NABP2 (SSB1) | Single-stranded DNA binding protein | 12q13.3 | NM_024068 |
| | (ATM/MRN pathway) | | |

Other conserved DNA damage response genes

| | | | |
|---|---|---|---|
| ATR | ATM- and PI-3K-like essential kinase | 3q23 | NM_001184 |
| ATRIP | ATR-interacting protein | 3p21.31 | NM_130384 |
| MDC1 | Mediator of DNA damage checkpoint | 6p21.3 | NM_014641 |
| PCNA | Sliding clamp for pol delta and pol epsilon | 20p12.3 | NM_002592 |
| RAD1 | subunits of PCNA-like sensor of damaged DNA | 5p13.2 | NM_002853 |
| RAD9A | | 11q13.2 | NM_004584 |
| HUS1 | | 7p12.3 | NM_004507 |
| RAD17 (RAD24) | RFC-like DNA damage sensor | 5q13.2 | NM_002873 |
| CHEK1 | Effector kinases | 11q24.2 | NM_001274 |
| CHEK2 | | 22q12.1 | NM_007194 |
| TP53 | Regulation of the cell cycle | 17p13.1 | NM_000546 |
| TP53BP1 (53BP1) | chromatin-binding checkpoint protein | 15q15-q21 | NM_001141980 |
| RIF1 | suppressor of 5′-end-resection | 2q23.3 | NM_001177665 |
| TOPBP1 | DNA damage checkpoint control | 3q22.1 | NM_007027 |
| CLK2 | S-phase check point and biological clock protein | 1q21 | NM_003993 |
| PER1 | S-phase check point and biological clock protein | 17p12 | NM_002616 |

*Some gene products act in more than one pathway, but each is listed only once.

The term “Pol θ inhibitor” as used herein refers to a compound that inhibits human Pol θ activity with a half-maximal inhibitory concentration (IC₅₀) of 100 μM or less. In one embodiment, a Pol θ inhibitor inhibits human Pol θ activity an IC₅₀ of 10 μM or less. In another embodiment, a Pol θ inhibitor inhibits human Pol θ activity with an IC₅₀ of 1 μM or less. In another embodiment, a Pol θ inhibitor inhibits human Pol θ activity with an IC₅₀ of 500 nM or less. In another embodiment, a Pol θ inhibitor inhibits human Pol θ activity with an IC₅₀ of 100 nM or less. In another embodiment, a Pol θ inhibitor inhibits human Pol θ activity with an IC₅₀ of 50 nM or less. In another embodiment, a Pol θ inhibitor inhibits human Pol θ activity with an IC₅₀ of 10 nM or less. A Pol θ inhibitor assay is disclosed in WO2021/123785. Likewise, the term “Pol η inhibitor” as used herein refers to a compound that inhibits human Pol η enzyme activity with a half-maximal inhibitory concentration (IC₅₀) of 100 μM or less, 10 μM or less, 1 μM or less, 500 nM or less, 100 nM or less, 50 nM or less, or 10 nM or less; the term “Pol μ inhibitor” as used herein refers to a compound that inhibits human Pol μ enzyme activity with a half-maximal inhibitory concentration (IC₅₀) of 100 μM or less, 10 μM or less, 1 μM or less, 500 nM or less, 100 nM or less, 50 nM or less, or 10 nM or less; etc.

In some embodiments, phosphorylated Compounds of the Disclosure, see Scheme 1, are DDR enzyme inhibitors. In some embodiments, the triphosphates of Compounds of the Disclosure are DDR enzyme inhibitors. Representative triphosphates (TP) of Compounds of the Disclosure are provided in Table 1A.

TABLE 1A

| Cpd. No. | Structure |
|---|---|
| 1-TP | 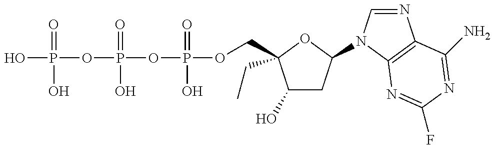 [SMILES: CC[C@]1(COP(=O)(O)OP(=O)(O)OP(=O)(O)O)O[C@@H](n2cnc3c(N)nc(F)nc32)C[C@@H]1O] |
| 2-TP | 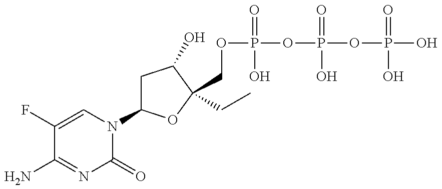 [SMILES: CC[C@]1(COP(=O)(O)OP(=O)(O)OP(=O)(O)O)O[C@@H](n2cc(F)c(N)nc2=O)C[C@@H]1O] |
| 3-TP | 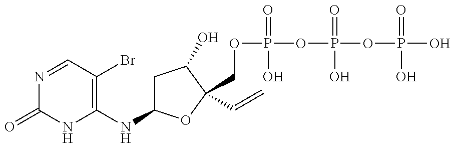 [SMILES: C=C[C@]1(COP(=O)(O)OP(=O)(O)OP(=O)(O)O)O[C@@H](Nc2[nH]c(=O)ncc2Br)C[C@@H]1O] |
| 4-TP | 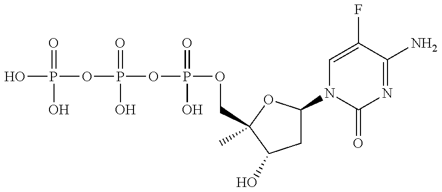 [SMILES: C[C@]1(COP(=O)(O)OP(=O)(O)OP(=O)(O)O)O[C@@H](n2cc(F)c(N)nc2=O)C[C@@H]1O] |
| 5-TP | 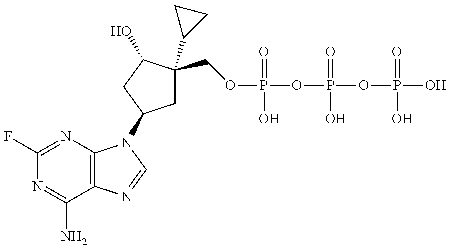 [SMILES: Nc1nc(F)nc2c1ncn2[C@H]1C[C@H](O)[C@](COP(=O)(O)OP(=O)(O)OP(=O)(O)O)(C2CC2)C1] |
| 6-TP | 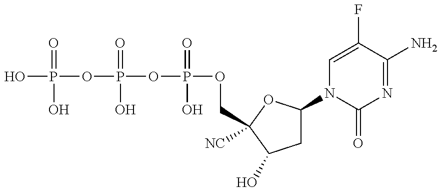 [SMILES: N#C[C@]1(COP(=O)(O)OP(=O)(O)OP(=O)(O)O)O[C@@H](n2cc(F)c(N)nc2=O)C[C@@H]1O] |

The terms “patient” and “subject” as used herein are synonymous terms referring to any human or animal that is in need of or might benefit from administration of a Compound of the Disclosure for treating cancer. Foremost among such subjects are mammals, e.g., humans, although the methods and compositions provided herein are not intended to be so limited. Other subjects include veterinary animals, e.g., cows, sheep, pigs, horses, dogs, cats and the like. In one embodiment, the subject is a human. In one embodiment, the subject is an animal.

The term “biological sample” as used herein refers any tissue or fluid from a subject that is suitable for detecting a biomarker. Examples of useful biological samples include, but are not limited to, biopsied tissues and/or cells, e.g., lymph gland, inflamed tissue, tissue and/or cells involved in a condition or disease, blood, plasma, serous fluid, cerebrospinal fluid, saliva, urine, lymph, cerebral spinal fluid, and the like. Other suitable biological samples will be familiar to those of ordinary skill in the relevant arts. A biological sample can be analyzed for the expression level of a biological compound, e.g., Pol θ, using any technique known in the art. Such techniques include, but are not limited to, polymerase chain reaction (PCR) methodology, reverse transcription-polymerase chain reaction (RT-PCR) methodology, or cytoplasmic light chain immunofluorescence combined with fluorescence in situ hybridization (cIg-FISH). A biological sample can be obtained using techniques that are well within the scope of ordinary knowledge of a clinical practitioner. In one embodiment of the disclosure, the biological sample comprises a tissue or blood sample.

The terms “a”, “an”, “the”, and similar referents in the context of describing the disclosure (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language, e.g., “such as,” provided herein, is intended to better illustrate the disclosure and is not a limitation on the scope of the disclosure unless otherwise claimed. No language in the specification should be construed as indicating any non-claimed element as essential to the practice of the disclosure.

The term “about,” as used herein, includes the recited number±10%. Thus, “about 10” means 9 to 11.

As used herein, the terms “treat,” “treating,” “treatment,” and the like refer to eliminating, reducing, or ameliorating cancer, and/or the symptoms associated therewith. Although not precluded, treating cancer does not require that the cancer, and/or symptom(s) associated therewith be completely eliminated. However, in one embodiment, administration of a Compound of the Disclosure leads to complete elimination of the cancer and associated symptoms.

As used herein, the terms “prevent,” “preventing,” “prevention” and the like refer to a method of preventing the onset of cancer and/or symptom(s) associated therewith, or barring a subject from acquiring the cancer. The terms “prevent,” “preventing,” and “prevention” also include delaying the onset of cancer and/or its attendant symptom(s), and reducing a subject's risk of acquiring the cancer. The terms “prevent,” “preventing” and “prevention” also includes “prophylactic treatment,” which refers to reducing the probability of redeveloping the cancer, or of a recurrence of a previously-controlled cancer, in a subject who does not have, but is at risk of or is susceptible to, redeveloping the cancer or a recurrence of the cancer. The terms “prevent,” “preventing” and “prevention” also include delaying or reversing the progression of the underlying pathology of the cancer.

The term “therapeutically effective amount,” as used herein, refers to that amount of a Compound of the Disclosure and, optionally, one or more optional therapeutic agents sufficient to result in amelioration of one or more symptoms of cancer, or prevent advancement of a cancer, or cause regression of cancer. For example, a therapeutically effective amount will refer to the amount of a Compound of the Disclosure that causes a therapeutic response or endpoint, e.g., delay the progression of cancer or increase relapse-free survival in subject by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, or more.

The term “container” means any receptacle and closure therefore suitable for storing, shipping, dispensing, and/or handling the Compound of the Disclosure. Non-limiting exemplary containers include vials, ampules, bottles, and syringes.

The term “insert” means information accompanying a pharmaceutical product that provides a description of how to administer the product, along with the safety and efficacy data required to allow the physician, pharmacist, and subject to make an informed decision regarding use of the product. The package insert generally is regarded as the “label” for a pharmaceutical product.

In some embodiments, when administered in combination, a Compound of the Disclosure and an optional therapeutic agent can have a synergistic effect. The terms “synergy,” “synergistic,” “synergistically” and derivations thereof, such as in a “synergistic effect” or a “synergistic combination” or a “synergistic composition” as used herein refer to circumstances under which the biological activity of a combination of an agent and at least one additional therapeutic agent is greater than the sum of the biological activities of the respective agents when administered individually. For example, the term “synergistically effective” as used herein refers to the interaction between a Compound of the Disclosure and another therapeutic agent that causes the total effect of the drugs to be greater than the sum of the individual effects of each drug. Berenbaum, Pharmacological Reviews 41:93-141 (1989).

The phrase “in combination” as used in connection with the administration of a Compound of the Disclosure and one or more optional therapeutic agents to a subject means that the Compound of the Disclosure and the one or more optional therapeutic agents can be administered to the subject together, e.g., as part of a single pharmaceutical composition or formulation, or separately, e.g., as part of two or more separate pharmaceutical compositions or formulations. The phrase “in combination” as used in connection with the administration of a Compound of the Disclosure and the one or more optional therapeutic agents to a subject is thus intended to embrace administration of the Compound of the Disclosure and the one or more optional therapeutic agents in a sequential manner, wherein the Compound of the Disclosure and the one or more optional therapeutic agents are administered to the subject at a different time, as well as administration concurrently, or in a substantially simultaneous manner, e.g., less than 30 minutes apart. Simultaneous administration can be accomplished, for example, by administering to the subject a single capsule having a fixed ratio of each of the Compound of the Disclosure and the one or more optional therapeutic agents or in multiple, single capsules for each of the Compound of the Disclosure and the one or more optional therapeutic agents. Sequential or substantially simultaneous administration of the Compound of the Disclosure and the one or more optional therapeutic agents can be accomplished by any appropriate route including, but not limited to, oral routes, intravenous routes, subcutaneous routes, intramuscular routes, etc. The Compound of the Disclosure and the one or more optional therapeutic agents can be administered by the same route or by different routes. For example, the one or more optional therapeutic agents and the Compound of the Disclosure of the combination may be administered orally. Alternatively, for example, the Compound of the Disclosure may be administered orally and the one or more optional therapeutic agents may be administered by intravenous injection. The Compound of the Disclosure and the one or more optional therapeutic agents may also be administered in alternation. In one embodiment, the Compound of the Disclosure and the one or more optional therapeutic agents are administered to a subject separately, e.g., as part of two or more separate pharmaceutical compositions or formulations.

### VII. Particular Embodiments

The present disclosure provides the following particular embodiments.

### A. Compounds of the Disclosure for Treating Cancer.

Embodiment 1. A method for treating cancer in subject in need thereof, the method comprising administering a therapeutically effective amount of a Compound of the Disclosure to the subject.

Embodiment 2. The method of Embodiment 1, wherein the Compound of the Disclosure is a compound of Table 1, or a pharmaceutically acceptable salt or solvate thereof, or tautomer thereof.

Embodiment 3. The method of Embodiment 1, wherein the Compound of the Disclosure is a compound of Table 2, or a pharmaceutically acceptable salt or solvate thereof, or tautomer thereof.

Embodiment 4. The method of Embodiment 1, wherein the Compound of the Disclosure is a compound of Table 3, or a pharmaceutically acceptable salt or solvate thereof, or tautomer thereof.

Embodiment 5. The method of any one of Embodiments 1-4, wherein the cancer is breast cancer, colon cancer, lung cancer, pancreatic ductal cancer, prostate cancer, ovarian cancer, or head and neck cancer.

Embodiment 6. The method of Embodiment 5, wherein the cancer is breast cancer.

Embodiment 7. The method of Embodiment 5, wherein the cancer is colon cancer.

Embodiment 8. The method of Embodiment 5, wherein the cancer is lung cancer.

Embodiment 9. The method of Embodiment 5, wherein the cancer is pancreatic ductal cancer.

Embodiment 10. The method of Embodiment 5, wherein the cancer is prostate cancer.

Embodiment 11. The method of Embodiment 5, wherein the prostate cancer is high-risk localized prostate cancer.

Embodiment 12. The method of Embodiment 5, wherein the cancer is ovarian cancer.

Embodiment 13. The method of Embodiment 5, wherein the cancer is head and neck cancer.

Embodiment 14. The method of any one of Embodiments 1-5, wherein the subject has prostate cancer and the Compound of the Disclosure is administered as an adjuvant therapy.

Embodiment 15. The method of any one of Embodiments 1-5, wherein the subject has prostate cancer and the Compound of the Disclosure is administered as a neoadjuvant therapy.

Embodiment 16. The method of any one of Embodiments 1-15 further comprising administering a therapeutically effective amount of at least one second therapeutic agent useful for treating the cancer.

Embodiment 17. The method of Embodiment 16, wherein the cancer is breast cancer and the at least one second therapeutic agent is Soltamox® (tamoxifen), Arimidex® (anastrozole), Femara® (letrozole), Aromasin® (exemestane), Herceptin® (trastuzumab), Abraxane® (paclitaxel), Cytoxan® (cyclophosphamide), Taxol® (paclitaxel), Afinitor® (everolimus), Taxotere® (docetaxel), Xeloda® (capecitabine), Trexall® (methotrexate), Faslodex (fulvestrant), Adriamycin® (doxorubicin), Perjeta® (pertuzumab), Gemzar (gemcitabine), Tykerb® (lapatinib), Adrucil® (fluorouracil), Ibrance® (palbociclib), Verzenio® (abemaciclib), Fareston® (toremifene), Halaven® (eribulin), Menest, Kadcyla® (ado-trastuzumab emtransine), Androxy® (fluoxymesterone), Avastin® (bevacizumab), esterified estrogens, Herzuma® (trastuzumab), Ixempra® (ixabepilone), Kanjinti® (trastuzumab), Kisqali® (ribociclib), Ogivri® (trastuzumab), Ontruzant® (trastuzumab), Tepadina® (thiotepa), Trazimera® (trastuzumab), Velban® (vinblastine), Piqray® (alpelisib), Tecentriq® (atezolizumab), Enhertu® (fam-trastuzumab deruxtecan), Herceptin, Hylecta™ (hyaluronidase/trastuzumab), Infugem® (gemcitabine), Kisqali® Femara® Co-Pack (ribociclib and letrozole), Talzenna® (talazoparib), Trodelvy® (sacituzumab) or Tukysa™ (tukatinib).

Embodiment 18. The method of Embodiment 16, wherein the cancer is colon cancer and the at least one second therapeutic agent is Xeloda® (capecitabine), Eloxatin® (oxaliplatin), fluorouracil, Avastin® (bevacizumab), leucovorin, Camptosar® (irinotecan), Stivarga® (regorafenib), Erbitux® (cetuximab), Vectibix® (panitumumab), Lonsurf® (tipiracil/trifluridine), Zaltrap® (ziv-aflibercept), Betaseron® (interferon beta-1b), Fusilev® (levoleucovorin), Wellcocorin® (methotrexate), Keytruda® (pembrolizumab), Mvasi® (bevacizumab-awwb), Cyramza® (ramucirumab), Yervoy® (ipilmumab), Opdivo® (nivolumab), Braflovi® (encorafenib), Khapzory® (levoleucovorin) orZirabev® (bevacizumab-bvzr).

Embodiment 19. The method of Embodiment 16, wherein the cancer is lung cancer and the at least one second therapeutic agent is Etopophos® (etoposide), Hycamtin® (topotecan), VePesid® (etoposide), Toposar® (etoposide), Opdivo® (nivolumab), Keytruda® (pembrolizumab), Tecentriq® (atezolizumab), Imfinizi® (durvalumab), methotrexate, cyclophosphamide, Carboplatin, Cisplatin, docetaxel, Gemcitabine, Irinotecan, Paclitaxel, Pemetrexed, Vinblastine, or Vinorelbine.

Embodiment 20. The method of Embodiment 16, wherein the cancer is pancreatic ductal cancer and the at least one second therapeutic agent is Gemzar® (Gemcitabine), fluorouracil, Afinitor® (everolimus), Tarceva® (erlotinib), Abraxane® (paclitaxel), capecitabine, Sutent® (sunitinib), pancreatin, methotrexate, Zanosar® (streptozocin), Mutamycin® (mitomycin), Onivyde® (irinotecan), bevacizumab, cetuximab, Infugem® (gemcitabine) or Lynparza® (olaparib).

Embodiment 21. The method of Embodiment 16, wherein the cancer is head and neck cancer and the at least one second therapeutic agent is Erbituz® (cetuximab), Taxotere® (docetaxel), Trexall® (methotrexate), Keytruda® (pembrolizumab) or Opdivo® (nivolumab).

Embodiment 22. The method of Embodiment 16, wherein the cancer is prostate cancer and the at least one second therapeutic agent is Suprefact® (buserelin), Firmagon® (degarelix), Zoladex® (goserelin), Vantas® (histrelin), Eligard® (leuprolide), Orgovyx® (relugolix), Trelstar® (triptorelin), Casodex® (bicalutamide), Eulexin® (flutamide), Nilandron® (nilutamide), Zytiga® (biraterone acetate), Erleada® (apalutamide), or Xtandi® (enzalutamide).

Embodiment 23. The method of Embodiment 16, wherein the at least one second therapeutic agent is a STING agonist.

Embodiment 24. The method of any one of Embodiments 1-23, wherein the subject is (a) not infected with the HIV virus, (b) not suspected of being infected with the HIV virus, (c) not being treated for the HIV virus, and/or (d) not being treated to prevent the HIV virus.

Embodiment 25. A kit for carrying out the method of any one Embodiments 1-24, the kit comprising (i) a Compound of the Disclosure; and (ii) and instructions for administering the Compound of the Disclosure to a subject having cancer.

Embodiment 26. The kit of Embodiment 25 further comprising at least one second therapeutic agent.

Embodiment 27. The method of any one of Embodiments 1-24, wherein the cells of the cancer are suspected to or exhibit deficiency of a DDR enzyme, e.g., one or more DDR enzymes of Table C, e.g., Pol η, Pol μ, and/or Pol θ, or the cells of the cancer are suspected to or exhibit amplification of a DDR gene, e.g., one or more DDR genes of Table C, e.g., POLH, POLM, and/or POLQ.

### B. Compounds of the Disclosure for Use in Treating Cancer

Embodiment 1. A Compound of the Disclosure for use in treating cancer in a subject.

Embodiment 2. The Compound of the Disclosure for use of Embodiment 1, wherein the Compound of the Disclosure is a compound of Table 1, or a pharmaceutically acceptable salt or solvate thereof, or tautomer thereof.

Embodiment 3. The Compound of the Disclosure for use of Embodiment 1, wherein the Compound of the Disclosure is a compound of Table 2, or a pharmaceutically acceptable salt or solvate thereof, or tautomer thereof.

Embodiment 4. The Compound of the Disclosure for use of Embodiment 1, wherein the Compound of the Disclosure is a compound of Table 3, or a pharmaceutically acceptable salt or solvate thereof, or tautomer thereof.

Embodiment 5. The Compound of the Disclosure for use of any one of Embodiments 1-4, wherein the cancer is breast cancer, colon cancer, lung cancer, pancreatic ductal cancer, prostate cancer, ovarian cancer, or head and neck cancer.

Embodiment 6. The Compound of the Disclosure for use of Embodiment 5, wherein the cancer is breast cancer.

Embodiment 7. The Compound of the Disclosure for use of Embodiment 5, wherein the cancer is colon cancer.

Embodiment 8. The Compound of the Disclosure for use of Embodiment 5, wherein the cancer is lung cancer.

Embodiment 9. The Compound of the Disclosure for use of Embodiment 5, wherein the cancer is pancreatic ductal cancer.

Embodiment 10. The Compound of the Disclosure for use of Embodiment 5, wherein the cancer is prostate cancer.

Embodiment 11. The Compound of the Disclosure for use of Embodiment 5, wherein the prostate cancer is high-risk localized prostate cancer.

Embodiment 12. The Compound of the Disclosure for use of Embodiment 5, wherein the cancer is ovarian cancer.

Embodiment 13. The Compound of the Disclosure for use of Embodiment 5, wherein the cancer is head and neck cancer.

Embodiment 14. The Compound of the Disclosure for use of any one of Embodiments 1-5, wherein the subject has prostate cancer and the Compound of the Disclosure is administered as an adjuvant therapy.

Embodiment 15. The Compound of the Disclosure for use of any one of Embodiments 1-5, wherein the subject has prostate cancer and the Compound of the Disclosure is administered as a neoadjuvant therapy.

Embodiment 16. The Compound of the Disclosure for use of any one of Embodiments 1-15 further comprising administering a therapeutically effective amount of at least one second therapeutic agent useful for treating the cancer.

Embodiment 17. The Compound of the Disclosure for use of Embodiment 16, wherein the cancer is breast cancer and the at least one second therapeutic agent is Soltamox® (tamoxifen), Arimidex® (anastrozole), Femara® (letrozole), Aromasin® (exemestane), Herceptin® (trastuzumab), Abraxane® (paclitaxel), Cytoxan® (cyclophosphamide), Taxol® (paclitaxel), Afinitor® (everolimus), Taxotere® (docetaxel), Xeloda® (capecitabine), Trexall® (methotrexate), Faslodex (fulvestrant), Adriamycin® (doxorubicin), Perjeta® (pertuzumab), Gemzar (gemcitabine), Tykerb® (lapatinib), Adrucil® (fluorouracil), Ibrance® (palbociclib), Verzenio® (abemaciclib), Fareston® (toremifene), Halaven® (eribulin), Menest, Kadcyla® (ado-trastuzumab emtransine), Androxy® (fluoxymesterone), Avastin® (bevacizumab), esterified estrogens, Herzuma® (trastuzumab), Ixempra® (ixabepilone), Kanjinti® (trastuzumab), Kisqali® (ribociclib), Ogivri® (trastuzumab), Ontruzant® (trastuzumab), Tepadina® (thiotepa), Trazimera® (trastuzumab), Velban® (vinblastine), Piaray® (alpelisib), Tecentriq® (atezolizumab), Enhertu® (fam-trastuzumab deruxtecan), Herceptin, Hylecta™ (hyaluronidase/trastuzumab), Infugem® (gemcitabine), Kisqali® Femara® Co-Pack (ribociclib and letrozole), Talzenna® (talazoparib), Trodelvy® (sacituzumab) or Tukysa™ (tukatinib).

Embodiment 18. The Compound of the Disclosure for use of Embodiment 16, wherein the cancer is colon cancer and the at least one second therapeutic agent is Xeloda® (capecitabine), Eloxatin® (oxaliplatin), fluorouracil, Avastin® (bevacizumab), leucovorin, Camptosar® (irinotecan), Stivarga® (regorafenib), Erbitux® (cetuximab), Vectibix® (panitumumab), Lonsurf® (tipiracil/trifluridine), Zaltrap® (ziv-aflibercept), Betaseron® (interferon beta-1b), Fusilev® (levoleucovorin), Wellcocorin® (methotrexate), Keytruda® (pembrolizumab), Mvasi® (bevacizumab-awwb), Cyramza® (ramucirumab), Yervoy® (ipilmumab), Opdivo® (nivolumab), Braftovi® (encorafenib), Khapzory® (levoleucovorin) or Zirabev® (bevacizumab-bvzr).

Embodiment 19. The Compound of the Disclosure for use of Embodiment 16, wherein the cancer is lung cancer and the at least one second therapeutic agent is Etopophos® (etoposide), Hycamtin® (topotecan), VePesid® (etoposide), Toposar® (etoposide), Opdivo® (nivolumab), Keytruda® (pembrolizumab), Tecentriq® (atezolizumab), Imfinizi® (durvalumab), methotrexate, cyclophosphamide, Carboplatin, Cisplatin, docetaxel, Gemcitabine, Irinotecan, Paclitaxel, Pemetrexed, Vinblastine, or Vinorelbine.

Embodiment 20. The Compound of the Disclosure for use of Embodiment 16, wherein the cancer is pancreatic ductal cancer and the at least one second therapeutic agent is Gemzar® (Gemcitabine), fluorouracil, Afinitor® (everolimus), Tarceva® (erlotinib), Abraxane® (paclitaxel), capecitabine, Sutent® (sunitinib), pancreatin, methotrexate, Zanosar® (streptozocin), Mutamycin® (mitomycin), Onivyde® (irinotecan), bevacizumab, cetuximab, Infugem® (gemcitabine) or Lynparza® (olaparib).

Embodiment 21. The Compound of the Disclosure for use of Embodiment 16, wherein the cancer is head and neck cancer and the at least one second therapeutic agent is Erbituz® (cetuximab), Taxotere® (docetaxel), Trexall® (methotrexate), Keytruda® (pembrolizumab) or Opdivo® (nivolumab).

Embodiment 22. The Compound of the Disclosure for use of Embodiment 16, wherein the cancer is prostate cancer and the at least one second therapeutic agent is Suprefact® (buserelin), Firmagon® (degarelix), Zoladex® (goserelin), Vantas® (histrelin), Eligard® (leuprolide), Orgovyx® (relugolix), Trelstar® (triptorelin), Casodex® (bicalutamide), Eulexin® (flutamide), Nilandron® (nilutamide), Zytiga® (biraterone acetate), Erleada® (apalutamide), or Xtandi® (enzalutamide).

Embodiment 23. The Compound of the Disclosure for use of Embodiment 16, wherein the at least one second therapeutic agent is a STING agonist.

Embodiment 24. The Compound of the Disclosure for use of any one of Embodiments 1-23, wherein the subject is (a) not infected with the HIV virus, (b) not suspected of being infected with the HIV virus, (c) not being treated for the HIV virus, and/or (d) not being treated to prevent the HIV virus.

Embodiment 25. The Compound of the Disclosure for use of any one of Embodiments 1-24, wherein the cells of the cancer are suspected to or exhibit deficiency of a DDR enzyme, e.g., one or more DDR enzymes of Table C, e.g., Pol η, Pol μ, and/or Pol θ, or the cells of the cancer are suspected to or exhibit amplification of a DDR gene, e.g., one or more DDR genes of Table C, e.g., POLH, POLM, and/or POLQ.

### C. Compounds of the Disclosure in the Manufacture of a Medicament to Treat Cancer

Embodiment 1. Use of a Compound of the Disclosure in the manufacture of a medicament for treating cancer in a subject.

Embodiment 2. The use of Embodiment 1, wherein the Compound of the Disclosure is a compound of Table 1, or a pharmaceutically acceptable salt or solvate thereof, or tautomer thereof.

Embodiment 3. The use of Embodiment 1, wherein the Compound of the Disclosure is a compound of Table 2, or a pharmaceutically acceptable salt or solvate thereof, or tautomer thereof.

Embodiment 4. The use of Embodiment 1, wherein the Compound of the Disclosure is a compound of Table 3, or a pharmaceutically acceptable salt or solvate thereof, or tautomer thereof.

Embodiment 5. The use of any one of Embodiments 1-4, wherein the cancer is breast cancer, colon cancer, lung cancer, pancreatic ductal cancer, prostate cancer, ovarian cancer, or head and neck cancer.

Embodiment 6. The use of Embodiment 5, wherein the cancer is breast cancer.

Embodiment 7. The use of Embodiment 5, wherein the cancer is colon cancer.

Embodiment 8. The use of Embodiment 5, wherein the cancer is lung cancer.

Embodiment 9. The use of Embodiment 5, wherein the cancer is pancreatic ductal cancer.

Embodiment 10. The use of Embodiment 5, wherein the cancer is prostate cancer.

Embodiment 11. The use of Embodiment 5, wherein the prostate cancer is high-risk localized prostate cancer.

Embodiment 12. The use of Embodiment 5, wherein the cancer is ovarian cancer.

Embodiment 13. The use of Embodiment 5, wherein the cancer is head and neck cancer.

Embodiment 14. The use of any one of Embodiments 1-5, wherein the subject has prostate cancer and the Compound of the Disclosure is administered as an adjuvant therapy.

Embodiment 15. The use of any one of Embodiments 1-5, wherein the subject has prostate cancer and the Compound of the Disclosure is administered as a neoadjuvant therapy.

Embodiment 16. The use of any one of Embodiments 1-15 further comprising administering a therapeutically effective amount of at least one second therapeutic agent useful for treating the cancer.

Embodiment 17. The use of Embodiment 16, wherein the cancer is breast cancer and the at least one second therapeutic agent is Soltamox® (tamoxifen), Arimidex® (anastrozole), Femara® (letrozole), Aromasin® (exemestane), Herceptin® (trastuzumab), Abraxane® (paclitaxel), Cytoxan® (cyclophosphamide), Taxol® (paclitaxel), Afinitor® (everolimus), Taxotere® (docetaxel), Xeloda® (capecitabine), Trexall® (methotrexate), Faslodex (fulvestrant), Adriamycin® (doxorubicin), Perjeta® (pertuzumab), Gemzar (gemcitabine), Tykerb® (lapatinib), Adrucil® (fluorouracil), Ibrance® (palbociclib), Verzenio® (abemaciclib), Fareston® (toremifene), Halaven® (eribulin), Menest, Kadcyla® (ado-trastuzumab emtransine), Androxy® (fluoxymesterone), Avastin® (bevacizumab), esterified estrogens, Herzuma® (trastuzumab), Ixempra® (ixabepilone), Kanjinti® (trastuzumab), Kisqali® (ribociclib), Ogivri® (trastuzumab), Ontruzant® (trastuzumab), Tepadina® (thiotepa), Trazimera® (trastuzumab), Velban® (vinblastine), Piqray® (alpelisib), Tecentriq® (atezolizumab), Enhertu® (fam-trastuzumab deruxtecan), Herceptin, Hylecta™ (hyaluronidase/trastuzumab), Infugem® (gemcitabine), Kisqali® Femara® Co-Pack (ribociclib and letrozole), Talzenna® (talazoparib), Trodelvy® (sacituzumab) or Tukysa™ (tukatinib).

Embodiment 18. The use of Embodiment 16, wherein the cancer is colon cancer and the at least one second therapeutic agent is Xeloda® (capecitabine), Eloxatin® (oxaliplatin), fluorouracil, Avastin® (bevacizumab), leucovorin, Camptosar® (irinotecan), Stivarga® (regorafenib), Erbitux® (cetuximab), Vectibix® (panitumumab), Lonsurf® (tipiracil/trifluridine), Zaltrap® (ziv-aflibercept), Betaseron® (interferon beta-1b), Fusilev® (levoleucovorin), Wellcocorin® (methotrexate), Keytruda® (pembrolizumab), Mvasi® (bevacizumab-awwb), Cyramza® (ramucirumab), Yervoy® (ipilmumab), Opdivo® (nivolumab), Braflovi® (encorafenib), Khapzory® (levoleucovorin) or Zirabev® (bevacizumab-bvzr).

Embodiment 19. The use of Embodiment 16, wherein the cancer is lung cancer and the at least one second therapeutic agent is Etopophos® (etoposide), Hycamtin® (topotecan), VePesid® (etoposide), Toposar® (etoposide), Opdivo® (nivolumab), Keytruda® (pembrolizumab), Tecentriq® (atezolizumab), Imfinizi® (durvalumab), methotrexate, cyclophosphamide, Carboplatin, Cisplatin, docetaxel, Gemcitabine, Irinotecan, Paclitaxel, Pemetrexed, Vinblastine, or Vinorelbine.

Embodiment 20. The use of Embodiment 16, wherein the cancer is pancreatic ductal cancer and the at least one second therapeutic agent is Gemzar® (Gemcitabine), fluorouracil, Afinitor® (everolimus), Tarceva® (erlotinib), Abraxane® (paclitaxel), capecitabine, Sutent® (sunitinib), pancreatin, methotrexate, Zanosar® (streptozocin), Mutamycin® (mitomycin), Onivyde® (irinotecan), bevacizumab, cetuximab, Infugem® (gemcitabine) or Lynparza® (olaparib).

Embodiment 21. The use of Embodiment 16, wherein the cancer is head and neck cancer and the at least one second therapeutic agent is Erbituz® (cetuximab), Taxotere® (docetaxel), Trexall® (methotrexate), Keytruda® (pembrolizumab) or Opdivo® (nivolumab).

Embodiment 22. The use of Embodiment 16, wherein the cancer is prostate cancer and the at least one second therapeutic agent is Suprefact® (buserelin), Firmagon® (degarelix), Zoladex® (goserelin), Vantas® (histrelin), Eligard® (leuprolide), Orgovyx® (relugolix), Trelstar® (triptorelin), Casodex® (bicalutamide), Eulexin® (flutamide), Nilandron® (nilutamide), Zytiga® (biraterone acetate), Erleada® (apalutamide), or Xtandi® (enzalutamide).

Embodiment 23. The use of Embodiment 16, wherein the at least one second therapeutic agent is a STING agonist.

Embodiment 24. The use of any one of Embodiments 1-23, wherein the subject is (a) not infected with the HIV virus, (b) not suspected of being infected with the HIV virus, (c) not being treated for the HIV virus, and/or (d) not being treated to prevent the HIV virus.

Embodiment 25. The use of any one of Embodiments 1-24, wherein the cells of the cancer are suspected to or exhibit deficiency of a DDR enzyme, e.g., one or more DDR enzymes of Table C, e.g., Pol η, Pol μ, and/or Pol θ, or the cells of the cancer are suspected to or exhibit amplification of a DDR gene, e.g., one or more DDR genes of Table C, e.g., POLH, POLM, and/or POLQ.

### D. Compounds of the Disclosure to Treat Metastatic Prostate Cancer

Embodiment 1. A method of treating metastatic prostate cancer in a subject, wherein the cancer is resistant or may become resistant to androgen deprivation therapy (ADT), comprising administering to the subject an effective amount of a Compound of the Disclosure.

Embodiment 2. The method of Embodiment 1, further comprising administering at least one second agent that is an ADT.

Embodiment 3. The method of Embodiment 2, wherein the ADT is administered before the Compound of the Disclosure is administered to the subject.

Embodiment 4. The method of Embodiment 2, wherein the ADT is administered after the Compound of the Disclosure is administered to the subject.

Embodiment 5. The method of Embodiment 2, wherein the ADT and the Compound of the Disclosure are administered concomitantly to the subject.

Embodiment 6. The method of any one of Embodiments 2-5, wherein the ADT comprises administration of buserelin, degarelix, goserelin, histrelin, leuprolide, relugolix, triptorelin, bicalutamide, enzalutamide, bicalutamide, apalutamide, darolutamide, nilutamide, flutamide, abiraterone acetate, topilutamide, cyproterone acetate, megestrol acetate, chlormadinone acetate, spironolactone, oxendolone, or osaterone acetate.

Embodiment 7. The method of any one of Embodiments 1-6, further comprising administering at least one third therapeutic agent for the treatment of metastatic prostate cancer.

Embodiment 8. The method of Embodiment 7, wherein the at least one third therapeutic agent that is useful for the treatment of metastatic prostate cancer is a poly ADP ribose polymerase (PARP) inhibitor, an ATM inhibitor, a wee1 inhibitor, or an ATR inhibitor.

Embodiment 9. The method of any one of Embodiments 1-8, wherein the metastatic prostate cancer cells are resistant to PARP inhibition.

Embodiment 10. The method of Embodiment 9, wherein the at least one third therapeutic agent is a PARP inhibitor.

Embodiment 11. The method of Embodiment 10, wherein the PARP inhibitor is olaparib, rucaparib, niraparib or talazoparib.

Embodiment 12. The method of any one of Embodiments 1-11, wherein the Compound of the Disclosure is a compound of Table 1, or a pharmaceutically acceptable salt or solvate thereof, or a tautomer thereof.

Embodiment 13. The method of any one of Embodiments 1-11, wherein the Compound of the Disclosure is a compound of Table 2, or a pharmaceutically acceptable salt or solvate thereof, or a tautomer thereof.

Embodiment 14. The method of any one of Embodiments 1-11, wherein the Compound of the Disclosure is a compound of Table 3, or a pharmaceutically acceptable salt or solvate thereof, or a tautomer thereof.

Embodiment 15. The method of Embodiments 1-14, wherein the cells of the metastatic prostate cancer exhibit deficiency or loss of function of one or more DNA damage repair genes.

Embodiment 16. The method of Embodiment 15, wherein the DNA damage repair genes are BRCA1, BRCA2, PARP2, or POLQ genes.

Embodiment 17. The method of any one of Embodiments 1-16, wherein the subject has metastatic prostate cancer with tumors distant from the prostate.

Embodiment 18. The method of any one of Embodiments 1-17, wherein the subject has previously been treated with ADT, has developed resistance to ADT, and is administered the Compound of the Disclosure.

Embodiment 19. The method of any one of Embodiments 1-18, wherein the subject has not previously been treated with ADT, and is administered the ADT and the Compound of the Disclosure.

Embodiment 20. A kit for carrying out the method of any one Embodiments 1-19, the kit comprising (i) a Compound of the Disclosure; and (ii) and instructions for administering the Compound of the Disclosure to a subject having metastatic prostate cancer, wherein the prostate cancer is resistant or may become resistant to ADT.

Embodiment 21. The kit of Embodiment 20, further comprising instructions for administering at least one second therapeutic agent for the treatment of metastatic prostate cancer comprising at least one ADT.

Embodiment 22. The kit of Embodiment 21, further comprising instructions for administering at least one third therapeutic agent for the treatment of metastatic prostate cancer.

Embodiment 23 The method of any one of Embodiments 1-22, wherein the subject is (a) not infected with the HIV virus, (b) not suspected of being infected with the HIV virus, (c) not being treated for the HIV virus, and/or (d) not being treated to prevent the HIV virus.

### E. Compounds of the Disclosure for Use in Treating Metastatic Prostate Cancer

Embodiment 1. A Compound of the Disclosure for use in treating metastatic prostate cancer in a subject, wherein the cancer is resistant or may become resistant to androgen deprivation therapy (ADT).

Embodiment 2. The Compound of the Disclosure for use of Embodiment 1, wherein the Compound of the Disclosure is to be administered to the subject with at least one second agent that is an ADT.

Embodiment 3. The Compound of the Disclosure for use of Embodiment 2, wherein the ADT is to be administered to the subject before the Compound of the Disclosure.

Embodiment 4. The Compound of the Disclosure for use of Embodiment 2, wherein the ADT is to be administered to the subject after the Compound of the Disclosure.

Embodiment 5. The Compound of the Disclosure for use of Embodiment 2, wherein the ADT and the Compound of the Disclosure are to be administered concomitantly to the subject.

Embodiment 6. The Compound of the Disclosure for use of any one of Embodiments 2-5, wherein the ADT comprises buserelin, degarelix, goserelin, histrelin, leuprolide, relugolix, triptorelin, bicalutamide, enzalutamide, bicalutamide, apalutamide, darolutamide, nilutamide, flutamide, abiraterone acetate, topilutamide, cyproterone acetate, megestrol acetate, chlormadinone acetate, spironolactone, oxendolone, or osaterone acetate.

Embodiment 7. The Compound of the Disclosure for use of any one of Embodiments 1-6, further comprising administering at least one third therapeutic agent for the treatment of metastatic prostate cancer.

Embodiment 8. The Compound of the Disclosure for use of Embodiment 7, wherein the at least one third therapeutic agent that is useful for the treatment of metastatic prostate cancer is a poly ADP ribose polymerase (PARP) inhibitor, an ATM inhibitor, a wee1 inhibitor, or an ATR inhibitor.

Embodiment 9. The Compound of the Disclosure for use of any one of Embodiments 1-8, wherein the metastatic prostate cancer cells are resistant to PARP inhibition.

Embodiment 10. The Compound of the Disclosure for use of Embodiment 9, wherein the at least one third therapeutic agent is a PARP inhibitor.

Embodiment 11. The Compound of the Disclosure for use of Embodiment 10, wherein the PARP inhibitor is olaparib, rucaparib, niraparib or talazoparib.

Embodiment 12. The Compound of the Disclosure for use of any one of Embodiments 1-11, wherein the Compound of the Disclosure is a compound of Table 1, or a pharmaceutically acceptable salt or solvate thereof, or a tautomer thereof.

Embodiment 13. The Compound of the Disclosure for use of any one of Embodiments 1-11, wherein the Compound of the Disclosure is a compound of Table 2, or a pharmaceutically acceptable salt or solvate thereof, or a tautomer thereof.

Embodiment 14. The Compound of the Disclosure for use of any one of Embodiments 1-11, wherein the Compound of the Disclosure is a compound of Table 3, or a pharmaceutically acceptable salt or solvate thereof, or a tautomer thereof.

Embodiment 15. The Compound of the Disclosure for use of Embodiments 1-14, wherein the cells of the metastatic prostate cancer exhibit deficiency or loss of function of one or more DNA damage repair genes.

Embodiment 16. The Compound of the Disclosure for use of Embodiment 15, wherein the DNA damage repair genes are BRCA1, BRCA2, PARP2, or POLQ genes.

Embodiment 17. The Compound of the Disclosure for use of any one of Embodiments 1-16, wherein the subject has metastatic prostate cancer with tumors distant from the prostate.

Embodiment 18. The Compound of the Disclosure for use of any one of Embodiments 1-17, wherein the subject has previously been treated with ADT, has developed resistance to ADT, and is administered the Compound of the Disclosure.

Embodiment 19. The Compound of the Disclosure for use of any one of Embodiments 1-18, wherein the subject has not previously been treated with ADT, and is to be administered the ADT and the Compound of the Disclosure.

### F. Compounds of the Disclosure in the Manufacture of a Medicament for Treating Metastatic Prostate Cancer

Embodiment 1. Use of a Compound of the Disclosure for the manufacture of a medicament for treating metastatic prostate cancer in a subject, wherein the cancer is resistant or may become resistant to androgen deprivation therapy (ADT).

Embodiment 2. The use of Embodiment 1, wherein the Compound of the Disclosure is to be administered to the subject with at least one second agent that is an ADT.

Embodiment 3. The use of Embodiment 2, wherein the ADT is to be administered to the subject before the Compound of the Disclosure.

Embodiment 4. The use of Embodiment 2, wherein the ADT is to be administered to the subject after the Compound of the Disclosure.

Embodiment 5. The use of Embodiment 2, wherein the ADT and the Compound of the Disclosure are to be administered concomitantly to the subject.

Embodiment 6. The use of any one of Embodiments 2-5, wherein the ADT comprises buserelin, degarelix, goserelin, histrelin, leuprolide, relugolix, triptorelin, bicalutamide, enzalutamide, bicalutamide, apalutamide, darolutamide, nilutamide, flutamide, abiraterone acetate, topilutamide, cyproterone acetate, megestrol acetate, chlormadinone acetate, spironolactone, oxendolone, or osaterone acetate.

Embodiment 7. The use of any one of Embodiments 1-6, further comprising administering at least one third therapeutic agent for the treatment of metastatic prostate cancer.

Embodiment 8. The use of Embodiment 7, wherein the at least one third therapeutic agent that is useful for the treatment of metastatic prostate cancer is a poly ADP ribose polymerase (PARP) inhibitor, an ATM inhibitor, a wee1 inhibitor, or an ATR inhibitor.

Embodiment 9. The use of any one of Embodiments 1-8, wherein the metastatic prostate cancer cells are resistant to PARP inhibition.

Embodiment 10. The use of Embodiment 9, wherein the at least one third therapeutic agent is a PARP inhibitor.

Embodiment 11. The use of Embodiment 10, wherein the PARP inhibitor is olaparib, rucaparib, niraparib or talazoparib.

Embodiment 12. The use of any one of Embodiments 1-11, wherein the Compound of the Disclosure is a compound of Table 1, or a pharmaceutically acceptable salt or solvate thereof, or a tautomer thereof.

Embodiment 13. The use of any one of Embodiments 1-11, wherein the Compound of the Disclosure is a compound of Table 2, or a pharmaceutically acceptable salt or solvate thereof, or a tautomer thereof.

Embodiment 14. The use of any one of Embodiments 1-11, wherein the Compound of the Disclosure is a compound of Table 3, or a pharmaceutically acceptable salt or solvate thereof, or a tautomer thereof.

Embodiment 15. The use of Embodiments 1-14, wherein the cells of the metastatic prostate cancer exhibit deficiency or loss of function of one or more DNA damage repair genes.

Embodiment 16. The use of Embodiment 15, wherein the DNA damage repair genes are BRCA1, BRCA2, PARP2, or POLQ genes.

Embodiment 17. The use of any one of Embodiments 1-16, wherein the subject has metastatic prostate cancer with tumors distant from the prostate.

Embodiment 18. The use of any one of Embodiments 1-17, wherein the subject has previously been treated with ADT, has developed resistance to ADT, and is administered the Compound of the Disclosure.

Embodiment 19. The use of any one of Embodiments 1-18, wherein the subject has not previously been treated with ADT, and is to be administered the ADT and the Compound of the Disclosure.

### EXAMPLES

The compounds of Tables 1-3 may be prepared as described in the EXAMPLES below, and, for example, as described in Nomura et al., J. Med. Chem. 42:2901-2908 (1999); Ohrui et al., J. Med. Chem. 43:4516-4525 (2000), JP Patent No. 6767011, and/or U.S. Pat. No. 10,933,067.

The abbreviations in Table 7 may be used in the EXAMPLES.

TABLE 7

| | |
|---|---|
| aq. | aqueous |
| ACN, MeCN, CH₃CN | acetonitrile |
| AcOH | acetic acid |
| AmF | ammonium formate |
| AmBic | ammonium bicarbonate |
| CDC1₃ | chloroform-d |
| calc. | calculated |
| conc. | concentrated |
| DCM | methylene chloride or dichloromethane |
| DMF | dimethylformamide |
| DMSO | dimethylsulfoxide |
| DMSO-d₆ | deuterated dimethylsulfoxide |
| Et | ethyl |
| Et₂O | diethyl ether |
| ESI-MS | electrospray ionisation mass spectrometry |
| EtOAc | ethyl acetate |
| H₂ | hydrogen gas |
| H₂O | water |
| HCl | hydrochloric acid |
| hex. | hexanes |
| hep. | heptanes |
| HPLC | high pressure liquid chromatography |
| K₂CO₃ | potassium carbonate |
| LCMS | liquid chromatography mass spectrometry |
| M | molar |
| Me | methyl |
| MHz | megahertz |
| mg | milligram(s) |
| min. | minutes |
| mL | milliliter(s) |
| mM | millimolar |
| Mmol | millimole |
| MeOH | methanol |
| MeOD | methanol-d₄ |
| MgSO₄ | magnesium sulfate |
| MS | mass spectrometer |
| μg | microgram(s) |
| μL | microliter(s) |
| μM | micromolar |
| μmol | micromole |
| N₂ | nitrogen gas |
| NaHCO₃ | sodium bicarbonate |
| NaI | Sodium iodide |
| Na₂CO₃ | sodium carbonate |
| Na₂SO₄ | sodium sulfate |
| NaOH | sodium hydroxide |
| NaOMe | sodium methoxide |
| Nap | 2-naphthyl |
| NH₄Cl | ammonium chloride |
| ¹H NMR | proton nuclear magnetic resonance |
| ON | overnight |
| Pd/C | palladium on carbon |
| Ph | phenyl |
| psi | pound per square inch |
| quant. | quantitative |
| RT | retention time |
| rt | room temperature |
| r.b.f. (rbf) | round bottom flask |
| sat | saturated |
| THF | tetrahydrofuran |
| TLC | thin layer chromatography |
| UPLC | ultra performance liquid chromatography |
| UV | ultraviolet |
| Prep-HPLC | Preparative HPLC |

The following LC-MS Methods may be used in the EXAMPLES.

Method A: UPLC-MS method: Waters Acquity UPLC CSH C18, 1.8 μm, 2.1×30 mm at 40° C.; 5% to 100%/B in 5.2 minutes; hold 1001/c B for 1.8 minutes, run time=7.0 min, flow 0.9 mL/min; Eluents: A=Milli-Q H₂O+10 mM Ammonium formate pH=3.8, B=MeCN. Waters Acquity UPLC system. UV Detector=Waters Acquity PDA, 198-360 nm. MS Detector=Waters SQD ESI.

Method B: Waters Acquity CSH C18, 3.5 μm, 4.6×30 mm at 40° C.; Iso 5% B for 0.5 min, 5% to 100% B in 5 minutes: hold 100% B for 1.5 minutes, run time=7.0 min, flow 0.9 mL/min; Eluents: A=Milli-Q H₂O+10 mM Ammonium formate pH=3.8; B=MeCN. Waters Alliance 2695 system. UV Detection: Waters 2996 PDA, 198-360 nm. MS Detector: Waters ZQ 2000, ESI

Method C: SHIMADZU LC20-MS2010: MERCK, RP-18e 25-2 mm at 50° C.; 1.5 ML/4 L TFA in water (solvent A) and 0.75 ML/4 LTFA in acetonitrile (solvent B), using the elution gradient 5/6-95% (solvent B) over 0.7 minutes and holding at 95% for 0.4 minutes at a flow rate of 1.5 ml/min, run time=1.5 min; UV Detector=220 nm, 254 nm: MS Detector=ESI

Method D: LCMS-BT: SHIMADZU LC20-MS2020: X bridge Shield RP-18.5 μm, 2.1*50 mm at 50° C.; 0.8 mL/4 L NH₃·H₂O in water (solvent A) and acetonitrile (solvent B), using the elution gradient 0/6-30% (solvent B) over 2 minutes and holding at 30% for 0.48 minutes at a flow rate of 1 ml/min, run time=3 min; UV Detector=220 nm, 254 nm; MS Detector=ESI

Method E: LCMS-AN: Agilent LC1200-MS6110: X timate C18 2.1*30 mm, 3 μm at 50° C.; 1.5 ML/4 L TFA in water (solvent A) and 0.75 ML/4 L TFA in acetonitrile (solvent B), using the elution gradient 0%-60% (solvent B) over 0.9 minutes and holding at 60% for 0.6 minutes at a flow rate of 1.2 ml/min, run time=2 min; UV Detector=220 nm; MS Detector=ESI.

Method F: LCMS-CI: Agilent LC1200-MS6110: Xbridge Shield RP-18.5 um, 2.1*50 mm; at 30° C.; water (4 L)+NH₃·H₂O (0.8 mL) (solvent A) and acetonitrile (solvent B), using the elution gradient 0%-60% (solvent B) over 2.0 minutes and holding at 60% for 0.48 minutes at a flow rate of 1 ml/min, run time=3.0 min; UV Detector=220 nm, 254 nm; MS Detector=ESI.

Method G: UPLC-MS Method: Waters Acquity UPLC CSH C18, 1.7 μm, 2.1×30 mm at 40° C.; 5% to 100% B in 5.2 minutes; hold 100% B for 1.8 minutes, run time=7.0 min, flow 0.9 mL/min; Eluents: A=Milli-Q H₂O+10 mM Ammonium Bicarbonate pH=10; B=MeCN. Waters Acquity UPLC system. UV Detector=Waters Acquity PDA, 198-360 nm. MS Detector=Waters SQD ESI.

Method H: UPLC-MS Method: Waters Acquity UPLC CSH C18, 1.8 μm, 2.1×30 mm at 40° C.; 5% to 100% B in 2.0 minutes; hold 100% B for 0.7 minutes, run time=2.7 min, flow 0.9 mL/min; Eluents: A=Milli-Q H₂O+10 mM Ammonium formate pH=3.8; B=MeCN. Waters Acquity UPLC system. UV Detector=Waters Acquity PDA, 198-360 nm. MS Detector=Waters SQD ESI.

Method I: UPLC-MS Method: Waters Acquity UPLC CSH C18, 1.8 μm, 2.1×30 mm at 40° C.; 5% to 100% B in 2.0 minutes; hold 100% B for 0.7 minutes, run time=2.7 min, flow 0.9 mL/min; Eluents: A=Milli-Q H₂O+10 mM Ammonium bicarbonate pH=10; B=MeCN. Waters Acquity UPLC system. UV Detector=Waters Acquity PDA, 198-360 nm. MS Detector=Waters SQD ESI.

Method J: UPLC-MS Method: Waters Acquity UPLC CSH C18, 1.8 μm, 2.1×30 mm at 40° C.; 5% to 100% B in 2.0 minutes; hold 100% B for 0.7 minutes, run time=2.7 min, flow 0.9 mL/min; Eluents: A=Milli-Q H₂O+10 mM Ammonium bicarbonate pH=10; B=MeCN. Waters Acquity UPLC system. UV Detector=Waters Acquity PDA, 198-360 nm. MS Detector=Waters 3100 ESI.

### Example 1

### Synthesis of (2R,3S,5R)-5-(6-amino-2-fluoro-9H-purin-9-yl)-2-ethyl-2-(hydroxymethyl)tetrahydrofuran-3-ol (Cpd. No. 1)

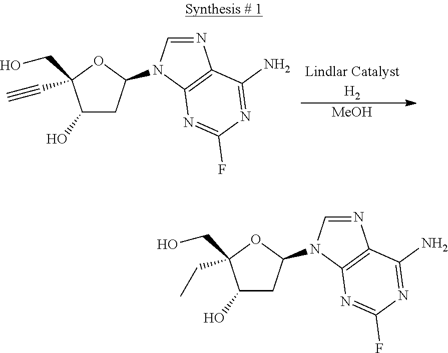 [SMILES: "MeOH" ; C#C[C@]1(CO)O[C@@H](n2cnc3c(N)nc(F)nc32)C[C@@H]1O ; "Lindlar CatalystH2" ; CC[C@]1(CO)O[C@@H](n2cnc3c(N)nc(F)nc32)C[C@@H]1O]

(2R,3S,5R)-5-(6-amino-2-fluoro-9H-purin-9-yl)-2-ethynyl-2-(hydroxymethyl)tetrahydrofuran-3-ol was purchased from MedChemExpess LLC.

(2R,3S,5R)-5-(6-amino-2-fluoro-9H-purin-9-yl)-2-ethynyl-2-(hydroxymethyl)tetrahydrofuran-3-ol (20.7 mg, 70.6 μmol) and Pd (0.416 μl, 48.6 μmol) (10% w/w on activated carbon) were dissolved in MeOH (2.00 mL) at room temperature. Nitrogen atmosphere was bubbled through the solution for 5 min and then hydrogen was bubbled through the solution for 30 min using a balloon. The reaction was sealed and stirred for 1 h at room temperature. Then the resulting mixture was filtered and rinsed with MeOH (10 mL). The solvent was concentrated under reduced pressure and the residue was purified by column chromatography on silica gel (MeOH/DCM 1% to 30%) affording (2R,3S,5R)-5-(6-amino-2-fluoro-9H-purin-9-yl)-2-ethyl-2-(hydroxymethyl)tetrahydrofuran-3-ol (19.0 mg, 91%) as white powder. LC-MS (ESI) m/z calcd for C₁₂H₁₇FN₅O₃: 298.12. Found 298.24 [M−H]−. ¹H NMR (400 MHZ, DMSO-d₆) δ 8.32 (s, 1H), 7.81 (s, 2H), 6.19 (t, J=6.9 Hz, 1H), 5.17 (d, J=4.9 Hz, 1H), 4.92 (t, J=5.5 Hz, 1H), 4.40 (dd, J=8.5, 5.3 Hz, 1H), 3.50 (dd, J=11.5, 5.3 Hz, 1H), 3.41 (dd, J=11.5, 6.0 Hz, 1H), 2.85-2.75 (m, 1H), 2.28 (ddd, J=13.1, 6.1, 3.4 Hz, 1H), 1.72-1.50 (m, 2H), 0.87 (t, J=7.5 Hz, 3H). LC-RT=0.74 min; Method A.

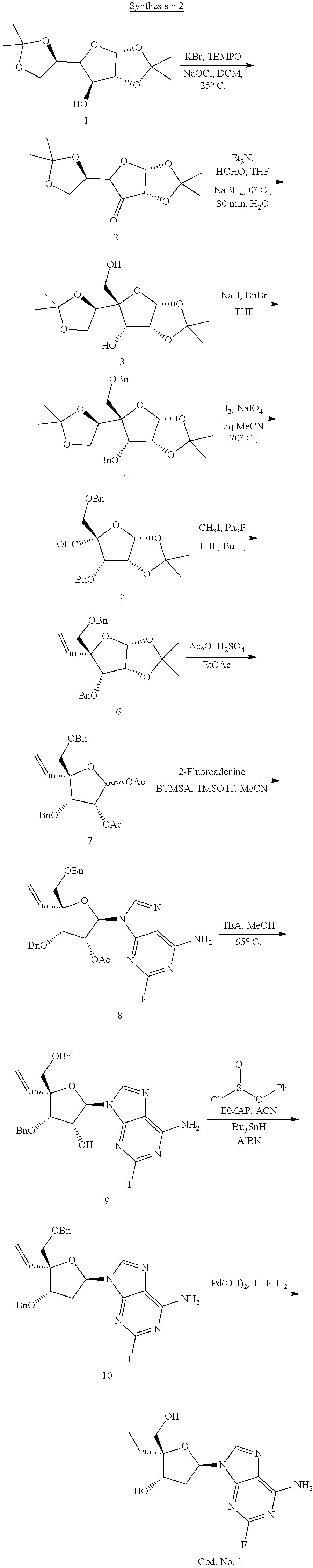

Step 1. Synthesis of (3aR,6aS)-5-((R)-2,2-dimethyl-1,3-dioxolan-4-yl)-2,2-dimethyldihydrofuro[2,3-d][1,3]dioxol-6 (3aH)-one (2). Diacetone-D-glucose (1) (110 g, 414 mmol) was dissolved in CH2Cl (547 mL). TEMPO (1.68 g, 10.6 mmol), potassium bromide (5.08 g, 42.2 mmol) were added to the reaction solution. The resulting mixture was stirred at 25° C. while being stirred, and NaOCl (˜15% aq, 256 mL, 530 mmol) was added over ˜1 h maintaining a temperature of 25-30° C. After finishing addition, the reaction was stirred for more 30 min at the same temperature. The layers were separated, and the organic layer was washed sequentially with a solution of KI (4.5 g in HCl (0.50 M aq, 500 mL), Na₂S₂O₃ (aq, sat, 500 mL), and NaHCO₃[˜5% (w/w) aq, 500 mL]. The combined organics were dried over MgSO₄ and concentrated under reduced pressure. The residue was azeotroped three times by evaporation from toluene (100 mL) to give crude ketone 2 (104 g, 97%) as a brown liquid. NMR (400 MHz, CDCl₃) δ 6.12 (d, J=4.5 Hz, 1H), 4.39-4.35 (m, 1H), 4.35-4.31 (m, 2H), 4.02 (s, 1H), 4.01-3.99 (m, 1H), 1.43 (s, 3H), 1.41 (s, 3H), 1.31 (s, 6H).

Step 2. Synthesis of (3aR,5R,6S,6aR)-5-((R)-2,2-dimethyl-1,3-dioxolan-4-yl)-5-(hydroxymethyl)-2,2-dimethyltetrahydrofuro[2,3-d][1,3]dioxol-6-ol (3). Compound 2 (101 g, 391 mmol) was dissolved in THF (800 mL). To the solution were added Triethylamine (551 mL, 3.91 mol) and formaldehyde (117 g, 3.91 mol, 37% w/w) at room temperature. The mixture was stirred at RT for 16 h. The mixture was adjusted to pH 4-5 using NH₄Cl (aq, sat). After the addition of EtOAc (300 mL) and H₂O (300 mL), the layers were separated. The aqueous layer was extracted twice using EtOAc (300 mL). The combined organics were dried over MgSO₄ and concentrated under reduced pressure. The residue was dissolved into EtOAc (250 mL) at 70° C., and hexane (900 mL) was added dropwise over 30 min. The resulting turbid solution was allowed to cool to RT and left to stirred for 16 h. The solid was collected via filtration, and the filter cake was washed twice with hexane (600 mL) and dried to give a brown solid which was dissolved in MeOH (600 mL). To the solution were added Sodium borohydride (38.3 g, 972 mmol) portion-wise at 0° C. temperature and the resulting mixture was stirred for 30 min. The mixture was quenched with water, remove the MeOH under the vacuum, then the aqueous layer was extracted with DCM (100 mL×3). The combined organic layers were dried by MgSO₄, filtered, concentrated in vacuum, and the residue was triturated with a mixture of hexane and DCM to give a white solid of 3 (71.0 g, 75%). NMR (400 MHz, CDCl₃/D₂O) δ 5.89 (d, J=4.0 Hz, 1H), 4.71 (dd, J=6.2, 4.0 Hz, 1H), 4.60 (dd, J=7.2, 6.8 Hz, 1H), 4.32 (d, J=6.3 Hz, 1H), 4.13 (dd, J=9.3, 7.5 Hz, 1H), 3.88 (dd, J=9.3, 6.5 Hz, 1H), 3.76 (d, J=11.8 Hz, 1H), 3.58 (d, J=11.9 Hz, 1H), 1.61 (s, 3H), 1.44 (s, 3H), 1.38 (s, 3H), 1.32 (s, 3H).

Step 3. Synthesis of (3aR,5S,6S,6aR)-6-(benzyloxy)-5-((benzyloxy)methyl)-5-((R)-2,2-dimethyl-1,3-dioxolan-4-yl)-2,2-dimethyltetrahydrofuro[2,3-d][1,3]dioxole (4). To a stirred suspension of Sodium hydride 60% in dispersion in mineral oil (13.7 g, 342 mmol) in THF (228 mL) was added dropwise a solution of 3 (33.0 g, 114 mmol) in THF (55 mL) at 0° C. under a nitrogen atmosphere. After 10 min of stirring, Benzyl bromide (41.3 mL, 341 mmol) was added and the resulting mixture was heated slowly to reflux temperature and stirred for 30 min. The mixture was poured into cold water slowly and extracted with EtOAc. The extract was washed with brine, dried (MgSO₄), and concentrated in vacuum. The residue was passed through a pad of silica gel using hexane (to remove BnBr) and then EtOAc (to get desired product) as eluents. The EtOAc solution was concentrated in vacuum, gives the desired product 4 (50.0 g, 93%). NMR (400 MHz, CDCl₃) δ 7.37-7.26 (m, 8H), 7.24 (t, J=4.1 Hz, 2H), 5.82 (d, J=3.8 Hz, 1H), 4.81-4.71 (m, 2H), 4.66 (dd, J=5.2, 3.9 Hz, 1H), 4.53 (d, J=11.8 Hz, 1H), 4.47-4.38 (m, 2H), 4.25 (d, J=5.3 Hz, 1H), 4.15 (d, J=1.5 Hz, 1H), 4.04 (dd, J=9.3, 7.6 Hz, 1H), 3.76 (dd, J=9.3, 6.4 Hz, 1H), 3.67 (d, J=10.5 Hz, 1H), 3.57 (d, J=10.5 Hz, 1H), 1.60 (s, 3H), 1.39 (s, 3H), 1.36 (s, 3H, 1.29 (s, 3H).

Step 4. Synthesis of ((3aR,5R,6S,6aR)-6-(benzyloxy)-5-((benzyloxy)methyl)-2,2-dimethyltetrahydrofuro[2,3-d][1,3]dioxole-5-carbaldehyde (5). Compound 4 (9.65 g, 20.5 mmol) was dissolved in ACN (615 mL). To the solution was successively added iodine (2.17 g, 8.53 mmol) and sodium periodate (11.6 g, 53.9 mmol) and H₂O (20.5 mL) at room temperature, and the resulting mixture was stirred at 70° C. for 5 h. The mixture was diluted with EtOAc (100 mL) and filtered through a filter. The filter cake was washed with EtOAc (100 mL) and the washing was combined with the filtrate. The resulting EtOAc solution was mixed with aq Na₂S₂O₃ (30 w/v %, 10 mL) and the organic layer was separated. The aqueous layer was extracted with EtOAc (20 mL×2), and the combined organic layer and extracts were washed with brine, dried (MgSO₄) and concentrated in vacuum to give yellow color oil of 5 (7.75 g, 95%). this material was carried over to the next step without further purification. NMR (400 MHz, CDCl₃) δ 9.90 (s, 1H), 7.34-7.28 (m, 8H), 7.24-7.20 (m, 2H), 5.83 (d, J=3.4 Hz, 1H), 4.70 (d, J=12.1 Hz, 1H), 4.58 (dd, J=7.8, 4.2 Hz, 2H), 4.48 (q, J=12.0 Hz, 2H), 4.36 (d, J=4.4 Hz, 1H), 3.67 (d, J=11.0 Hz, 1H), 3.60 (d, J=11.0 Hz, 1H), 1.59 (s, 3H), 1.33 (s, 3H).

Step 5. Synthesis of (3aR,5R,6S,6aR)-6-(benzyloxy)-5-((benzyloxy)methyl)-2,2-dimethyl-5-vinyltetrahydrofuro[2,3-d][1,3]dioxole (6). Butyllithium (2.71 mmol, 2.5 M in hexane) was added into the solution of Methyltriphenylphosphonium iodide (2.9 g, 6.78 mmol) in dry THF (25 mL) at 25° C., after addition, the reaction mixture was stirred at 40° C. for 2 hrs, then the solution was cooled to 0° C. and 5 (900 mg, 2.26 mmol in 5 ml THF) was added into the mixture, removed the ice-bath, the reaction was stirred at 30° C. After 2 h, the reaction mixture was quenched by saturated aqueous NH₄CI solution (10 mL), extracted with ether, washed with water, brine solution, dried (Na₂SO₄) and evaporated to dryness under reduced pressure. Chromatography (hexane: ethyl acetate 20:1 to 15:1) afforded 6 (685 mg, 75%) as a colorless oil. NMR (400 MHz, CDCl₃) δ 7.39-7.18 (m, 10H), 6.26-6.10 (m, 1H), 5.83-5.69 (m, 1H), 5.51 (dd, J=17.6, 1.8 Hz, 1H), 5.38-5.17 (m, 1H), 4.75 (d, J=12.3 Hz, 1H), 4.57 (dd, J=11.4, 7.0 Hz, 2H), 4.51 (d, J=12.1 Hz, 1H), 4.39 (d, J=12.1 Hz, 1H), 4.29-4.10 (m, 1H), 3.32 (s, 2H), 1.52 (s, 3H), 1.28 (s, 3H).

Step 6. Synthesis of (3R,4S,5R)-4-(benzyloxy)-5-((benzyloxy)methyl)-5-vinyltetrahydrofuran-2,3-diyl diacetate (7). To a solution of 6 (600 mg, 1.52 mmol) in EtOAc (3.00 mL) was added Acetic anhydride (431 uL, 4.56 mmol), and the solution was cooled to 0° C. After the addition of sulfuric acid (304 umol), the reaction mixture was allowed to warm to RT and stirred for 16 h. After the addition of NaHCO₃ (aq, sat, 2.00 mL), the mixture was stirred at RT for 10 min. The layers were separated, and the aqueous layer was extracted three times with EtOAc (5.00 mL). The combined organics were dried over MgSO₄ and concentrated under reduced pressure to give colorless oil 7 (600 mg, 90%). this material was carried over to the next step without further purification. NMR (400 MHz, CDCl₃) δ 7.34-7.26 (m, 8H), 7.21-7.16 (m, 2H), 6.17 (s, 1H), 5.28 (d, J=4.7 Hz, 1H), 4.70 (d, J=12.2 Hz, 1H), 4.61-4.44 (m, 4H), 4.38 (d, J=11.8 Hz, 1H), 3.66 (d, J=10.9 Hz, 1H), 3.57-3.53 (m, 1H), 2.64 (s, 1H), 2.13 (d, J=6.7 Hz, 3H), 1.79 (s, 3H).

Step 7. Synthesis of (2R,3R,4S,5R)-2-(6-amino-2-fluoro-9H-purin-9-yl)-4-(benzyloxy)-5-((benzyloxy)methyl)-5-vinyltetrahydrofuran-3-yl acetate (8). To a flame dried flask under nitrogen was added 2-Fluoroadenine (1.59 g, 10.4 mmol) and it was dissolved in MeCN (94.1 mL). To this was added 7 (3.80 g, 8.63 mmol) and this mixture was stirred for 1 h at 70° C. Then the flask was cooled and N,O-Bis-(trimethylsilyl)acetamide (6.68 mL, 25.9 mmol) solid Trimethylsilyl trifluormethansulfonate (1.89 mL, 10.4 mmol) was added followed by N,O-Bis-(trimethylsilyl)acetamide (6.68 mL, 25.9 mmol) and this mixture was stirred at 70° C. for 1 h. The reaction was quenched with 50 mL of sat NaHCO₃. The mixture was diluted with 50 mL of EtOAc and extracted 3 times. The organic layer was separate, dried over Na₂SO₄, filtered and concentrated under reduced pressure. The crude was purified by column chromatography on SiO₂ EtOAc 0-100% in Hexane to give 8 (3.50 g, 76%) as a white solid. NMR (400 MHz, CDCl₃) δ 8.10 (s, 1H), 7.37-7.27 (m, 8H), 7.23 (s, 2H), 6.22 (d, J=3.5 Hz, 1H), 5.98 (dd, J=17.4, 11.0 Hz, 3H), 5.62 (dd, J=5.6, 3.5 Hz, 1H), 5.50 (dd, J=17.4, 1.3 Hz, 1H), 5.34-5.24 (m, 1H), 4.70 (d, J=5.6 Hz, 1H), 4.59 (d, J=11.6 Hz, 1H), 4.59 (d, J=11.6 Hz, 1H), 4.48 (dd, J=11.7, 4.8 Hz, 2H), 4.40 (d, J=11.8 Hz, 1H), 3.51-3.44 (m, 2H), 2.04 (s, 3H).

Step 8. (2R,3R,4S,5R)-2-(6-amino-2-fluoro-9H-purin-9-yl)-4-(benzyloxy)-5-((benzyloxy)methyl)-5-vinyltetrahydrofuran-3-ol (9). To a flame dried flask under nitrogen was added 8 (3.50 g, 6.56 mmol) dissolved in MeOH (100 mL). To the solution was added Triethylamine (16.4 mL, 117 mmol) at room temperature and the mixture was stirred at reflux for 18 h. The mixture was concentrated in vacuum and diluted with EtOAc (15 mL). The solution was successively washed with water (5 mL×3) and brine, dried (MgSO₄), and concentrated in vacuum to the 9 (3.01 g, 93%) as white solid. NMR (400 MHz, DMSO-d6) δ 8.23 (s, 1H), 7.88 (d, J=30.8 Hz, 2H), 7.43-7.23 (m, 10H), 6.05 (dd, J=17.4, 10.9 Hz, 1H), 5.87 (d, J=6.4 Hz, 1H), 5.58 (d, J=6.3 Hz, 1H), 5.37 (dd, J=17.4, 1.8 Hz, 1H), 5.20 (dd, J=10.9, 1.8 Hz, 1H), 4.90 (dd, J=11.6, 6.2 Hz, 1H), 4.81 (d, J=12.0 Hz, 1H), 4.62 (d, J=12.0 Hz, 1H), 4.52 (s, 2H), 4.30 (d, J=5.2 Hz, 1H), 3.55 (q, J=10.2 Hz, 2H).

Step 9. Synthesis of 9-((2R,4S,5R)-4-(benzyloxy)-5-((benzyloxy)methyl)-5-vinyltetrahydrofuran-2-yl)-2-fluoro-9H-purin-6-amine derivatives (10). Step-1: To an acetonitrile solution (63.7 mL) of 9 (1.34 g, 2.73 mmol) and 4-Dimethylaminopyridine (999 mg, 8.01 mmol) was added O-Phenyl chlorothionoformate (457 uL, 3.27 mmol) at room temperature under an argon atmosphere, and the mixture was stirred at the same temperature for 1 h. After the solvent was removed under reduced pressure, the residue was partitioned between ethyl acetate and water. The organic phase was washed with water and brine and dried over sodium sulfate. The filtrate was concentrated under reduced pressure, and the residue was passed through a short silica gel column with EtOAc to give an oily colourless liquid of 9a (50-67%). Step-2: Compound 9a (1.00 g, 1.55 mmol) and n-Bu3SnH (515 uL, 1.86 mmol) and Azobisisobutyronitrile (127 mg, 774 umol) were dissolved in toluene at room temperature under nitrogen atmosphere. Then the mixture was stirred at 85° C. for 1 h. After finishing the reaction, directly purified with silica gel column on flash chromatography using 50% EtOAc in hexane to afford the white solid of 10 (48-63%).

Step 10. Synthesis of (2R,3S,5R)-5-(6-amino-2-fluoro-9H-purin-9-yl)-2-ethyl-2-(hydroxymethyl)tetrahydrofuran-3-ol (Cpd. No. 1). A flame-dried RBF equipped with 10 (89.0 mg, 180 umol) and Pd(OH)₂/C, 20% loading (133 mg) [substrate and catalyst as 1:1.5 ratio] in THF (7.09 mL). Hydrogen balloon was added under nitrogen, then the reaction mixture was bubbled with hydrogen gas from a balloon for 1 min. The reaction mixture was then allowed to stir under hydrogen (1 atm) at ambient temperature overnight. after 18 h. Pd(OH)₂/C was removed by filtration and concerted the reaction mixture giving Cpd. No. 1 (65-85%).

### Example 2

### Synthesis of 4-amino-1-((2R,4S,5R)-5-ethyl-4-hydroxy-5-(hydroxymethyl)tetrahydrofuran-2-yl)-5-fluoropyrimidin-2(1H)-one (Cpd. No. 2)

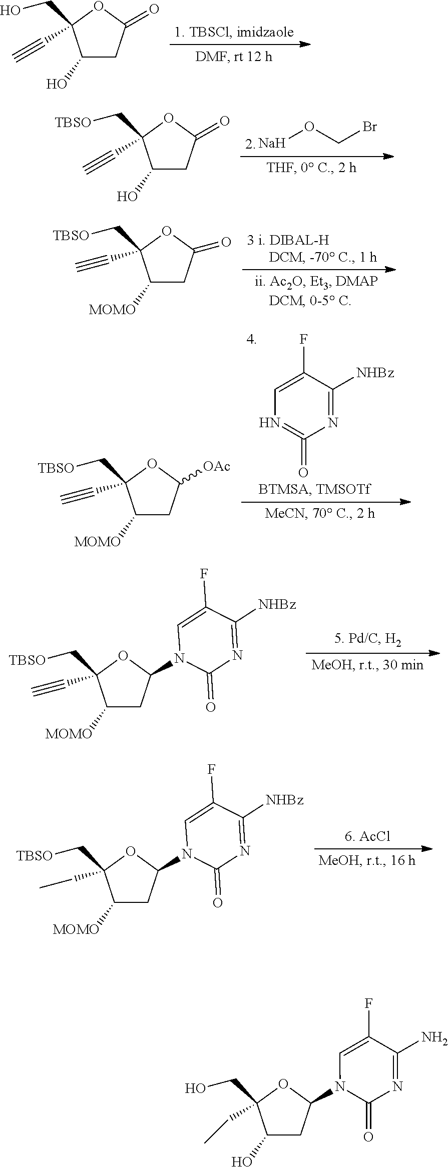

(4S,5R)-5-ethynyl-4-hydroxy-5-(hydroxymethyl)tetrahydrofuran-2-one was synthesized according the procedure described in WO 2007/038507 A2.

Step 1. To a solution of (4S,5R)-5-ethynyl-4-hydroxy-5-(hydroxymethyltetrahydrofuran-2-one (1 g, 6.4 mmol) and imidazole (1.31 g, 19.2 mmol) in DMF (30 mL) was added TBSCl (1.06 g, 7.1 mmol) at 20° C. The resulting mixture was stirred at 20° C. for 16 hours. After that, the reaction was diluted with EtOAc (80 mL) and washed with brine (50 mL×2). The layers were separated, the organic layer was concentrated. The residue was purified by column chromatography on silica gel (20% to 30% EtOAc in petroleum ether) to afford the compound (0.7 g, 40%) as a white solid. ¹H NMR (400 MHz, CDCl₃) δ 4.53-4.50 (m, 1H), 3.95-3.87 (m, 2H), 2.99-2.93 (m, 1H), 2.83 (s, 1H), 2.61-2.55 (m, 1H), 2.39-2.37 (m, 1H), 0.89 (s, 9H), 0.10 (s, 6H).

Step 2. To a solution of (4S,5R)-5-(((tert-butyldimethylsilyl)oxy)methyl)-5-ethynyl-4 hydroxydihydrofuran-2(3H)-one (3 g, 11 mmol) in THF (50 mL) was added NaH (665 mg, 16 mmol, 60% dispersion in mineral oil) at 0° C. After stirring 0° C. for 10 min, MOMBr (2.77 g, 22 mmol) was added. The resulting mixture was stirred at 25° C. for additional 3 hours. After that, the reaction mixture was poured into water (50 mL) and extracted with DCM (50 mL×2). The organic layer was washed with brine (50 mL×2), concentrated. The residue was purified by column chromatography on silica gel (10% to 20% EtOAc in petroleum ether) to afford the compound (2.1 g, 60%) as a colorless oil. ¹H NMR (400 MHz, CDCl₃) δ 4.81-4.69 (m, 2H), 4.56-4.53 (m, 1H), 3.95-3.85 (m, 2H), 3.42 (s, 3H), 2.97-2.91 (m, 1H), 2.72 (s, 1H), 2.68-2.62 (m, 1H), 0.89 (s, 9H), 0.10 (s, 6H).

Step 3. To a solution of (4S,5R)-5-[[tert-butyl(dimethyl)silyl]oxymethyl]-5-ethynyl-4-(methoxymethoxy)tetrahydrofuran-2-one (1.1 g, 3.5 mmol) in DCM (30 mL) was added DIBAL-H (4.2 mL, 4.2 mmol) dropwise at −70° C. The mixture was stirred for 30 min. After that, the reaction was quenched with methanol (5 mL), washed with aqueous citric acid solution (10 wt/o, 30 mL) and brine (30 mL×2), concentrated to give 1.1 g crude (4S,5R)-5-[[tert-butyl(dimethyl)silyl]oxymethyl]-5-ethynyl-4-(methoxymethoxy)tetrahydrofuran-2-ol as a yellow oil. It was dissolved in DCM (20 mL). Et₃N (457 mg, 4.52 mmol), DMAP (42 mg) and Ac₂O (426 mg, 4.17 mmol) were added sequentially at 0° C. After stirring at 25° C. for 1 hour, the reaction mixture was diluted with MTBE (100 mL), washed with aqueous citric acid solution (10 wt %, 50 mL) and brine (50×2 mL), concentrated under reduce pressure. The residue was purified by column chromatography on silica gel (10% to 30% EtOAc in petroleum ether) to afford the compound (0.9 g, 72%) as a white solid.

Step 4. A mixture of N-(5-fluoro-2-hydroxy-pyrimidin-4-yl)benzamide (100 mg, 0.43 mmol) and BTMSA (219 mg, 1.29 mmol) in MeCN (10 mL) was stirred at 70° C. for 1 hour, then cooled to 20° C., TMSOTf (148 mg, 0.66 mmol) and a solution of [(4S,5R)-5-[[tert-butyl(dimethyl)silyl]oxymethyl]-5-ethynyl-4-(methoxymethoxy)tetrahydrofuran-2-yl]acetate (0.12 g, 0.33 mmol) in MeCN (5 mL) were added sequentially. After stirring at 20° C. for 3 hours, the reaction mixture was poured into water (50 mL), extracted with EtOAc (50 mL×2), concentrated. The residue was purified by pre-TLC (30% EtOAc in petroleum ether) to afford the compound (40 mg, 22%) as a white solid. ¹H NMR (400 MHz, CDCl₃) δ 13.10 (s, 1H), 8.30-8.19 (m, 3H), 7.58-7.54 (m, 1H), 7.48-7.44 (m, 2H), 6.28-6.26 (m, 1H), 4.75-4.71 (m, 2H), 4.43 (t, J=7.6 Hz, 1H), 4.10-4.07 (m, 1H), 3.94-3.91 (m, 1H), 3.42 (s, 3H), 2.76-2.71 (m, 1H), 2.67 (s, 1H), 2.40-2.35 (m, 1H), 0.97 (s, 9H), 0.18 (s, 6H). LCMS (ESI): m/z 532.5 (M+H)+.

Step 5. A mixture of N-[1-[(2R,4S,5R)-5-[[tert-butyl(dimethyl)silyl]oxymethyl]-5-ethynyl-4-(methoxymethoxy)tetrahydrofuran-2-yl]-5-fluoro-2-oxo-pyrimidin-4-yl]benzamide (70 mg, 131 μmol) and 10% palladium on carbon (10 mg) in MeOH (5 mL) was stirred at 20° C. for 2 hours under H₂ (15 psi). Then the reaction mixture was filtrated. The filtrate was concentrated to afford the compound (70 mg, 99%) as a white solid. LCMS (ESI): m/z 536.1 (M+H)+.

Step 6. To a solution of N-[1-[(2R,4S,5R)-5-[[tert-butyl(dimethyl)silyl]oxymethyl]-5-ethyl-4-(methoxymethoxy)tetrahydrofuran-2-yl]-5-fluoro-2-oxo-pyrimidin-4-yl]benzamide (70 mg, 0.13 mmol) in MeOH (5 mL) was added acetyl chloride (102 mg, 1.31 mmol) at 20° C. After stirring at 20° C. for 16 hours, the reaction mixture was concentrated. The residue was purified by prep-HPLC (basic) to afford the title compound (1.75 mg, 5%) as a white solid. ¹H NMR (400 MHz, MeOD) δ8.34 (d, J=7.2 Hz, 1H), 6.12-6.09 (m, 1H), 4.44-4.41 (m, 1H), 3.73-3.70 (m, 1H), 3.61-3.56 (m, 1H), 2.42-2.24 (m, 2H), 1.78-1.56 (m, 2H), 0.97 (t, J=7.6 Hz, 3H). LCMS (ESI): m/z 296.1 (M+Na)⁺. LC-RT=0.83 min; Method D.

### Example 3

### Synthesis of (2R,3S,5R)-5-(6-amino-2-fluoro-9H-purin-9-yl)-2-cyclopropyl-2-(hydroxymethyl)tetrahydrofuran-3-ol (Cpd. No. 5)

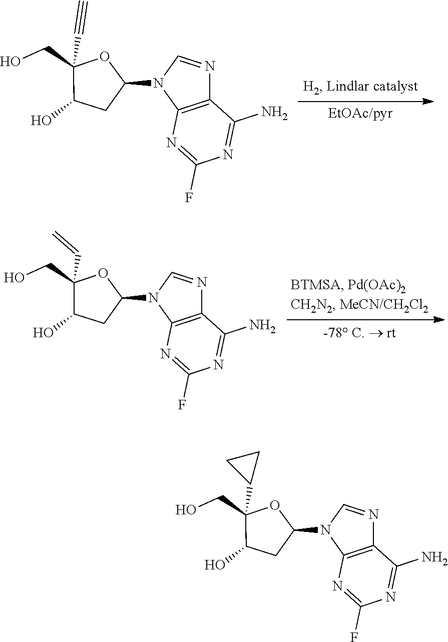 [SMILES: C#C[C@]1(CO)O[C@@H](n2cnc3c(N)nc(F)nc32)C[C@@H]1O ; C=C[C@]1(CO)O[C@@H](n2cnc3c(N)nc(F)nc32)C[C@@H]1O ; Nc1nc(F)nc2c1ncn2[C@H]1C[C@H](O)[C@](CO)(C2CC2)O1]

Step 1. Commercially available (2R,3S,5R)-5-(6-amino-2-fluoro-9H-purin-9-yl)-2-ethynyl-2-(hydroxymethyl)tetrahydrofuran-3-ol (127 mg, 0.43 mmol) and Lindlar catalyst (127 mg, 1.0 equiv by weight) were dissolved in 9:1 EtOAc/pyr solution (4.3 mL, 0.1 M). Then hydrogen was bubbled into the solution with a balloon for 0.5 h. The reaction was stirred a rt for 6 h. Then, the reaction mixture was filtered through a pad of celite and washed with MeOH. The filtrate was concentrated under reduced pressure to obtain (2R,3S,5R)-5-(6-amino-2-fluoro-9H-purin-9-yl)-2-(hydroxymethyl)-2-vinyltetrahydrofuran-3-ol (128 mg, 99%) as a white foam. This material was used for step 2 without further purification.

Step 2. (2R,3S,5R)-5-(6-amino-2-fluoro-9H-purin-9-yl)-2-(hydroxymethyl)-2-vinyltetrahydrofuran-3-ol (128 mg, 0.43 mmol) was dissolved in MeCN (434 μL, 0.5M) under inert atmosphere. Then N,O-Bis-(trimethylsilyl)acetamide (446 μL, 4 equiv, 1.73 mmol) was added dropwise into the reaction mixture. The obtained mixture was heated at 70° C. for 0.5 h. Then the solvent was thoroughly removed under reduced pressure. The obtained thick oil was redissolved in CH2C12 (434 μL, 0.5M) and Palladium (II) Acetate (9.73 mg, 0.1 equiv, 43.4 μmol) was added, forming solution A.

In a separate flask, a solution of diazomethane in Et₂O (˜30%, 4 mL) was cooled down to −78° C. To the latter, solution A was slowly added via syringe. During the addition, some white solid was observed. After the completion of the addition of solution A, ˜1 mL of dry MeCN was added to fully dissolve any solid formed in the process. The reaction was allowed to warm up to rt and it was stirred for 18 h. Then, a solution 1:1 CH2Ch/AcOH (˜ 5 mL) was added into the reaction mixture. The obtained solution was stirred at rt for further 2 h. Then, the solvent was removed under reduced pressure and the obtained yellow oil was purified by prep-HPLC column using the following conditions: XBridge Prep C18, 5 μm 19×10 mm, CSH Prep C18 OBD, 5 μm, 30×75 mm, Isocratic at 10% B for Imin, 10% B isocratic for 1 min, 10% B to 30% B for 11 minutes, 30% B to 100% B for 0.1 minute, hold 100% B for 2.9 minutes, with flow 45 mL/min. After drying, (2R,3S,5R)-5-(6-amino-2-fluoro-9H-purin-9-yl)-2-cyclopropyl-2-(hydroxymethyl)tetrahydrofuran-3-ol (3.65 mg, 6.5%) was obtained as a white solid. 1H-NMR (400 MHZ, CD3OD) δ 8.29 (s, 1H), 6.14 (t, J=6.1 Hz, 1H), 4.75 (t, J=6.3 Hz, 1H), 3.73-3.47 (m, 2H), 2.75 (ddd, J=13.2, 6.6, 5.7 Hz, 1H), 2.64-2.34 (m, 1H), 0.95 (tt, J=8.4, 5.6 Hz, 1H), 0.65-0.38 (m, 2H), 0.30 (ddtd, J=13.3, 11.7, 5.7, 4.0 Hz, 2H). 19F-NMR (376, CD3OD) −49.65 (s). LCMS ([M−H]⁺=308).

### Example 4

### Synthesis of 4-amino-5-fluoro-1-((2R,4S,5R)-4-hydroxy-5-(hydroxymethyl)-5-methyltetrahydrofuran-2-yl)pyrimidin-2(1H)-one (Cpd. No. 4)

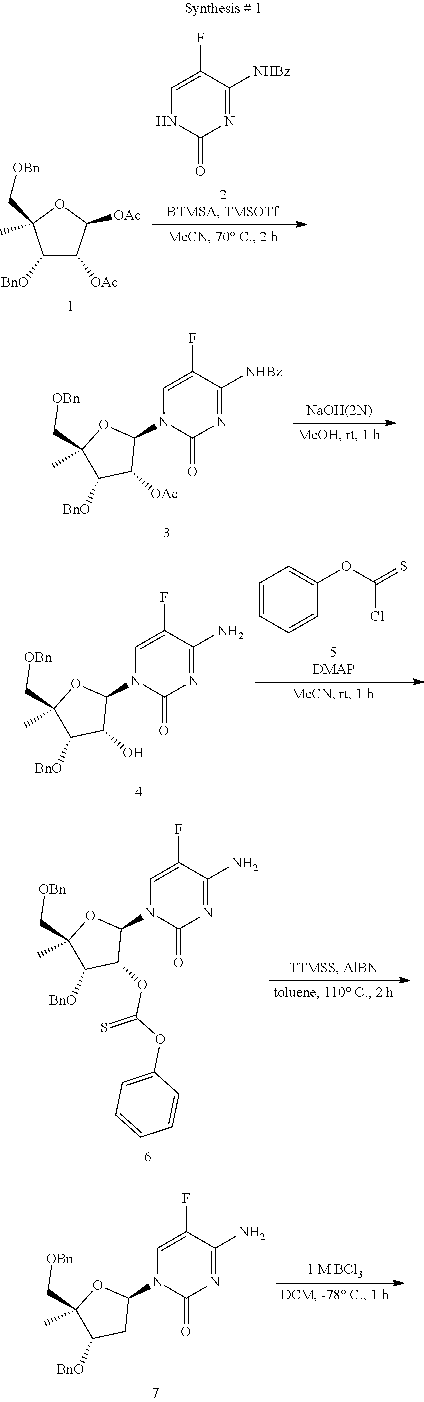

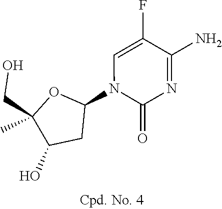 [SMILES: C[C@]1(CO)O[C@@H](n2cc(F)c(N)nc2=O)C[C@@H]1O]

Step 1. To a mixture of N-(5-fluoro-2-oxo-1H-pyrimidin-4-yl)benzamide (330 mg, 1.42 mmol) in MeCN (15 mL) was added BTMSA (603 mg, 3.54 mmol) in one portion at 25° C. under N2. Then the mixture was stirred at 70° C. for 1 hour. After cooling to 25° C., the TMSOTf (389 mg, 1.75 mmol) and [(2S,3R,4S,5R)-2-acetoxy-4-benzyloxy-5-(benzyloxymethyl)-5-methyl-tetrahydrofuran-3-yl]acetate (0.5 g, 1.17 mmol) was added dropwise at 25′C. The resulting mixture was stirred at 25° C. for 16 hours. Then the reaction was quenched by addition of 10 wt % of aqueous citric acid solution (10 mL) and then extracted by EtOAc (20 mL). The organic layer was washed with brine (15 mL), dried over Na₂SO₄, filtered and concentrated. The residue was purified by silica gel chromatography (column height: 250 mm, diameter: 100 mm, 100-200 mesh silica gel, Petroleum ether/Ethyl acetate=3/1) to give the title compound (0.65 g, 93% yield) as yellow oil. LC-RT=1.036 min, [M+Na]⁺624.3; Method C.

Step 2. To a mixture of [(2R,3R,4S,5R)-2-(4-benzamido-5-fluoro-2-oxo-pyrimidin-1-yl)-4-benzyloxy-5-(benzyloxymethyl)-5-methyl-tetrahydrofuran-3-yl]acetate (0.65 g, 1.08 mmol) in MeOH (15 mL) was added NaOH (2 M, 1.62 mL) aqueous solution in one portion at 25° C. The mixture was stirred at 25° C. for 30 min. The reaction mixture was concentrated directly. The residue was purified by silica gel chromatography (column height: 250 mm, diameter: 100 mm, 100-200 mesh silica gel, Dichloromathane/MeOH=100/0, 15/1) to give the title compound (0.35 g, 71% yield) as a white solid. ¹H NMR (400 MHz, DMSO-d6) δ 7.91 (d, J=6.8 Hz, 1H), 7.75 (s, 1H), 7.50 (s, 1H), 7.36-7.27 (m, 10H), 5.86-5.81 (m, 1H), 5.48 (d, J=6.0 Hz, 1H), 4.78 (d, J=12.0 Hz, 1H), 4.56-4.48 (m, 3H), 4.26 (q, J=5.6 Hz, 1H), 3.97 (d, J=5.2 Hz, 1H), 3.54 (d, J=10.0 Hz, 1H), 3.42 (d, J=10.4 Hz, 1H), 1.22 (s, 3H)

Step 3. To a mixture of 4-amino-1-[(2R,3R,4S,5R)-4-benzyloxy-5-(benzyloxymethyl)-3-hydroxy-5-methyl-tetrahydrofuran-2-yl]-5-fluoro-pyrimidin-2-one (0.35 g, 768.42 umol) in MeCN (15 mL) was added DMAP (282 mg, 2.31 mmol), then 0-phenyl chloromethanethioate (199 mg, 1.15 mmol) was added dropwise at 25° C. under N2. After stirring at 25° C. for 1 hour, the mixture was concentrated directly. The residue was purified by silica gel chromatography (column height: 250 mm, diameter: 100 mm, 100-200 mesh silica gel, Dichloromathane/MeOH=100/0) to give the title compound (0.32 g, 70% yield) as a light yellow solid.

Step 4: To a mixture of 4-amino-1-[(2R,3R,4S,5R)-4-benzyloxy-5-(benzyloxymethyl)-5-methyl-3-phenoxycarbothioyloxy-tetrahydrofuran-2-yl]-5-fluoro-pyrimidin-2-one (0.32 g, 540.86 umol) in toluene (12 mL) was added AIBN (44 mg, 270.43 umol) and TTMSS (672 mg, 2.70 mmol) in one portion at 25° C. under N2. The mixture was heated to 110° C. and stirred for 2 hours. Then the reaction mixture was concentrated directly. The residue was purified by pre-TLC (Dichloromathane/MeOH=15/1) to give the title compound (110 mg, 46% yield) as colorless oil.

Step 5. To a mixture of 4-amino-1-[(2R,4S,5R)-4-benzyloxy-5-(benzyloxymethyl)-5-methyl-tetrahydrofuran-2-yl]-5-fluoro-pyrimidin-2-one (0.09 g, 204.79 umol) in DCM (9 mL) was added BCl₃ (1 M, 1.43 mL) dropwise at −78° C. under N2. The reaction mixture was stirred at −78° C. for 15 min, then allowed to warm up to −40° C. and stirred for another 0.5 h. Then the reaction was quenched by addition of MeOH (1 mL) and NH₄OH (1 mL) at −40° C. After stirring −40° C. for another 10 min, the mixture was warmed to room temperature and stirred for 10 min. After that, the reaction mixture was concentrated directly. The residue was purified by pre-HPLC (basic) to give the title compound (3.2 mg, 6% yield) as a white solid. ¹H NMR (400 MHz, CD₃OD) δ 8.37 (d, J=7.2 Hz, 1H), 6.15-6.05 (m, 1H), 4.36 (t, J=6.0 Hz, 1H), 3.67-3.53 (m, 2H), 2.51-2.39 (m, 1H), 2.30-2.20 (m, 1H), 1.17 (s, 3H). LC-RT=0.791 min, [2M+H]⁺519.2, Method D.

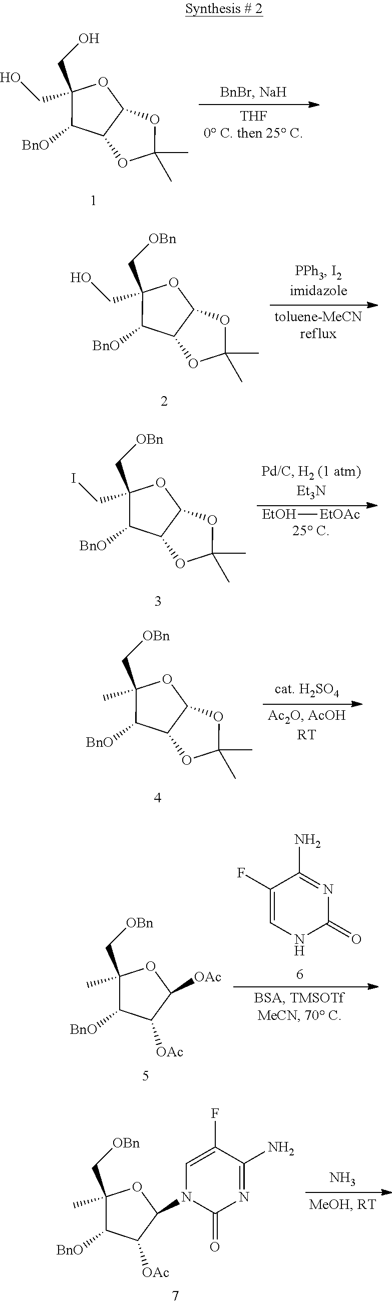

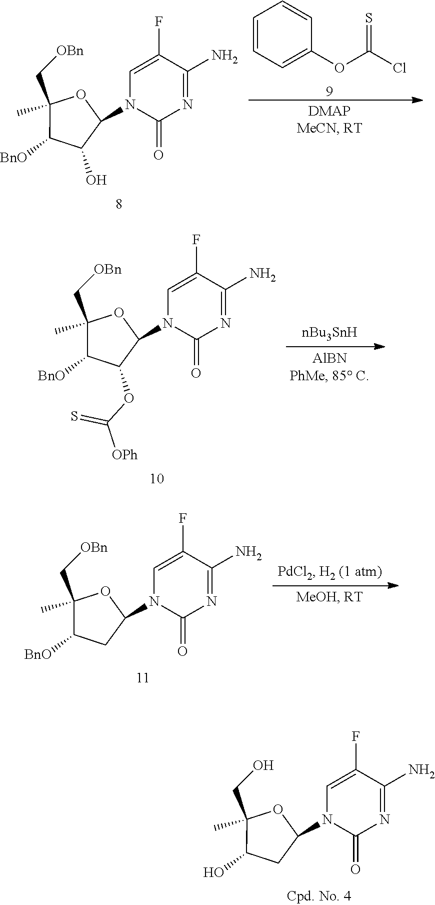

Step 1: Synthesis of ((3aR,5R,6S,6aR)-6-(benzyloxy)-5-((benzyloxy)methyl)-2,2-dimethyltetrahydrofuro[2,3-d][1,3]dioxol-5-yl)methanol. To a mixture of NaH (3.54 g, 88.61 mmol, 60% purity) in THF (120 mL) was added [(3aR,6S,6aR)-6-benzyloxy-5-(hydroxymethyl)-2,2-dimethyl-6,6a-dihydro-3aH-furo[2,3-d][1,3]dioxol-5-yl]methanol 1 (25 g, 80.56 mmol, 1 eq) in THF (120 mL) dropwise at 0° C. under N2. The mixture was stirred at 0° C. for 30 min, then benzyl bromide (13.78 g, 80.56 mmol) was added dropwise. The resulting mixture was allowed to warm up to 25° C. and stirred for 2 hours. After that, the reaction was quenched by addition of sat. aq. NH₄Cl (50 mL) at 0° C. and washed with EtOAc (200 mL). The organic phase was washed with brine (100 mL), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=100/1, 5/1) to give the title compound 2 (17 g, 53% yield) as light yellow oil. ¹H NMR (400 MHz, CDCl₃) δ 7.40-7.27 (m, 10H), 5.80 (d, J=3.6 Hz, 1H), 4.79 (d, J=12.0 Hz, 1H), 4.68-4.63 (m, 1H), 4.58-4.43 (m, 3H), 4.28 (d, J=5.2 Hz, 1H), 3.98-3.90 (m, 1H), 3.88-3.79 (m, 1H), 3.64-3.58 (m, 1H), 3.57-3.51 (m, 1H), 2.43-2.35 (m, 1H), 1.64 (s, 3H), 1.36 (s, 3H)

Step 2: Synthesis of (3aR,5R,6S,6aR)-6-(benzyloxy)-5-((benzyloxy)methyl)-5-(iodomethyl)-2,2-dimethyltetrahydrofuro[2,3-d][1,3]dioxole. To a mixture of [(3aR,5R,6S,6aR)-6-benzyloxy-5-(benzyloxymethyl)-2,2-dimethyl-6,6a-dihydro-3aH-furo[2,3-d][1,3]dioxol-5-yl]methanol 2 (28 g, 69.92 mmol) in toluene (250 mL) and dioxane (60 mL) was added PPh₃ (73.36 g, 279.68 mmol), imidazole (19 g, 279.68 mmol) and 12 (41 g, 160.81 mmol) in one portion at 25° C. The mixture was stirred at 100° C. for 16 hours. After that, the reaction was quenched by addition of 200 mL of sat. aq. Na₂SO₃, then extracted by EtOAc (400 mL). The organic layer was washed with brine (200 mL), dried over anhydrous Na₂SO₄, filtered, concentrated. The residue was purified by silica gel chromatography (column height: 250 mm, diameter: 100 mm, 100-200 mesh silica gel, Petroleum ether/Ethyl acetate=10/1) to give the title compound 3 (31 g, 87% yield) as yellow oil. ¹H NMR (400 MHz, CDCl₃) δ 7.38-7.27 (m, 10H), 5.74 (d, J=3.6 Hz, 1H), 4.76 (d, J=12.0 Hz, 1H), 4.65-4.54 (m, 3H), 4.51-4.46 (m, 1H), 4.27 (d, J=4.8 Hz, 1H), 3.97 (d, J=11.2 Hz, 1H), 3.61-3.55 (m, 1H), 3.53-3.44 (m, 2H), 1.64 (s, 3H), 1.34 (s, 3H).

Step 3: Synthesis of (3aR,5R,6S,6aR)-6-(benzyloxy)-5-((benzyloxy)methyl)-2,2,5-trimethyltetrahydrofuro[2,3-d][1,3]dioxole. To a mixture of (3aR,5R,6S,6aR)-6-benzyloxy-5-(benzyloxymethyl)-5-(iodomethyl)-2,2-dimethyl-6,6a-dihydro-3aH-furo[2,3-d][1,3]dioxole 3 (10 g, 19.59 mmol) in EtOH (50 mL) was added TEA (4.56 g, 45.07 mmol) and 10% palladium on carbon (6.26 g, 5.88 mmol) in EtOAc (50 mL) at 25° C. under H₂. After stirring at 25° C. for 2 hour, the mixture was filtered and concentrated directly. The residue was purified by silica gel chromatography (column height. 250 mm, diameter: 100 mm, 100-200 mesh silica gel, Petroleum ether/Ethyl acetate=10/1) to give the title compound 4 (7 g, 93% yield) as yellow oil. ¹H NMR (400 MHz, CDCl₃) δ 7.38-7.24 (m, 10H), 5.75 (d, J=4.0 Hz, 1H), 4.77 (d, J=12.4 Hz, 1H), 4.65-4.61 (m, 1H), 4.57 (d, J=12.4 Hz, 1H), 4.54-4.49 (m, 1H), 4.47-4.41 (m, 1H), 4.10 (d, J=5.2 Hz, 1H), 3.39 (d, J=10.4 Hz, 1H), 3.24 (d, J=10.4 Hz, 1H), 1.64 (s, 3H), 1.40 (s, 3H), 1.34 (s, 3H)

Step 4: Synthesis of (2S,3R,4S,5R)-4-(benzyloxy)-5-((benzyloxy)methyl)-5-methyltetrahydrofuran-2,3-diyl diacetate. To a mixture of (3aR,5R,6S,6aR)-6-benzyloxy-5-(benzyloxymethyl)-2,2,5-trimethyl-6,6a-dihydro-3aH-furo[2,3-d][1,3]dioxole 4 (10 g, 26.01 mmol) in Ac₂O (12 mL) and AcOH (120 mL) was added H₂SO₄ (10 drops, 98% purity) in one portion at 25° C. under N2. The mixture was stirred for 1 hour. The residue was poured into ice-water (20 mL) and stirred for 5 min. The aqueous phase was extracted with ethyl acetate (100 mL×2). The combined organic phase was washed with brine (100 mL), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=10/1) to give the title compound 5 (7.7 g, 69% yield) as colorless oil. ¹H NMR (400 MHz, CDCl₃) δ 7.36-7.27 (m, 10H), 6.10 (s, 1H), 5.35 (d, J=5.2 Hz, 1H), 4.67-4.61 (m, 1H), 4.51 (s, 1H), 4.48 (s, 1H), 4.46-4.41 (m, 1H), 4.31 (d, J=5.2 Hz, 1H), 3.43-3.38 (m, 1H), 3.35-3.30 (m, 1H), 2.13 (s, 3H), 1.86 (s, 3H), 1.32 (s, 3H).

Step 5: Synthesis of (2R,3R,4S,5R)-2-(4-amino-5-fluoro-2-oxopyrimidin-1(2H)-yl)-4-(benzyloxy)-5-((benzyloxy)methyl)-5-methyltetrahydrofuran-3-yl acetate. A flame-dried three-necked round-bottomed flask equipped with a magnetic stirrer bar and fitted with a reflux condenser was charged with a suspension of 5-fluorocytosine 6 (775 mg, 6.00 mmol) in dry acetonitrile (45.0 mL). N,O-Bis(trimethylsilyl)acetamide (3.90 mL, 15.1 mmol) was added dropwise, and the reaction mixture heated to 70° C. with stirring for ca. 1.5 h. A solution of (2S,3R,4S,5R)-4-(benzyloxy)-5-((benzyloxy)methyl)-5-methyltetrahydrofuran-2,3-diyl diacetate 5 (2.14 g, 5.00 mmol) in dry acetonitrile (5.00 mL) was added dropwise, followed by dropwise addition of trimethylsilyl trifluoromethanesulfonate (1.10 mL, 6.0 mmol) over ca. 5 mins. The reaction mixture was then stirred at 70° C. for ca. 1 h, then cooled to 0° C. with stirring and quenched by careful addition of saturated aqueous NaHCO₃ solution (40 mL). Water (50 mL) was added, the mixture poured into a separatory funnel, and the organics extracted with a 4:1 mixture of CHCl₃-IPA (3×50 mL). The combined organics were washed with water (50 mL) and saturated aqueous NaCl solution, dried (anhyd. Na₂SO₄), filtered and concentrated in vacuo to afford crude 7 (2.49 g, 5.00 mmol, quant.) as a white foam, which was utilized directly in the next step without further purification. ¹H NMR (400 MHz, CDCl₃) δ 8.13 (d, J=6.7 Hz, 1H), 7.37-7.21 (m, 10H), 6.13 (dd, J=2.4, 1.8 Hz, 1H), 5.40 (dd, J=5.7, 2.7 Hz, 1H), 5.31 (br. s, 2H), 4.63 (d, J=11.8 Hz, 1H), 4.45 (d, J=11.5 Hz, 1H), 4.36 (d, J=11.8 Hz, 1H), 4.35 (d, J=11.5 Hz, 1H), 4.22 (d, J=5.7 Hz, 1H), 3.59 (d, J=10.4 Hz, 1H), 3.31 (d, J=10.4 Hz, 1H), 2.12 (s, 3H), 1.27 (s, 3H).

Step 6: Synthesis of 4-amino-1-((2R,3R,4S,5R)-4-(benzyloxy)-5-((benzyloxy)methyl)-3-hydroxy-5-methyltetrahydrofuran-2-yl)-5-fluoropyrimidin-2(1H)-one. An RBF equipped with a magnetic stirrer bar was charged with crude (2R,3R,4S,5R)-2-(4-amino-5-fluoro-2-oxopyrimidin-1(2H)-yl)-4-(benzyloxy)-5-((benzyloxy)methyl)-5-methyltetrahydrofuran-3-yl acetate 7 (2.49 g, 5.00 mmol) and a solution of ammonia (7N in MeOH, 50 mL, 360 mmol) added in one portion. The reaction mixture was stirred at ambient temperature for ca. 19 h. The reaction mixture was then sparged with nitrogen for ca. 60 minutes to remove excess ammonia, then concentrated in vacuo to afford crude 8 (2.28 g, 5 mmol, quant.) as a white solid, which was utilized directly without further purification.

Step 7: Synthesis of O-((2R,3R,4S,5R)-2-(4-amino-5-fluoro-2-oxopyrimidin-1(2H)-yl)-4-(benzyloxy)-5-((benzyloxy)methyl)-5-methyltetrahydrofuran-3-yl)O-phenyl carbonothioate. To a stirring suspension of crude 4-amino-1-((2R,3R,4S,5R)-4-(benzyloxy)-5-((benzyloxy)methyl)-3-hydroxy-5-methyltetrahydrofuran-2-yl)-5-fluoropyrimidin-2(1H)-one 8 (2.28 g, 5.00 mmol) and 4-dimethylaminopyridine (1.92 g, 15.7 mmol) in dry MeCN (105 mL) at ambient temperature was added dropwise O-phenyl chlorothionoformate 9 (1.00 mL, 7.23 mmol) under nitrogen, forming a bright yellow mixture with concomitant evolution of white fumes (vented with extra needle through septum). The reaction mixture was stirred at ambient temperature for ca. 1 h. The reaction mixture was concentrated in vacuo to remove the volatiles, and the residue partitioned between 4:1 CHCl₃-IPA (150 mL) and 10% aqueous citric acid solution (100 mL). The organics were then washed with water (100 mL) and saturated aqueous NaCl solution, dried (anhyd. Na₂SO₄), filtered and concentrated in vacuo. The residue was purified by FCC on SiO₂ (70-100% EtOAc/hexanes, then 5-10% IPA/EtOAc) affording the title compound 10 (1.95 g, 3.30 mmol, 63%) as a cream solid. ¹H NMR (400 MHz, DMSO-d6) δ 7.96 (d, J=7.0 Hz, 1H), 7.92 (br. s, 1H), 7.64 (br. s, 1H), 7.47-7.41 (m, 2H), 7.40-7.26 (m, 11H), 7.08-7.03 (m, 2H), 6.08 (dd, J=4.3, 1.4 Hz, 1H), 5.94 (dd, 0.1=5.8, 4.4 Hz, 1H), 4.61 (s, 2H), 4.58-4.51 (m, 3H), 3.61 (d, J=10.3 Hz, 1H), 3.50 (d, J=10.3 Hz, 1H), 1.23 (s, 3H).

Step 8: Synthesis of 4-amino-1-((2R,4S,5R)-4-(benzyloxy)-5-((benzyloxy)methyl)-5-methyltetrahydrofuran-2-yl)-5-fluoropyrimidin-2(1H)-one. To a stirring suspension of 0-((2R,3R,4S,5R)-2-(4-amino-5-fluoro-2-oxopyrimidin-1(2H)-yl)-4-(benzyloxy)-5-((benzyloxy)methyl)-5-methyltetrahydrofuran-3-yl)O-phenyl carbonothioate 10 (1.95 g, 3.30 mmol) in toluene (16.5 mL) were added AIBN (271 mg, 1.65 mmol) and trbutyltin hydride (1.10 mL, 4.09 mmol). The reaction mixture was heated to 85° C. for ca. 1 h, then cooled to ambient temperature. The reaction mixture was diluted with EtOAc (40 mL) and washed with 0.5 M aqueous KF solution (3×20 mL). The organics were then washed with saturated aqueous NaCl solution, dried (anhyd. Na₂SO₄), filtered and concentrated in vacuo. The residue was purified by flash chromatography on SiO₂ (0-10% IPA/CHCl₃) affording the title compound 11 (869 mg, 1.98 mmol, 60%) as a yellow solid. ¹H NMR (400 MHz, CDCl₃) δ 8.22 (d, J=6.7 Hz, 1H), 7.38-7.26 (m, 10H), 6.14 (ddd, J=6.7, 4.1, 1.8 Hz, 1H), 5.87 (br. s, 1H), 5.31 (br. s, 1H), 4.58 (d, J=12.0 Hz, 1H), 4.56 (d, J=11.6 Hz, 1H), 4.48 (d, J=11.6 Hz, 1H), 4.40 (d, J=12.0 Hz, 1H), 4.22 (app. t, J=6.9 Hz, 1H), 3.65 (d, J=10.3 Hz, 1H), 3.46 (d, J=10.3 Hz, 1H), 2.64 (dt, J=13.8, 6.9 Hz, 1H), 2.27 (ddd, J=13.8, 6.8, 4.1 Hz, 1H), 1.23 (s, 3H).

Step 9: Synthesis of 4-amino-5-fluoro-1-((2R,4S,5R)-4-hydroxy-5-(hydroxymethyl)-5-methyltetrahydrofuran-2-yl)pyrimidin-2(1H)-one (Cpd. No. 4). Palladium(II) chloride (159 mg, 897 μmol) was added in one portion to a stirring solution of 4-amino-1-((2R,4S,5R)-4-(benzyloxy)-5-((benzyloxy)methyl)-5-methyltetrahydrofuran-2-yl)-5-fluoropyrimidin-2(1H)-one 11 (394 mg, 897 μmol) in MeOH (36.0 mL) under nitrogen. The reaction mixture was sparged with hydrogen gas from a balloon for ca. 60 seconds, then the mixture was then allowed to stir under hydrogen atmosphere for ca. 30 mins. The hydrogen balloon was removed and the reaction was quenched by addition of Et₃N (5.00 mL). The reaction mixture was then filtered through a short pad of Celite, rinsing with MeOH as necessary, and the filtrate was concentrated in vacuo. The residue was purified by reverse-phase FCC on C18 (Biotage Isolera Sfar 25 g, 1-25% ACN/AmB) affording the title Cpd. No. 4 (88 mg, 0.34 mmol, 38%) as an amorphous white solid after lyophilization.

### Example 5

### Synthesis of (2R,3S,5R)-5-(4-amino-5-fluoro-2-oxopyrimidin-1(2H)-yl)-3-hydroxy-2-(hydroxymethyl)tetrahydrofuran-2-carbonitrile (Cpd. No. 6)

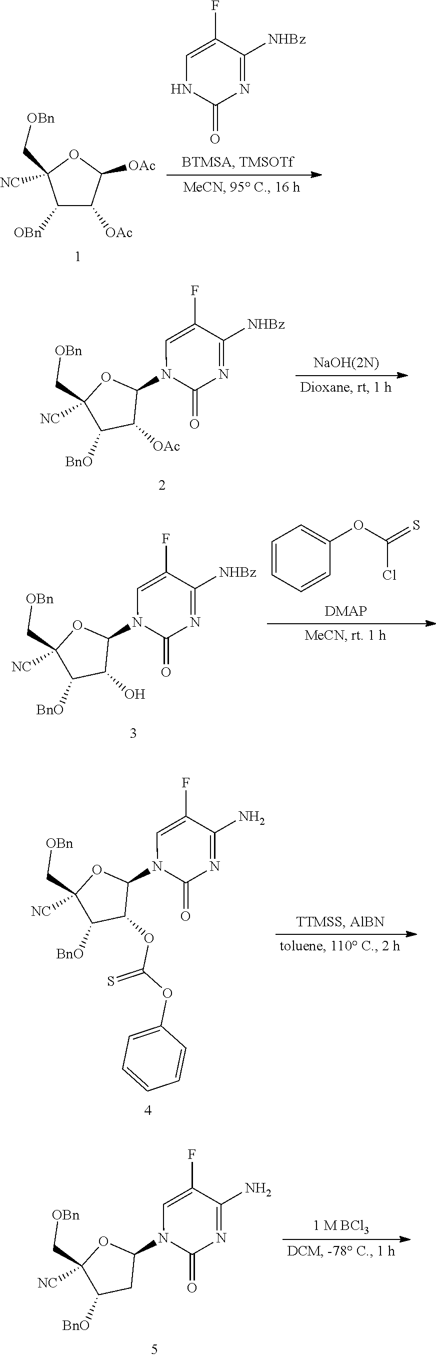

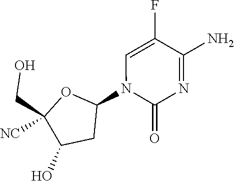 [SMILES: N#C[C@]1(CO)O[C@@H](n2cc(F)c(N)nc2=O)C[C@@H]1O]

Step 1. A solution of N-(5-fluoro-2-oxo-1H-pyrimidin-4-yl) benzamide (0.8 g, 3.4 mmol) in acetonitrile (15 mL) was degassed with N2 for three times. Then trimethylsilyl (1E)-N-trimethylsilylethanimidate (4.2 mL, 17.15 mmol) was added. The resulting reaction mixture was stirred at 70° C. for 1 h, then cooled to room temperature. Trimethylsilyl trifluoromethanesulfonate (822 uL, 4.6 mmol) was added, followed by addition of a solution of [(2S,3R,4S,5R)-2-acetoxy-4-benzyloxy-5-(benzyloxymethyl)-5-cyano-tetrahydrofuran-3-yl]acetate (1.0 g, 2.3 mmol) in acetonitrile (8 mL). The reaction mixture was stirred at 105° C. for 16 h. After cooling to room temperature, the reaction mixture was diluted with water (20 mL), extracted with EtOAc (30 mL). The organic layer was washed with brine (20 mL) dried over Na₂SO₄, filtered, concentrated to give the crude product which was purified by silica gel chromatography (solvent gradient: 0-3% MeOH in DCM) to afford the title product (0.28 g, 20% yield) as brown oil. ¹H NMR (400 MHz, CDCl₃): δ 8.28 (d, J=7.6 Hz, 2H), 7.79 (d, J=5.6 Hz, 1H), 7.61-7.55 (m, 1H), 7.51-7.45 (m, 2H), 7.39-7.32 (m, 9H), 7.27-7.21 (m, 2H), 6.13 (d, 1=2.8 Hz, 1H), 5.45-5.36 (m, 1H), 4.69 (d, J=11.6 Hz, 1H), 4.59-4.53 (m, 1H), 4.52-4.42 (m, 4H), 3.93 (d, J=10.8 Hz, 1H), 3.63 (d, J=10.8 Hz, 1H), 2.18 (s, 3H).

Step 2. To a solution of [(2R,3R,4S,5R)-2-(4-benzamido-5-fluoro-2-oxo-pyrimidin-1-yl)-4-benzyloxy-5-(benzyloxymethyl)-5-cyano-tetrahydrofuran-3-yl]acetate (0.11 g, 179.6 umol) in dioxane (3 mL) was added aqueous NaOH solution (1 M, 0.5 mL). The reaction mixture was stirred at 25° C. for 1 h. Then the reaction mixture was diluted with water (5 mL), extracted with EtOAc (10 mL), washed with brine (10 mL), dried over Na₂SO₄, filtered and concentrated. The residue was purified by silica gel chromatography (solvent gradient: 0-3% MeOH in DCM) to afford the title product (95 mg, 93% yield) as a white solid. ¹H NMR (400 MHz, CDCl₃): δ 8.28 (d, J=7.6 Hz, 2H), 7.70 (s, 1H), 7.61-7.53 (m, 1H), 7.50-7.45 (m, 2H), 7.44-7.30 (m, 10H), 5.95 (s, 1H), 4.85 (d, J=11.2 Hz, 1H), 4.70 (d, J=11.2 Hz, 1H), 4.63-4.54 (m, 2H), 4.41-4.26 (m, 2H), 3.92 (d, J=10.4 Hz, 1H), 3.75-3.69 (m, 1H).

Step 3. To a solution of N-[1-[(2R,3R,4S,5R)-4-benzyloxy-5-(benzyloxymethyl)-5-cyano-3-hydroxy-tetrahydrofuran-2-yl]-5-fluoro-2-oxo-pyrimidin-4-yl]benzamide (0.21 g, 368.1 umol) in acetonitrile (5 mL) degassed with N2 for three times. Then DMAP (90.2 mg, 736.2 umol) and O-phenyl chloromethanethioate (127.1 mg, 736.1 umol) were added. The resulting reaction mixture was stirred at 25° C. for 1 h. After that, the reaction mixture was diluted with water (5 mL), extracted with EtOAc (10 mL). The organic layer was washed with brine (10 mL), dried over Na₂SO₄, filtered and concentrated. The residue was purified by silica gel chromatography (solvent gradient: 0-3% MeOH in DCM) to afford the title product (0.25 g, 96% yield) as brown oil. LCMS (ESI): m/z 707.1 (M+H)⁺.

Step 4. A solution of N-[1-[(2R,3R,4S,5R)-4-benzyloxy-5-(benzyloxymethyl)-5-cyano-3-phenoxycarbothioyloxy-tetrahydrofuran-2-yl]-5-fluoro-2-oxo-pyrimidin-4-yl]benzamide (0.25 g, 353.7 umol) in toluene (3 mL) was purged with N2 for 5 min. Bis(trimethylsilyl)silyl-trimethyl-silane (263.8 mg, 1.06 mmol) and AIBN (29.0 mg, 176.8 umol) was added. The resulting reaction mixture was stirred at 110° C. for 2 h. After cooling to room temperature, the reaction mixture was concentrated to residue which was purified by silica gel chromatography (solvent gradient: 0-3% MeOH in DCM) to afford the title product (100 mg, 51% yield) as a white solid. ¹H NMR (400 MHz, CDCl₃): S 8.28 (d, J=7.6 Hz, 2H), 7.84 (d, J=6.0 Hz, 1H), 7.57 (d, J=7.6 Hz, 1H), 7.48 (d, J=7.6 Hz, 2H), 7.41-7.33 (m, 11H), 6.41 (t, J=6.4 Hz, 1H), 4.66 (d, J=3.6 Hz, 2H), 4.59 (d, J=8.0 Hz, 2H), 4.40 (t, J=6.0 Hz, 1H), 3.97 (d, J=10.4 Hz, 1H), 3.77 (d, J=10.4 Hz, 1H), 2.71-2.56 (m, 1H), 2.34-2.20 (m, 1H).

Step 5: A solution of N-[1-[(2R,4S,5R)-4-benzyloxy-5-(benzyloxymethyl)-5-cyano-tetrahydrofuran-2-yl]-5-fluoro-2-oxo-pyrimidin-4-yl]benzamide (40 mg, 72.1 umol) in DCM (2 mL) was degassed with N2 for three times and cooled to −78° C., and then BCl₃ in DCM (1 M, 0.2 mL) was added dropwise. The reaction mixture was stirred at −45° C. for 0.5 h. The reaction mixture was quenched with MeOH (1 mL) at −45° C. and warmed to 0° C. 1 mL NH₃H₂O (28% purity) was added to adjust pH=8 and concentrated to residue. The residue was purified by pre-HPLC (acetonitrile 0-30% /NH₃H₂O+NH₄HCO₃ in water) to afford the title compound (3.0 mg, 29/6 yield) as a white solid. ¹H NMR (400 MHz, MeOD-d4): δ 8.02 (d, 0.1=6.8 Hz, 1H), 6.35 (t, J=5.6 Hz, 1H), 4.58 (t, J=6.8 Hz, 1H), 3.97-3.83 (m, 2H), 2.53-2.34 (m, 2H). LC-RT=0.839 min, [M+H]+ 271.1; Method F.

### Example 6

### Synthesis of 5-fluoro-1-((2R,4S,5R)-4-hydroxy-5-(hydroxymethyl)-5-methyltetrahydrofuran-2-yl)pyrimidine-2,4(1H,3H)-dione (Cpd. No. 105)

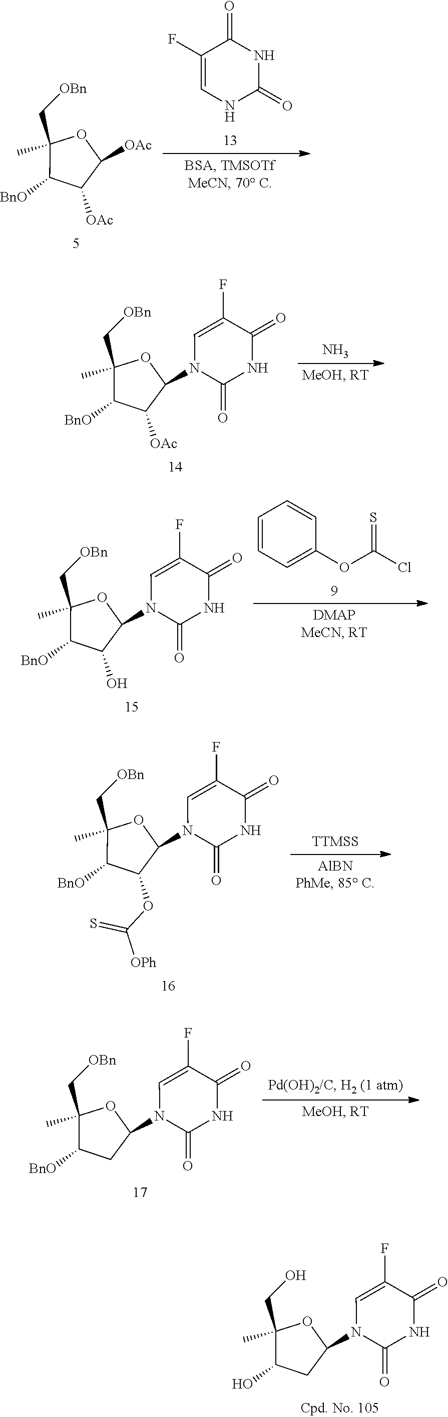

Step 1: (2R,3R,4S,5R)-4-(benzyloxy)-5-((benzyloxy)methyl)-2-(5-fluoro-2,4-dioxo-3,4-dihydropyrimidin-1(2H)-yl)-5-methyltetrahydrofuran-3-yl acetate. A flame-dried three-necked round-bottomed flask equipped with a magnetic stirrer bar and fitted with a reflux condenser was charged with a suspension of 5-fluorouracil 13 (1.53 g, 11.7 mmol) in dry acetonitrile (105 mL). NO-Bis(trimethylsilyl)acetamide (4.51 mL, 17.5 mmol) was added dropwise, and the reaction mixture heated to 70° C. with stirring for ca. 1 h. A solution of (2S,3R,4S,5R)-4-(benzyloxy)-5-((benzyloxy)methyl)-5-methyltetrahydrofuran-2,3-diyl diacetate 5 (5.00 g, 11.7 mmol) in dry acetonitrile (11.7 mL) was added dropwise, followed by dropwise addition of trimethylsilyl trifluoromethanesulfonate (2.58 mL, 14.2 mmol). The reaction mixture was then stirred at 70° C. for ca. 1.5 h, then cooled to 0° C. with stirring and quenched by careful addition of saturated aqueous NaHCO₃ solution (40 mL). Water (50 mL) was added, the mixture poured into a separatory funnel, and the organics extracted with a 4:1 mixture of CHCl₃-IPA (3×50 mL). The combined organics were washed with water (50 mL) and saturated aqueous NaCl solution, dried (anhyd. Na₂SO₄), filtered and concentrated in vacuo to afford crude 14 (5.42 g, 10.9 mmol, 93%) as a colourless oil, which was utilized directly in the next step without further purification.

Step 2: 1-((2R,3R,4S,5R)-4-(benzyloxy)-5-((benzyloxy)methyl)-3-hydroxy-5-methyltetrahydrofuran-2-yl)-5-fluoropyrimidine-2,4(1H,3H)-dione. To a stirring solution of (2R,3R,4S,5R)-4-(benzyloxy)-5-((benzyloxy)methyl)-2-(5-fluoro-2,4-dioxo-3,4-dihydropyrimidin-1(2H)-yl)-5-methyltetrahydrofuran-3-yl acetate 14 (5.42 g, 10.9 mmol) in methanol (146 mL) was added triethylamine (27.1 mL, 192 mmol) at ambient temperature. The reaction mixture was then heated to 65° C. with stirring for ca. 18 h before cooling to ambient temperature. The reaction mixture was concentrated in vacuo to remove the volatiles, then the residue was taken up in EtOAc (100 mL). The organics were washed with water (3×20 mL) and saturated aqueous NaCl solution, dried (anhyd. Na₂SO₄), filtered and concentrated in vacuo to afford the crude product. Purification by flash chromatography on SiO₂ (50-100% EtOAc/hexanes) furnished the title compound 15 (3.10 g, 6.79 mmol, 62%) as a yellow solid.

Step 3: 0-((2R,3R,4S,5R)-4-(benzyloxy)-5-((benzyloxy)methyl)-2-(5-fluoro-2,4-dioxo-3,4-dihydropyrimidin-1(2H)-yl)-5-methyltetrahydrofuran-3-yl) 0-phenyl carbonothioate. To a stirring solution of 1-((2R,3R,4S,5R)-4-(benzyloxy)-5-((benzyloxy)methyl)-3-hydroxy-5-methyltetrahydrofuran-2-yl)-5-fluoropyrimidine-2,4(1H,3H)-dione 15 (1.25 g, 2.74 mmol) in acetonitrile (27.4 mL) at ambient temperature was added 4-dimethylaminopyridine (1.00 g, 8.05 mmol), followed by O-phenyl chlorothionoformate 9 (383 μL, 2.74 mmoL) dropwise. The reaction mixture was stirred at ambient temperature for ca. 1 h, then the solvent was removed under reduced pressure to afford the crude product. Purification by flash chromatography on SiO₂ (25-100% EtOAc/hexanes) afforded the title compound 16 (740 mg, 1.25 mmol, 46%) as a yellow solid. ¹H NMR (400 MHZ, CDCl3) δ 8.91 (s, 1H, NH), 8.01 (d, J=6.3 Hz, 1H), 7.44-7.27 (m, 13H), 7.05-6.97 (m, 2H), 6.36 (dd, J=5.6, 1.6 Hz, 1H), 5.88 (t, J=5.6 Hz, 1H), 4.71 (t, J=7.5 Hz, 1H), 4.65-4.46 (m, 3H), 4.42 (t, J=6.6 Hz, 1H), 3.62 (d, J=10.2 Hz, 1H), 3.41-3.31 (m, 1H).

Step 4: 1-((2R,4S,5R)-4-(benzyloxy)-5-((benzyloxy)methyl)-5-methyltetrahydrofuran-2-yl)-5-fluoropyrimidine-2,4(1H,3H)-dione. Tris(trimethylsilyl)silane (794 μL, 2.50 mmol) and AIBN (103 mg, 624 μmol) were added to a solution of O-((2R,3R,4S,5R)-4-(benzyloxy)-5-((benzyloxy)methyl)-2-(5-fluoro-2,4-dioxo-3,4-dihydropyrimidin-1(2H)-yl)-5-methyltetrahydrofuran-3-yl) 0-phenyl carbonothioate 16 (740 mg, 1.25 mmol) in toluene (12.5 mL) at room temperature, and the resulting mixture heated to 85° C. with stirring for ca. 2 h. The reaction mixture was then cooled to ambient temperature and the solvent removed under reduced pressure. The residue was purified by flash chromatography on SiO₂ (100% EtOAc then 100% IPA) affording the title compound 17 (469 mg, 1.07 mmol, 85%) as a white solid.

Step 5: 5-fluoro-1-((2R,4S,5R)-4-hydroxy-5-(hydroxymethyl)-5-methyltetrahydrofuran-2-yl)pyrimidine-2,4(1H,3H)-dione (Cpd. No. 105). To a suspension of 1-((2R,4S,5R)-4-(benzyloxy)-5-((benzyloxy)methyl)-5-methyltetrahydrofuran-2-yl)-5-fluoropyrimidine-2,4(1H,3H)-dione 17 (230 mg, 522 μmol) in methanol (29.3 mL) was added palladium hydroxide (20 wt. % on carbon, wet; 230 mg) with stirring. The vessel was purged with hydrogen gas from a balloon, then the reaction mixture allowed to stir under hydrogen (1 atm) at ambient temperature for ca. 18 h. The vessel was then purged under positive flow of nitrogen and the reaction mixture filtered through a short plug of Celite, rinsing with methanol as necessary. The filtrate was concentrated in vacuo and the residue purified by reverse-phase chromatography on C18 (10% acetonitrile in 10 mM aqueous ammonium formate) to afford Cpd. No. 105 (7.76 mg, 29.8 μmol, 5.7%) as an off-white powder after lyophilization. ¹H NMR (400 MHZ, CD3OD) δ 8.49 (s, 1H), 8.34 (d, J=7.0 Hz, 1H), 6.14 (td, J=6.1, 1.6 Hz, 1H), 4.36 (t, J=6.0 Hz, 1H), 3.57 (q, J=11.7 Hz, 2H), 2.38-2.26 (m, 2H), 1.12 (s, 3H).

### Example 7

### Synthesis of (2R,3S,5R)-5-(6-amino-2-fluoro-9H-purin-9-yl)-2-(hydroxymethyl)-2-methyltetrahydrofuran-3-ol (Cpd. No. 126)

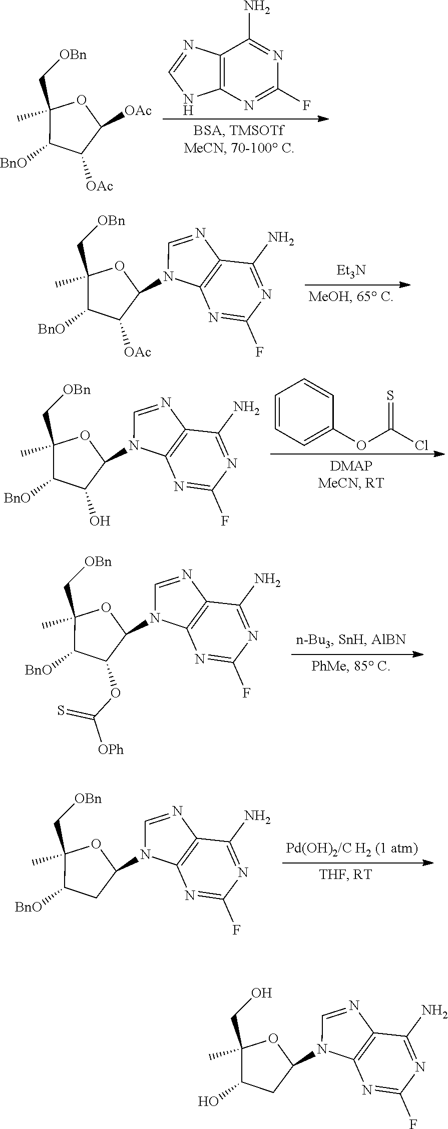

Step 1: (2R,3R,4S,5R)-2-(6-amino-2-fluoro-9H-purin-9-yl)-4-(benzyloxy)-5-((benzyloxy) methyl)-5-methyltetrahydrofuran-3-yl acetate. A flame-dried three-necked round-bottomed flask, equipped with a magnetic stirrer bar and fitted with a reflux condenser, was charged with a suspension of 2-fluoroadenine (1.12 g, 7.29 mmol) in anhydrous MeCN (50.0 mL). N,O-Bis (trimethylsilyl) acetamide (4.50 mL, 17.5 mmol) was added, and the reaction mixture heated to 70° C. with stirring for ca. 1.5 h before cooling to ambient temperature. A solution of (2S,3R,4S,5R)-4-(benzyloxy)-5-((benzyloxy) methyl)-5-methyltetrahydrofuran-2,3-diyl diacetate (2.50 g, 5.83 mmol) in anhydrous MeCN (6.10 mL) was then added dropwise, followed by dropwise addition of trimethylsilyl trifluoromethanesulfonate (1.30 mL, 7.18 mmol) over ca. 5 mins. The reaction mixture was then heated to 100° C. with stirring for co. 18 h. The mixture was then cooled to 0° C. with stirring and quenched with saturated aqueous NaHCO₃ solution (40 mL). Water (50 mL) was added, the mixture poured into a separatory funnel, and the organics extracted with a 4:1 mixture of CHCl₃-IPA (3×50 mL). The combined organics were washed with water (50 mL) and saturated aqueous NaCl solution, dried (anhyd. Na₂SO₄), filtered and concentrated in vacuo to afford the title compound (3.04 g, 5.83 mmol, quant) as a yellow foam, which was utilized in the next reaction without further purification. LC-MS (ESI) m/z 522.3 [M+H]+. LC-MS RT=1.42 min; Method H.

Step 2. (2R,3R,4S,5R)-2-(6-amino-2-fluoro-9H-purin-9-yl)-4-(benzyloxy)-5-((benzyloxy)methyl)-5-methyltetrahydrofuran-3-ol. A round-bottomed flask equipped with a magnetic stirrer bar was charged with a solution of (2R,3R,4S,5R)-2-(6-amino-2-fluoro-9H-purin-9-yl)-4-(benzyloxy)-5-((benzyloxy)methyl)-5-methyltetrahydrofuran-3-yl acetate (3.04 g, 5.83 mmol) in MeOH (77.0 mL). Triethylamine (14.0 mL, 100 mmol) was added, and the reaction mixture was heated to 65° C. with stirring for ca. 4 h. The reaction mixture was then cooled to ambient temperature, concentrated in vacuo to remove the volatiles, and the residue taken up in EtOAc (80 mL) The organics were washed with water (3×30 mL) and saturated aqueous NaCl solution, then dried (anhyd. Na₂SO₄), filtered and concentrated in vacuo to afford the title compound (2.67 g, 5.56 mmol, 95%) as a yellow solid. LC-MS (ESI) m/z 480.2 [M+H]+. LC-MS RT=1.29 min; Method H.

Step 3. O-((2R,3R,4S,5R)-2-(6-amino-2-fluoro-9H-purin-9-yl)-4 (benzyloxy)-5-((benzyloxy) methyl). S-methyltetrahydrofuran-3-yl) O)-phenyl carbonothioate. To a stirring suspension of (2R,3R,4S,5R)-2-(6-amino-2-fluoro-9H-purin-9-yl)-4-(benzyloxy)-5-((benzyloxy) methyl)-5-methyltetrahydrofuran-3-ol (2.67 g, 5.29 mmol) and 4-dimethylaminopyridine (2.05 g, 16.8 mmol) in dry MeCN (112 mL) was added dropwise O-phenyl chlorothionoformate (1.10 mL, 7.93 mmol) at ambient temperature under nitrogen. The reaction mixture was stirred at ambient temperature for ca. 1 h, then the mixture was concentrated in vacuo to remove the volatiles. The residue was taken up in EtOAc (150 mL) and washed with 10% aqueous citric acid solution (100 mL) and saturated aqueous NaCl solution. The organics were then dried (anhyd Na₂SO₄), filtered and concentrated in vacuo. The residue was purified by flash chromatography on silica gel (eluent: 50-100% EtOAc in hexanes) to furnish the title compound (2.30 g, 3.73 mmol, 71%) as an orange foam. LC-MS (ESI) m/z 616.3 [M+H]⁺. LC-MS RT=1.64 min; Method H.

Step 4: 9-(2R,4S,5R)-4-(benzyloxy)-5-((benzyloxy) methyl)-5-methyltetrahydrofuran-2-yl)-2-fluoro-9H-purin-6-amine. AIBN (307 mg, 1.87 mmol) and tributyltin hydride (1.20 mL, 4.45 mmol) were added to a stirring suspension of O-((2R,3R,4S,5R)-2-(6-amino-2-fluoro-9H-purin-9-yl)-4-(benzyloxy)-5-((benzyloxy) methyl)-5-methyltetrahydrofuran-3-yl)O-phenyl carbonothioate (2.30 g, 3.74 mmol) in toluene (37.1 mL). The reaction mixture was heated to 85° C. for ca. 1.5 h, then cooled to ambient temperature. The reaction mixture was then diluted with EtOAc (100 mL) and washed with 0.5 M aqueous KF solution (3×25 mL). The organics were then washed with saturated aqueous NaCl solution and dried (anhyd. Na₂SO₄), filtered, and concentrated in vacuo. The residue was purified by flash chromatography on SiO₂ (eluent: 0-4% MeOH in DCM) to furnish the title compound (295 mg, 638 umol, 17%) as a light tan film. LC-MS (ESI) m/z 464.3 [M+H]+. LC-MS RT=1.41 min; Method H.

Step 5: (2R,3S,5R)-5-(6-amino-2-fluoro-9H-purin-9-yl)-2-(hydroxymethyl)-2-methyltetrahydrofuran-3-ol (Cpd. No. 126). A flame-dried RBF equipped with a magnetic stirrer bar was charged with 9-((2R,4S,5R)-4-(benzyloxy)-5-((benzyloxy)methyl)-5-methyltetrahydrofuran-2-yl)-2-fluoro-9H-purin-6-amine (25.0 mg, 53.9 μmol) and anhydrous THF (2.00 mL). Palladium hydroxide (20 wt. % on carbon, 25.0 mg) was added under nitrogen atmosphere, then the reaction mixture was sparged with hydrogen gas from a balloon for ca. 30 seconds. The reaction mixture was then allowed to stir under hydrogen (1 atm) at ambient temperature for ca. 38 h. The hydrogen balloon was then removed, and the vessel was purged with nitrogen. The reaction mixture was filtered and concentrated in vacuo. The residue was then dissolved in a minimum volume of DMF, loaded onto a pre-packed C18 cartridge, and purified by reverse-phase chromatography (eluent: 5-20% ACN in 10 mM aqueous AmB) afforded Cpd. No. 126 (7.00 mg, 24.7 μmol, 46%) as an amorphous white solid. ¹H NMR (400 MHz, DMSO-d6) δ 8.32 (s, 1H), 7.83 (br. s, 2H), 6.19 (app. t, J=6.6 Hz, 1H), 5.21 (d, J=4.9 Hz, 1H), 5.01 (t, J=5.7 Hz, 1H), 4.39-4.34 (m, 1H), 3.49 (dd, J=11.4, 5.4 Hz, 1H), 3.37-3.29 (m, 1H), 2.75 (dt, J=13.1, 6.4 Hz, 1H), 2.32 (ddd, J=13.2, 6.4, 4.4 Hz, 1H), 1.11 (s, 3H). ¹⁹F NMR (376 MHz, DMSO-d6) δ −52.09 (s, 1F), uncalibrated. LC-MS (ESI) m/z 284.2 [M+H]+. LC-MS RT=0.52 min; Method G.

### Example 8

### Synthesis of (2R,3S,5R)-5-(6-amino-2-fluoro-9H-purin-9-yl)-2-(hydroxymethyl)-2-propyltetrahydrofuran-3-ol (Cpd. No. 129)

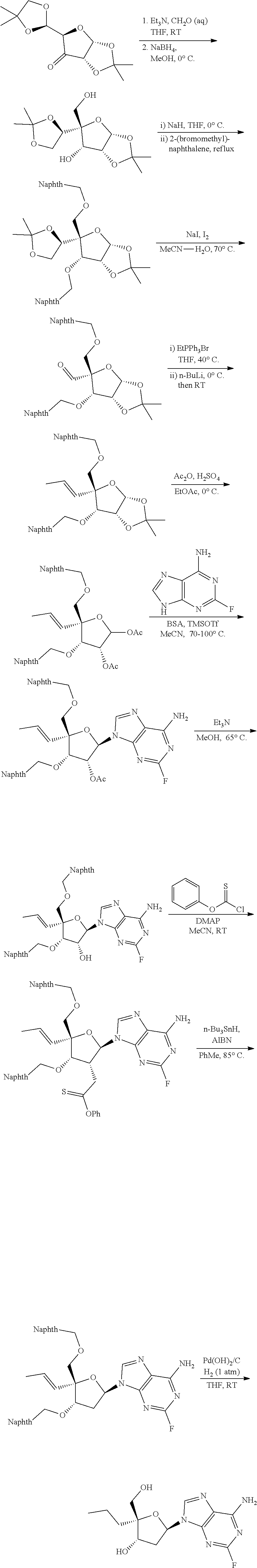

Step 1: (3aR,5R,6S,6aR)-5-((R)-2,2-dimethyl-1,3-dioxolan-4-yl)-5-(hydroxymethyl)-2,2-dimethyltetrahydrofuro[2,3-d][1,3]dioxol-6-ol. To a solution of (3aR,5R,6aS)-5-((S)-2,2-dimethyl-1,3-dioxolan-4-yl)-2,2-dimethyldihydrofuro[2,3-d][1,3]dioxol-6(5H)-one (101 g, 391 mmol) in THF (1.59 L) were added triethylamine (551 mL, 3.91 mol) and 37% aqueous formaldehyde solution (107 mL, 3.91 mol) at ambient temperature with stirring. The reaction mixture was stirred at ambient temperature for ca. 16 h, then the mixture was adjusted to pH 4 using saturated aqueous NH₄Cl solution. The mixture was then diluted with EtOAc (300 mL) and water (300 mL), and the phases were separated. The aqueous phase was extracted with EtOAc (2×300 mL) and the combined organics dried (anhyd. MgSO₄), filtered and concentrated in vacuo. The residue was dissolved in EtOAc (250 mL) at 70° C., and hexane (900 mL) was added dropwise over ca. 30 mins. The resulting turbid solution was allowed to cool to ambient temperature and stirred for ca. 16 h. The resulting precipitate was collected by filtration, and the filter cake washed with hexane (2×600 mL) and allowed to dry. The resulting brown solid was dissolved in MeOH (601 mL) and cooled to 0° C. with stirring, then sodium borohydride (38.3 g, 972 mmol) was added portion-wise to the mixture. Following complete addition, the mixture was allowed to stir at 0° C. for ca. 30 mins, then the reaction was quenched with water. The aqueous phase was extracted with DCM (3×100 mL) and the combined organics dried (MgSO₄), filtered and concentrated in vacuo. The residue was taken up in EtOAc and filtered through a short pad of silica gel. The filtrate was concentrated in vacuo and the residue triturated with a mixture of Et₂O and hexanes to afford the title compound (81.0 g, 279 mmol, 85%) as a white solid. ¹H NMR (400 MHz, CDCl₃) δ 5.91 (d, J=4.1 Hz, 1H), 4.72 (dd, J=6.3, 4.1 Hz, 1H), 4.59 (dd, J=7.2, 6.7 Hz, 1H), 4.32 (t, J=6.5 Hz, 1H), 4.15 (dd, J=9.3, 7.5 Hz, 1H), 3.90 (dd, J=9.3, 6.5 Hz, 1H), 3.78 (dd, J=11.8, 3.0 Hz, 1H), 3.60 (dd, J=11.7, 7.1 Hz, 1H), 2.81 (d, J=6.7 Hz, 1H), 2.14 (dd, J=6.3, 4.2 Hz, 1H), 1.62 (s, 3H), 1.45 (s, 3H), 1.40 (s, 3H), 1.33 (s, 3H).

Step 2: (3aR,5S,6S,6aR)-5-((R)-2,2-dimethyl-1,3-dioxolan-4-yl)-2,2-dimethyl-6-(naphthalen-2-ylmethoxy)-5-((naphthalen-2-ylmethoxy)methyl)tetrahydrofuro[2,3-d][1,3]dioxole. A solution of (3aR,5R,6S,6aR)-5-((R)-2,2-dimethyl-1,3-dioxolan-4-yl)-5-(hydroxymethyl)-2,2-dimethyltetrahydrofuro[2,3-d][1,3]dioxol-6-ol (10.0 g, 34.4 mmol) in anhydrous THF (15.0 mL) was added dropwise to a stirring suspension of sodium hydride (60 wt. % dispersion in mineral oil, 4.15 g, 104 mmol) in anhydrous THF (69.0 mL) at 0° C. The mixture was allowed to stir at 0° C. for ca. 10 mins, then 2-(bromomethyl)naphthalene (23.3 g, 103 mmol) was added, and the reaction mixture was heated to reflux for ca. 30 mins before cooling to ambient temperature. The reaction mixture was then partitioned between EtOAc and water. The organics were washed with saturated aqueous NaCl solution, dried (anhyd. MgSO₄), filtered and concentrated in vacuo. The residue was purified by flash chromatography on SiO₂ (eluent: 100% hexane, then 100% EtOAc) to provide the title compound (10.0 g, 17.2 mmol, 50%). NMR (400 MHz, CDCl₃) δ 7.85-7.70 (m, 7H), 7.66-7.64 (m, 1H), 7.52-7.44 (m, 4H), 7.43 (dd, J=8.4, 1.7 Hz, 1H), 7.31 (dd, J=8.4, 1.6 Hz, 1H), 5.85 (d, J=3.8 Hz, 1H), 4.91 (d, J=11.7 Hz, 1H), 4.85 (dd, J=7.5, 6.5 Hz, 1H), 4.69 (dd, J=5.2, 3.8 Hz, 1H), 4.66 (d, J=11.9 Hz, 1H), 4.59 (d, J=11.6 Hz, 1H), 4.56 (d, J=11.8 Hz, 1H), 4.35 (d, J=5.2 Hz, 1H), 4.10 (dd, J=9.2, 7.6 Hz, 1H), 3.85 (dd, J=9.2, 6.5 Hz, 1H), 3.74 (d, J=10.5 Hz, 1H), 3.64 (d, J=10.5 Hz, 1H), 1.65 (s, 3H), 1.40 (s, 3H), 1.39 (s, 3H), 1.31 (s, 3H). LC-MS (ESI) m/z 588.4 [M+H₂O]+. LC-MS RT=2.12 min; Method J.

Step 3: (3aR,5R,6S,6aR)-2,2-dimethyl-6-(naphthalen-2-ylmethoxy)-5-((naphthalen-2-ylmethoxy)methyl)tetrahydrofuro[2,3-d][1,3]dioxole-5-carbaldehyde. (3aR,5S,6S,6aR)-5-((R)-2,2-dimethyl-1,3-dioxolan-4-yl)-2,2-dimethyl-6-(naphthalen-2-ylmethoxy)-5-((naphthalen-2-ylmethoxy)methyl)tetrahydrofuro[2,3-d][1,3]dioxole (11.0 g, 19.3 mmol) was dissolved in MeCN (272 mL) with stirring, and to the resulting solution were added iodine (3.13 g, 12.3 mmol), NaIO₄ (12.4 g, 57.8 mmol) and water (9.07 mL) at ambient temperature. The resulting mixture was then heated to 70° C. with stirring for ca. 2 h, then cooled to ambient temperature. The volume was partially reduced in vacuo, and the residue was diluted with EtOAc (200 mL) and filtered to remove precipitates. The filter cake was washed with EtOAc (20 mL) and the washings combined with the filtrate. The combined EtOAc solution was washed with 30% saturated aqueous Na₂S₂O₃ solution (100 mL), and the aqueous phase was extracted with EtOAc (3×50 mL). The combined EtOAc phases were washed with saturated aqueous NaCl solution and dried (anhyd. MgSO₄), filtered and concentrated in vacuo to afford the title compound (9.60 g, 19.3 mmol, assumed quant) as a yellow oil. LC-MS (ESI) m/z 516.4 [M+H₂O]+. LC-MS RT=1.99 min; Method J.

Step 4: (3aR,5R,6S,6aR)-2,2-dimethyl-6-(naphthalen-2-ylmethoxy)-5-((naphthalen-2-ylmethoxy)methyl)-5-((E)-prop-1-en-1-yl)tetrahydrofuro[2,3-d][1,3]dioxole. Ethyltriphenylphosphonium bromide (13.8 g, 36.1 mmol) was added to a solution of (3aR,5R,6S,6aR)-2,2-dimethyl-6-(naphthalen-2-ylmethoxy)-5-((naphthalen-2-ylmethoxy)methyl)tetrahydrofuro[2,3-d][1,3]dioxole-5-carbaldehyde (9.00 g, 18.1 mmol) in anhydrous THF (180° C.) at ambient temperature. The mixture was heated to 40° C. with stirring for ca. 2 h, then cooled to 0° C. in an ice bath with stirring. A solution of n-butyllithium in hexanes (2.5 M, 11.2 mL, 28.0 mmol) was then added dropwise. The ice bath was removed, and the reaction mixture warmed to 30° C. with stirring for ca. 2 h. The reaction was quenched with saturated NH₄Cl solution and the organics extracted with Et₂O. The combined organics were washed with water and saturated NaCl solution, then the organics were dried (anhyd. Na₂SO₄), filtered and concentrated in vacuo. The residue was purified by flash chromatography on SiO₂ (eluent: 5-6% EtOAc in hexanes) furnished the title compound as a colourless oil. LC-MS (ESI) m/z 528.4 [M+H₂O]+. LC-MS RT=2.18 min; Method J.

Step 5: (3R,4S,5R)-4-(naphthalen-2-ylmethoxy)-5-((naphthalen-2-ylmethoxy)methyl)-5-((E)-prop-1-en-1-yl)tetrahydrofuran-2,3-diyl diacetate. Acetic anhydride (5.00 mL, 52.9 mmol) was added to a solution of (3aR,5R,6S,6aR)-2,2-dimethyl-6-(naphthalen-2-ylmethoxy)-5-((naphthalen-2-ylmethoxy)methyl)-5-((E)-prop-1-en-1-yl)tetrahydrofuro[2,3-d][1,3]dioxole (9.00 g, 17.6 mmol) in EtOAc (34.7 mL) at ambient temperature, and the resulting mixture cooled to 0° C. with stirring. Concentrated H₂SO₄ (0.19 mL, 3.53 mmol) was added, and the reaction mixture was allowed to warm to ambient temperature with stirring for ca. 16 h. The reaction was then quenched by addition of saturated aqueous NaHCO₃ solution (20 mL) and the phases were separated. The aqueous phase was extracted with EtOAc (3×50 mL) and the combined organics were dried (anhyd. MgSO₄), filtered and concentrated in vacuo to afford the title compound (9.78 g, 17.6 mmol, assumed quant) as a brown oil, as a mixture of anomers. For the major anomer, LC-MS (ESI) m/z 495.3 [M-OAc]+. LC-MS RT=2.15 min; Method J.

Step 6: (2R,3R,4S,5R)-2-(6-amino-2-fluoro-9H-purin-9-yl)-4-(naphthalen-2-ylmethoxy)-5-((naphthalen-2-ylmethoxy)methyl)-5-((E)-prop-1-en-1-yl)tetrahydrofuran-3-yl acetate. N,O-Bis(trimethylsilyl)acetamide (13.3 mL, 51.4 mmol) was added to a suspension of 2-fluoroadenine (3.15 g, 20.6 mmol) in anhydrous MeCN (187 mL), and the mixture heated to 70° C. with stirring for ca. 1 h. The mixture was cooled to ambient temperature, then (3R,4S,5R)-4-(naphthalen-2-ylmethoxy)-5-((naphthalen-2-ylmethoxy)methyl)-5-((E)-prop-1-en-1-yl)tetrahydrofuran-2,3-diyl diacetate (9.50 g, 17.1 mmol) and trimethylsilyl trifluoromethanesulfonate (3.76 mL, 20.6 mmol) were added. The mixture was then heated to 70° C. with stirring until the reaction was determined to be complete by LCMS. The mixture was cooled to ambient temperature, quenched with saturated aqueous NaHCO₃ solution, and the organics extracted with EtOAc. The combined organics were dried (anhyd. Na₂SO₄), filtered, and concentrated in vacuo to furnish the title compound (11.1 g, 17.1 mmol, assumed quant). LC-MS (ESI) m/z 648.3 [M+H]+. LC-MS RT=2.07 min: Method J.

Step 7: (2R,3R,4S,5R)-2-(6-amino-2-fluoro-9H-purin-9-yl)-4-(naphthalen-2-ylmethoxy)-5-((naphthalen-2-ylmethoxy)methyl)-5-((E)-prop-1-en-1-yl)tetrahydrofuran-3-ol. A flame-dried round-bottomed flask equipped with a magnetic stirrer bar was charged with a solution of (2R,3R,4S,5R)-2-(6-amino-2-fluoro-9H-purin-9-yl)-4-(naphthalen-2-ylmethoxy)-5-((naphthalen-2-ylmethoxy)methyl)-5-((E)-prop-1-en-1-yl)tetrahydrofuran-3-yl acetate (9.00 g, 13.9 mmol) in MeOH (187 mL). Tirethylamine (34.6 mL, 246 mmol) was added at ambient temperature, and the reaction mixture was heated to 65° C. with stirring for ca. 18 h. The volatiles were then removed in vacuo and the residue taken up in EtOAc. The organics were washed with water (3×) and saturated aqueous NaCl solution, then dried (anhyd. MgSO), filtered, and concentrated in vacuo. The residue was purified by flash chromatography on SiO₂ (eluent: 50% EtOAc in hexanes) to furnish the title compound (5.50 g, 9.08 mmol, 65%). LC-MS (ESI) m/z 606.4 [M+H]+. LC-MS RT=1.84 min; Method J.

Step 8: O-((2R,3R,4S,5R)-2-(6-amino-2-fluoro-9H-purin-9-yl)-4-(naphthalen-2-ylmethoxy)-5-((naphthalen-2-ylmethoxy)methyl)-5-((E)-prop-1-en-1-yl)tetrahydrofuran-3-yl) 0-phenyl carbonothioate. To a solution of (2R,3R,4S,5R)-2-(6-amino-2-fluoro-9H-purin-9-yl)-4-(naphthalen-2-ylmethoxy)-5-((naphthalen-2-ylmethoxy)methyl)-5-((E)-prop-1-en-1-yl)tetrahydrofuran-3-ol (3.50 g, 5.78 mmol) and 4-dimethylaminopyridine (2.12 g, 17.0 mmol) in acetonitrile (135 mL) was added dropwise O-phenyl chlorothionoformate (969 μL, 6.93 mmol) at ambient temperature. The reaction mixture was stirred at ambient temperature for ca. 1 h, then the volatiles were removed in vacuo. The residue was partitioned between EtOAc and water, and the organics were washed with water and saturated aqueous NaCl solution, then dried (anhyd. Na₂SO₄), filtered and concentrated in vacuo. The residue was purified by flash chromatography on SiO₂ (eluent: 50% EtOAc in hexanes) to afford the title compound (2.50 g, 3.37 mmol, 58%) as a solid. LC-MS (ESI) m/z 742.4 [M+H]+. LC-MS RT=2.16 min; Method J.

Step 9: 2-fluoro-9-((2R,4S,5R)-4-(naphthalen-2-ylmethoxy)-5-((naphthalen-2-ylmethoxy)methyl)-5-((E)-prop-1-en-1-yl)tetrahydrofuran-2-yl)-9H-purin-6-amine. O-((2R,3R,4S,5R)-2-(6-amino-2-fluoro-9H-purin-9-yl)-4-(naphthalen-2-ylmethoxy)-5-((naphthalen-2-ylmethoxy)methyl)-5-((E)-prop-1-en-1-yl)tetrahydrofuran-3-yl) 0-phenyl carbonothioate (1.00 g, 1.35 mmol), AIBN (111 mg, 675 μmol) and tributyltin hydride (449 μL, 1.62 mmol) were dissolved in toluene (9.10 mL) at ambient temperature. The reaction mixture was then heated to 85° C. with stirring for ca. 30 mins before cooling to ambient temperature. The reaction mixture was then directly purified by flash chromatography on SiO₂ (eluent: 50% EtOAc in hexanes) to furnish the title compound (380 mg, 644 μmol, 48%) as a ca. 4:1 mixture of anomers. For the major β-anomer, LC-MS (ESI) m/z 590.5 [M+H]+. LC-MS RT=1.78 min; Method H.

Step 10: (2R,3S,5R)-5-(6-amino-2-fluoro-9H-purin-9-yl)-2-(hydroxymethyl)-2-propyltetrahydrofuran-3-ol (Cpd. No. 129). A flame-dried RBF equipped with a magnetic stirrer bar was charged with 2-fluoro-9-((2R,4S,5R)-4-(naphthalen-2-ylmethoxy)-5-((naphthalen-2-ylmethoxy)methyl)-5-((E)-prop-1-en-1-yl)tetrahydrofuran-2-yl)-9H-purin-6-amine (152 mg, 258 μmol) and anhydrous THF (10.1 mL). Palladium hydroxide (20 wt. % on carbon, 228 mg) was added under nitrogen atmosphere, then the reaction mixture was sparged with hydrogen gas from a balloon for ca. 30 seconds. The reaction mixture was then allowed to stir under hydrogen (1 atm) at ambient temperature for ca. 38 h. The hydrogen balloon was then removed, and the vessel was purged with nitrogen. The reaction mixture was filtered and concentrated in vacuo. The residue was then purified by flash chromatography on SiO₂ (eluent: 10% MeOH in DCM) affording Cpd. No. 129 (36.0 mg, 116 umol, 44%) as an amorphous white solid. ¹H NMR (400 MHZ, DMSO-d6) δ 8.32 (s, 1H), 7.82 (s, 2H), 6.18 (t, J=6.9 Hz, 1H), 5.17 (d, J=4.9 Hz, 1H), 4.92 (t, J=5.5 Hz, 1H), 4.39 (dd, J=8.7, 5.4 Hz, 1H), 3.50 (dd, J=11.5, 5.3 Hz, 1H), 3.39 (dd, J=11.5, 6.1 Hz, 1H), 2.93-2.74 (m, 1H), 2.27 (ddd, J=13.2, 6.1, 3.4 Hz, 1H), 1.67-1.46 (m, 2H), 1.35 (dt, J=10.9, 8.2 Hz, 2H), 0.88 (t, J=7.2 Hz, 3H) LC-MS (ESI) m/z 312.1 [M+H]+LC-MS RT=0.95 min; Method B.

### Example 9

### Synthesis of (2R,3S′,5R)-5-(6-amino-2-fluoro-9H-purin-9-yl)-2-(cyclopropylmethyl)-2-(hydroxymethyl)tetrahydrofuran-3-ol (Cpd. No. 130)

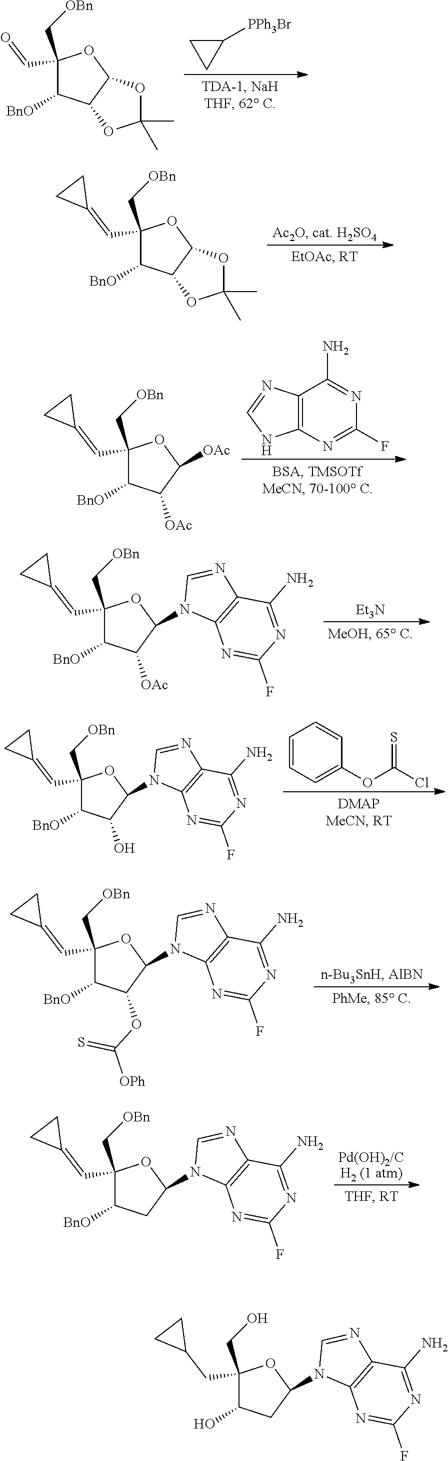

Step 1: (3aR,5R,6S,6aR)-6-(benzyloxy)-5-((benzyloxy)methyl)-5-(cyclopropylidenemethyl)-2,2-dimethyltetrahydrofuro[2,3-d][1,3]dioxole. A flame-dried three-necked round bottomed flask, equipped with a magnetic stirrer bar and fitted with a reflux condenser, was charged with a suspension of sodium hydride (60 wt. % dispersion in mineral oil, 652 mg, 16.3 mmol) and cyclopropyltriphenylphosphonium bromide (6.25 g, 16.3 mmol) in anhydrous THF (54.0 mL) under nitrogen. The reaction mixture was stirred at ambient temperature until a uniform heterogenous mixture was obtained. In the meantime, a separate flame-dried round-bottomed flask, equipped with a magnetic stirrer bar, was charged with a solution of (3aR,5R,6S,6aR)-6-(benzyloxy)-5-((benzyloxy)methyl)-2,2-dimethyltetrahydroftxro[2,3-d][1,3]dioxole-5-carbaldehyde (5.00 g, 12.5 mmol) and tris(3,6-dioxalieptyl)amine (TDA-1, 0.40 mL, 1.25 mmol) in anhydrous THF (18.0 mL). The solution containing the aldehyde was added dropwise to the suspension of the phosphorus ylide, and the reaction mixture heated to 62° C. with stirring for ca. 22 h. The mixture was then cooled to ambient temperature, diluted with Et₂O (200 mL) and filtered through a short pad of silica gel. The filtrate was concentrated in vacuo and the residue was purified by flash chromatography on SiO₂ (eluent: 2-20% EtOAc in hexanes) to furnish the title compound (765 mg, 1.80 mmol, 14%) as a colourless oil. ¹H NMR (400 MHz, CDCl₃) δ 7.37-7.26 (m, 8H), 7.25-7.21 (m, 2H), 6.36 (app p, J=19 Hz, 1H), 5.76 (d, J=3.9 Hz, 1H), 4.73 (d, A of AB, J_{AB}=12.3 Hz, 1H), 4.61 (app. t, J=4.4 Hz, 1H), 4.60 (d, B of AB, J_{AB}=12.2 Hz, 1H), 4.55 (d, A of AB, J_{AB}=12 1 Hz, 1H), 4.41 (d, B of AB, J_{AB}=12.1 Hz, 1H), 4.31 (d, J=4.9 Hz, 1H), 3.62 (d, A of AB, J_{AB}=11.1 Hz, 1H), 3.52 (d, B of AB, J_{AB}=11.1 Hz, 1H), 1.46 (s, 3H), 1.29 (s, 3H), 1.21 (dddd, J=9.8, 8.4, 6.1, 2.3 Hz, 1H), 1.15-1.08 (m, 1H), 1.04-0.91 (m, 2H). LC-MS (ESI) m/z 440.4 [M+H₂O]+LC-MS RT=1.64 min; Method H.

Step 2: (2S,3R,4S,5R)-4-(benzyloxy)-5-((benzyloxy)methyl)-5-(cyclopropylidenemethyl)tetrahydrofuran-2,3-diyl diacetate. A solution of (3aR,5R,6S,6aR)-6-(benzyloxy)-5-((benzyloxy)methyl)-5-(cyclopropylidenemethyl)-2,2-dimethyltetrahydrofuro[2,3-d][1,3]dioxole (765 mg, 1.81 mmol) and acetic anhydride (0.51 mL, 5.40 mmol) in EtOAc (3.60 mL) was cooled to 0° C. with stirring, and concentrated sulfuric acid (20 μL, 357 μmol) was added dropwise. The reaction mixture was then warmed to ambient temperature and stirred overnight. The mixture was then cooled to 0° C. with stirring and quenched by dropwise addition of saturated aqueous NaHCO₃ solution (10 mL). The mixture was warmed to ambient temperature, diluted with EtOAc (10 mL), and the phases separated. The aqueous phase was extracted with EtOAc (3×10 mL) and the combined organics washed with saturated aqueous NaCl solution, dried (anhyd. Na₂SO₄), filtered and concentrated in vauco to furnish the title compound (845 mg, 1.81 mmol, quant.) as a brown oil, as a ca. 4:1 mixture of anomers. For the major anomer: LC-MS (ESI) m/z 484.5 [M+H₂O]+. LC-MS RT=1.60 min; Method H.

Step 3: (2R,3R,4S,5R)-2-(6-amino-2-fluoro-9H-purin-9-yl)-4-(benzyloxy)-5-((benzyloxy)methyl)-5-(cyclopropylidenemethyl)tetrahydrofuran-3-yl acetate. A flame-dried three-necked round-bottomed flask, equipped with a magnetic stirrer bar and fitted with a reflux condenser, was charged with a suspension of 2-fluoroadenine (333 mg, 2.17 mmol) in anhydrous MeCN (16.0 mL). N,O-Bis(trimethylsilyl)acetamide (1.40 mL, 5.45 mmol) was added, and the reaction mixture heated to 70° C. with stirring for ca. 1.5 h. A solution of (2S,3R,4S,5R)-4-(benzyloxy)-5-((benzyloxy)methyl)-5-(cyclopropylidenemethyl)tetrahydrofuran-2,3-diyl diacetate (844 mg, 1.81 mmol) in anhydrous MeCN (4.00 mL) was added dropwise at the same temperature, followed by dropwise addition of trimethylsilyl trifluoromethane sulfonate (400 μL, 2.21 mmol) over ca. 5 mins. The reaction mixture was then heated to 100° C. with stirring for ca. 19 h before cooling to 0° C. The reaction was quenched with saturated aqueous NaHCO₃ solution (20 mL) and diluted with water (10 mL) and saturated aqueous NH₄Cl solution (20 mL). The organics were extracted with a 4:1 mixture of CHCl₃-IPA (3×20 mL), then the combined organics were washed with water (50 mL) and saturated aqueous NaCl solution, dried (anhyd. Na₂SO₄), filtered and concentrated in vacuo. The crude product was purified by flash chromatography on SiO₂ (eluent: 1-3% MeOH in DCM) to furnish the title compound (509 mg, 912 umol, 50%) as an orange foam. ¹H NMR (400 MHZ, CDCl₃) δ 8.10 (s, 1H), 7.39-7.26 (m, 10H), 6.26 (d, J=4.4 Hz, 1H), 6.07 (app. p. J=1.9 Hz, 1H), 5.71 (br. s, 2H), 5.66 (dd, J=5.6, 4.4 Hz, 1H), 4.66 (d, J=5.7 Hz, 1H), 4.55 (d, A of AB, J_{AB}=11.5 Hz, 1H), 4.54 (d, A of AB, J_{AB}=11.8 Hz, 1H), 4.47 (d, B of AB, J_{AB}=11.5 Hz, 1H), 4.46 (d, B of AB, J_{AB}=11.8 Hz, 1H), 3.73 (d, A of AB, J_{AB}=10.6 Hz, 1H), 3.68 (d, B of AB, J_{AB}=10.6 Hz, 1H), 2.04 (s, 3H), 1.27-1.19 (m, 1H), 1.19-1.11 (m, 1H), 1.07-1.01 (m, 2H). LC-MS (ESI) m/z 560.4 [M+H]+. LC-MS RT=1.51 min; Method H.

Step 4: (2R,3R,4S,5R)-2-(6-amino-2-fluoro-9H-purin-9-yl)-4-(benzyloxy)-5-((benzyloxy)methyl)-5-(cyclopropylidenemethyl)tetrahydrofuran-3-ol. Triethylamine (2.29 mL, 16.5 mmol) was added in one portion to a stirring suspension of (2R,3R,4S,5R)-2-(6-amino-2-fluoro-9H-purin-9-yl)-4-(benzyloxy)-5-((benzyloxy)methyl)-5-(cyclopropylidenemethyl)tetrahydrofuran-3-yl acetate (509 mg, 910 μmol) in MeOH (12.0 mL) at ambient temperature. The mixture was heated to 65° C. with stirring for ca. 18 h, then the mixture was cooled to ambient temperature and the volatiles removed in vacuo. The residue was taken up in EtOAc (50 mL) and washed with water (3×25 mL) and saturated aqueous NaCl solution, dried, filtered and concentrated in vacuo to afford the title compound (410 mg, 793 μmol, 87%) as a brown foam. LC-MS (ESI) m/z 518.4 [M+1]+LC-MS RT=1.40 min; Method H.

Step 5: O-(2R,3R,4S,5R)-2-(6-amino-2-fluoro-9H-purin-9-yl)-4-(benzyloxy)-5-((benzyloxy) methyl)-S-(cyclopropylidenemethyl)tetrahydrofuran-3-yl)O-phenyl carbonothioate. A flame-dried round-bottomed flask, equipped with a magnetic stirrer bar and capped with a rubber septum, was charged with a mixture of (2R,3R,4S,5R)-2-(6-amino-2-fluoro-9H-purin-9-yl)-4-(benzyloxy)-5-((benzyloxy) methyl)-5-(cyclopropylidenemethyl)tetrahydrofuran-3-ol (461 mg, 891 umol) and 4-dimethylaminopyridine (326 mg, 2.67 mmol) in anhydrous MeCN (17.9 mL). The mixture was cooled to 0° C. with stirring, then O-phenyl chlorothionoformate (180 uL, 1.30 mmol) was added dropwise. The reaction mixture was warmed to ambient temperature and stirred for ca. 30 mins, then the volatiles were removed in vacuo. The residue was taken up in EtOAc (30 mL), and the organics washed with water (20 mL). The aqueous phase was extracted with EtOAc (3×10 mL), then the combined organics were washed with saturated aqueous NaCl solution, dried (anhyd. Na₂SO₄), filtered and concentrated in vacuo. The residue was purified by flash chromatography on SiO₂ (eluent: 40-100% EtOAc in hexanes) to furnish the title compound (302 mg, 462 umol, 52%) as a pale yellow foam. LC-MS (ESI) m/z 654.5 [M+H]+. LC-MS RT=1.73 min; Method H

Step 6: 9-((2R,4S,5R)-4-(benzyloxy)-S-((benzyloxy) methyl)-5-methyltetrahydrofuran-2-yl)-2-fluoro-9H-purin-6-amine. AIBN (37.9 mg, 231 umol) and tributyltin hydride (150 μL, 559 μmol) were added to a mixture of O-((2R,3R,4S,5R)-2-(6-amino-2-fluoro-9H-purin-9-yl)-4-(benzyloxy)-5-((benzyloxy) methyl)-5-(cyclopropylidenemethyl)tetrahydrofuran-3-yl)O-phenyl carbonothioate (302 mg, 462 umol) in toluene (3.10 mL). The reaction mixture was heated to 85° C. for ca. 1 h, then cooled to ambient temperature. The reaction mixture was then diluted with EtOAc (25 mL) and washed with 0.5 M aqueous KF solution (3×10 mL). The organics were then washed with saturated aqueous NaCl solution and dried (anhyd. Na₂SO₄), filtered, and concentrated in vacuo. The residue was purified by flash chromatography on SiO₂ (eluent: 0-10% MeOH in DCM) to furnish the title compound (295 mg, 638 umol, 17%) as a pale yellow film. LC-MS (ESI) m/z 502.3 [M+H]+. LC-MS RT=1.51 min; Method H.

Step 7: (2R,3S,5R)-5-(6-amino-2-fluoro-9H-purin-9-yl)-2-(cyclopropylmethyl)-2-(hydroxymethyl)tetrahydrofuran-3-ol. A flame-dried RBF equipped with a magnetic stirrer bar was charged with 9-((2R,4S,5R)-4-(benzyloxy)-5-((benzyloxy)methyl)-5-(cyclopropylidenemethyl)tetrahydrofuran-2-yl)-2-fluoro-9H-purin-6-amine (30.0 mg, 59.8 μmol) and THF (2.40 mL). Palladium hydroxide (20 wt. % on carbon, 30.0 mg) was added under nitrogen, then the reaction mixture was sparged with hydrogen gas from a balloon for ca. 30 seconds. The reaction mixture was then allowed to stir under hydrogen (1 atm) at ambient temperature for ca. 19 h. The reaction vessel was then purged with nitrogen, and the mixture filtered. The filtrate was concentrated in vacuo to afford the crude product. Purification of the residue by reverse-phase chromatography on C18 (eluent: 5-100% MeCN in 10% aq. ammonium bicarbonate) to afford Cpd. No. 130 (1.10 mg, 3.41 μmol, 5.7%) as an amorphous white solid. ¹H NMR (400 MHz, CD3OD) δ 8.30 (s, 1H), 6.31 (app. t, J=6.7 Hz, 1H), 4.63 (dd, J=6.2, 4.2 Hz, 1H), 3.88 (d, A of AB, J_{AB}=11.8 Hz, 1H), 3.74 (d, B of AB, J_{AB}=11.9 Hz, 1H), 2.85 (dt, J=13.3, 6.7 Hz, 1H), 2.47 (ddd, J=13.5, 6.5, 4.4 Hz, 1H), 1.71 (dd, J=14.4, 6.2 Hz, 1H), 1.48 (dd, J=14.4, 7.2 Hz, 1H), 0.91-0.83 (m, 1H), 0.50-0.44 (m, 2H), 0.15-0.09 (m, 2H). LC-MS (ESI) m/z 324.3 [M+H]+. LC-MS RT=1.03 min; Method G.

### Example 10

### Synthesis of (2R,3S,5R)-5-(6-amino-2-fluoro-9H-purin-9-yl)-2-(hydroxymethyl)-2-(2-methylprop-1-en-1-yl)tetrahydrofuran-3-ol (Cpd. No. 133) and (2R,3S,5R)-5-(6-amino-2-fluoro-9H-purin-9-yl)-2-(hydroxymethyl)-2-(2-methylprop-1-en-1-yl)tetrahydrofuran-3-ol (Cpd. No. 132)

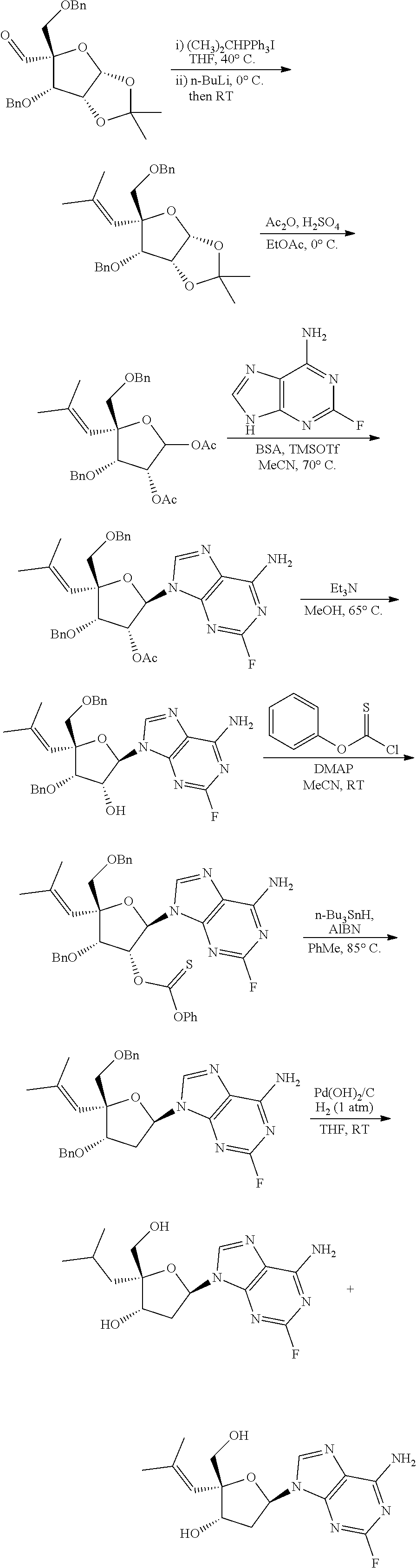

Step 1: (3aR,5R,6S,6aR)-6-(benzyloxy)-5-((benzyloxy)methyl)-2,2-dimethyl-5-(2-methylprop-1-en-1-yl)tetrahydrofuro[2,3-d][1,3]dioxole. (3aR,5R,6S,6aR)-6-(benzyloxy)-5-((benzyloxy)methyl)-2,2-dimethyltetrahydrofuro[2,3-d][1,3]dioxole-5-carbaldehyde (4.25 g, 10.7 mmol) was added to a suspension of isopropyltriphenylphosphonium iodide (8.00 g, 18.1 mmol) in anhydrous THF (35.6 mL) at ambient temperature. The mixture was heated to 40° C. with stirring for ca. 2 h, then cooled to 0° C. with stirring. A solution of n-butyllithium (2.5 M in hexanes, 8.53 mL, 21.3 mmol) was added dropwise, then the mixture was warmed to 30° C. for ca. 2 h. The reaction mixture was then quenched with saturated aqueous NH₄Cl solution (10 mL), the phases separated, and the aqueous phase extracted with Et₂O. The combined organics were washed with water and saturated aqueous NaCl solution, then the organics were dried (anhyd. Na₂SO₄), filtered and concentrated in vacuo to furnish the title compound (4.22 g, 9.97 mmol, 93%) as a yellow oil. The material was utilized without further purification.

Step 2x: (3R,4S,5R)-4-(benzyloxy)-5-((benzyloxy)methyl)-5-(2-methylprop-1-en-1-yl)tetrahydrofuran-2,3-diyl diacetate. To a solution of (3aR,5R,6S,6aR)-6-(benzyloxy)-5-((benzyloxy)methyl)-2,2-dimethyl-5-(2-methylprop-1-en-1-yl)tetrahydrofuro[2,3-d][1,3]dioxole (1.01 g, 2.38 mmol) in EtOAc (9.52 mL) was added acetic anhydride (675 μL, 7.14 mmol), and the resulting solution cooled to 0° C. with stirring. Concentrated H₂SO₄ (70.0 μL, 26.6 mmol) was added, and the reaction mixture warmed to ambient temperature with stirring for ca. 24 h. The reaction was quenched with saturated aqueous NaHCO₃ solution (2 mL) and the mixture stirred vigorously at ambient temperature for ca. 10 mins. The phases were then separated and the aqueous phase extracted with EtOAc (3×5 mL). The combined organic extracts were dried (anhyd. MgSO₄), filtered and concentrated in vacuo to furnish the title compound (1.11 g, 2.38 mmol, assumed quant). ¹H NMR (400 MHz, CDCl₃) δ 7.36-7.26 (m, 10H), 6.14 (d, J=0.6 Hz, 1H), 5.41-5.39 (m, 1H), 5.29 (dd, J=5.0, 0.7 Hz, 1H), 4.64 (d, J=11.7 Hz, 1H), 4.56 (d, J=12.0 Hz, 1H), 4.48 (d, J=11.8 Hz, 1H), 4.45 (d, J=5.0 Hz, 1H), 4.38 (d, J=12.0 Hz, 1H), 3.55 (d, J=11.1 Hz, 1H), 3.39 (d, J=11.1 Hz, 1H), 2.06 (s, 3H), 1.91 (s, 3H), 1.84 (d, J=1.2 Hz, 3H), 1.72 (d, 0.1=1.3 Hz, 3H). LC-MS (ESI) m/z 486.3 [M+H₂O]⁺. LC-MS RT=1.99 min; Method J.

Step 3x: (2R,3R,4S,5R)-2-(6-amino-2-fluoro-9H-purin-9-yl)-4-(benzyloxy)-5-((benzyloxy)methyl)-5-(2-methylprop-1-en-1-yl)tetrahydrofuran-3-yl acetate. A flame-dried three-necked round-bottomed flask, equipped with a magnetic stirrer bar and fitted with a reflux condenser, was charged with a suspension of 2-fluoroadenine (381 mg, 2.49 mmol) in anhydrous MeCN (23.7 mL) at ambient temperature. N,O-Bis(trimethylsilyl)acetamide (1.83 mL, 7.11 mmol) was added, and the mixture heated to 70° C. with stirring for ca. 1 h before cooling to 0° C. A solution of (3R,4S,5R)-4-(benzyloxy)-5-((benzyloxy)methyl)-5-(2-methylprop-1-en-1-yl)tetrahydrofuran-2,3-diyl diacetate (1.11 g, 2.37 mmol) in anhydrous MeCN (4.00 mL) was added, followed by dropwise addition of trimethylsilyl trifluoromethanesulfonate (455 uL, 2.49 mmol). The reaction mixture was then stirred at ambient temperature for ca. 15 mins and then warmed to 70° C. with stirring for ca. 1 h. The mixture was cooled to ambient temperature and quenched with saturated aqueous NaHCO₃ solution (30 mL). The mixture was diluted with saturated aqueous NaCl solution and the organics were extracted with EtOAc (3×30 mL). The combined organics were dried (anhyd. Na₂SO₄), filtered and concentrated in vacuo to furnish the title compound (470 mg, 838 μmol, 35%) as a brown oil. LC-MS (ESI) m/z 562.3 [M+H]⁺. LC-MS RT=1.91 min; Method J.

Step 4x: (2R,3R,4S,5R)-2-(6-amino-2-fluoro-9H-purin-9-yl)-4-(benzyloxy)-5-((benzyloxy)methyl)-5-(2-methylprop-1-en-1-yl)tetrahydrofuran-3-ol. (2R,3R,4S,5R)-2-(6-amino-2-fluoro-9H-purin-9-yl)-4-(benzyloxy)-5-((benzyloxy)methyl)-5-(2-methylprop-1-en-1-yl)tetrahydrofuran-3-yl acetate (470 mg, 838 μmol) was dissolved in MeOH (11.3 mL) at ambient temperature with stirring, and triethylamine (2.09 mL, 14.8 mmol) was added. The mixture was heated to reflux for ca. 18 h, then the mixture was cooled to ambient temperature and concentrated to remove the volatiles. The residue was taken up in EtOAc (15 mL) and washed with water (3×5 mL) and saturated aqueous NaCl solution. The organics were then dried (anhyd. MgSO₄), filtered and concentrated in vacuo to furnish the title compound (273 mg, 525 μmol, 63%) as an oil. LC-MS (ESI) m/z 520.3 [M+H]⁺. LC-MS RT=1.78 min: Method J.

Step 5x: O-((2R,3R,4S,5R)-2-(6-amino-2-fluoro-9H-purin-9-yl)-4-(benzyloxy)-5-((benzyloxy)methyl)-5-(2-methylprop-1-en-1-yl)tetrahydrofuran-3-yl) 0-phenyl carbonothioate. To a stirring solution of (2R,3R,4S,5R)-2-(6-amino-2-fluoro-9H-purin-9-yl)-4-(benzyloxy)-5-((benzyloxy)methyl)-5-(2-methylprop-1-en-1-yl)tetrahydrofuran-3-ol (512 mg, 985 μmol) and 4-dimethylaminopyridine (361 mg, 2.90 mmol) in MeCN (9.85 mL) was added O-phenyl chlorothionoformate (138 μL, 985 μmol) dropwise at ambient temperature. The mixture was then stirred at ambient temperature for ca. 1 h, and the volatiles were removed in vacuo. The residue was partitioned between EtOAc and water, and the organic phase washed with water and saturated aqueous NaCl solution. The organics were then dried (anhyd. Na₂SO₄), filtered and concentrated in vacuo. The residue was filtered through a short silica gel column, eluting with EtOAc, to afford the title compound (231 mg, 352 μmol, 36%) as an off-white solid. LC-MS (ESI) m/z 656.3 [M+H]⁺. LC-MS RT=2.01 min: Method J.

Step 6x: 9-((2R,4S,5R)-4-(benzyloxy)-5-((benzyloxy)methyl)-5-(2-methylprop-1-en-1-yl)tetrahydrofuran-2-yl)-2-fluoro-9H-purin-6-amine. O-((2R,3R,4S,5R)-2-(6-amino-2-fluoro-9H-purin-9-yl)-4-(benzyloxy)-5-((benzyloxy)methyl)-5-(2-methylprop-1-en-1-yl)tetrahydrofuran-3-yl) 0-phenyl carbonothioate (231 mg, 352 μmol), AIBN (28.9 mg, 176 μmol) and tributyltin hydride (117 μL, 423 μmol) were dissolved in toluene (2.38 mL) at ambient temperature under nitrogen. The reaction mixture was heated to 85° C. with stirring for ca. 2 h, then cooled to ambient temperature. The residue was adsorbed onto a mixture of 10% K₂CO₃/SiO₂ and purified by flash chromatography on SiO₂ (eluent: 2-20% MeOH in DCM) to furnish the title compound (31.0 mg, 61.6 μmol, 17%) as a colorless oil. LC-MS (ESI) m/z 504.3 [M+H]+. LC-MS RT=1.84 min; Method J.

Step 7x: (2R,3S,5R)-5-(6-amino-2-fluoro-9H-purin-9-yl)-2-(hydroxymethyl)-2-isobutyltetrahydrofuran-3-ol (Cpd. No. 133) and (2R,3S,5R)-5-(6-amino-2-fluoro-9H-purin-9-yl)-2-(hydroxymethyl)-2-(2-methylprop-1-en-1-yl)tetrahydrofuran-3-ol (Cpd. No. 132). An round-bottomed flask equipped with a magnetic stirrer bar was charged with 9-((2R,4S,5R)-4-(benzyloxy)-5-((benzyloxy)methyl)-5-(2-methylprop-1-en-1-yl)tetrahydrofuran-2-yl)-2-fluoro-9H-purin-6-amine (23.3 mg, 46.3 μmol) and EtOAc (2.60 mL). Palladium hydroxide (20 wt. % on carbon, 23.3 mg) was added under nitrogen, then the reaction mixture was sparged with hydrogen gas from a balloon for ca. 30 seconds. The reaction mixture was then allowed to stir under hydrogen (1 atm) at ambient temperature for ca. 18 h. The reaction vessel was then purged with nitrogen, and the mixture filtered through a plug of Celite®. The filtrate was concentrated in vacuo to afford the crude product. Purification of the residue by reverse-phase chromatography on C18 (eluent: 5-100% MeCN in 10% aq. ammonium formate solution) to afford Cpd. No. 133 (1.30 mg, 3.97 μmol, 8.6%) as an amorphous white solid. LC-MS (ESI) m/z 324.3 [M−H]⁻. LC-MS RT=1.62 min; Method A. Cpd. No. 132 (1.20 mg, 3.70 μmol, 8.0%) was also recovered from this experiment as a white amorphous solid. NMR (400 MHz, CD3OD) δ 8.34 (s, 1H), 6.23 (dd, J=6.6, 5.6 Hz, 1H), 5.42 (s, 1H), 4.68 (t, J=6.4 Hz, 1H), 3.74 (d, J=12.4 Hz, 1H), 3.60 (d, 0.1=12.3 Hz, 1H), 2.73-2.66 (m, 1H), 2.43-2.36 (m, 1H), 1.88 (d, J=1.1 Hz, 3H), 1.80 (d, J=1.2 Hz, 3H). LC-MS (ESI) m/z 324.2 [M+H]+. LC-MS RT=1.50 min; Method A.

### Example 11

### Synthesis of (2S,3S,5R)-5-(6-amino-2-fluoro-9H-purin-9-yl)-2-(hydroxymethyl)-2-(methoxymethyl)tetrahydrofuran-3-ol (Cpd. No. 140)

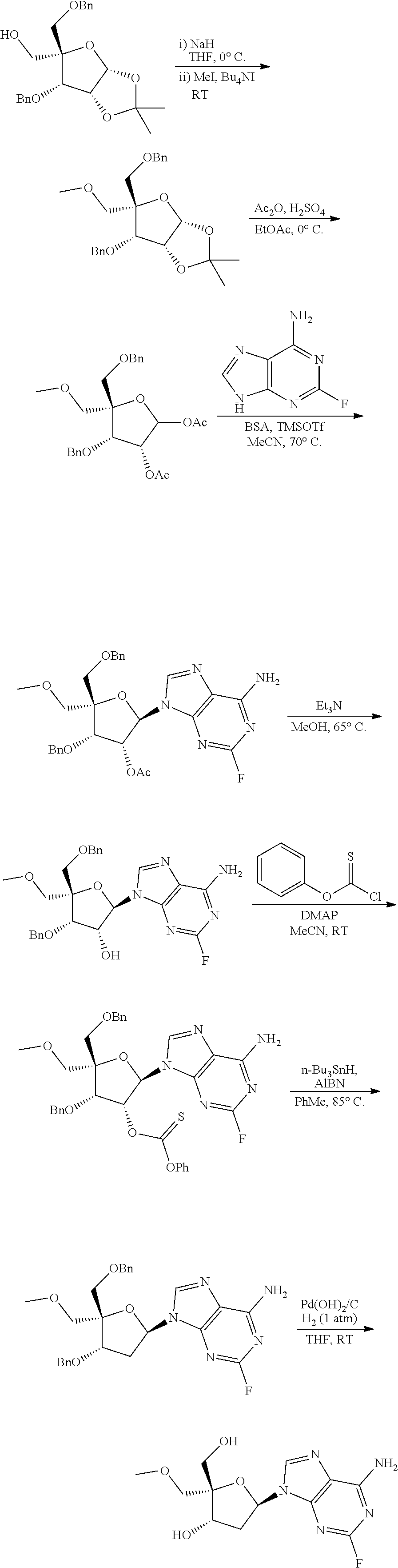

Step 1: (3aR,5R,6S,6aR)-6-(benzyloxy)-5-((benzyloxy)methyl)-5-(methoxymethyl)-2,2-dimethyltetrahydrofuro[2,3-d][1,3]dioxole. To a stirring suspension of sodium hydride (60 wt. % dispersion in mineral oil, 1.20 g, 30.0 mmol) in anhydrous THF (39.1 mL) was added dropwise a solution of ((3aR,5R,6S,6aR)-6-(benzyloxy)-5-((benzyloxy)methyl)-2,2-dimethyltetrahydrofuro[2,3-d][1,3]dioxol-5-yl)methanol (6.00 g, 15.0 mmol) in anhydrous THF (5.00 mL) at 0° C. The mixture was stirred at 0° C. for ca. 15 mins, then neat iodomethane (1.88 mL, 30.0 mmol) was added dropwise to the reaction mixture, followed by tetrabutylammonium iodide (565 mg, 1.50 mmol) in one portion. The mixture was warmed to ambient temperature with stirring for ca. 2 h, then cooled to 0° C. and quenched by addition of saturated aqueous NH₄Cl solution. The volatiles were removed in vacuo, and the organics were extracted with EtOAc. The combined organics were washed with water and saturated aqueous NaCl solution, then dried (anhyd. Na₂SO₄), filtered and concentrated in vacuo. The residue was purified by flash chromatography on SiO₂ (eluent: 16-18% EtOAc in hexanes) to furnish the title compound (5.90 g, 14.3 mmol, 95%) as a colorless oil. LC-MS (ESI) m/z 432.4 [M+H₂O]⁺. LC-MS RT=1.51 min; Method H.

Step 2: (3R,4S,5R)-4-(benzyloxy)-5-((benzyloxy)methyl)-5-(methoxymethyl)tetrahydrofuran-2,3-diyl diacetate. To a solution of (3aR,5R,6S,6aR)-6-(benzyloxy)-5-((benzyloxy)methyl)-5-(methoxymethyl)-2,2-dimethyltetrahydrofuro[2,3-d][1,3]dioxole (6.00 g, 14.5 mmol) in EtOAc (28.5 mL) was added acetic anhydride (4.10 mL, 43.4 mmol), and the resulting solution cooled to 0° C. with stirring. Concentrated H₂SO₄ (155 μL, 2.90 mmol) was added, and the reaction mixture warmed to ambient temperature with stirring for ca. 24 h. The reaction was quenched with saturated aqueous NaHCO₃ solution (20 mL) and the mixture stirred vigorously at ambient temperature for ca. 10 mins. The phases were then separated and the aqueous phase extracted with EtOAc (3×50 mL). The combined organic extracts were dried (anhyd. MgSO₄), filtered and concentrated in vacuo to furnish the title compound (5.02 g, 11.0 mmol, 76%) as a brown oil. LC-MS (ESI) m/z 476.4 [M+H₂O]⁺. LC-MS RT=1.50 min; Method H.

Step 3: (2R,3R,4S,5R)-2-(6-amino-2-fluoro-9H-purin-9-yl)-4-(benzyloxy)-5-((benzyloxy)methyl)-5-(methoxymethyl)tetrahydrofuran-3-yl acetate. A flame-dried three-necked round-bottomed flask, equipped with a magnetic stirrer bar and fitted with a reflux condenser, was charged with a suspension of 2-fluoroadenine (2.40 g, 15.7 mmol) in anhydrous MeCN (143 mL) at ambient temperature. N,O-Bis(trimethylsilyl)acetamide (10.1 mL, 39.3 mmol) was added, and the mixture heated to 70° C. with stirring for ca. 1 h before cooling to ambient temperature. To the cooled reaction mixture was added (3R,4S,5R)-4-(benzyloxy)-5-((benzyloxy)methyl)-5-(methoxymethyl)tetrahydrofuran-2,3-diyl diacetate (6.00 g, 13.1 mmol), followed by dropwise addition of trimethylsilyl trifluoromethanesulfonate (2.87 mL, 15.7 mmol). The reaction mixture was then warmed to 70° C. with stirring for ca. 15 mins. The mixture was cooled to ambient temperature and quenched with saturated aqueous NaHCO₃ solution (50 mL). The organics were extracted with EtOAc (4×50 mL). The combined organics were dried (anhyd. Na₂SO₄), filtered and concentrated in vacuo to furnish the title compound (7.20 g, 13.1 mmol, assumed quant.). LC-MS (ESI) m/z 552.3 [M+H]⁺. LC-MS RT=1.65 min; Method J.

Step 4: (2R,3R,4S,5R)-2-(6-amino-2-fluoro-9H-purin-9-yl)-4-(benzyloxy)-5-((benzyloxy)methyl)-5-(methoxymethyl)tetrahydrofuran-3-ol. (2R,3R,4S,5R)-2-(6-amino-2-fluoro-9H-purin-9-yl)-4-(benzyloxy)-5-((benzyloxy)methyl)-5-(methoxymethyl)tetrahydrofuran-3-yl acetate (7.00 g, 12.7 mmol) was dissolved in MeOH (171 mL) at ambient temperature with stirring, and triethylamine (31.6 mL, 225 mmol) was added. The mixture was heated to reflux for ca. 18 h, then the mixture was cooled to ambient temperature and concentrated to remove the volatiles. The residue was taken up in EtOAc and washed with water and saturated aqueous NaCl solution. The organics were then dried (anhyd. MgSO₄), filtered and concentrated in vacuo to furnish the title compound (6.47 g, 12.7 mmol, assumed quant). LC-MS (ESI) m/z 510.4 [M+H]⁺. LC-MS RT=1.30 min; Method H.

Step 5: O-((2R,3R,4S,5R)-2-(6-amino-2-fluoro-9H-purin-9-yl)-4-(benzyloxy)-5-((benzyloxy)methyl)-5-(methoxymethyl)tetrahydrofuran-3-yl) 0-phenyl carbonothioate. To a stirring solution of (2R,3R,4S,5R)-2-(6-amino-2-fluoro-9H-purin-9-yl)-4-(benzyloxy)-5-((benzyloxy)methyl)-5-(methoxymethyl)tetrahydrofuran-3-ol (2.00 g, 3.93 mmol) and 4-dimethylaminopyridine (1.44 g, 11.5 mmol) in MeCN (91.7 mL) was added O-phenyl chlorothionoformate (658 μL, 4.71 mmol) dropwise at ambient temperature. The mixture was then stirred at ambient temperature for ca. 1 h, and the volatiles were removed in vacuo. The residue was partitioned between EtOAc and water, and the organic phase washed with water and saturated aqueous NaCl solution. The organics were then dried (anhyd. Na₂SO₄), filtered and concentrated in vacuo. The residue was filtered through a short silica gel column, eluting with EtOAc, to afford the title compound (1.60 g, 2.49 mmol, 63%) as an oily colourless liquid. LC-MS (ESI) m/z 646.3 [M+H]⁺. LC-MS RT=1.95 min; Method J.

Step 6: 9-((2R,4S,5R)-4-(benzyloxy)-5-((benzyloxy)methyl)-5-(methoxymethyl)tetrahydrofuran-2-yl)-2-fluoro-9H-purin-6-amine. O-((2R,3R,4S,5R)-2-(6-amino-2-fluoro-9H-purin-9-yl)-4-(benzyloxy)-5-((benzyloxy)methyl)-5-(methoxymethyl)tetrahydrofuran-3-yl) 0-phenyl carbonothioate (1.00 g, 1.55 mmol), AIBN (127 mg, 724 μmol) and tributyltin hydride (515 μL, 1.86 mmol) were dissolved in toluene (10.4 mL) at ambient temperature under nitrogen. The reaction mixture was heated to 85° C. with stirring for ca. 30 mins, then cooled to ambient temperature. The reaction mixture was purified directly by flash chromatography on SiO₂ (eluent: 50% EtOAc in hexanes) to furnish the title compound (480 mg, 0.97 mmol, 63%) as a white solid. LC-MS (ESI) m/z 494.3 [M+H]+. LC-MS RT=1.40 min; Method H.

Step 7: (2R,3S,5R)-5-(6-amino-2-fluoro-9H-purin-9-yl)-2-(hydroxymethyl)-2-isobutyltetrahydrofuran-3-ol (Cpd. No. 140). An round-bottomed flask equipped with a magnetic stirrer bar was charged with 9-((2R,4S,5R)-4-(benzyloxy)-5-((benzyloxy)methyl)-5-(methoxymethyl)tetrahydrofuran-2-yl)-2-fluoro-9H-purin-6-amine (100 mg, 203 umol) and THF (8.00 mL). Palladium hydroxide (20 wt. % on carbon, 150 mg) was added under nitrogen, then the reaction mixture was sparged with hydrogen gas from a balloon for ca. 30 seconds. The reaction mixture was then allowed to stir under hydrogen (1 atm) at ambient temperature for ca. 18 h. The reaction vessel was then purged with nitrogen, and the mixture filtered through a plug of Celite®. The filtrate was concentrated in vacuo to afford the crude product. Purification of the residue by flash chromatography on SiO₂ (eluent: 10% MeOH in DCM) to afford Cpd. No. 140 (36 mg, 115 μmol, 56%) as an amorphous white solid. NMR (400 MHz, DMSO-d6) δ 8.32 (s, 1H), 7.85 (s, 1H), 6.24 (t, J=6.9 Hz, 1H), 5.24 (d, 0.1=5.0 Hz, 1H), 4.94 (t, J=5.7 Hz, 1H), 4.50-4.43 (m, 3H), 3.52 (dd, J=7.9, 5.5 Hz, 1H), 3.45 (d, J=10.2 Hz, 1H), 3.26 (s, 3H), 2.78 (ddd, J=13.4, 7.4, 6.2 Hz, 1H), 2.31 (ddd, J=13.1, 6.3, 3.5 Hz, 1H). LC-MS (ESI) m/z 314.2 [M−H]⁻. LC-MS RT=0.58 min; Method A.

### Example 12

### Synthesis of 4-amino-5-fluoro-1-((2R,4S,5R)-5-(fluoromethyl)-4-hydroxy-5-(hydroxymethyl)tetrahydrofuran-2-yl)pyrimidin-2(1H)-one (Cpd. No. 197)

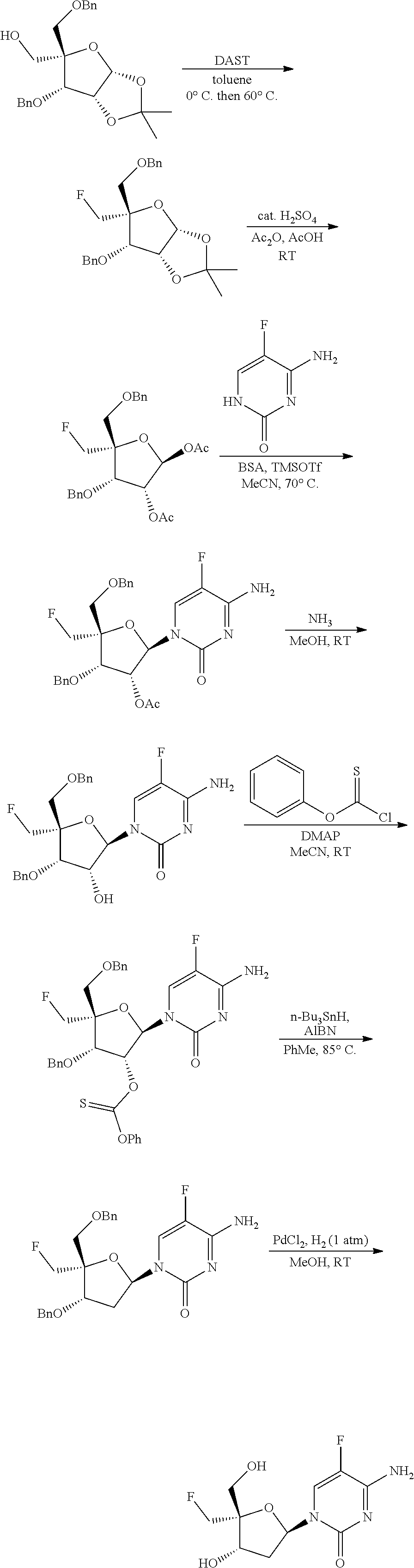

Step 1: (3aR,5R,6S,6aR)-6-(benzyloxy)-5-((benzyloxy)methyl)-5-(fluoromethyl)-2,2-dimethyltetrahydrofuro[2,3-d][1,3]dioxole. A flame-dried round-bottomed flask, equipped with a magnetic stirrer bar and capped with a rubber septum, was charged with a solution of (diethylamino)sulfur trifluoride (7.00 mL, 56.6 mmol) in anhydrous toluene (19.0 mL) under nitrogen atmosphere, and the solution cooled to 0° C. with stirring. A solution of ((3aR,5R,6S,6aR)-6-(benzyloxy)-5-((benzyloxy)methyl)-2,2-dimethyltetrahydrofuro[2,3-d][1,3]dioxol-5-yl)methanol (7.55 g, 18.9 mmol) in anhydrous toluene (75.0 mL), prepared in a separate flame-dried round-bottomed flask, was added dropwise via stainless steel cannula over ca. 15 minutes. The reaction mixture was then heated to 60° C. with stirring for ca. 19 h. The reaction mixture was then cooled to 0° C. and quenched by portion-wise addition of saturated aqueous NaHCO₃ solution (100 mL), pausing to allow for cessation of effervescence between additions. The quenched reaction mixture was then poured into water (200 mL), EtOAc (100 mL) added, and the mixture stirred vigorously for ca. 10 mins at ambient temperature. The mixture was then poured into a separatory funnel, the phases separated, and the aqueous phase extracted with EtOAc (2×50 mL). The combined organics were washed with saturated aqueous NaCl solution, dried (anhyd. Na₂S04), filtered and concentrated in vacuo. The residue was purified by flash chromatography on SiO₂ (10-30% EtOAc in hexanes) to furnish the title compound (4.62 g, 11.5 mmol, 61%) as a yellow oil. ¹H NMR (400 MHz, CDCl₃) δ 7.37-7.24 (m, 10H), 5.77 (d, J=3.6 Hz, 1H), 4.83 (dd, J=79.8, 10.1 Hz, 1H), 4.73 (d, J=12.2 Hz, 1H), 4.72 (dd, J=78.0, 10.2 Hz, 1H), 4.61 (ddd, J=4.8, 3.7, 1.4 Hz, 1H), 4.56 (d, J=12.0 Hz, 1H), 4.54 (d, J=12.1 Hz, 1H), 4.49 (d, J=12.0 Hz, 1H), 4.26 (dd, J=5.1, 1.7 Hz, 1H), 3.61 (dd, J=10.4, 2.0 Hz, 1H), 3.55 (dd, J=10.4, 1.7 Hz, 1H), 1.63 (s, 3H), 1.35 (s, 3H). LC-MS (ESI) m/z 420.5 [M+H₂O]+. LC-MS RT=1.52 min; Method H.

Step 2: (2S,3R,4S,5R)-4-(benzyloxy)-5-((benzyloxy)methyl)-5-(fluoromethyl)tetrahydrofuran-2,3-diyl diacetate. A round-bottomed flask, equipped with a magnetic stirrer bar and capped with a rubber septum, was charged with a solution of (3aR,5R,6S,6aR)-6-(benzyloxy)-5-((benzyloxy)methyl)-5-(fluoromethyl)-2,2-dimethyltetrahydrofuro[2,3-d][1,3]dioxole (4.62 g, 11.5 mmol) and acetic anhydride (4.39 mL, 46.5 mmol) in AcOH (57.0 mL) at ambient temperature. Concentrated sulfuric acid (0.12 mL, 2.25 mmol) was added dropwise with stirring, and the reaction mixture stirred at ambient temperature for ca. 1 h. The reaction mixture was then poured slowly into ice water (200 mL), EtOAc (250 mL) and saturated aqueous NaCl (100 mL) added, and the mixture poured into a separatory funnel. The phases were separated, and the organics washed with saturated aqueous NaHCO₃ solution (2×100 mL) and saturated aqueous NaCl solution, dried (anhyd. Na₂SO₄), filtered and concentrated in vacuo to afford the crude product. Purification by flash chromatography on SiO₂ (10-30/o EtOAc in hexanes) furnished the title compound (2.88 g, 6.46 mmol, 56%) as a pale yellow oil. NMR (400 MHz, CDCl₃) as a ca. 10:1 mixture of diastereomers; for the major diastereomer, δ 7.37-7.22 (m, 10H), 6.17 (s, 1H), 5.34 (d, 1=4.9 Hz, 1H), 4.72-4.64 (m, 1H), 4.60-4.47 ((m, 5H), 4.43 (dd, J=5.0, 1.0 Hz, 1H), 3.69 (dd, J=9.8, 1.7 Hz, 1H), 3.50 (dd, J=9.8, 2.3 Hz, 1H), 2.11 (s, 3H), 1.89 (s, 1H). LC-MS (ESI) m/z 464.5 [M+H₂O]+. LC-MS RT=1.51 min; Method H.

Step 3: (2R,3R,4S,5R)-2-(4-amino-5-fluoro-2-oxopyrimidin-1(2H)-yl)-4-(benzyloxy)-5-((benzyloxy)methyl)-5-(fluoromethyl)tetrahydrofuran-3-yl acetate. A flame-dried three-necked round-bottomed flask, equipped with a magnetic stirrer bar and fitted with a reflux condenser, was charged with a suspension of 5-fluorocytosine (312 mg, 2.42 mmol) in anhydrous acetonitrile (18.0 mL) under nitrogen atmosphere. N,O-Bis(trimethylsilyl)acetamide (1.60 mL, 6.21 mmol) was added, and the reaction mixture heated to 70° C. with stirring for ca. 45 mins. A solution of (2S,3R,4S,5R)-4-(benzyloxy)-5-((benzyloxy)methyl)-5-(fluoromethyl)tetrahydrofuran-2,3-diyl diacetate (900 mg, 2.02 mmol) in anhydrous acetonitrile (2.00 mL), prepared in a separate flame-dried round-bottomed flask, was then added dropwise, followed by dropwise addition of trimethylsilyl trifluoromethanesulfonate (0.44 mL, 2.44 mmol). The reaction mixture was then stirred at 70° C. for ca. 20 mins. The reaction mixture was then cooled to 0° C. with stirring and quenched by slow addition of saturated aqueous NaHCO₃ solution (30 mL). Water (50 mL) was added, the mixture poured into a separatory funnel, and the organics extracted with a 4:1 mixture of CHCl₃-IPA (3×25 mL). The combined organics were washed with water (2×40 mL) and saturated aqueous NaCl solution, dried (anhyd. Na₂SO₄), filtered and concentrated in vacuo to afford the title compound (1.04 g, 2.02 mmol, quant.) as a white foam. The material was utilized directly in the next reaction without further purification. ¹H NMR (400 MHz, CDCl₃) δ 7.96 (d, J=6.5 Hz, 1H), 7.39-7.24 (m, 10H), 6.22 (dd, J=4.3, 1.5 Hz, 1H), 5.56 (br. s, 1H), 5.45 (br. s, 1H), 5.40 (dd, J=5.2, 4.7 Hz, 1H), 4.69-4.38 (m, 7H), 3.86 (dd, J=10.3, 1.8 Hz, 1H), 3.57 (d, J=10.4, 1.8 Hz, 1H), 2.09 (s, 3H). LC-MS (ESI) m/z 516.5 [M+H]+. LC-MS RT=1.27 min; Method H.

Step 4: 4-amino-1-((2R,3R,4S,5R)-4-(benzyloxy)-5-((benzyloxy)methyl)-5-(fluoromethyl)-3-hydroxytetrahydrofuran-2-yl)-5-fluoropyrimidin-2(1H)-one. A round-bottomed flask equipped with a magnetic stirrer bar was charged with (2R,3R,4S,5R)-2-(4-amino-5-fluoro-2-oxopyrimidin-1(2H)-yl)-4-(benzyloxy)-5-((benzyloxy)methyl)-5-(fluoromethyl)tetrahydrofuran-3-yl acetate (1.57 g, 3.05 mmol) and a solution of ammonia in methanol (7N, 50.0 mL, 219 mmol). The reaction mixture was stirred at ambient temperature for ca. 19 h, then sparged with nitrogen gas for ca. 2 h to remove excess ammonia. The reaction mixture was then concentrated in vacuo to furnish the title compound (1.44 g, 3.05 mmol, quant.) as a yellow solid. The material was utilized directly in the next reaction without further purification. NMR (400 MHz, DMSO-d6) δ 7.88 (d, 1=7.1 Hz, 1H), 7.84 (br. s, 1H), 7.59 (br. s, 1H), 7.39-7.25 (m, 10H), 5.92 (dd, J=6.0, 1.9 Hz, 1H), 5.67 (d, J=5.9 Hz, 1H), 4.81 (d, J=11.9 Hz, 1H), 4.65 (dd, J=24.2, 10.1 Hz, 1H), 4.58-4.49 (m, 3H), 4.51 (d, J=11.9 Hz, 1H), 4.34 (dd, J=11.2, 5.8 Hz, 1H), 4.17 (d, J=5.1 Hz, 1H), 3.72 (dd, J=10.1, 2.0 Hz, 1H), 3.64 (dd, J=10.1, 0.8 Hz, 1H). LC-MS (ESI) m/z 474.5 [M+H]+. LC-MS RT=1.18 min; Method H.

Step 5: O-((2R,3R,4S,5R)-2-(4-amino-5-fluoro-2-oxopyrimidin-1(2H)-yl)-4-(benzyloxy)-5-((benzyloxy)methyl)-5-(fluoromethyl)tetrahydrofuran-3-yl) 0-phenyl carbonothioate. To a stirring suspension of 4-amino-1-((2R,3R,4S,5R)-4-(benzyloxy)-5-((benzyloxy)methyl)-5-(fluoromethyl)-3-hydroxytetrahydrofuran-2-yl)-5-fluoropyrimidin-2(1H)-one (1.44 g, 3.05 mmol) in anhydrous acetonitrile (61.0 mL) were added successively DMAP (758 mg, 6.08 mmol) and O-phenyl chlorothionoformate (0.63 mL, 4.56 mmol) at ambient temperature. The reaction mixture was stirred at ambient temperature for ca. 1 h, then the mixture was concentrated in vacuo to remove the volatiles. The residue was taken up in a 4:1 mixture of CHCl₃-IPA (150 mL) and the solution washed successively with 5% aqueous citric acid solution (100 mL) and saturated aqueous NaHCO₃ solution (100 mL). The organics were dried (anhyd. Na₂SO₄), filtered and concentrated in vacuo to furnish the crude product. Purification by flash chromatography on SiO₂ (0-5% MeOH in DCM) afforded the title compound (1.30 g, 2.14 mmol, 70%) as a yellow foam. ¹H NMR (400 MHz, CDCl₃) δ 8.05 (br. s, 1H), 7.87 (d, J=6.3 Hz, 1H), 7.40-7.24 (m, 13H), 7.02-6.98 (m, 2H), 6.41 (dd, J=5.3, 1.5 Hz, 1H), 5.96 (app. t, J=5.4 Hz, 1H), 5.54 (br. s, 1H), 4.74-4.48 (m, 7H), 3.86 (dd, J=10.2, 1.8 Hz, 1H), 3.67 (dd, J=10.3, 2.2 Hz, 1H). LC-MS (ESI) m/z 610.5 [M+H]+. LC-MS RT 1.51 min; Method H.

Step 6. 4-amino-1-((2R,4S,5R)-4-(benzyloxy)-5-((benzyloxy)methyl)-5-(fluoromethyl)tetrahydrofuran-2-yl)-5-fluoropyrimidin-2(1H)-one. A flame-dried three-necked round bottomed flask, equipped with a magnetic stirrer bar and fitted with a reflux condenser, was charged with a suspension of O-((2R,3R,4S,5R)-2-(4-amino-5-fluoro-2-oxopyrimidin-1(2H)-yl)-4-(benzyloxy)-5-((benzyloxy)methyl)-5-(fluoromethyl)tetrahydrofuran-3-yl) O-phenyl carbonothioate (1.30 g, 2.13 mmol) in anhydrous toluene (14.2 mL). AIBN (175 mg, 1.07 mmol) and tributyltin hydride (0.69 mL, 2.56 mmol) were added at ambient temperature with stirring, then the resulting mixture was heated to 85° C. with stirring for ca. 1 h before cooling to ambient temperature. The reaction mixture was then diluted with EtOAc (40 mL) and washed with 0.5M aqueous KF solution (3×20 mL). The organics were then washed with saturated aqueous NaCl solution, dried (anhyd. Na₂SO₄), filtered and concentrated in vacuo to afford the crude product. Purification by flash chromatography on SiO₂ (0-10% MeOH in DCM) furnished the title compound (339 mg, 0.74 mmol, 35%) as a yellow foam. LC-MS (ESI) m/z 458.5 [M+H]+. LC-MS RT 1.56 min; Method 1.

Step 7: 4-amino-5-fluoro-1-((2R,4S,5R)-5-(fluoromethyl)-4-hydroxy-5-(hydroxymethyl)tetrahydrofuran-2-yl)pyrimidin-2(1H)-one (Cpd. No. 197). A flame-dried round-bottomed flask equipped with a magnetic stirrer bar was charged with a solution of 4-amino-1-((2R,4S,5R)-4-(benzyloxy)-5-((benzyloxy)methyl)-5-(fluoromethyl)tetrahydrofuran-2-yl)-5-fluoropyrimidin-2(1H)-one (289 mg, 632 μmol) in methanol (25.0 mL) under nitrogen atmosphere. Palladium(II) chloride (117 mg, 657 μmol) was added in one portion, then the reaction mixture was sparged with hydrogen gas from a balloon for ca. 30 seconds. The reaction mixture was then allowed to stir under hydrogen (1 atm) for ca. 1 h. The hydrogen balloon was then removed, and the vessel purged under positive pressure of nitrogen for ca. 30 seconds. Triethylamine (ca. 2.00 mL) was added dropwise to quench HCl by-products, then the reaction mixture was filtered through a short pad of Celite®, rinsing with small portions of MeOH, and the filtrate concentrated in vacuo. The residue was purified by reverse-phase flash chromatography on C18 (1-25% MeCN in 10 mM aqueous ammonium bicarbonate solution) to furnish Cpd. No. 197 (158 mg, 571 μmol, 90%) as a white amorphous solid after lyophilization. ¹H NMR (400 MHz, DMSO-d6) S 8.03 (d, J=7.2 Hz, 1H), 7.77 (br. s, 1H), 7.53 (br. s, 1H), 6.20 (td, J=6.6, 1.7 Hz, 1H), 5.38 (d, J=4.3 Hz, 1H), 5.28 (t, 0.1=4.7 Hz, 1H), 4.60-4.53 (m, 1H), 4.47-4.41 (m, 1H), 4.37 (app. q, J=4.4 Hz, 1H), 3.57 (dd, J=11.2, 4.0 Hz, 1H), 3.53 (dd, J=11.4, 3.7 Hz, 1H), 2.17-2.13 (m, 2H). ¹⁹F NMR (376 MHz, DMSO-d6) δ −166.82 (dd, J=7.2, 2.0 Hz, 1F), −234.82 (t, J=47.9 Hz, 1F), uncalibrated. LC-MS (ESI) m/z 278.3 [M+H]+. LC-MS RT=0.24 min: Method G.

### Example 13

### Synthesis of 4-amino-1-((2R,4S,5R)-5-(fluoromethyl)-4-hydroxy-5-(hydroxymethyl)tetrahydrofuran-2-yl)pyrimidin-2(1H)-one (Cpd. No. 198)

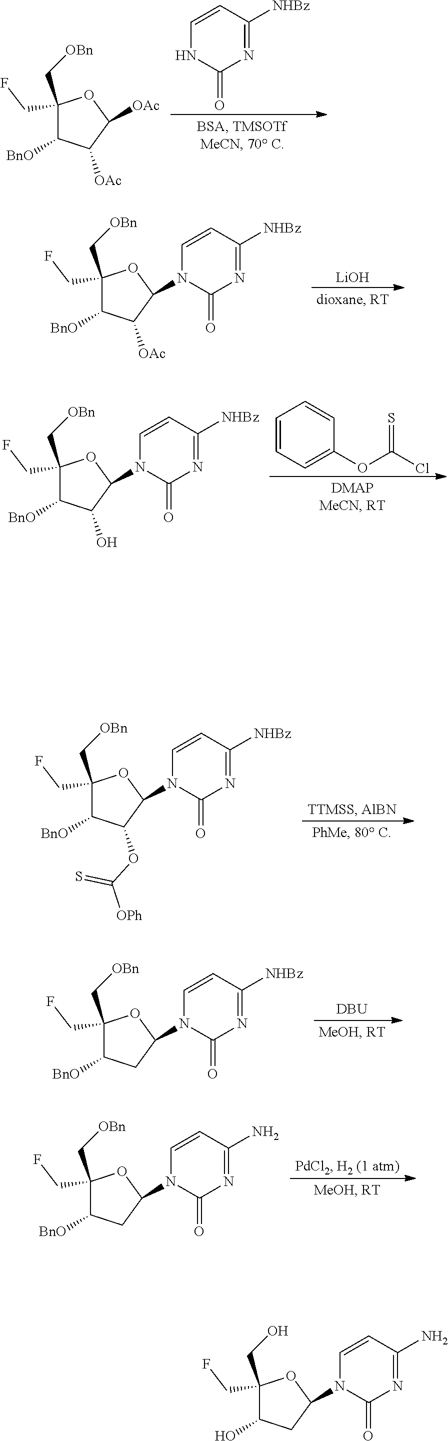

Step 1: (2R,3R,4S,5R)-2-(4-benzamido-2-oxopyrimidin-1(2H)-yl)-4-(benzyloxy)-5-((benzyloxy)methyl)-5-(fluoromethyl)tetrahydrofuran-3-yl acetate. A flame-dried three-necked round-bottomed flask, equipped with a magnetic stirrer bar and fitted with a reflux condenser, was charged with a suspension of N⁴-benzoylcytosine (810 mg, 3.76 mmol) in anhydrous MeCN (28.0 mL) under nitrogen. N,O-Bis(trimethylsilyl)acetamide (1.20 mL, 4.70 mmol) was added, and the reaction mixture heated to 70° C. with stirring for ca. 45 mins. A solution of (2S,3R,4S,5R)-4-(benzyloxy)-5-((benzyloxy)methyl)-5-(fluoromethyl)tetrahydrofuran-2,3-diyl diacetate (1.40 g, 3.14 mmol) in anhydrous MeCN (3.00 mL) was added dropwise, followed by dropwise addition of trimethylsilyl trifluoromethanesulfonate (690 μL, 3.79 mmol) over ca. 5 mins. The reaction mixture was then stirred at ambient temperature for ca. 20 mins, then cooled to ambient temperature. The reaction was quenched with saturated aqueous NaHCO₃ solution (5 mL) and diluted with water. The resulting precipitate was collected by filtration using a Buchner funnel. The filter cake was washed with water (80 mL), then with a 4:1 mixture of CHCl₃-IPA (100 mL, portion-wise). The filtrate was then poured into a separatory funnel, the phases separated, and the aqueous phase extracted with a 4:1 mixture of CHCl₃-IPA (2×25 mL). The combined organics were washed with water (2×50 mL) and saturated aqueous NaCl solution, dried (anhyd. Na₂SO₄), filtered and concentrated in vacuo to furnish the title compound (1.60 g, 2.66 mmol, 85%) as a pale yellow solid. ¹H NMR (400 MHz, CDCl₃) δ 8.90 (br. s, 1H), 8.26 (d, J=7.5 Hz, 1H), 7.90 (d, J=7.5 Hz, 2H), 7.64-7.59 (m, 1H), 7.54-7.49 (m, 2H), 7.46-7.26 (m, 1H), 6.28 (d, J=3.7 Hz, 1H), 5.48 (dd, J=5.5, 3.8 Hz, 1H), 4.68 (dd, J=40.3, 10.3 Hz, 1H), 4.61 (d, J=11.6 Hz, 1H), 4.56 (dd, J=39.4, 10.1 Hz, 1H), 4.51 (d, J=11.6 Hz, 1H), 4.46-4.38 (m, 3H), 3.91 (dd, J=10.4, 1.9 Hz, 1H), 3.60 (dd, J=10.4, 1.7 Hz, 1H), 2.12 (s, 3H). LC-MS (ESI) m/z 602.6 [M+H]+. LC-MS RT=1.53 min; Method H.

Step 2: N-(1-((2R,3R,4S,5R)-4-(benzyloxy)-5-((benzyloxy)methyl)-5-(fluoromethyl)-3-hydroxytetrahydrofuran-2-yl)-2-oxo-1,2-dihydropyrimidin-4-yl)benzamide. To a stirring cloudy solution of (2R,3R,4S,5R)-2-(4-amino-2-oxopyrimidin-1(2H)-yl)-4-(benzyloxy)-5-((benzyloxy)methyl)-5-(fluoromethyl)tetrahydrofuran-3-yl acetate (1.07 g, 1.78 mmol) in 1,4-dioxane (36.0 mL) was added aqueous LiOH solution (2M, 7.10 mL, 3.56 mmol) at ambient temperature. The reaction mixture was stirred at ambient temperature for ca. 90 mins, then the volatiles were removed in vacuo. The residue was partitioned between EtOAc and water, and the combined organics washed with saturated aqueous NaCl solution, dried, filtered and concentrated in vacuo to furnish the title compound (958 mg, 1.71 mmol, 96%) as a pale yellow foam. LC-MS (ESI) m/z 560.5 [M+H]+. LC-MS RT=1.45 min; Method H.

Step 3: O-((2R,3R,4S,5R)-2-(4-benzamido-2-oxopyrimidin-1(2H)-yl)-4-(benzyloxy)-5-((benzyloxy)methyl)-5-(fluoromethyl)tetrahydrofuran-3-yl)O-phenyl carbonothioate. To a stirring suspension of N-(1-((2R,3R,4S,5R)-4-(benzyloxy)-5-((benzyloxy)methyl)-5-(fluoromethyl)-3-hydroxytetrahydrofuran-2-yl)-2-oxo-1,2-dihydropyrimidin-4-yl)benzamide (1.19 g, 2.12 mmol) and 4-dimethylaminopyridine (529 mg, 4.25 mmol) in MeCN (42.5 mL) was added dropwise O-phenyl chlorothionoformate (440 μL, 3.18 mmol) at ambient temperature. The reaction mixture was stirred at ambient temperature for ca. 1 h, then the volatiles were removed in vacuo. The residue was taken up in a 4:1 mixture of CHCl₃-IPA (150 mL) and the organics washed with 5% aqueous citric acid solution (100 mL) and saturated aqueous NaHCO₃ solution (100 mL). The organics were then dried (anhyd. Na₂SO₄), filtered and concentrated in vacuo. The crude product was purified by flash chromatography on SiO₂ (eluent: 0-5% MeOH in DCM) to afford the title compound (1.39 g, 1.99 mmol, 94%) as a yellow foam. ¹H NMR (400 MHz, CDCl₃) δ 8.16 (d, J=7.5 Hz, 1H), 7.90 (d, J=7.4 Hz, 2H), 7.64-7.59 (m, 1H), 7.54-7.49 (m, 2H), 7.44-7.27 (m, 15H), 7.04-7.00 (m, 2H), 6.47 (d, J=4.8 Hz, 1H), 6.06 (t, J=5.1 Hz, 1H), 4.75-4.50 (m, 7H), 3.90 (dd, J=10.2, 1.8 Hz, 1H), 3.70 (dd, J=10.2, 2.0 Hz, 1H). LC-MS (ESI) m/z 696.6 [M+H]+. LC-MS RT=1.71 min; Method H.

Step 4: N-(1-((2R,4S,5R)-4-(benzyloxy)-5-((benzyloxy)methyl)-5-(fluoromethyl)tetrahydrofuran-2-yl)-2-oxo-1,2-dihydropyrimidin-4-yl)benzamide. A round-bottomed flask, equipped with a magnetic stirrer bar and fitted with a reflux condenser, was charged with a solution of O-((2R,3R,4S,5R)-2-(4-benzamido-2-oxopyrimidin-1(2H)-yl)-4-(benzyloxy)-5-((benzyloxy)methyl)-5-(fluoromethyl)tetrahydrofuran-3-yl)O-phenyl carbonothioate (1.39 g, 1.99 mmol) in toluene (13.5 mL) at ambient temperature. AIBN (163 mg, 995 mol) was added in a single portion with stirring, followed by TTMSS (3.10, 10.1 mmol) in one portion. The reaction mixture was then heated to 80° C. with stirring for ca. 1 h. Additional TTMSS (1.65 mL, 5.55 mmol) was added in one portion, and the mixture was stirred at 80° C. for a further ca. 30 mins. The mixture was then cooled to ambient temperature and concentrated in vacuo. The residue was taken up in EtOAc (50 mL), and the organics washed with water (3×20 mL) and saturated aqueous NaCl solution. The organics were then dried (anhyd. Na₂SO₄), filtered and concentrated in vacuo. The residue was purified by flash chromatography on SiO₂ (eluent: 0-4% MeOH in DCM) to furnish the title compound (889 mg, 1.64 mmol, 82%) as a pale yellow foam. ¹H NMR (400 MHz, DMSO-d6) δ 11.25 (s, 1H), 8.25 (d, J=7.5 Hz, 1H), 8.03-7.97 (m, 2H), 7.66-7.60 (m, 1H), 7.55-7.49 (m, 2H), 7.41-7.24 (m, 11H), 6.20 (app. t, J=6.2 Hz, 1H), 4.76-4.47 (m, 6H), 4.42 (dd, J=6.2, 5.0 Hz, 1H), 3.73-3.69 (m, 2H), 2.66-2.60 (m, 1H), 2.32 (dt, J=13.8, 6.3 Hz, 1H). LC-MS (ESI) m/z 544.5 [M+H]+. LC-MS RT=1.53 min; Method H.

Step 5: 4-amino-1-((2R,4S,5R)-4-(benzyloxy)-5-((benzyloxy)methyl)-5-(fluoromethyl)tetrahydrofuran-2-yl)pyrimidin-2(1H)-one. DBU (321 μL, 2.15 mmol) was added to a stirring suspension of N-(1-((2R,4,5R)-4-(benzyloxy)-5-((benzyloxy)methyl)-5-(fluoromethyl)tetrahydrofuran-2-yl)-2-oxo-1,2-dihydropyrimidin-4-yl)benzamide (789 mg, 1.45 mmol) in MeOH (14.5 mL) at ambient temperature. The reaction mixture was then stirred at ambient temperature for ca. 2 h, then the mixture was concentrated in vacuo to remove the volatiles. The residue was purified by reverse-phase chromatography on C18 (eluent: 25-55% MeCN in 10 mM aqueous ammonium bicarbonate solution) to furnish the title compound (281 mg, 639 μmol, 44%) as an amorphous white solid. LC-MS (ESI) m/z 440.5 [M+H]+. LC-MS RT=1.19 min; Method H.

Step 6: 4-amino-1-((2R,4S,5R)-5-(fluoromethyl)-4-hydroxy-5-(hydroxymethyl)tetrahydrofuran-2-yl)pyrimidin-2(1H)-one (Cpd. No. 198). 4-amino-5-fluoro-1-((2R,4S,5R)-5-(fluoromethyl)-4-hydroxy-5-(hydroxymethyl)tetrahydrofuran-2-yl)pyrimidin-2(1H)-one (Cpd. No. 197). A flame-dried round-bottomed flask equipped with a magnetic stirrer bar was charged with a solution of 4-amino-1-((2R,4S,5R)-4-(benzyloxy)-5-((benzyloxy)methyl)-5-(fluoromethyl)tetrahydrofuran-2-yl)pyrimidin-2(1H)-one (280 mg, 637 μmol) in methanol (25.0 mL) under nitrogen atmosphere. Palladium(II) chloride (113 mg, 657 μmol) was added in one portion, then the reaction mixture was sparged with hydrogen gas from a balloon for ca. 30 seconds. The reaction mixture was then allowed to stir under hydrogen (1 atm) for ca. 45 mins. The hydrogen balloon was then removed, and the vessel purged under positive pressure of nitrogen for ca. 30 seconds. Triethylamine (ca. 3.50 mL) was added dropwise to quench HCl by-products, then the reaction mixture was filtered through a short pad of Celite®, rinsing with small portions of MeOH, and the filtrate concentrated in vacuo. The residue was purified by reverse-phase flash chromatography on C18 (1-25% MeCN in 10 mM aqueous ammonium bicarbonate solution) to furnish Cpd. No. 198 (76 mg, 293 μmol, 46%) as a white amorphous solid after lyophilization. NMR (400 MHz, CD3OD) δ 7.97 (d, J=7.5 Hz, 1H), 6.31 (t, J=6.6 Hz, 1H), 5.89 (d, J=7.5 Hz, 1H), 4.59 (d, J=47.5 Hz, 2H), 4.50 (dd, J=6.6, 4.1 Hz, 1H), 3.73 (dd, J=11.7, 1.6 Hz, 1H), 3.68 (dd, J=11.7, 1.6 Hz, 1H), 2.47-2.39 (m, 1H), 2.27 (dt, 1=13.5, 6.7 Hz, 1H). LC−MS (ESI) m/z 258.3 [M−H]−. LC-MS RT=0.16 min; Method G.

### Example 14

### Synthesis of (2R,3S,5R)-5-(6-amino-2-fluoro-9H-purin-9-yl)-2-(1-fluoroethyl)-2-(hydroxymethyl)tetrahydrofuran-3-ol (Cpd. No. 128)

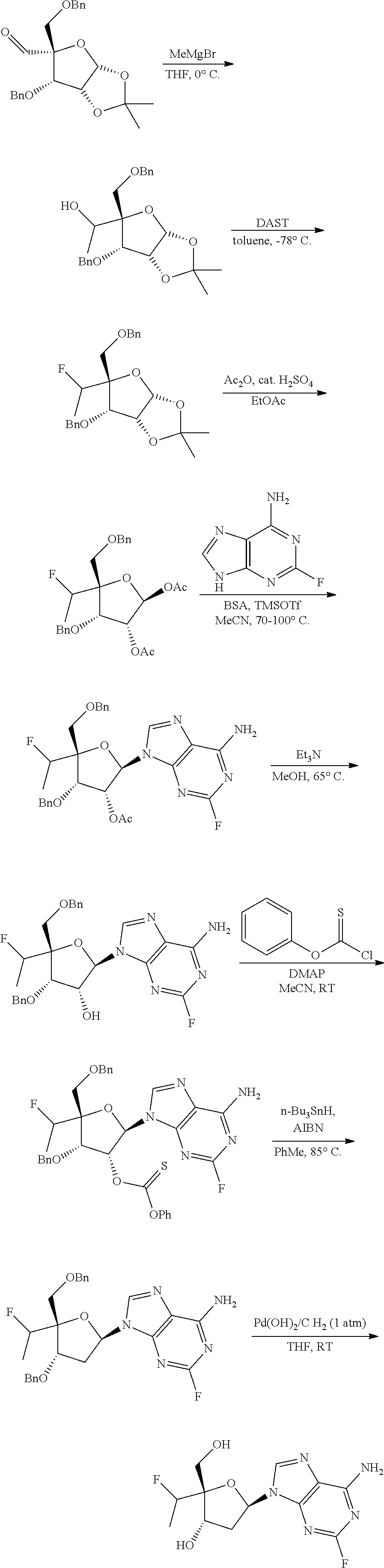

Step 1: 1-((3aR,5S,6S,6aR)-6-(benzyloxy)-5-((benzyloxy)methyl)-2,2-dimethyltetrahydrofuro[2,3-d][1,3]dioxol-5-yl)ethan-1-ol. A flame-dried round-bottomed flask equipped with a magnetic stirrer bar was charged with a solution of (3aR,5R,6S,6aR)-6-(benzyloxy)-5-((benzyloxy)methyl)-2,2-dimethyltetrahydrofuro[2,3-d][1,3]dioxole-5-carbaldehyde (3.00 g, 7.53 mmol) in anhydrous THF (38.0 mL) under nitrogen atmosphere. The solution was cooled to 0° C. with stirring, then a solution of methylmagnesium bromide (3.0 M in Et₂O, 7.50 mL, 22.5 mmol) was added dropwise. The reaction mixture was then warmed to ambient temperature with stirring for ca. 2 h. After this time, the solution was cooled to 0° C., and an additional portion of methylmagnesium bromide solution (3.0 M in Et₂O, 2.50 mL, 7.50 mmol) was added dropwise with stirring. The reaction mixture was then warmed to ambient temperature with stirring for ca. 18 h. The mixture was again cooled to 0° C. with stirring and quenched by addition of saturated aqueous NH₄Cl solution (20 mL). Water (10 mL) was added to dissolve precipitated inorganic salts, and the organics were extracted with EtOAc (3×60 mL). The combined organics were washed with water (3×100 mL) and saturated aqueous NaCl solution, then the organics were dried (anhyd. Na₂SO₄), filtered and concentrated in vacuo. The residue was purified by flash chromatography on SiO₂ (eluent. 2-25% EtOAc in heptanes) to afford the title compound (1.95 g, 4.71 mmol, 62%) as a viscous pale yellow oil. 1H NMR. (400 MHZ, CDCl3) δ 7.38-7.27 (m, 8H), 7.25-7.22 (m, 2H), 5.79 (d, J=3.9 Hz, 1H), 4.86 (d, J=11.4 Hz, 1H), 4.69-4.65 (m, 1H), 4.59 (qd, J=6.3, 3.2 Hz, 1H), 4.54 (d, J=12.1 Hz, 1H), 4.48 (d, J=11.4 Hz, 1H), 4.45-4.39 (m, 2H), 3.79 (d, J=10.7 Hz, 1H), 3.63 (d, J=10.7 Hz, 1H), 3.29 (br. 5s, 1H), 1.59 (s, 3H), 1.35 (s, 3H), 1.20 (d, J=6.7 Hz, 3H) LC-MS (ESI) m/z 432.4 [M+H₂O]+. LC-MS RT=1.45 min; Method H.

Step 2: (3aR,5R,6S,6aR)-6-(benzyloxy)-5-((benzyloxy) methyl)-5-(1-fluoroethyl)-2,2-dimethyltetrahydrofuro[2,3-d][1,3]dioxole. A solution of 1-((3aR,5S,6S,6aR)-6-(benzyloxy)-5-((benzyloxy) methyl)-2,2-dimethyltetrahydrofuro[2,3-d][1,3]dioxol-5-yl) ethan-1-ol (1.85 g, 4.46 mmol) in anhydrous toluene (3.70 mL) was added dropwise to a solution of DAST (1.10 mL, 8.93 mmol) in anhydrous toluene (14.3 mL) at −78° C. with stirring. The reaction mixture was then allowed to slowly warm to ambient temperature with stirring overnight. The mixture was then cooled to 0° C. with stirring and quenched by dropwise addition of saturated aqueous NaHCO₃ solution (20 mL). The reaction mixture was then warmed to ambient temperature, diluted with water (20 mL), and the organics extracted with EtOAc (3×20 mL). The combined organics were washed with saturated aqueous NaCl solution, dried (anhyd. Na₂SO₄), filtered and concentrated in vacuo. The residue was purified by flash chromatography on SiO₂ (eluent: 5-15% EtOAc in hexanes) to furnish the title compound (1.40 g, 3.36 mmol, 75%) as a colorless oil. ¹H NMR (400 MHZ, CDCl3) δ 7.38-7.28 (m, 8H), 7.26-7.23 (m, 2H), 5.78 (dd, J=3.9, 1.6 Hz, 1H), 5.36 (dq, ²J_{H-F}=46.3, J=6.6 Hz, 1H), 4.74 (d, J=12.0 Hz, 1H), 4.68 (dd, J=5.5, 4.0 Hz, 1H), 4.57 (d, J=12.2 Hz, 1H), 4.54 (d, J=12.2 Hz, 1H), 4.45 (d, J=11.9 Hz, 1H), 4.28 (d, J=5.5 Hz, 1H), 3.65 (d, ⁴J_{H-F}=3.0 Hz, 2H), 1.54 (s, 3H), 1.45 (dd, ³J_{H-F}=25.4, J=6.6 Hz, 3H), 1.34 (s, 3H), ¹⁹F NMR (376 MHz, CDCl₃) δ−183.52-−183.88 (m, 1F), uncalibrated. LC-MS (ESI) m/z 434.4 [M+H₂O]+. LC-MS RT=1.64 min; Method H.

Step 3: (2S,3R,4S,5R)-4-(benzyloxy)-5-((benzyloxy)methyl)-5-(1-fluoroethyl)tetrahydrofuran-2,3-diyl diacetate. A solution of (3aR,5R,6S,6aR)-6-(benzyloxy)-5-((benzyloxy)methyl)-5-(1-fluoroethyl)-2,2-dimethyltetrahydrofuro[2,3-d][1,3]dioxole (1.49 g, 3.57 mmol) and acetic anhydride (1.00 mL, 10.6 mmol) in EtOAc (7.10 mL) was cooled to 0° C. with stirring, and concentrated sulfuric acid (40 μL, 714 μmol) added dropwise. The reaction mixture was then warmed to ambient temperature and stirred overnight. The reaction mixture was cooled to 0° C. and quenched by dropwise addition of saturated aqueous NaHCO₃ solution (10 mL). The mixture was then warmed to ambient temperature, diluted with EtOAc (10 mL), and the phases separated. The aqueous phase was extracted with EtOAc (3×10 mL) and the combined organics washed with saturated aqueous NaCl solution, dried (anhyd. Na₂SO₄), filtered and concentrated in vacuo to furnish the title compound (1.50 g, 3.27 mmol, 91%), as a ca. 4:1 mixture of anomers, as a brown waxy solid. For the major anomer, LC-MS (ESI) m/z 478.4 [M+H₂O]+. LC-MS RT=1.59 min; Method H.

Step 4: (2R,3R,4S,5R)-2-(6-amino-2-fluoro-9H-purin-9-yl)-4-(benzyloxy)-5-((benzyloxy)methyl)-5-(1-fluoroethyl)tetrahydrofuran-3-yl acetate. A flame-dried three-necked round-bottomed flask, equipped with a magnetic stirrer bar and fitted with a reflux condenser, was charged with a suspension of 2-fluoroadenine (569 mg, 3.72 mmol) in anhydrous MeCN (29.0 mL). N,O-Bis(trimethylsilyl)acetamide was added, and the mixture heated to 70° C. with stirring for ca. 1.5 h. After this time, a solution of (2S,3R,4S,5R)-4-(benzyloxy)-5-((benzyloxy)methyl)-5-(1-fluoroethyl)tetrahydrofuran-2,3-diyl diacetate (1.50 g, 3.10 mmol) in anhydrous MeCN (4.00 mL) was added dropwise at the same temperature, followed by trimethylsilyl trifluoromethanesulfonate (0.68 mL, 3.76 mmol). The reaction mixture was then heated to 100° C. with stirring for ca. 19 h. The reaction mixture was then cooled to 0° C. and quenched by addition of saturated aqueous NaHCO₃ solution (10 mL). The mixture was warmed to ambient temperature with stirring. A 4:1 mixture of CHCl₃-IPA (40 mL), water (10 mL), saturated aqueous NaHCO₃ solution (10 mL) and saturated aqueous NH₄C1 (20 mL) were added, the entire mixture poured into a separatory funnel, and the phases separated. The aqueous phase was extracted with a 4:1 mixture of CHCl₃-IPA (2×25 mL), and the combined organics were washed with water (50 mL) and saturated aqueous NaCl solution. The organics were then dried (anhyd. Na₂SO₄), filtered and concentrated in vacuo to furnish the title compound (1.65 g, 2.98 mmol, 96%) as a foam. LC-MS (ESI) m/z 554.4 [M+H]+. LC-MS RT=1.49 min; Method H.

Step 5: (2R,3R,4S,5R)-2-(6-amino-2-fluoro-9H-purin-9-yl)-4-(benzyloxy)-5-((benzyloxy)methyl)-5-(1-fluoroethyl)tetrahydrofuran-3-ol. Triethylamine (7.50 mL, 53.8 mmol) was added in one portion to a stirring suspension of (2R,3R,4S,5R)-2-(6-amino-2-fluoro-9H-purin-9-yl)-4-(benzyloxy)-5-((benzyloxy)methyl)-5-(1-fluoroethyl)tetrahydrofuran-3-yl acetate (1.64 g, 2.97 mmol) in MeOH (40.0 mL) at ambient temperature. The mixture was then heated to 65° C. with stirring for ca. 18 h. The mixture was then cooled to ambient temperature and the volatiles removed in vacuo. The residue was taken up in EtOAc (50 mL) and washed with water (3×25 mL) and saturated aqueous NaCl solution. The organics were then dried (anhyd. Na₂SO₄), filtered and concentrated in vacuo to furnish the title compound (1.26 g, 2.46 mmol, 83%) as a beige solid. LC-MS (ESI) m/z 512.4 [M+H]+. LC-MS RT=1.38 min; Method H.

Step 6: 9-((2R,4S,5R)-4-(benzyloxy)-5-((benzyloxy)methyl)-5-(1-fluoroethyl)tetrahydrofuran-2-yl)-2-fluoro-9H-purin-6-amine. A flame-dried round-bottomed flask equipped with a magnetic stirrer bar was charged with a mixture of (2R,3R,4S,5R)-2-(6-amino-2-fluoro-9H-purin-9-yl)-4-(benzyloxy)-5-((benzyloxy)methyl)-5-(1-fluoroethyl)tetrahydrofuran-3-ol (1.16 g, 2.04 mmol) and 4-dimethylaminopyridine (749 mg, 6.13 mmol) in anhydrous MeCN (41.0 mL). The mixture was cooled to 0° C. with stirring, then O-phenyl chlorothionoformate (0.43 mL, 3.11 mmol) was added dropwise. The reaction mixture was then warmed to ambient temperature and stirred for ca. 30 mins. The reaction mixture was then concentrated in vacuo to remove the volatiles, and the residue partitioned between EtOAc (40 mL) and water (40 mL). The phases were separated, and the aqueous phase extracted with EtOAc (3×20 mL) and a 4:1 mixture of CHCl₃-IPA (2×10 mL). The combined organics were washed with saturated aqueous NaCl solution, dried (anhyd. Na₂SO₄), filtered and concentrated in vacuo. The residue was then dissolved in anhydrous toluene (14.0 mL) with stirring, and tris(trimethylsilyl)silane (0.75 mL, 2.43 mmol) and AIBN (167 mg, 1.02 mmol) were added. The reaction vessel was immersed in a pre-heated oil bath and stirred at 85° C. for ca. 2 h. After this time, additional portions of AIBN (167 mg, 1.02 mmol) and tris(trimethylsilyl)silane (0.75 mL, 2.43 mmol) were added, and the mixture heated at 85° C. for ca. 1 h. The mixture was then cooled to ambient temperature, the volatiles removed in vacuo and the residue partitioned between DCM and water. The organics were washed with saturated aqueous NaCl solution, dried (anhyd. Na₂SO₄), filtered and concentrated in vacuo. The residue was then dissolved in anhydrous toluene (14.0 mL), tris(trimethylsilyl)silane (0.75 mL, 2.43 mmol) and AIBN (167 mg, 1.02 mmol) were added, and the mixture was heated to 110° C. for ca. 1 h. The mixture was then cooled to ambient temperature, and the reaction mixture partitioned between EtOAc and water. The organics were washed with saturated aqueous NaCl solution, dried (anhyd. Na₂SO₄), filtered and concentrated in vacuo. Purification of the residue by flash chromatography on SiO₂ (eluent: 10-50% EtOAc/DCM) furnished the title compound (92.0 mg, 186 μmol, 9.1%) as a tan foam. LC-MS (ESI) m/z 496.4 [M+H]+. LC-MS RT=1.47 min; Method H.

Step 7: (2R,3S,5R)-5-(6-amino-2-fluoro-9H-purin-9-yl)-2-(1-fluoroethyl)-2-(hydroxymethyl)tetrahydrofuran-3-ol (Cpd. No. 128). A flame-dried round-bottomed flask equipped with a magnetic stirrer bar was charged with 9-((2R,4S,5R)-4-(benzyloxy)-5-((benzyloxy)methyl)-5-(1-fluoroethyl)tetrahydrofuran-2-yl)-2-fluoro-9H-purin-6-amine (30.0 mg, 60.5 μmol) and anhydrous THF (2.40 mL) under nitrogen. Palladium hydroxide (20 wt. % on carbon, 30.0 mg) was added in one portion, then the reaction mixture was sparged with hydrogen gas from a balloon for ca. 30 seconds. The reaction mixture was then allowed to stir under hydrogen (1 atm) at ambient temperature for ca. 19 h. After this time, fresh palladium hydroxide (20 wt. % on carbon, 15.0 mg) was added, a fresh hydrogen balloon was affixed to the reaction vessel, and the mixture stirred under hydrogen (1 atm) at ambient temperature for ca. 22 h. The reaction vessel was purged with nitrogen, and the reaction mixture filtered. The filtrate was concentrated in vacuo and the residue purified by reverse-phase chromatography on C18 (eluent: 5-25% MeCN in 10 mM aqueous ammonium bicarbonate solution) to furnish Cpd. No. 128 (5.76 mg, 18.2 μmol, 30%) as an amorphous white solid. ¹H NMR (400 MHz, DMSO-d6) δ 8.32 (s, 1H), 7.88 (br. s, 1H), 7.85 (br. s, 1H), 6.27 (app. t, J=6.9 Hz, 1H), 5.49 (d, J=4.9 Hz, 1H), 5.11 (t, 0.1=5.6 Hz, 1H), 4.94 (dq, 1=46.3, 6.4 Hz, 1H), 4.68-4.63 (m, 1H), 3.63-3.53 (m, 2H), 2.84 (dt, J=13.6, 6.8 Hz, 1H), 2.32 (ddd, J=13.7, 6.3, 3.9 Hz, 1H), 1.41 (dd, J=25.7, 6.4 Hz, 3H). ¹⁹F NMR (376 MHz, DMSO-d6) δ −51.98 (s, 1F), −183.22-−183.60 (m, 1F), uncalibrated. LC-MS (ESI) m/z 316.7 [M+H]+. LC-MS RT=0.76 min; Method G.

### Example 15

### Synthesis of (2R,3S,5R)-5-(6-amino-2-chloro-9H-purin-9-yl)-2-ethyl-2-(hydroxymethyl)tetrahydrofuran-3-ol (Cpd. No. 176)

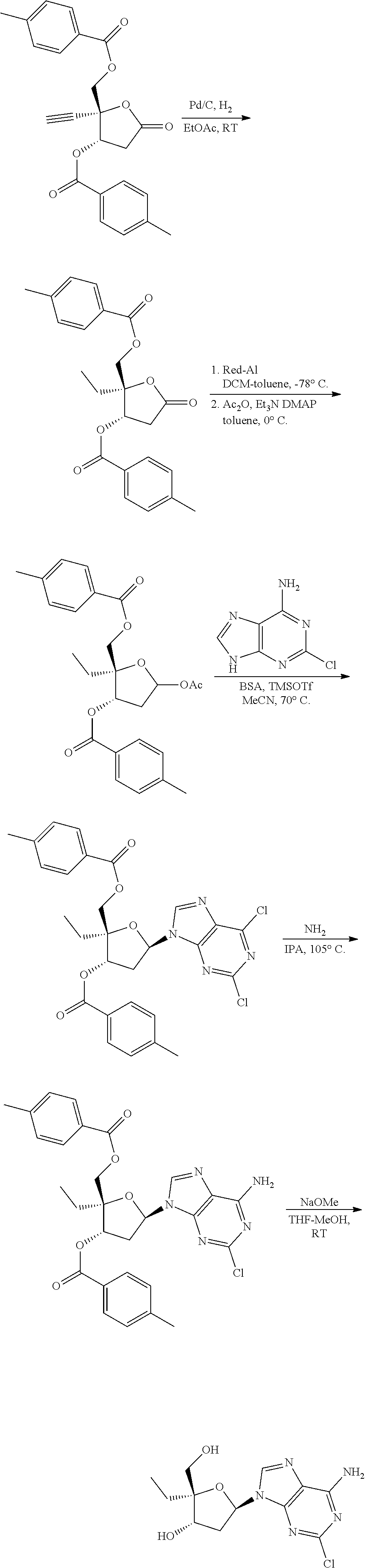

Step 1: (2R,3S)-2-ethyl-2-(((4-methylbenzoyl)oxy)methyl)-5-oxotetrahydrofuran-3-yl 4-methylbenzoate. A round-bottomed flask equipped with a magnetic stirrer bar was charged with a solution of (2R,3S)-2-ethynyl-2-(((4-methylbenzoyl)oxy)methyl)-5-oxotetrahydrofuran-3-yl 4-methylbenzoate (5.00 g, 12.7 mmol) in EtOAc (51.0 mL), to which palladium on carbon (10 wt. % loading, 5.00 g) was added in one portion under nitrogen. The reaction vessel was then purged with hydrogen gas from a balloon, and the reaction mixture allowed to stir under hydrogen (1 atm) for ca. 22 h. The reaction vessel was then purged with nitrogen and the mixture filtered through a short pad of Celite®. The filter cake was rinsed with EtOAc and the filtrate concentrated in vacuo to furnish the title compound (5.05 g, 12.7 mmol, assumed quant) as a light grey solid. LC-MS (ESI) m/z 397.3 [M+H]+. LC-MS RT=1.58 min; Method H.

Step 2: (2R,3S)-5-acetoxy-2-ethyl-2-(((4-methylbenzoyl)oxy)methyl)tetrahydrofuran-3-yl 4-methylbenzoate. A flame-dried round-bottomed flask equipped with a magnetic stirrer bar was charged with a solution of (2R,3S)-2-ethyl-2-(((4-methylbenzoyl)oxy)methyl)-5-oxotetrahydrofuran-3-yl 4-methylbenzoate (5.03 g, 12.7 mmol) in a mixture of toluene (75.0 mL) and DCM (34.0 mL). The solution was cooled to −78° C. with stirring, then a solution of Red-Al (60 wt. % in toluene, 4.54 mL, 14.0 mmol) was added dropwise over ca. 15 mins. The reaction mixture was stirred at −78° C. for ca. 1 h, then quenched by dropwise addition of a solution of AcOH (7.30 mL, 127 mmol) in DCM (15 mL) over ca. 15 mins, maintaining the internal temperature below −60° C. until addition was complete. The mixture was then warmed to ambient temperature and washed sequentially with 1M aqueous H₂SO₄ solution (50 mL) and 50% saturated aqueous NaCl solution (50 mL). The organics were then dried (anhyd. Na₂SO₄), filtered, and partially concentrated in vacuo to remove the majority of the DCM. The residual toluene solution (ca. 42 mL) was cooled to 0° C. with stirring, and 4-dimethylaminopyridine (310 mg, 2.54 mmol) was added in small portions, followed by triethylamine (2.20 mL, 15.6 mmol) dropwise. Acetic anhydride (1.20 mL, 12.7 mmol) was then added slowly dropwise, and the reaction mixture stirred at 0° C. for ca. 1 h before quenching with water (5 mL). The mixture was warmed to ambient temperature, and the organics washed with aqueous 10% citric acid solution (2×20 mL), 50% saturated aqueous NaHCO₃ solution (2×20 mL) and water (20 mL). The organics were then washed with saturated aqueous NaCl solution and dried (anhyd. Na₂SO₄), filtered, and concentrated in vacuo. The crude product was purified by flash chromatography on SiO₂ (eluent: 5-40% EtOAc in hexanes) to furnish the title compound (2.49 g, 5.37 mmol, 42%) as a viscous colorless oil. ¹H NMR (400 MHZ, CDCl3) as a ca. 4:1 mixture of anomers; for the major anomer, δ 8 7.93 (d, J=8.2 Hz, 2H), 7.93 (d, J=8.2 Hz, 2H), 7.28-7.26 (m, 2B), 7.19 (d, J=8.0 Hz, 2H), 6.45 (dd, J=5.8, 1.9 Hz, 1H), 5.81 (t, J=6.8 Hz, 1H), 4.46 (d, J=11.6 Hz, 1H), 4.40 (d, J=11.6 Hz, 1H), 2.70 (ddd, J=14.3, 7.2, 2.0 Hz, 1H), 2.50-2.44 (m, 1H), 2.43 (s, 3H), 2.39 (s, 3H), 1.96 (s, 3H), 1.92-1.81 (m, 2H), 1.06 (t, J=7.5 Hz, 3H).

Step 3: (2R,3S,5R)-5-(2,6-dichloro-9H-purin-9-yl)-2-ethyl-2-(((4-methylbenzoyl)oxy)methyl)tetrahydrofuran-3-yl 4-methylbenzoate. A flame-dried round-bottomed flask, equipped with a magnetic stirrer bar and fitted with a reflux condenser, was charged with a suspension of 2,6-dichloropurine (263 mg, 1.36 mmol) in anhydrous MeCN (10.0 mL). N,O-Bis(trimethylsilyl)acetamide (880 μL, 3.42 mmol) was added, and the mixture heated to 70° C. with stirring for ca. 1.5 h. A solution of (2R,3S)-5-acetoxy-2-ethyl-2-(((4-methylbenzoyl)oxy)methyl)tetrahydrofuran-3-yl 4-methylbenzoate (500 mg, 1.14 mmol) in anhydrous MeCN (1.30 mL) was added dropwise, followed by dropwise addition of trimethylsilyl trifluoromethanesulfonate (250 μL, 1.38 mmol) over ca. 5 min. The reaction mixture was then stirred at 70° C. overnight. The mixture was then cooled to ambient temperature, quenched with saturated aqueous NaHCO₃ solution (25 mL), and diluted with water (40 mL). The organics were extracted with a 4:1 mixture of CHCl₃-IPA (3×25 mL), and the combined organics washed with saturated aqueous NaCl solution, dried (anhyd. Na₂SO₄), filtered, and concentrated in vacuo. Purification of the residue by flash chromatography on SiO₂ (eluent: 5% EtOAc in CHCl₃) furnished the title compound (33 mg, 58 μmol, 5.1%) as an amorphous white foam. NMR (400 MHz, CDCl₃) δ 8.51 (s, 1H), 7.99-7.95 (m, 2H), 7.65-7.60 (m, 2H), 7.31-7.27 (m, 2H), 7.24-7.19 (m, 2H), 6.54 (dd, J=7.2, 2.0 Hz, 1H), 5.82 (dd, J=5.9, 1.3 Hz, 1H), 4.51 (d, J=11.8 Hz, 1H), 4.40 (d, J=11.8 Hz, 1H), 3.29 (ddd, J=15.5, 7.1, 6.3 Hz, 1H), 3.05-3.00 (m, 1H), 2.44 (s, 3H), 2.41 (s, 3H), 2.13-1.95 (m, 2H), 1.09 (t, J=7.5 Hz, 3H).

Step 4: (2R,3S,5R)-5-(6-amino-2-chloro-9H-purin-9-yl)-2-ethyl-2-(((4-methylbenzoyl)oxy)methyl)tetrahydrofuran-3-yl 4-methylbenzoate. A pressure tube equipped with a magnetic stirrer bar was charged with (2R,3S,5R)-5-(2,6-dichloro-9H-purin-9-yl)-2-ethyl-2-(((4-methylbenzoyl)oxy)methyl)tetrahydrofuran-3-yl 4-methylbenzoate (33 mg, 58 μmol) and a solution of ammonia in IPA (2M, 2.90 mL, 5.80 mmol). The vessel was sealed with a Teflon screw cap and the mixture heated to 105° C. with stirring for ca. 18 h. The mixture was then cooled to ambient temperature and the volatiles removed in vacuo to afford the title compound (31.9 mg, 58 μmol, assumed quant). LC-MS (ESI) m/z 550.4 [M+H]+. LC-MS RT=1.53 min; Method H.

Step 5: (2R,3S,5R)-5-(6-amino-2-chloro-9H-purin-9-yl)-2-ethyl-2-(hydroxymethyl)tetrahydrofuran-3-ol (Cpd. No. 176). (2R,3S,5R)-5-(6-amino-2-chloro-9H-purin-9-yl)-2-ethyl-2-(((4-methylbenzoyl)oxy)methyl)tetrahydrofuran-3-yl 4-methylbenzoate (32 mg, 58 μmol) was dissolved in a mixture of THF (320 μL) and MeOH (160 μL), A solution of NaOMe (25 wt. % in MeOH, 50 μL) was added dropwise, and the reaction mixture stirred at ambient temperature for ca. 20 h. The reaction was quenched with a few drops of AcOH and the mixture concentrated in vacuo. The residue was purified by reverse-phase chromatography on C18 (eluent: 5-20% MeCN in 10 mM aqueous ammonium bicarbonate solution) to furnish Cpd. No. 176 (8.75 mg, 27.9 μmol, 48%) as an amorphous white solid. ¹NMR (400 MHz, DMSO-d6) δ 8.37 (s, 1H), 7.82 (br. s, 2H), 6.20 (dd, J=8.2, 2.7 Hz, 1H), 5.53 (d, J=5.0 Hz, 1H), 4.86 (t, J=5.7 Hz, 1H), 4.27 (ddd, J=6.3, 5.0, 1.6 Hz, 1H), 3.37 (dd, J=11.4, 5.9 Hz, 1H), 3.33-3.28 (m, 1H), 2.95 (ddd, J=14.4, 8.1, 6.3 Hz, 1H), 2.25 (ddd, J=14.4, 2.4, 1.9 Hz, 1H), 1.76-1.61 (m, 2H), 0.84 (t, J=7.5 Hz). LC-MS (ESI) m/z 314.2 [M+H]+. LC-MS RT=0.50 min: Method G.

### Example 16

### Synthesis of (2R,3S,4R,5R)-5-(6-amino-2-fluoro-9H-purin-9-yl)-2-(hydroxymethyl)-2-methyltetrahydrofuran-3,4-diol (Cpd. No. 178)

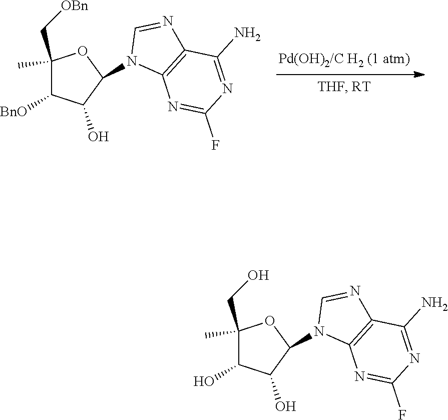 [SMILES: C[C@]1(COCc2ccccc2)O[C@@H](n2cnc3c(N)nc(F)nc32)[C@H](O)[C@@H]1OCc1ccccc1 ; C[C@]1(CO)O[C@@H](n2cnc3c(N)nc(F)nc32)[C@H](O)[C@@H]1O]

Step 1: (2R,3S,4R,5R)-5-(6-amino-2-fluoro-9H-purin-9-yl)-2-(hydroxymethyl)-2-methyltetrahydrofuran-3,4-diol (Cpd. No. 178). A flame-dried round-bottomed flask equipped with a magnetic stirrer bar was charged with a solution of (2R,3R,4S,5R)-2-(6-amino-2-fluoro-9H-purin-9-yl)-4-(benzyloxy)-5-((benzyloxy)methyl)-5-methyltetrahydrofuran-3-ol (220 mg, 459 μmol) in THF (18.0 mL) under nitrogen. Palladium hydroxide (20 wt. % on carbon, 330 mg) was added in one portion, then the reaction mixture was sparged with hydrogen gas from a balloon for ca. 30 seconds. The reaction mixture was then allowed to stir under hydrogen (1 atm) at ambient temperature for ca. 18 h. The reaction mixture was filtered through Celite and the filtrate was concentrated in vacuo to provide Cpd. No. 178 (70 mg, 235 μmol, 51%) as an amorphous white solid. ¹H NMR (400 MHz, DMSO-d6) δ 8.34 (s, 1H), 7.86 (s, br. 1H), 5.76 (d, J=7.3 Hz, 1H), 5.33 (d, J=6.9 Hz, 1H), 5.16 (dd, J=6.5, 5.2 Hz, 1H), 5.13 (d, J=4.9 Hz, 1H), 4.76 (td, J=7.1, 5.2 Hz, 1H), 4.03 (t, J=5.0 Hz, 1H), 3.52 (dd, J=11.5, 5.2 Hz, 1H), 3.37-3.32 (m), 1.15 (s, 3H). LC-MS (ESI) m/z 300.2 [M+H]+. LC-MS RT=0.53 min; Method A.

### Example 17

### Synthesis of 4-amino-1-((2R,3S,4S,5R)-3,4-dihydroxy-5-(hydroxymethyl)-5-methyltetrahydrofuran-2-yl)pyrimidin-2(1H)-one (Cpd. No. 193)

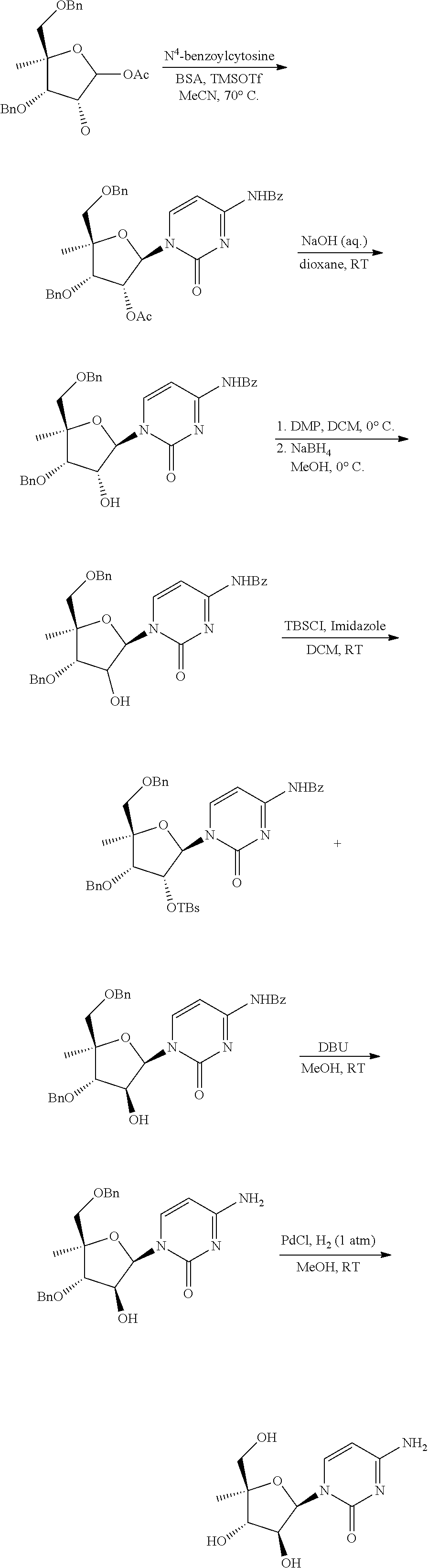

Step 1: (2R,3R,4S,5R)-2-(4-benzamido-2-oxopyrimidin-1(2H)-yl)-4-(benzyloxy)-5-((benzyloxy)methyl)-5-methyltetrahydrofuran-3-yl acetate. A flame-dried three-necked round-bottomed flask, equipped with a magnetic stirrer bar and fitted with a reflux condenser, was charged with a suspension of N⁴-benzoylcytosine (3.37 g, 15.7 mmol) in anhydrous acetonitrile (116 mL) under nitrogen atmosphere. N,O-Bis(trimethylsilyl)acetamide (10.0 mL, 38.9 mmol) was added, and the reaction mixture heated to 70° C. with stirring for ca. 1 h. A solution of (3R,4S,5R)-4-(benzyloxy)-5-((benzyloxy)methyl)-5-methyltetrahydrofuran-2,3-diyl diacetate (5.50 g, 12.8 mmol) in anhydrous acetonitrile (12.80 mL), prepared in a separate flame-dried round-bottomed flask, was then added dropwise, followed by dropwise addition of trimethylsilyl trifluoromethanesulfonate (2.83 mL, 15.7 mmol). The reaction mixture was then stirred at 50° C. for ca. 30 mins. The reaction mixture was then cooled to 0° C. with stirring and quenched by slow addition of saturated aqueous NaHCO₃ solution (40 mL). Water (50 mL) was added, the mixture poured into a separatory funnel, and the organics extracted with EtOAc (3×50 mL). The combined organics were washed with water (50 mL) and saturated aqueous NaCl solution, dried (anhyd. Na₂SO₄), filtered and concentrated in vacuo. The residue was purified by flash chromatography on SiO₂ (eluent: 50% EtOAc in DCM) to afford the title compound (4.05 g, 6.92 mmol, 54%) as a white solid. ¹H NMR (400 MHz, CDCl₃) δ 8.67 (s, 1H), 8.43 (d, J=6.7 Hz, 1H), 7.90 (d, J=7.0 Hz, 2H), 7.61 (t, J=7.4 Hz, 1H), 7.52 (t, J=7.6 Hz, 2H), 7.45-7.27 (m, 8H), 7.24 (dd, J=7.7, 1.5 Hz, 2H), 6.20 (d, J=2.3 Hz, 1H), 5.45 (dd, J=5.7, 2.4 Hz, 1H), 4.64 (d, J=11.8 Hz, 1H), 4.37 (dt, J=14.3, 11.2 Hz, 3H), 4.24 (d, J=5.7 Hz, 1H), 3.62 (d, J=10.4 Hz, 1H), 3.34 (d, J=10.4 Hz, 1H), 2.15 (s, 3H), 1.31 (s, 3H). LC-MS (ESI) m/z 570.5 [M+H]⁺. LC-MS RT=1.57 min; Method H.

Step 2: N-(1-((2R,3R,4S,5R)-4-(benzyloxy)-5-((benzyloxy)methyl)-3-hydroxy-5-methyltetrahydrofuran-2-yl)-2-oxo-1,2-dihydropyrimidin-4-yl)benzamide. A solution of (2R,3R,4S,5R)-2-(4-benzamido-2-oxopyrimidin-1(2H)-yl)-4-(benzyloxy)-5-((benzyloxy)methyl)-5-methyltetrahydrofuran-3-yl acetate (2.10 g, 3.60 mmol) in dioxane (105 mL) was treated with a solution of aqueous NaOH (1M, 11.0 mL, 11.0 mmol) at ambient temperature with stirring for ca. 3 h. The mixture was adjusted to pH 7 by addition of 1M aqueous HCl solution, then the mixture was diluted with water and the organics extracted with EtOAc. The organics were washed with saturated aqueous NaCl solution and dried (anhyd. Na₂SO₄), filtered and concentrated in vacuo to furnish the title compound (1.76 g, 3.24 mmol, 90%). LC-MS (ESI) m/z 542.5 [M+H]⁺. LC-MS RT=1.45 min; Method H.

Step 3: N-(1-((2R,3S,4S,5R)-4-(benzyloxy)-5-((benzyloxy)methyl)-3-hydroxy-5-methyltetrahydrofuran-2-yl)-2-oxo-1,2-dihydropyrimidin-4-yl)benzamide. A solution of N-(1-((2R,3R,4S,5R)-4-(benzyloxy)-5-((benzyloxy)methyl)-3-hydroxy-5-methyltetrahydrofuran-2-yl)-2-oxo-1,2-dihydropyrimidin-4-yl)benzamide (1.00 g, 1.85 mmol) in DCM (10.0 mL) was cooled to 0° C. with stirring, and Dess-Martin periodinane (1.57 g, 3.71 mmol) was added portion-wise to the reaction mixture. The mixture was then warmed to ambient temperature and stirred for ca. 16 h. The reaction mixture was then diluted with Et₂O, and the mixture washed successively with saturated aqueous NaHCO₃ solution and 10% aqueous Na₂S₂O₃ solution. The organics were dried (anhyd. Na₂SO₄), filtered and concentrated in vacuo. The residue was then dissolved in MeOH (10.0 mL), the resulting solution cooled to 0° C. with stirring, and sodium borohydride (71.5 mg, 1.85 mmol) added. The mixture was stirred at 0° C. for ca. 30 mins, then the reaction was quenched with water and the organics extracted with DCM. The combined organics were dried (anhyd. Na₂SO₄), filtered and concentrated in vacuo to afford the crude product (1.00 g, 1.85 mmol, assumed quant) as a 1:1 mixture of 2′-epimers. LC-MS (ESI) m/z 656.7 [M+H]⁺. LC-MS RT=1.98 min: Method H. To resolve the epimers, the entire crude product (1.00 g, 1.85 mmol) was dissolved in anhydrous DCM (15.0 mL) and treated with imidazole (382 mg, 5.56 mmol) and TBSCl (846 mg, 5.56 mmol) at 0° C. with stirring, leading to selective silylation of the 2′-(R)-epimer. The reaction was quenched with saturated aqueous NaHCO₃ solution, and the phases separated. The organics were dried (anhyd. Na₂SO₄), filtered and concentrated in vacuo. The residue was purified by flash chromatography on SiO₂ (eluent: 20-60% EtOAc in hexanes). The TBS-protected 2′-(R)-epimer, N-(1-((2R,3R,4S,5R)-4-(benzyloxy)-5-((benzyloxy)methyl)-3-((tert-butyldimethylsilyl)oxy)-5-methyltetrahydrofuran-2-yl)-2-oxo-1,2-dihydropyrimidin-4-yl)benzamide (490 mg, 747 μmol, 81%) was recovered and set aside. NMR (400 MHz, CDCl₃) δ 8.58 (d, J=5.6 Hz, 1H), 7.89 (d, J=6.6 Hz, 2H), 7.59 (d, J=7.4 Hz, 1H), 7.50 (t, J=7.6 Hz, 2H), 7.44-7.35 (m, 3H), 7.36-7.26 (m, 6H), 7.24-7.18 (m, 3H), 7.08 (br. s, 1H), 5.92 (d, J=1.3 Hz, 1H), 4.72 (d, J=12.0 Hz, 1H), 4.44 (s, 1H), 4.39-4.20 (m, 3H), 4.00 (d, J=4.8 Hz, 1H), 3.66 (d, J=10.5 Hz, 1H), 3.41 (d, J=10.4 Hz, 1H), 1.40 (s, 3H), 0.90 (s, 9H), 0.20 (s, 3H), 0.10 (s, 3H). LC-MS (ESI) m/z 656.7 [M+H]+. LC-MS RT=1.98 min; Method H. The title compound (435 mg, 803 μmol, 86%) was recovered as a light yellow foam. ¹H NMR (400 MHz, CDCl₃) δ 8.86 (br. s, 1H), 8.31 (d, J=7.4 Hz, 1H), 7.91 (d, J=7.6 Hz, 2H), 7.61 (dd, J=8.4, 6.4 Hz, 1H), 7.51 (t, J=7.6 Hz, 2H), 7.43-7.27 (m, 10H), 6.20 (d, J=4.6 Hz, 1H), 4.81 (d, 0.1=12.1 Hz, 1H), 4.68 (s, 1H), 4.53 (dt, J=17.1, 7.9 Hz, 3H), 4.07 (s, 1H), 3.65 (d, J=10.2 Hz, 1H), 3.46 (d, J=10.2 Hz, 1H), 1.28 (s, 3H). LC-MS (ESI) m/z 542.5 [M+H]+. LC-MS RT=1.46 min; Method H.

Step 4: 4-amino-1-((2R,3S,4S,5R)-4-(benzyloxy)-5-(benzyloxy)methyl)-3-hydroxy-5-methyltetrahydrofuran-2-yl)pyrimidin-2(1H)-one. DBU (0.18 mL, 1.18 mmol) was added dropwise to a stirring solution of N-(1-((2R,3S,4S,5R)-4-(benzyloxy)-5-((benzyloxy)methyl)-3-hydroxy-5-methyltetrahydrofuran-2-yl)-2-oxo-1,2-dihydropyrimidin-4-yl)benzamide (427 mg, 788 μmol) in MeOH (8.30 mL) at ambient temperature, and the mixture stirred at ambient temperature for ca. 30 min. The reaction mixture was adsorbed onto silica gel and purified by flash chromatography on SiO₂ (eluent: 15% MeOH in DCM) to furnish the title compound (200 mg, 457 μmol, 57%). LC-MS (ESI) m/z 438.5 [M+H]+. LC-MS RT=1.14 min; Method H.

Step 5: 4-amino-1-((2R,3S,4S,5R)-3,4-dihydroxy-5-(hydroxymethyl)-5-methyltetrahydrofuran-2-yl)pyrimidin-2(1H)-one (Cpd. No. 193). Palladium(II) chloride (60.9 mg, 343 μmol) was added under nitrogen to a solution of 4-amino-1-((2R,3S,4S,5R)-4-(benzyloxy)-5-((benzyloxy)methyl)-3-hydroxy-5-methyltetrahydrofuran-2-yl)pyrimidin-2(1H)-one (150 mg, 343 μmol) in MeOH (13.8 mL) at ambient temperature. The mixture was sparged with hydrogen from a balloon for ca. 1 min, then the reaction mixture was stirred under hydrogen (1 atm) for ca. 1 h. Et₃N (ca. 5 mL) was added, and the mixture was filtered through a short pad of Celite®. The filtrate was concentrated in vacuo and the residue purified by flash chromatography on SiO₂ (eluent: 30% MeOH in DCM) to furnish Cpd. No. 193 (65 mg, 252 μmol, 73%) as an amorphous white solid. NMR (400 MHz, DMSO-d6) δ 7.64 (d, J=7.4 Hz, 1H), 7.05 (br. s, 1H), 6.96 (br. s, 1H), 6.03 (d, J=4.8 Hz), 5.63 (d, J=7.4 Hz, 1H), 5.40 (d, J=5.6 Hz, 1H), 5.30 (d, J=4.8 Hz, 1H), 5.05 (br. app. t, J=5.2 Hz, 1H), 4.05 (dd, J=9.1, 4.9 Hz, 1H), 3.89 (app. t, J=4.1 Hz, 1H), 3.47 (dd, J=11.2, 4.5 Hz, 1H), 3.41 (dd, J=10.9, 4.4 Hz, 1H), 1.07 (s, 3H). LC-MS (ESI) m/z 258.2 [M+H]+. LC-MS RT=0.19 min; Method A.

### Example 18

### Synthesis of 4-amino-1-((2R,3R,4S,5R)-3,4-dihydroxy-5-(hydroxymethyl)-5-methyltetrahydrofuran-2-yl)pyrimidin-2(1H)-one (Cpd. No. 188)

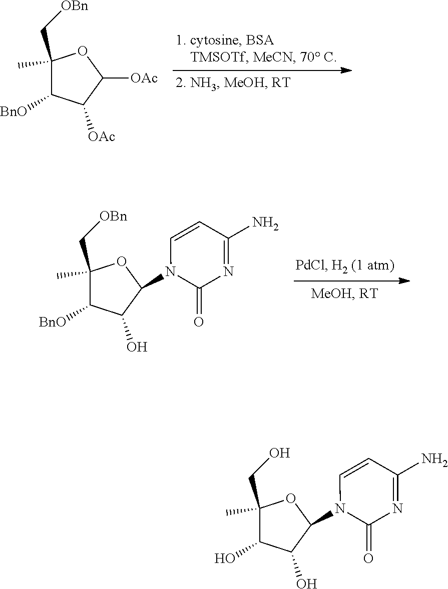 [SMILES: CC(=O)OC1O[C@](C)(COCc2ccccc2)[C@@H](OCc2ccccc2)[C@H]1OC(C)=O ; C[C@]1(COCc2ccccc2)O[C@@H](n2ccc(N)nc2=O)[C@H](O)[C@@H]1OCc1ccccc1 ; C[C@]1(CO)O[C@@H](n2ccc(N)nc2=O)[C@H](O)[C@@H]1O]

Step 1: 4-amino-1-((2R,3R,4S,5R)-4-(benzyloxy)-5-((benzyloxy)methyl)-3-hydroxy-5-methyltetrahydrofuran-2-yl)pyrimidin-2(1H)-one. A flame-dried three-necked round-bottomed flask, equipped with a magnetic stirrer bar and fitted with a reflux condenser, was charged with a suspension of cytosine (953 mg, 8.40 mmol) in anhydrous acetonitrile (63.0 mL) under nitrogen atmosphere. N,O-Bis(trimethylsilyl)acetamide (5.46 mL, 21.2 mmol) was added, and the reaction mixture heated to 70° C. with stirring for ca. 1 h. A solution of (3R,4S,5R)-4-(benzyloxy)-5-((benzyloxy)methyl)-5-methyltetrahydrofuran-2,3-diyl diacetate (3.53 g, 7.00 mmol) in anhydrous acetonitrile (7.00 mL), prepared in a separate flame-dried round-bottomed flask, was then added dropwise, followed by dropwise addition of trimethylsilyl trifluoromethanesulfonate (1.55 mL, 8.54 mmol). The reaction mixture was then stirred at 50° C. for ca. 30 mins. The reaction mixture was then cooled to 0° C. with stirring and quenched by slow addition of saturated aqueous NaHCO₃ solution (40 mL). Water (50 mL) was added, the mixture poured into a separatory funnel, and the organics extracted with EtOAc (3×50 mL). The combined organics were washed with water (50 mL) and saturated aqueous NaCl solution, dried (anhyd. Na₂SO₄), filtered and concentrated in vacuo. The residue was dissolved in a solution of ammonia in MeOH (7N, 100 mL) and stirred at ambient temperature overnight. The volatiles were removed in vacuo and the residue purified by flash chromatography on SiO₂ (eluent: 7% IPA in DCM) to afford the title compound (2.50 g, 5.72 mmol, 82%) as a brown semi-solid. ¹H NMR (400 MHz, CDCl₃) 7.93 (d, J=7.6 Hz, 1H), 7.41-7.27 (m, 9H), 7.23 (d, 1=7.0 Hz, 2H), 5.88 (d, 1=3.9 Hz, 1H), 5.70 (d, 1=7.6 Hz, 1H), 4.73 (d, J=11.8 Hz, 1H), 4.54-4.49 (m, 1H), 4.42 (q, J=11.3 Hz, 2H), 4.34-4.27 (m, 1H), 4.05 (d, J=5.6 Hz, 1H), 3.53 (d, J=10.2 Hz, 1H), 3.33 (d, 0.1=10.1 Hz, 1H), 1.27 (s, 3H). LC-MS (ESI) m/z 438.5 [M+H]⁺. LC-MS RT=1.10 min; Method H.

Step 2: 4-amino-1-((2R,3R,4S,5R)-3,4-dihydroxy-5-(hydroxymethyl)-5-methyltetrahydrofuran-2-yl)pyrimidin-2(1H)-one (Cpd. No. 188). A flame-dried round-bottomed flask equipped with a magnetic stirrer bar was charged with a solution of 4-amino-1-((2R,3R,4S,5R)-4-(benzyloxy)-5-((benzyloxy)methyl)-3-hydroxy-5-methyltetrahydrofuran-2-yl)pyrimidin-2(1H)-one (100 mg, 229 μmol) in MeOH (9.18 mL) under nitrogen atmosphere. Palladium(II) chloride (40.6 mg, 229 μmol) was added in one portion, then the reaction mixture was sparged with hydrogen gas from a balloon for ca. 30 seconds. The reaction mixture was then allowed to stir under hydrogen (1 atm) for ca. 1 h. The hydrogen balloon was then removed, and the vessel purged under positive pressure of nitrogen for ca. 30 seconds. The reaction mixture was filtered through a short pad of Celite®, rinsing with small portions of MeOH, and the filtrate concentrated in vacuo. The residue was purified by flash chromatography on SiO₂ (eluent: 30% MeOH in DCM) to furnish Cpd. No. 188 (14 mg, 53 μmol, 23%) as a white amorphous solid after lyophilization. ¹H NMR (400 MHz, DMSO-d6) δ 8.15 (d, J=7.3 Hz, 1H), 7.77 (br. S, 1H), 7.53 (br. S, 1H), 5.80 (dd, J=6.1, 2.1 Hz, 1H), 5.27 (t, J=5.2 Hz, 1H), 5.20 (d, J=6.3 Hz, 1H), 4.94 (d, J=5.4 Hz, 1H), 4.10 (dd, J=11.8, 6.0 Hz, 1H), 3.93 (t, J=5.4 Hz, 1H), 3.39 (tt, J=11.6, 5.6 Hz, 2H), 1.08 (s, 3H). LC-MS (ESI) m/z 258.3 [M+H]⁺. LC-MS RT=0.18 min; Method A.

### Example 19

### Synthesis of 4-amino-5-chloro-1-((2R,4S,5R)-5-ethynyl-4-hydroxy-5-(hydroxymethyl)tetrahydrofuran-2-yl)pyrimidin-2(1H)-one (Cpd. No. 109)

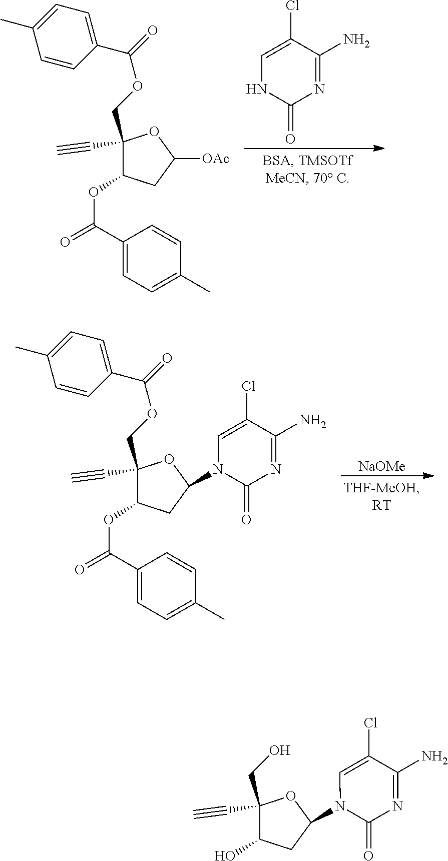

Step 1: (2R,3S)-5-(4-amino-5-chloro-2-oxopyrimidin-1(2H)-yl)-2-ethynyl-2-(((4-methylbenzoyl)oxy)methyl)tetrahydrofuran-3-yl 4-methylbenzoate. A flame-dried round-bottomed flask, equipped with a magnetic stirrer bar and fitted with a reflux condenser, was charged with 5-chlorocytosine (258 mg, 1.68 mmol) and anhydrous MeCN (17.5 mL). N,O-Bis(trimethylsilyl)acetamide (1.24 mL, 4.81 mEmol) was added, and the mixture heated to 70° C. with stirring for ca. 1 h before cooling to 0° C. A solution of (2R,3S)-5-acetoxy-2-ethynyl-2-(((4-methylbenzoyl)oxy)methyl)tetrahydrofuran-3-yl 4-methylbenzoate (700 mg, 1.60 mmol) in anhydrous MeCN (4.00 mL) was added dropwise, followed by dropwise addition of trimethylsilyl trifluoromethanesulfonate (326 μL, 1.76 mmol) over ca. 5 min. The reaction mixture was then warmed to ambient temperature with stirring for ca. 15 mins, then warmed to 70° C. with stirring for ca. 1 h. The mixture was then cooled to ambient temperature, quenched with saturated aqueous NaHCO₃ solution (30 mL), and diluted with saturated aqueous NaCl solution (12 mL). The organics were extracted with EtOAc (3×30 mL), and the combined organics were dried (anhyd. Na₂SO₄), filtered, and concentrated in vacuo. Purification of the residue by flash chromatography on SiO₂ (eluent: 5% MeOH in DCM) furnished the title compound (600 mg, 1.15 mmol, 72%) as an amorphous white solid, as a ca. 3:2 mixture of anomers by LCMS. For the major anomer, LC-MS (ESI) m/z 520.3 [M−H]⁻. LC-MS RT=1.35 min; Method H. For the minor anomer, LC-MS (ESI) m/z 520.4 [M−H]⁻. LC-MS RT=1.31 min; Method H.

Step 2: 4-amino-5-chloro-1-((2R,4S,5R)-5-ethynyl-4-hydroxy-5-(hydroxymethyl)tetrahydrofuran-2-yl)pyrimidin-2(1H)-one (Cpd. No. 109). (2R,3S,5R)-5-(4-amino-5-chloro-2-oxopyrimidin-1(2H)-yl)-2-ethynyl-2-(((4-methylbenzoyl)oxy)methyl)tetrahydrofuran-3-yl 4-methylbenzoate (600 mg, 1.15 mmol) was dissolved in a mixture of THF (7.66 mL) and MeOH (3.83 mL), A solution of NaOMe (25 wt. % in MeOH, 526 μL, 2.30 mmol) was added dropwise, and the reaction mixture stirred at ambient temperature for ca. 1 h. The volatiles were then removed in vacuo. The residue was purified by flash chromatography on SiO₂ (eluent: 20% IPA in DCM) to afford the deprotected nucleoside as a ca. 3:2 mixture of anomers. Further purification by preparative LCMS furnished Cpd. No. 109 (130 mg, 456 μmol, 79%). NMR (400 MHz, CD₃OD) δ 8.36 (s, 1H), 6.12 (dd, J=7.2, 3.4 Hz, 1H), 4.44 (t, J=7.9 Hz, 1H), 3.85 (d, J=12.2 Hz, 1H), 3.76 (d, J=12.2 Hz, 1H), 3.05 (s, 1H), 2.62-2.46 (m, 1H), 2.25 (ddd, J=13.4, 7.2, 3.4 Hz, 1H). LC-MS (ESI) m/z 286.1 [M+H]+. LC-MS RT=0.35 min; Method A.

### Example 20

### Synthesis of 5-fluoro-1-((2R,4S,5R)-5-(fluoromethyl)-4-hydroxy-5-(hydroxymethyl)tetrahydrofuran-2-yl)pyrimidine-2,4(1H,3H)-dione (Cpd. No. 112)

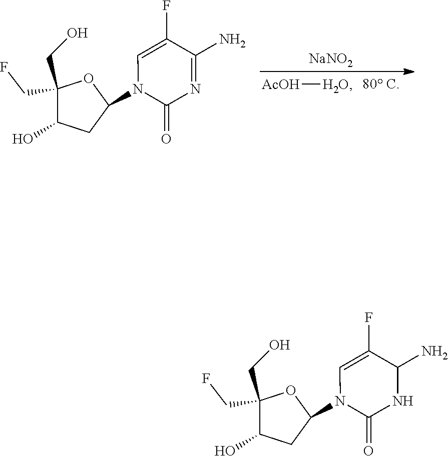

Step 1: 5-fluoro-1-((2R,4S,5R)-5-(fluoromethyl)-4-hydroxy-5-(hydroxymethyl)tetrahydrofuran-2-yl)pyrimidine-2,4(1H,3H)-dione (Cpd. No. 112). 4-amino-5-fluoro-1-((2R,4S,5R)-5-(fluoromethyl)-4-hydroxy-5-(hydroxymethyl)tetrahydrofuran-2-yl)pyrimidin-2(1H)-one (Cpd. No. 197, 80.3 mg, 290 μmol) was dissolved in AcOH (29.0 mL) at ambient temperature with stirring. A solution of sodium nitrite (303 mg, 4.34 mmol) in water (12.8 mL) was added dropwise, then the reaction mixture was heated to 80° C. with stirring for ca. 4 h. The reaction mixture was then cooled to ambient temperature and the volatiles removed in vacuo. The crude product was purified by reverse-phase chromatography on C18 (eluent: 10% MeCN in 10 mM aqueous ammonium formate solution; then 10% MeCN in 10 mM aqueous ammonium bicarbonate solution). Further purification by flash chromatography on SiO₂ (eluent: 5-30% MeOH in DCM) and semi-preparative chromatography on C18 (eluent: MeCN in 10 mM aqueous ammonium bicarbonate solution) afforded Cpd. No. 112. ¹H NMR (400 MHz, CD₃OD) δ 8.19 (d, J=6.8 Hz, 1H), 6.32 (td, J=6.7, 1.7 Hz, 1H), 4.56 (dd, J=47.5, 0.9 Hz, 2H), 4.53 (app. t, J=5.3 Hz, 1H), 3.73-3.72 (m, 2H), 2.38-2.33 (m, 2H). LC-MS (ESI) m/z 277.2 [M−H]−. LC-MS RT=0.29 min; Method A.

### Example 21

### Synthesis of (2R,3S,4R,5R)-5-(6-amino-2-fluoro-9H-purin-9-yl)-2-ethyl-2-(hydroxymethyl)tetrahydrofuran-3,4-diol (Cpd. No. 173)

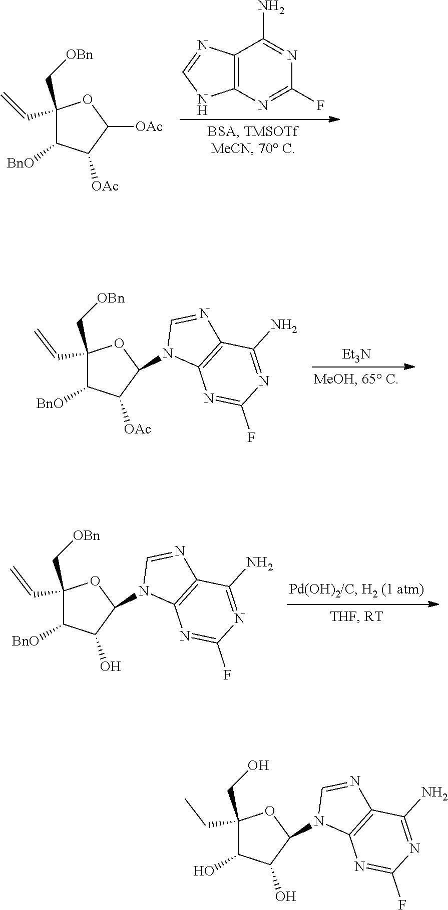 [SMILES: "Et3N" ; C=C[C@]1(COCc2ccccc2)OC(OC(C)=O)[C@H](OC(C)=O)[C@@H]1OCc1ccccc1 ; Nc1nc(F)nc2[nH]cnc12 ; C=C[C@]1(COCc2ccccc2)O[C@@H](n2cnc3c(N)nc(F)nc32)[C@H](OC(C)=O)[C@@H]1OCc1ccccc1 ; C=C[C@]1(COCc2ccccc2)O[C@@H](n2cnc3c(N)nc(F)nc32)[C@H](O)[C@@H]1OCc1ccccc1 ; CC[C@]1(CO)O[C@@H](n2cnc3c(N)nc(F)nc32)[C@H](O)[C@@H]1O]

Step 1: (2R,3R,4S,5R)-2-(6-amino-2-fluoro-9H-purin-9-yl)-4-(benzyloxy)-5-((benzyloxy)methyl)-5-vinyltetrahydrofuran-3-yl acetate. A flame-dried three-necked round-bottomed flask, equipped with a magnetic stirrer bar and fitted with a reflux condenser, was charged with a suspension of 2-fluoroadenine (1.59 g, 10.4 mmol) in anhydrous MeCN (94.1 mL) at ambient temperature. N,O-Bis(trimethylsilyl)acetamide (6.68 mL, 25.9 mmol) was added, and the mixture heated to 70° C. with stirring for ca. 1 h before cooling to ambient temperature. To the cooled reaction mixture was added (3R,4S,5R)-4-(benzyloxy)-5-((benzyloxy)methyl)-5-vinyltetrahydrofuran-2,3-diyl diacetate (3.80 g, 8.63 mmol), followed by dropwise addition of trimethylsilyl trifluoromethanesulfonate (1.89 mL, 10.4 mmol). The reaction mixture was then warmed to 70° C. with stirring for ca. 1 h. The mixture was cooled to ambient temperature and quenched with saturated aqueous NaHCO₃ solution (50 mL). The organics were extracted with EtOAc (4×50 mL). The combined organics were dried (anhyd. Na₂SO₄), filtered and concentrated in vacuo. The residue was purified by flash chromatography on SiO₂ (eluent: 0-100% EtOAc in hexanes) to furnish the title compound (3.50 g, 6.57 mmol, 76%) as a white solid. ¹H NMR (400 MHz, CDCl₃) δ 8.12 (s, 1H), 7.38-7.27 (m, 8H), 7.26-7.22 (m, 2H), 6.23 (d, J=3.5 Hz, 1H), 6.03 (br. S, 2H), 5.99 (dd, J=17.4, 11.0 Hz, 1H), 5.64 (dd, J=5.6, 3.5 Hz, 1H), 5.51 (dd, J=17.4, 1.3 Hz, 1H), 5.31 (dd, J=11.0, 1.3 Hz, 1H), 4.71 (d, J=5.6 Hz, 1H), 4.60 (d, J=11.6 Hz, 1H), 4.50 (d, J=11.9 Hz, 1H), 4.49 (d, J=11.6 Hz, 1H), 4.42 (d, J=11.8 Hz, 1H), 3.52-3.45 (m, 2H), 2.06 (s, 3H). LC-MS (ESI) m/z 534.4 [M+H]⁺. LC-MS RT=1.44 min: Method H.

Step 2: (2R,3R,4S,5R)-2-(6-amino-2-fluoro-9H-purin-9-yl)-4-(benzyloxy)-5-((benzyloxy)methyl)-5-vinyltetrahydrofuran-3-ol. Triethylamine (16.4 mL) was added to a solution of (2R,3R,4S,5R)-2-(6-amino-2-fluoro-9H-purin-9-yl)-4-(benzyloxy)-5-((benzyloxy)methyl)-5-vinyltetrahydrofuran-3-yl acetate (3.50 g, 6.56 mmol) in MeOH (100 mL) with stirring, and the mixture heated to 65° C. with stirring for ca. 18 h before cooled to ambient temperature. The volatiles were then removed in vacuo and the residue taken up in EtOAc. The organics were washed with water and saturated aqueous NaCl solution, then dried (anhyd. MgSO₄), filtered and concentrated to furnish the title compound (3.01 g, 6.13 mmol, 93%), which was utilized without further purification. LC-MS (ESI) m/z 492.4 [M+H]+. LC-MS RT=1.31 min; Method H.

Step 3: (2R,3S,4R,5R)-5-(6-amino-2-fluoro-9H-purin-9-yl)-2-ethyl-2-(hydroxymethyl)tetrahydrofuran-3,4-diol (Cpd. No. 173). A flame-dried round-bottomed flask equipped with a magnetic stirrer bar was charged with a solution of (2R,3R,4S,5R)-2-(6-amino-2-fluoro-9H-purin-9-yl)-4-(benzyloxy)-5-((benzyloxy)methyl)-5-vinyltetrahydrofuran-3-ol (100 mg, 203 μmol) in THF (7.97 mL) under nitrogen. Palladium hydroxide (20 wt. % on carbon, 150 mg) was added in one portion, then the reaction mixture was sparged with hydrogen gas from a balloon for ca. 1 min. The reaction mixture was then allowed to stir under hydrogen (1 atm) at ambient temperature overnight. The reaction mixture was filtered through Celite and the filtrate was concentrated in vacuo to provide Cpd. No. 173 (58 mg, 185 μmol, 91%) as an amorphous white solid. ¹H NMR (400 MHz, DMSO-d6) δ 8.34 (s, 1H), 7.86 (br. S, 2H), 5.72 (d, J=7.9 Hz, 1H), 5.30 (d, J=7.2 Hz, 1H), 5.09 (d, J=4.7 Hz, 1H), 5.07 (dd, J=6.4, 5.2 Hz, 1H), 4.81 (td, J=7.6, 5.1 Hz, 1H), 4.05 (t, J=4.9 Hz, 1H), 3.53 (dd, J=11.6, 5.1 Hz), 3.46 (dd, J=11.6, 6.5 Hz, 1H), 1.74-1.58 (m, 2H), 0.85 (t, J=7.5 Hz, 3H). LC-MS (ESI) m/z 314.1 [M+H]+. LC-MS RT=0.75 min: Method A.

### Example 22

### Synthesis of (2R,3S,5R)-5-(5-fluoro-2,4-dioxo-3,4-dihydropyrimidin-1(2H)-yl)-3-hydroxy-2-(hydroxymethyl)tetrahydrofuran-2-carbonitrile (Cpd. No. 107)

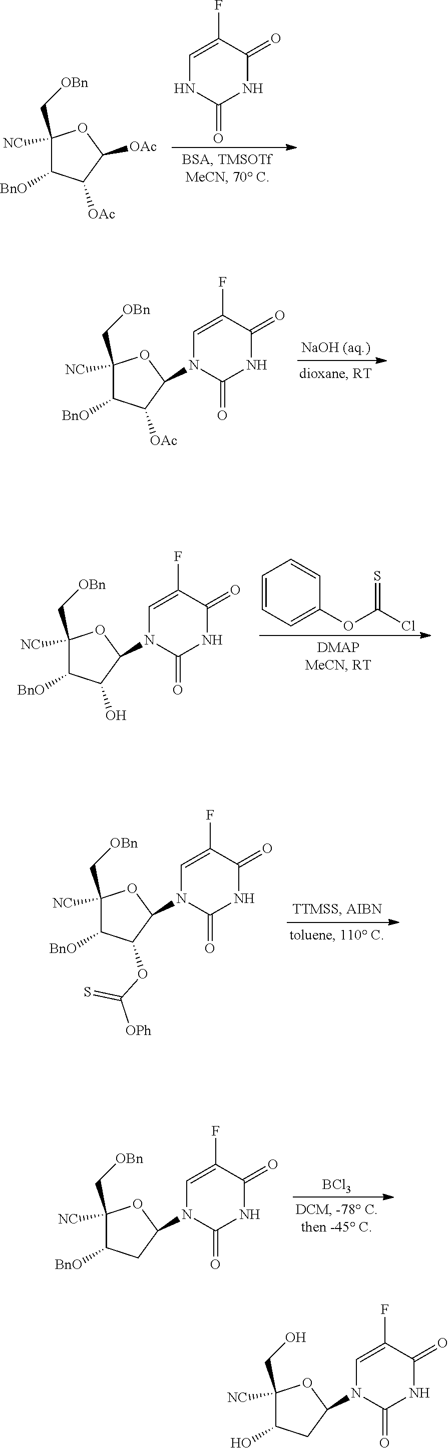

Step 1: (2R,3R,4S,5R)-4-(benzyloxy)-5-((benzyloxy)methyl)-5-cyano-2-(5-fluoro-2,4-dioxo-3,4-dihydropyrimidin-1(2H)-yl)tetrahydrofuran-3-yl acetate. A solution of 5-fluorouracil (200 mg, 1.54 mmol) in acetonitrile (10.0 mL) was degassed with N2 (3×). Then N,O-bis(trimethylsilyl)acetamide (1.90 mL, 7.77 mmol) was added. The resulting reaction mixture was stirred at 70° C. for 1 h and cooled to room temperature. Trimethylsilyl trifluoromethanesulfonate (411 μL, 1.33 mmol) was added and then (2S,3R,4S,5R)-4-(benzyloxy)-5-((benzyloxy)methyl)-5-cyanotetrahydrofuran-2,3-diyl diacetate (500 mg, 1.14 mmol) in acetonitrile (6.00 mL) was added. After stirring at 105° C. for 16 h, the reaction mixture was cooled to room temperature, diluted with water (20 mL), extracted with EtOAc (30 mL), washed with brine (20 mL), dried over Na₂SO₄, filtered, and concentrated. The residue was purified by silica gel chromatography eluting with 0-3% MeOH in DCM to afford the title compound (503 mg, 987 μmol, 87%) as a brown oil. ¹H NMR (400 MHz, CDCl₃) δ 8.52 (br s, 1H), 7.65 (d, J=6.0 Hz, 1H), 7.37-7.28 (m, 10H), 6.12 (d, J=2.8 Hz, 1H), 5.33 (d, J=5.2 Hz, 1H), 4.70-4.64 (m, 1H), 4.58-4.44 (m, 4H), 3.91 (d, J=10.8 Hz, 1H), 3.65-3.60 (m, 1H), 2.18 (s, 3H).

Step 2: (2R,3S,4R,5R)-3-(benzyloxy)-2-((benzyloxy)methyl)-5-(5-fluoro-2,4-dioxo-3,4-dihydropyrimidin-1(2H)-yl)-4-hydroxytetrahydrofuran-2-carbonitrile. To a solution of (2R,3R,4S,5R)-4-(benzyloxy)-5-((benzyloxy)methyl)-5-cyano-2-(5-fluoro-2,4-dioxo-3,4-dihydropyrimidin-1(2H)-yl)tetrahydrofuran-3-yl acetate (500 mg, 981 μmol) in dioxane (8 mL) was added aqueous NaOH (1 M, 1.00 mL). The resulting reaction mixture was stirred at 25° C. for 1 h. Then reaction mixture was diluted with water (5 mL), extracted with EtOAc (10 mL), washed with brine (10 mL), dried over Na₂SO₄, filtered and concentrated. The residue was purified by silica gel chromatography eluting with 0-3% MeOH in DCM to afford the title product (300 mg, 642 μmol 65%) as a white solid. ¹H NMR (400 MHz, DMSO-d6) δ 12.02 (br s, 1H), 8.00 (d, J=7.2 Hz, 1H), 7.39-7.31 (m, 10H), 5.96 (d, J=5.2 Hz, 1H), 5.90 (d, J=4.8 Hz, 1H), 4.82 (d, J=11.6 Hz, 1H), 4.67 (d, J=12.0 Hz, 1H), 4.59 (s, 2H), 4.50-4.41 (m, 1H), 4.25 (d, J=5.2 Hz, 1H), 3.92-3.86 (m, 1H), 3.83-3.77 (m, 1H).

Step 3: O-((2R,3R,4S,5R)-4-(benzyloxy)-5-((benzyloxy)methyl)-5-cyano-2-(5-fluoro-2,4-dioxo-3,4-dihydropyrimidin-1(2H)-yl)tetrahydrofuran-3-yl)O-phenyl carbonothioate. A solution of (2R,3S,4R,5R)-3-(benzyloxy)-2-((benzyloxy)methyl)-5-(5-fluoro-2,4-dioxo-3,4-dihydropyrimidin-1(2H)-yl)-4-hydroxytetrahydrofuran-2-carbonitrile (300 mg, 642 μmol) in acetonitrile (10 mL) was degassed with N2 (3×). Then DMAP (235 mg, 1.92 mmol) and O-phenyl chlorothionoformate (166 mg, 962 umol) were added. The resulting reaction mixture was stirred at 25° C. for 1 h. After that, the reaction mixture was diluted with water (5 mL), extracted with EtOAc (10 mL), washed with brine (10 mL), dried over Na₂SO₄, filtered and concentrated. The residue was purified by silica gel chromatography eluting with 0-3% MeOH in DCM to afford the title product (301.0 mg, 499 μmol, 78% yield) as a white solid.

Step 4: (2R,3S,5R)-3-(benzyloxy)-2-((benzyloxy)methyl)-5-(5-fluoro-2,4-dioxo-3,4-dihydropyrimidin-1(2H)-yl)tetrahydrofuran-2-carbonitrile. A solution of O-((2R,3R,4S,5R)-4-(benzyloxy)-5-((benzyloxy)methyl)-5-cyano-2-(5-fluoro-2,4-dioxo-3,4-dihydropyrimidin-1(2H)-yl)tetrahydrofuran-3-yl) 0-phenyl carbonothioate (300.0 mg, 497 μmol) in toluene (5 mL) was purged with N2 for 5 min. TTMSS (371 mg, 1.49 mmol) and AIBN (29.0 mg, 177 μmol) were added. The resulting reaction mixture was stirred at 110° C. for 2 h. After cooling to room temperature, the reaction mixture was concentrated. The residue was purified by silica gel chromatography eluting with 0-3% MeOH in DCM to afford the title product (90.0 mg, 199 μmol, 40% yield) as a white solid. LC-MS (ESI) m/z 474.0 [M+Na]⁺. LC-MS RT=0.890 min; Method C.

Step 5: (2R,3S,5R)-5-(5-fluoro-2,4-dioxo-3,4-dihydropyrimidin-1(2H)-yl)-3-hydroxy-2-(hydroxymethyl)tetrahydrofuran-2-carbonitrile (Cpd. No. 107). A solution of (2R,3S,5R)-3-(benzyloxy)-2-((benzyloxy)methyl)-5-(5-fluoro-2,4-dioxo-3,4-dihydropyrimidin-1(2H)-yl)tetrahydrofuran-2-carbonitrile (50.0 mg, 111 umol) in DCM (2 mL) was degassed with N2 (3×) and cooled to −78° C. with stirring, and then a solution of BCl₃ (1 M in DCM, 0.50 mL, 0.50 mmol) was added dropwise. The resulting reaction mixture was stirred at −45° C. for 0.5 h. Then the reaction mixture was quenched with MeOH (0 mL) at −45° C. and warmed to 0° C. The mixture was adjusted to pH >7 with 28% aqueous NH₄₀H solution (1.0 mL) and concentrated to residue. The residue was purified by prep-HPLC (acetonitrile 0-30%/NH₃·H₂O+NH₄HCO₃ in water) to afford Cpd. No. 107 (13.0 mg, 47.9 μmol, 43% yield) as a white solid. LC-MS (ESI) m/z 272.0 [M+H]⁺. LC-MS RT=0.170 min; Method F.

### Example 23

### Synthesis of 2-amino-9-((2R,4S,5R)-4-hydroxy-5-(hydroxymethyl)-5-methyltetrahydrofuran-2-yl)-9H-purin-6-ol (Cpd. No. 115)

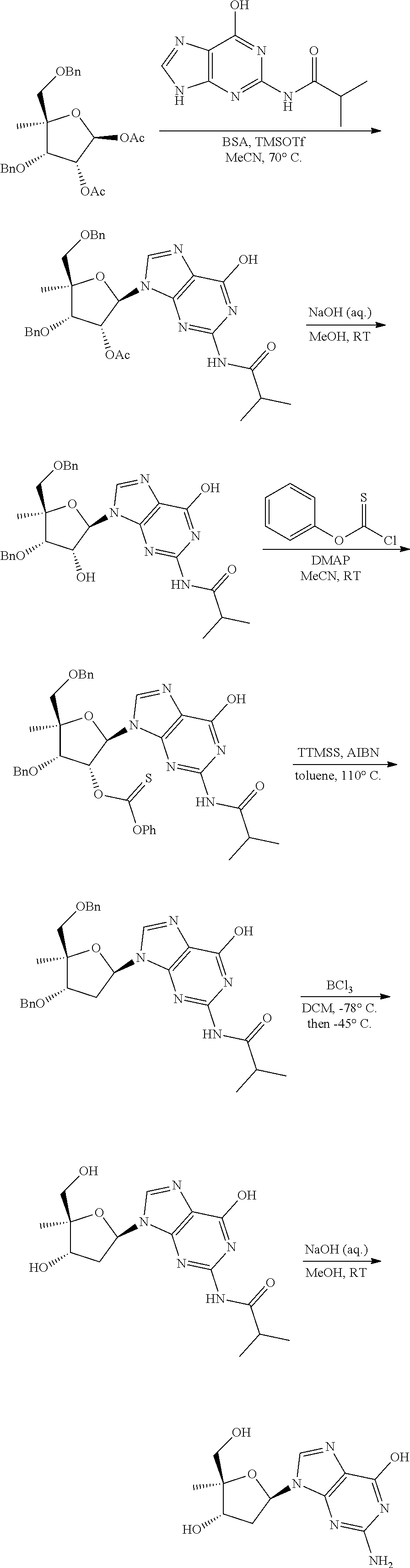

Step 1: (2R,3R,4S,5R)-4-(benzyloxy)-5-((benzyloxy)methyl)-2-(6-hydroxy-2-isobutyramido-9H-purin-9-yl)-5-methyltetrahydrofuran-3-yl acetate. A solution of N-(6-hydroxy-9H-purin-2-yl)isobutyramide (3.10 g, 14.0 mmol) in acetonitrile (15 mL) was degassed with N₂ (3×). Then N,O-bis(trimethylsilyl)acetamide (8.50 g, 41.8 mmol) was added. The reaction mixture was stirred at 70° C. for 1 h and then cooled to room temperature. Trimethylsilyl trifluoromethanesulfonate (3.90 g, 17.5 mmol) and (2S,3R,4S,5R)-4-(benzyloxy)-5-((benzyloxy)methyl)-5-methyltetrahydrofuran-2,3-diyl diacetate (5.00 g, 11.7 mmol) in acetonitrile (15 mL) were added. After stirring at 75° C. for 16 h, the reaction mixture was quenched with water (30 mL), extracted with EtOAc (40 mL), washed with brine (30 mL), dried over Na₂SO₄, filtered, and concentrated in vacuo. The residue was purified by silica gel chromatography eluting with 0-3% MeOH in DCM to afford the title product (3.00 g, 5.09 mmol, 44% yield) as a yellow solid. ¹H NMR (400 MHz, CDCl₃) δ 11.95 (s, 1H), 8.13 (s, 1H), 7.96 (s, 1H), 7.39-7.28 (m, 10H), 6.06 (d, J=4.8 Hz, 1H), 5.73 (t, J=5.2 Hz, 1H), 4.59-4.44 (m, 5H), 3.52 (d, J=10.0 Hz, 1H), 3.39 (d, J=10.4 Hz, 1H), 2.61-2.48 (m, 1H), 2.08 (s, 3H), 1.32 (s, 3H), 1.25 (d, J=7.2 Hz, 6H).

Step 2: N-(9-((2R,3R,4S,5R)-4-(benzyloxy)-5-((benzyloxy)methyl)-3-hydroxy-5-methyltetrahydrofuran-2-yl)-6-hydroxy-9H-purin-2-yl)isobutyramide. To a solution of (2R,3R,4S,5R)-4-(benzyloxy)-5-((benzyloxy)methyl)-2-(6-hydroxy-2-isobutyramido-9H-purin-9-yl)-5-methyltetrahydrofuran-3-yl acetate (100 mg, 170 μmol) in MeOH (5 mL) was added a solution of aqueous NaOH (2 M, 0.5 mL) at 25° C. The resulting reaction mixture was stirred at 25° C. for 1 h. Then the reaction mixture was adjusted to pH=7 with aqueous HCl (1 M) and then concentrated in vacuo. The residue was purified by silica gel chromatography eluting with 0-3% MeOH in DCM to afford the title product (50 mg, 91.3 μmol, 54% yield) as a white solid.

Step 3: O-((2R,3R,4S,5R)-4-(benzyloxy)-5-((benzyloxy)methyl)-2-(6-hydroxy-2-isobutyramido-9H-purin-9-yl)-5-methyltetrahydrofuran-3-yl) 0-phenyl carbonothioate. To a solution of N-(9-((2R,3R,4S,5R)-4-(benzyloxy)-5-((benzyloxy)methyl)-3-hydroxy-5-methyltetrahydrofuran-2-yl)-6-hydroxy-9H-purin-2-yl)isobutyramide (0.5 g, 913 μmol) in acetonitrile (15 mL) was added DMAP (328 mg, 2.68 mmol). The reaction mixture was degassed with N2 for three times and then O-phenyl chlorothionoformate (232 mg, 1.34 mmol) was added dropwise at 25° C. After stirring at 25° C. for 1 h, the reaction was quenched with water (10 mL), extracted with EtOAc (20 mL), washed with brine (20 mL), dried over Na₂SO₄, filtered and concentrated. The residue was purified by silica gel chromatography eluting with 0-3% MeOH in DCM to afford the title product (525 mg, 768 μmol, 84% yield) as a yellow solid. ¹H NMR (400 MHz, CDCl₃) δ 5 11.96 (s, 1H), 8.18-8.03 (m, 2H), 7.42-7.33 (m, 13H), 6.98 (d, J=8.0 Hz, 2H), 6.31-6.23 (m, 2H), 4.77-4.71 (m, 1H), 4.67-4.59 (m, 3H), 4.53-4.48 (m, 1H), 3.59 (d, J=10.4 Hz, 1H), 3.47 (d, J=10.4 Hz, 1H), 2.60-2.50 (m, 1H), 1.36 (s, 3H), 1.28-1.24 (m, 6H).

Step 4: N-(9-((2R,4S,5R)-4-(benzyloxy)-5-((benzyloxy)methyl)-5-methyltetrahydrofuran-2-yl)-6-hydroxy-9H-purin-2-yl)isobutyramide. A solution of O-((2R,3R,4S,5R)-4-(benzyloxy)-5-((benzyloxy)methyl)-2-(6-hydroxy-2-isobutyramido-9H-purin-9-yl)-5-methyltetrahydrofuran-3-yl) O-phenyl carbonothioate (1.00 g, 1.46 mmol) in toluene (24 mL) was purged with N2 for 5 min. AIBN (123 mg, 749 μmol) and TTMSS (1.90 g, 7.64 mmol) were added. The reaction mixture was stirred at 110° C. for 2 h. After cooling to room temperature, the reaction mixture was concentrated. The residue was purified by silica gel chromatography eluting with 0-3% MeOH in DCM to afford the title product (540 mg, 1.02 mmol, 70% yield) as a colorless oil.

Step 5: N-(6-hydroxy-9-((2R,4S,5R)-4-hydroxy-5-(hydroxymethyl)-5-methyltetrahydrofuran-2-yl)-9H-purin-2-yl)isobutyramide. A solution of N-(9-((2R,4S,5R)-4-(benzyloxy)-5-((benzyloxy)methyl)-5-methyltetrahydrofuran-2-yl)-6-hydroxy-9H-purin-2-yl)isobutyramide (100 mg, 188 umol) in DCM (5 mL) was degassed with N2 (3×) and cooled to −78° C. with stirring, and then a solution of BCl₃ (1 M in DCM, 0.60 mL, 0.60 mmol) was added dropwise at −78° C. Then the reaction mixture was stirred at −45° C. for 0.5 h. The reaction mixture was quenched with MeOH (1 mL) at −45° C., then allowed to warm to 0° C. The reaction mixture was adjusted to pH >7 with 28% aqueous NH₄OH solution (1.0 mL) and concentrated to residue. The residue was purified by prep-HPLC (acetonitrile 0-30%/NH₃·H₂O+NH₄HCO₃ in water) to afford the title compound (32 mg, 91.1 μmol, 39% yield) as a white solid. ¹H NMR (400 MHz, MeOD-d4) δ 8.27 (s, 1H), 6.32 (t, J=6.4 Hz, 1H), 4.57-4.52 (m, 1H), 3.58 (d, J=4.4 Hz, 2H), 2.82-2.68 (m, 2H), 2.55-2.46 (m, 1H), 1.24 (s, 3H), 1.22 (s, 6H).

Step 6: 2-amino-9-((2R,4S,5R)-4-hydroxy-5-(hydroxymethyl)-5-methyltetrahydrofuran-2-yl)-9H-purin-6-ol (Cpd. No. 115). A solution of N-(6-hydroxy-9-((2R,4S,5R)-4-hydroxy-5-(hydroxymethyl)-5-methyltetrahydrofuran-2-yl)-9H-purin-2-yl)isobutyramide (20.0 mg, 56.9 μmol) in MeOH (3 mL) was added aqueous NaOH solution (2 M, 0.5 mL). After stirring at 25° C. for 16 h, the reaction mixture was concentrated to residue and purified by prep-HPLC (acetonitrile 0-30%/NH₃·H₂O+NH₄HCO₃ in water) to afford Cpd. No. 115 (5.7 mg, 20.3 μmol, 36% yield) as a white solid. ¹H NMR (400 MHz, MeOD-d4) δ 7.96 (s, 1H), 6.22 (t, 0.1=6.8 Hz, 1H), 4.57-5.46 (m, 1H), 3.70-3.48 (m, 2H), 2.85-2.78 (m, 1H), 2.46-2.36 (m, 1H), 1.21 (s, 3H). LC-MS (ESI) m/z 304.1 [M+Na]⁺. LC-MS RT=0.179 min; Method F.

### Example 24

### Synthesis of 4-amino-1-((2R,4R,5R)-3,3-difluoro-4-hydroxy-5-(hydroxymethyl)-5-methyltetrahydrofuran-2-yl)pyrimidin-2(1H)-one (Cpd. No. 190)

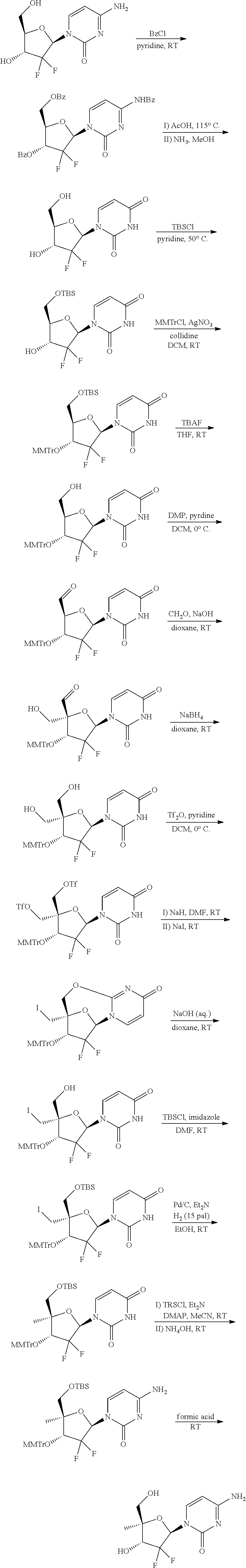

Step 1: (2R,3R,5R)-5-(4-benzamido-2-oxopyrimidin-1(2H)-yl)-2-((benzoyloxy)methyl)-4,4-difluorotetrahydrofuran-3-yl benzoate. To a mixture of 4-amino-1-((2R,4R,5R)-3,3-difluoro-4-hydroxy-5-(hydroxymethyl)tetrahydrofuran-2-yl)pyrimidin-2(1H)-one (20.0 g, 76.0 mmol) in pyridine (250 mL) was added in BzCl (44.0 mL, 379 mmol) at 0° C. under N₂. The mixture was stirred at 25° C. for 15 hours. Then the reaction mixture was concentrated. The residue was dissolved in EtOAc (300 mL), washed with brine (200 mL×3), dried over Na₂SO₄, filtered and concentrated to give the crude product which was purified by silica gel chromatography (solvent gradient: 0-30% EtOAc in Petroleum ether) to afford the title product (39.2 g, 68.1 mmol, 90% yield) as a white solid.

Step 2: 1-((2R,4R,5R)-3,3-difluoro-4-hydroxy-5-(hydroxymethyl)tetrahydrofuran-2-yl)pyrimidine-2,4(1H,3H)-dione. To a solution of (2R,3R,5R)-5-(4-benzamido-2-oxopyrimidin-1(2H)-yl)-2-((benzoyloxy)methyl)-4,4-difluorotetrahydrofuran-3-yl benzoate (20.0 g, 34.8 mmol) in AcOH (130 mL) was added H₂O (13 mL). The reaction mixture was stirred at 110° C. for 16 h. After that, the reaction mixture was concentrated under reduced pressure. The residue was taken up in EtOAc (300 mL), washed with saturated NaHCO₃ (200 mL×3), dried over Na₂SO₄, filtered and concentrated to residue. The residue was treated with 7 M NH₃/MeOH solution (100.0 mL, 0.7 mol), stirred at 25° C. for 16 h. Then the reaction mixture was concentrated to residue which was purified by silica gel chromatography (solvent gradient: 0-5% MeOH in Dichloromethane) to afford the title product (7.9 g, 29.9 mmol, 86% yield over two steps) as a white solid.

Step 3: 1-((2R,4R,5R)-5-(((tert-butyldimethylsilyl)oxy)methyl)-3,3-difluoro-4-hydroxytetrahydrofuran-2-yl)pyrimidine-2,4(1H,3H)-dione. To a solution of 1-((2R,4R,5R)-3,3-difluoro-4-hydroxy-5-(hydroxymethyl)tetrahydrofuran-2-yl)pyrimidine-2,4(1H,3H)-dione (14.5 g, 54.9 mmol) in pyridine (200 mL) was added imidazole (9.40 g, 138 mmol) and TBSCl (10.8 g, 71.7 mmol). The reaction mixture was stirred at 25° C. for 16 h. After that, the reaction mixture was concentrated to residue. The residue was dissolved in EtOAc (300 mL), washed with brine (200 mL×3), dried over Na₂SO₄, filtered and concentrated. The residue was purified by silica gel chromatography (solvent gradient: 0-3% MeOH in Dichloromethane) to afford the title product (16.5 g, 43.6 mmol, 79% yield) as colorless oil. ¹H NMR (400 MHz, CDCl₃) δ 8.63 (br s, 1H), 7.71 (d, J=8.0 Hz, 1H), 6.28-6.20 (m, 1H), 5.72 (d, J=8.0 Hz, 1H), 4.42-4.36 (m, 1H), 4.07-3.96 (m, 2H), 3.95-3.86 (m, 1H), 0.92 (s, 9H), 0.13 (d, J=2.4 Hz, 6H).

Step 4: 1-((2R,4R,5R)-5-(((tert-butyldimethylsilyl)oxy)methyl)-3,3-difluoro-4-((4-methoxyphenyl)diphenylmethoxy)tetrahydrofuran-2-yl)pyrimidine-2,4(1H,3H)-dione. To a solution of 1-((2R,4R,5R)-5-(((tert-butyldimethylsilyl)oxy)methyl)-3,3-difluoro-4-hydroxytetrahydrofuran-2-yl)pyrimidine-2,4(1H,3H)-dione (13.5 g, 35.7 mmol) in DCM (100 mL) was added AgNO₃ (12.2 g, 71.8 mmol), 2,4,6-trimethylpyridine (11.8 mL, 89.3 mmol) and MMTrCl (22.0 g, 71.2 mmol). The resulting reaction mixture was stirred at 25° C. for 16 h. After that, the reaction mixture was filtered, and the filtrate was concentrated to residue which was purified by silica gel chromatography (solvent gradient: 0-20% ethyl acetate in petroleum ether) to afford the title product (11.6 g, 17.8 mmol, 50% yield) as a yellow solid. ¹H NMR (400 MHz, CDCl₃-d) δ 8.03 (br s, 1H), 7.49-7.42 (m, 5H), 7.37-7.27 (m, 8H), 6.85 (d, J=8.8 Hz, 2H), 6.31-6.19 (m, 1H), 5.61 (dd, J=2.4, 8.0 Hz, 1H), 4.53-4.38 (m, 1H), 4.13-4.03 (m, 1H), 3.81 (s, 3H), 3.70-3.55 (m, 1H), 3.25 (dd, J=11.8, 2.8 Hz, 1H), 0.80 (s, 9H), −0.04 (d, J=2.4 Hz, 6H).

Step 5: 1-((2R,4R,5R)-3,3-difluoro-5-(hydroxymethyl)-4-((4-methoxyphenyl)diphenylmethoxy)tetrahydrofuran-2-yl)pyrimidine-2,4(1H,3H)-dione. To a solution of 1-((2R,4R,5R)-5-(((tert-butyldimethylsilyl)oxy)methyl)-3,3-difluoro-4-((4-methoxyphenyl)diphenylmethoxy)tetrahydrofuran-2-yl)pyrimidine-2,4(1H,3H)-dione (10.0 g, 15.4 mmol) in THE (100 mL) was added a solution of TBAF (1M in THF, 46.0 mL, 46.0 mmol). The reaction mixture was stirred at 25° C. for 2 h. Then the reaction mixture was diluted with water (100 mL), extracted with EtOAc (200 mL), washed with brine (150 mL×2), dried over Na₂SO₄, filtered and concentrated to residue which was purified by silica gel chromatography (solvent gradient: 0-35% ethyl acetate in petroleum ether) to afford the title product (8.20 g, 14.6 mmol, 99% yield) as yellow oil. ¹H NMR (400 MHz, CDCl₃-d) δ 8.18 (br s, 1H), 7.55-7.47 (m, 4H), 7.43-7.36 (m, 3H), 7.37-7.28 (m, 6H), 6.93-6.84 (m, 2H), 6.23 (t, J=8.4 Hz, 1H), 5.67 (dd, J=2.4, 8.4 Hz, 1H), 4.34-4.28 (m, 1H), 3.99-3.92 (m, 1H), 3.82 (s, 3H), 3.53-3.46 (m, 1H), 3.05-2.97 (m, 1H).

Step 6: 1-((2R,4R)-3,3-difluoro-5,5-bis(hydroxymethyl)-4-((4-methoxyphenyl)diphenylmethoxy)tetrahydrofuran-2-yl)pyrimidine-2,4(1H,3H)-dione. To a solution of 1-((2R,4R,5R)-3,3-difluoro-5-(hydroxymethyl)-4-((4-methoxyphenyl)diphenylmethoxy)tetrahydrofuran-2-yl)pyrimidine-2,4(1H,3H)-dione (8.00 g, 14.9 mmol) in DCM (60 mL) was added pyridine (3.6 mL, 44.5 mmol) and DMP (19.0 g, 44.8 mmol). The reaction mixture was stirred at 25° C. for 3 h. Then the reaction mixture was diluted with DCM (50 mL), quenched with saturated Na₂S₂O₃ (20 mL) and NaHCO₃ (20 mL). The resulting mixture was stirred at 25° C. for 15 min and then the organic layer was separated, washed with brine (50 mL), dried over Na₂SO₄ and concentrated to afford the crude aldehyde (7.80 g, 98% yield) as a yellow solid. To a solution of the above aldehyde (7.80 g, 14.6 mmol) in dioxane (60 mL) was added 37% HCHO (5.4 mL, 73.0 mmol) and 2 M KOH aqueous solution (16.0 mL, 32.0 mmol). The reaction was stirred at 25° C. for 16 h and then neutralized with AcOH to pH=7. The reaction mixture was then diluted with EtOH (20 mL), NaBH₄ (2.9 g, 76.7 mmol) was added, then the reaction mixture was stirred at 25° C. for 0.5 h. The reaction mixture was quenched with saturated NH₄Cl aqueous (30 mL), extracted with EtOAc (60 mL×2), washed with brine (50 mL×2). The organic layer was dried over Na₂SO₄, filtered and concentrated to residue which was purified by column chromatography on silica gel (solvent gradient: 0-5% MeOH in Dichloromethane) to afford the title compound (4.3 g, 7.59 mmol, 51% yield) as a white solid. ¹H NMR (400 MHz, DMSO-d₆) δ 11.50 (d, J=1.6 Hz, 1H), 7.57 (d, J=8.0 Hz, 1H), 7.48-7.40 (m, 4H), 7.37-7.25 (m, 8H), 6.91 (d, J=8.8 Hz, 2H), 6.30 (t, 0.1=8.0 Hz, 1H), 5.61 (dd, J=2.0, 8.0 Hz, 1H), 5.03 (t, J=5.6 Hz, 1H), 4.87 (t, J=4.8 Hz, 1H), 4.64-4.50 (m, 1H), 3.87-3.81 (m, 1H), 3.74 (s, 3H), 3.49-3.42 (m, 1H), 3.32-3.24 (m, 2H).

Step 7: ((3R,5R)-5-(2,4-dioxo-3,4-dihydropyrimidin-1(2H)-yl)-4,4-difluoro-3-((4-methoxyphenyl)diphenylmethoxy)tetrahydrofuran-2,2-diyl)bis(methylene) bis(trifluoromethanesulfonate). To a solution of 1-((2R,4R)-3,3-difluoro-5,5-bis(hydroxymethyl)-4-((4-methoxyphenyl)diphenylmethoxy)tetrahydrofuran-2-yl)pyrimidine-2,4(1H,3H)-dione (700 mg, 1.24 mmol) in DCM (10 mL) was added pyridine (1.50 mL, 18.5 mmol). The reaction mixture was degassed with N2 (3×) and cooled to 0° C. Tf₂O (509 uL, 3.03 mmol) was added dropwise and the reaction mixture was stirred at 0° C. for 30 min. Then the reaction mixture was concentrated to residue which was purified by silica gel chromatography (solvent gradient: 0-3% MeOH in Dichloromethane) to afford the title product (0.53 g, 0.64 mmol, 52% yield) as a yellow oil.

Step 8: (6R,8R,9R)-7,7-difluoro-9-(iodomethyl)-8-((4-methoxyphenyl)diphenylmethoxy)-7,8,9,10-tetrahydro-2H,6H-6,9-epoxypyrimido[2,1-b][1,3]oxazocin-2-one. A solution of ((3R,5R)-5-(2,4-dioxo-3,4-dihydropyrimidin-1(2H)-yl)-4,4-difluoro-3-((4-methoxyphenyl)diphenylmethoxy)tetrahydrofuran-2,2-diyl)bis(methylene) bis(trifluoromethanesulfonate)(520 mg, 626 μmol) in THF (5 mL) was degassed with N2 (3×) and then cooled to 0° C. NaH (60 wt. % dispersion in mineral oil, 33.0 mg, 825 μmol) was added and the reaction mixture was stirred at 25° C. for 4 h. After that, NaI (277 mg, 1.85 mmol) was added and the reaction mixture was stirred at 25° C. for 16 h. The reaction mixture was quenched with saturated NH₄Cl solution (10 mL), extracted with EtOAc (20 mL), washed with brine (20 mL), dried over Na₂SO₄ and concentrated to give the crude title product (400.0 mg, 97% yield) as yellow oil. LC-MS (ESI) m/z 659.1 [M+H]⁺. LC-MS RT=0.908 min; Method C.

Step 9: 1-((2R,4R,5R)-3,3-difluoro-5-(hydroxymethyl)-5-(iodomethyl)-4-((4-methoxyphenyl)diphenylmethoxy)tetrahydrofuran-2-yl)pyrimidine-2,4(1H,3H)-dione. To a solution of (6R,8R,9R)-7,7-difluoro-9-(iodomethyl)-8-((4-methoxyphenyl)diphenylmethoxy)-7,8,9,10-tetrahydro-2H,6H-6,9-epoxypyrimido[2,1-b][1,3]oxazocin-2-one (0.40 g, 0.61 mmol) in THF (5 mL) was added aqueous NaOH solution (2 M, 1 mL). The resulting reaction mixture was stirred at 25° C. for 1 h. Then the reaction mixture was diluted with water (10 mL), extracted with EtOAc (20 mL), washed with brine (20 mL), dried over Na₂SO₄, filtered and concentrated to residue which was purified by column chromatography on silica gel (solvent gradient: 0-5% MeOH in Dichloromethane) to afford the title compound (225 mg, 333 μmol, 55% yield) as a white solid. ¹H NMR (400 MHz, CDCl₃-d) δ 8.09 (s, 1H), 7.55-7.32 (m, 13H), 6.89 (d, J=8.8 Hz, 2H), 6.20 (d, J=12.4 Hz, 1H), 5.65 (dd, J=2.0, 8.2 Hz, 1H), 4.68 (br t, J=12.8 Hz, 1H), 3.82 (s, 3H), 3.77-3.69 (m, 2H), 3.29 (d, J=12.0 Hz, 1H), 2.34 (d, J=12.4 Hz, 1H).

Step 10: 1-((2R,4R,5R)-5-(((tert-butyldimethylsilyl)oxy)methyl)-3,3-difluoro-5-(iodomethyl)-4-((4-methoxyphenyl)diphenylmethoxy)tetrahydrofuran-2-yl)pyrimidine-2,4(1H,3H)-dione. To a solution of 1-((2R,4R,5R)-3,3-difluoro-5-(hydroxymethyl)-5-(iodomethyl)-4-((4-methoxyphenyl)diphenylmethoxy)tetrahydrofuran-2-yl)pyrimidine-2,4(1H,3H)-dione (220 mg, 325 μmol) in DMF (5 mL) was added imidazole (221.0 mg, 3.25 mmol) and TBSCl (245.0 mg, 1.63 mmol). The reaction mixture was stirred at 25° C. for 16 h. The reaction mixture was quenched with water (10 mL), extracted with EtOAc (20 ml), washed with brine (20 mL×2), dried over Na₂SO₄, filtered and concentrated to residue which was purified by column chromatography on silica gel (solvent gradient: 0-20% ethyl acetate in petroleum ether) to afford the title compound (230 mg, 291 μmol, 90% yield) as colorless oil. ¹H NMR (400 MHz, CDCl₃-d) δ 8.03 (s, 1H), 7.46-7.37 (m, 4H), 7.35-7.28 (m, 9H), 6.89-6.79 (m, 2H), 6.16 (dd, 1=5.2, 10.0 Hz, 1H), 5.59 (dd, J=2.4, 8.4 Hz, 1H), 4.89 (dd, J=9.6, 14.0 Hz, 1H), 3.94 (d, J=11.2 Hz, 1H), 3.81 (s, 3H), 3.77 (d, J=12.4 Hz, 1H), 3.51 (d, J=12.4 Hz, 1H), 3.26 (d, J=11.2 Hz, 1H), 0.79 (s, 9H), −0.04 (d, J=2.4 Hz, 6H).

Step 11: 1-((2R,4R,5R)-5-(((tert-butyldimethylsilyl)oxy)methyl)-3,3-difluoro-4-((4-methoxyphenyl)diphenylmethoxy)-5-methyltetrahydrofuran-2-yl)pyrimidine-2,4(1H,3H)-dione. To a solution of -((2R,4R,5R)-5-(((tert-butyldimethylsilyl)oxy)methyl)-3,3-difluoro-5-(iodomethyl)-4-((4-methoxyphenyl)diphenylmethoxy)tetrahydrofuran-2-yl)pyrimidine-2,4(1H,3H)-dione (0.22 g, 0.28 mmol) in MeOH (2 mL) was added 10 wt. % palladium on carbon (20 mg) and Et₃N (84.0 uL, 603 μmol). The reaction mixture was degassed with H₂ (3×) and stirred at 25° C. for 16 h under H₂ (15 psi). After that, the reaction mixture was filtered and the filtrate was concentrated to dryness. The residue was dissolved in EtOAc (15 mL), washed with water (10 mL) and brine (10 mL), dried over Na₂SO₄, filtered and concentrated to give the crude title compound (150 mg, 226 μmol, 81% yield) as a white solid. ¹H NMR (400 MHz, CDCl₃-d) δ 7.89 (br s, 1H), 7.45 (br d, J=7.6 Hz, 4H), 7.37-7.28 (m, 9H), 6.83 (d, J=8.8 Hz, 2H), 6.14-6.06 (m, 1H), 5.57 (dd, J=2.0, 8.0 Hz, 1H), 4.69-4.61 (m, 1H), 3.80 (s, 3H), 3.48 (d, J=11.2 Hz, 1H), 3.34 (d, 1=11.2 Hz, 1H), 1.47 (s, 3H), 0.79 (s, 9H), −0.04 (d, J=2.4 Hz, 6H).

Step 12: 4-amino-1-((2R,4R,5R)-5-(((tert-butyldimethylsilyl)oxy)methyl)-3,3-difluoro-4-((4-methoxyphenyl)diphenylmethoxy)-5-methyltetrahydrofuran-2-yl)pyrimidin-2(1H)-one. To a solution of 1-((2R,4R,5R)-5-(((tert-butyldimethylsilyl)oxy)methyl)-3,3-difluoro-4-((4-methoxyphenyl)diphenylmethoxy)-5-methyltetrahydrofuran-2-yl)pyrimidine-2,4(1H,3H)-dione (150 mg, 226 μmol) in MeCN (5 mL) was added Et₃N (94.0 μL, 674 μmol), DMAP (27 mg, 221 μmol) and 2,4,6-triisopropylbenzenesulfonyl chloride (205 mg, 677 μmol) at 25° C. The reaction mixture was stirred at 25° C. for 16 h, then aqueous NH₄₀H (1.0 mL, 28 wt. %) was added. The reaction mixture was stirred at 25° C. for another 16 h. After that, the reaction mixture was quenched with water (10 mL), extracted with EtOAc (20 mL), washed with brine (10 mL), dried over Na₂SO₄ and concentrated to residue which was purified by column chromatography on silica gel (solvent gradient: 0-5% MeOH in Dichloromethane) to afford the title compound (80.0 mg, 121 μmol, 54% yield) as a white solid. LC-MS (ESI) m/z 664.3 [M+H]⁺. LC-MS RT=1.003 min; Method C.

Step 13: 4-amino-1-((2R,4R,5R)-3,3-difluoro-4-hydroxy-5-(hydroxymethyl)-5-methyltetrahydrofuran-2-yl)pyrimidin-2(1H)-one (Cpd. No. 190). A solution of 4-amino-1-((2R,4R,5R)-5-(((tert-butyldimethylsilyl)oxy)methyl)-3,3-difluoro-4-((4-methoxyphenyl)diphenylmethoxy)-5-methyltetrahydrofuran-2-yl)pyrimidin-2(1H)-one (70 mg, 105 umol) in a mixture of formic acid (2 mL) and water (0.4 mL) was stirred at 25° C. for 16 h. The reaction mixture was concentrated to residue which was purified by prep-HPLC (acetonitrile 10-40%/NH₃H₂O+NH₄HCO₃ in water) to afford Cpd. No. 190 (9.2 mg, 33.2 μmol, 32% yield) as a white solid. ¹H NMR (400 MHz, MeOD-d4) δ 7.94 (d, J=7.6 Hz, 1H), 6.24-6.18 (m, 1H), 5.91 (d, J=7.6 Hz, 1H), 4.43 (t, J=12.0 Hz, 1H), 3.67 (d, J=12.0 Hz, 1H), 3.57 (d, J=12.0 Hz, 1H), 1.21 (s, 3H). LC-MS (ESI) m/z 555.1 [2M+H]⁺. LC-MS RT=0.256 min; Method C.

### Example 25

### Synthesis of 5-bromo-6-(((2R,4S,5R)-4-hydroxy-5-(hydroxymethyl)-5-vinyltetrahydrofuran-2-yl)amino)pyrimidin-2(1H)-one (Cpd. No. 3)

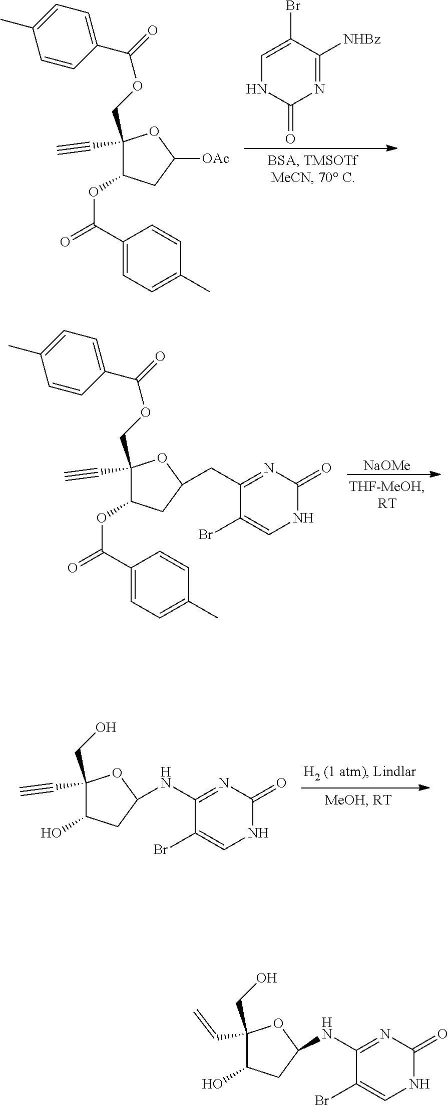

Step 1: (2R,3S)-5-((5-bromo-2-oxo-1,2-dihydropyrimidin-4-yl)amino)-2-ethynyl-2-(((4-methylbenzoyl)oxy)methyl)tetrahydrofuran-3-yl 4-methylbenzoate. A flame-dried round-bottomed flask, equipped with a magnetic stirrer bar and fitted with a reflux condenser, was charged with N-(5-bromo-2-oxo-1,2-dihydropyrimidin-4-yl)benzamide (6.06 g, 20.6 mmol) and anhydrous MeCN (150 mL). N,O-Bis(trimethylsilyl)acetamide (3.89 mL, 25.8 mmol) was added, and the mixture heated to 70° C. with stirring for ca. 1 h before cooling to ambient temperature. Trimethylsilyl trifluoromethanesulfonate (3.77 mL, 20.6 mmol) and (2R,3S)-5-acetoxy-2-ethynyl-2-(((4-methylbenzoyl)oxy)methyl)tetrahydrofuran-3-yl 4-methylbenzoate (7.50 g, 17.2 mmol) were then added, and the reaction mixture was stirred at ambient temperature overnight. The reaction was partitioned between EtOAc and water, then the organics were dried (anhyd. Na₂SO₄), filtered, and concentrated in vacuo. Purification of the residue by flash chromatography on SiO₂ (eluent: 0-90% EtOAc in hexanes) furnished the title compound (5.04 g, 8.91 mmol, 52%) as an amorphous white solid, as a ca. 1:1 mixture of anomers by LCMS. LC-MS (ESI) m/z 565.3 [M−H]⁻. LC-MS RT=1.43 min; Method H.

Step 2: 5-bromo-4-(((4S,5R)-5-ethynyl-4-hydroxy-5-(hydroxymethyl)tetrahydrofuran-2-yl)amino)pyrimidin-2(1H)-one. (2R,3S)-5-((5-bromo-2-oxo-1,2-dihydropyrimidin-4-yl)amino)-2-ethynyl-2-(((4-methylbenzoyl)oxy)methyl)tetrahydrofuran-3-yl 4-methylbenzoate (1.50 g, 2.65 mmol) was dissolved in a mixture of THF (17.6 mL) and MeOH (8.82 mL) with stirring at ambient temperature. A solution of sodium methoxide (25 wt. % in MeOH, 2.12 mL, 7.94 mmol) was added, and the reaction mixture was stirred at ambient temperature for ca. 18 h. The mixture was then adsorbed onto silica gel and purified by flash chromatography on SiO₂ (eluent: 20% IPA in DCM) to furnish the title compound (750 mg, 2.27 mmol, 86%). LC-MS (ESI) m/z 328.2 [M−H]⁻. LC-MS RT=0.45 min; Method A.

Step 3: 5-bromo-4-(((2R,4S,5R)-4-hydroxy-5-(hydroxymethyl)-5-vinyltetrahydrofuran-2-yl)amino)pyrimidin-2(1H)-one (Cpd. No. 3). 5-bromo-4-(((4S,5R)-5-ethynyl-4-hydroxy-5-(hydroxymethyl)tetrahydrofuran-2-yl)amino)pyrimidin-2(1H)-one (200 mg, 606 μmol) was dissolved in MeOH (6.06 mL) in a screw-top glass vial, equipped with a magnetic stirrer bar and a rubber septum, at ambient temperature, then Lindlar catalyst (200 mg) was added in one portion. The vessel was flushed with hydrogen from a balloon for ca. 6 mins, then the reaction mixture was stirred under hydrogen (1 atm) for ca. 24 h. The reaction mixture was then filtered through Celite®, the filter cake rinsed with MeOH, and the filtrate concentrated in vacuo. The residue was purified by flash chromatography on SiO₂ (eluent: 6% IPA in DCM) to furnish Cpd. No. 3 (42.0 mg, 126 μmol, 21%) as a white powder. NMR (400 MHz, DMSO-d6) δ 11.72 (s, 1H), 8.31 (s, 1H), 6.20 (dd, J=8.0, 1.8 Hz, 1H), 6.04 (dd, J=17.5, 11.1 Hz, 1H), 5.38 (d, J=3.4 Hz, 1H), 5.20 (ddd, 0.1=14.3, 13.2, 2.1 Hz, 2H), 5.03 (t, J=5.8 Hz, 1H), 4.27 (dd, J=5.1, 3.6 Hz, 1H), 3.23 (dd, J=11.4, 5.1 Hz, 2H), 2.79 (ddd, J=14.2, 7.8, 6.2 Hz, 1H), 1.88 (d, J=14.3 Hz, 1H). LC-MS (ESI) m/z 333.2 [M+2H]⁺. LC-MS RT=0.62 min: Method A.

### Example 26

### Synthesis of 2-amino-9-((2R,4S,5R)-5-ethyl-4-hydroxy-5-(hydroxymethyl)tetrahydrofuran-2-yl)-9H-purin-6-ol (Cpd. No. 223)

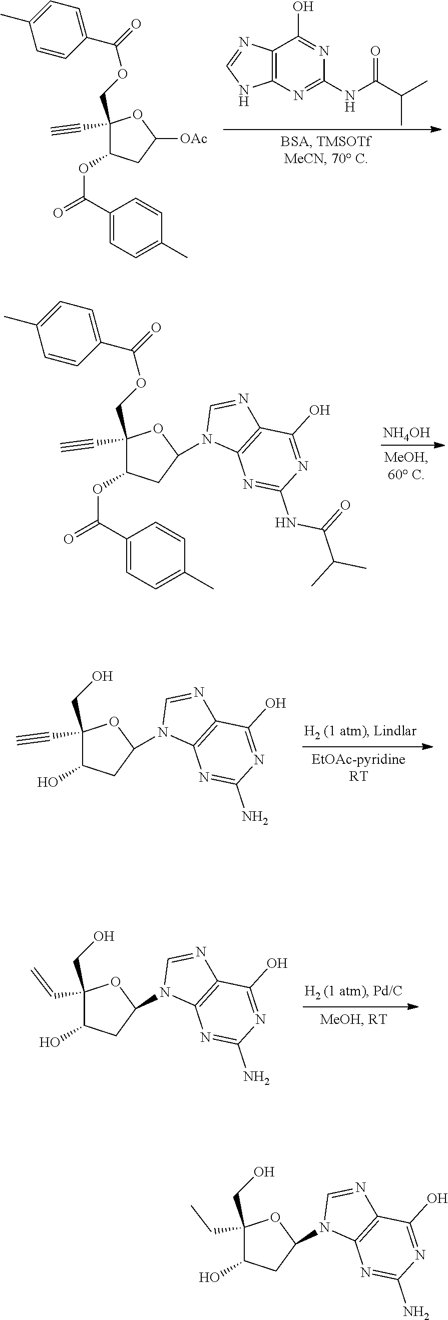

Step 1: (2R,3S)-2-ethynyl-5-(6-hydroxy-2-isobutyramido-9H-purin-9-yl)-2-(((4-methylbenzoyl)oxy)methyl)tetrahydrofuran-3-yl 4-methylbenzoate. A round-bottomed flask equipped with a magnetic stirrer bar was charged with a suspension of N-(6-hydroxy-9H-purin-2-yl)isobutyramide (1.77 g, 7.77 mL) in MeCN (58.2 mL). N,O-Bis(trimethylsilyl)acetamide (8.85 mL, 34.4 mL) was added, and the mixture heated to 70° C. with stirring for ca. 1 h. The mixture was then cooled to 0° C. with stirring, and a solution of (2R,3S)-5-acetoxy-2-ethynyl-2-(((4-methylbenzoyl)oxy)methyl)tetrahydrofuran-3-yl 4-methylbenzoate (3.00 g, 6.87 mmol) in MeCN (4.00 mL) was added, followed by trimethylsilyl trifluoromethanesulfonate (1.90 mL, 10.3 mmol). The resulting mixture was stirred at ambient temperature for ca. 15 mins, then warmed to 70° C. with stirring for ca. 18 h. The reaction mixture was then cooled to ambient temperature, quenched with saturated aqueous NaHCO₃ solution (30 mL) and diluted with saturated aqueous NaCl solution (12 mL). The organics were then extracted with EtOAc (3×30 mL), and the combined organics dried (anhyd. Na₂SO₄), filtered, and concentrated in vacuo. The residue was purified by flash chromatography on SiO₂ (eluent: 0-20% EtOAc in DCM) to afford the title compound (3.75 g, 6.27 mmol, 91%) as a white solid, as a mixture of anomers. LC-MS (ESI) m/z 598.3 [M+H]⁺. LC-MS RT=1.71 min; Method H.

Step 2: 2-amino-9-((4S,5R)-5-ethynyl-4-hydroxy-5-(hydroxymethyl)tetrahydrofuran-2-yl)-9H-purin-6-ol. A 28% aqueous solution of NH₄OH (1.22 mL) was added dropwise to a solution of (2R,3S)-2-ethynyl-5-(6-hydroxy-2-isobutyramido-9H-purin-9-yl)-2-(((4-methylbenzoyl)oxy)methyl)tetrahydrofuran-3-yl 4-methylbenzoate (3.75 g, 6.27 mmol) in MeOH (187 mL), and the reaction mixture heated to 60° C. with stirring for ca. 18 h. After this time, an additional portion of 28% aqueous NH₄OH solution (1.22 mL) was added, and the mixture stirred at 60° C. for a further ca. 3 h. The reaction mixture was then adsorbed onto silica gel and purified by flash chromatography on SiO₂ (eluent: 0-20% MeOH in DCM) to furnish the title compound (749 mg, 2.57 mmol, 41%) as a white powder, as a mixture of anomers. LC-MS (ESI) m/z 292.1 [M+H]⁺. LC-MS RT=0.26 min; Method H.

Step 3: 2-amino-9-((2R,4S,5R)-4-hydroxy-5-(hydroxymethyl)-5-vinyltetrahydrofuran-2-yl)-9H-purin-6-ol. Lindlar catalyst (960 mg) was added to a solution of 2-amino-9-((4S,5R)-5-ethynyl-4-hydroxy-5-(hydroxymethyl)tetrahydrofuran-2-yl)-9H-purin-6-ol (960 mg, 3.30 mmol) in a mixture of EtOAc (31.9 mL) and pyridine (1.10 mL). The reaction mixture was stirred under hydrogen (1 atm) for ca. 18 h, then the mixture was filtered through Celite® and the filtrate concentrated in vacuo. The residue was purified by prep-HPLC (eluent: 25% MeOH+0.1% NH₄OH/75% C02) to furnish the title compound (24.0 mg, 81.8 μmol, 5.0%). LC-MS (ESI) m/z 294.1 [M+H]⁺. LC-MS RT=0.30 min, Method A.

Step 4: 2-amino-9-((2R,4S,5R)-5-ethyl-4-hydroxy-5-(hydroxymethyl)tetrahydrofuran-2-yl)-9H-purin-6-ol (Cpd. No. 223). 2-amino-9-((2R,4S,5R)-4-hydroxy-5-(hydroxymethyl)-5-vinyltetrahydrofuran-2-yl)-9H-purin-6-ol (2.30 mg, 7.84 μmol) was dissolved in MeOH (78.4 μL) under nitrogen. 10 wt. % palladium on carbon (2.30 mg) was added, and the reaction mixture stirred under hydrogen (1 atm) for ca. 1 h. The mixture was then filtered through Celite®, rinsing the filter cake with MeOH, and the filtrate concentrated in vacuo to afford Cpd. No. 223 (1.20 mg, 4.06 μmol, 52%) as a white solid. ¹H NMR (400 MHz, CD₃OD) δ 7.91 (s, 1H), 6.22 (t, J=6.8 Hz, 1H), 4.57 (dd, J=6.3, 3.9 Hz, 1H), 3.69 (d, J=11.7 Hz, 1H), 3.61 (d, J=11.3 Hz, 1H), 2.85-2.78 (m, 1H), 2.38 (ddd, J=13.4, 6.3, 3.9 Hz, 1H), 1.76 (dt, J=15.3, 7.7 Hz, 1H), 1.71-1.63 (m, 1H), 0.99 (t, J=7.6 Hz, 3H). LC-MS (ESI) m/z 296.2 [M+H]⁺. LC-MS RT=0.65 min; Method A.

### Example 27

### Synthesis of 4-amino-1-((2R,3S,4S,5R)-3,4-dihydroxy-5-(hydroxymethyl)-5-methyltetrahydrofuran-2-yl)-5-fluoropyrimidin-2(1H)-one (Cpd. No. 212)

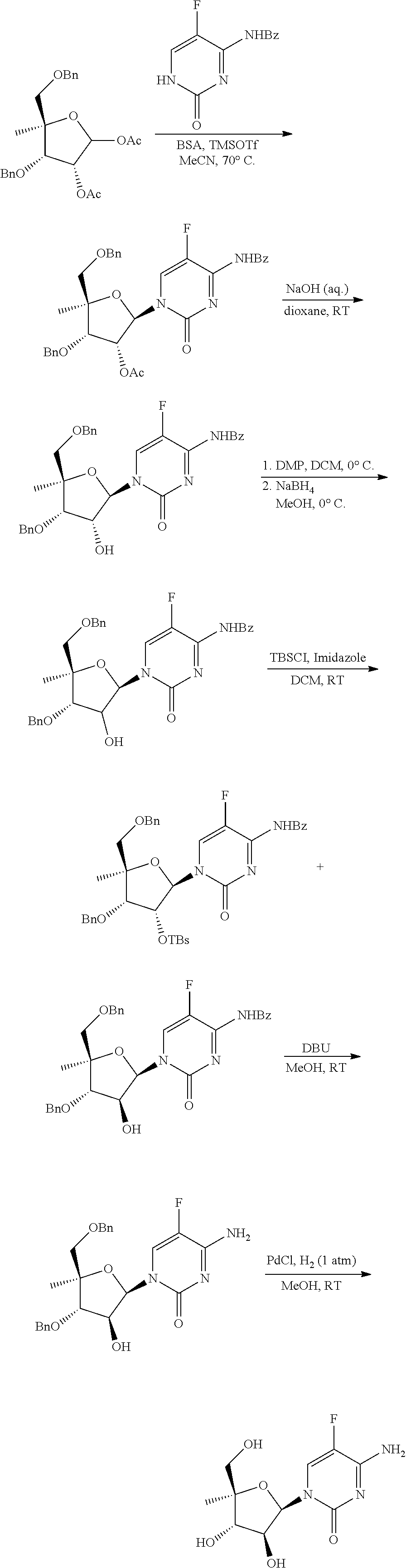

Step 1: (2R,3R,4S,5R)-2-(4-benzamido-5-fluoro-2-oxopyrimidin-1(2H)-yl)-4-(benzyloxy)-5-((benzyloxy)methyl)-5-methyltetrahydrofuran-3-yl acetate. A flame-dried three-necked round-bottomed flask, equipped with a magnetic stirrer bar and fitted with a reflux condenser, was charged with a suspension of N-(5-fluoro-2-oxo-1,2-dihydropyrimidin-4-yl)benzamide (1.99 g, 8.54 mmol) in anhydrous acetonitrile (63.0 mL) under nitrogen atmosphere. N,O-Bis(trimethylsilyl)acetamide 5.46 ML, 21.2 mmol) was added, and the reaction mixture heated to 70° C. with stirring for ca. 1 h. A solution of (3R,4S,5R)-4-(benzyloxy)-5-((benzyloxy)methyl)-5-methyltetrahydrofuran-2,3-diyl diacetate (3.00 g, 7.00 mmol) in anhydrous acetonitrile (7.00 mL), prepared in a separate flame-dried round-bottomed flask, was then added dropwise, followed by dropwise addition of trimethylsilyl trifluoromethanesulfonate (1.55 mL, 8.54 mmol). The reaction mixture was then stirred at 50° C. for ca. 30 mins. The reaction mixture was then cooled to 0° C. with stirring and quenched by slow addition of saturated aqueous NaHCO₃ solution (40 mL). Water (50 mL) was added, the mixture poured into a separatory funnel, and the organics extracted with EtOAc (3×50 mL). The combined organics were washed with water (50 mL) and saturated aaueous NaCl solution, dried (anhyd. Na₂SO₄) filtered and concentrated in vacuo to afford the crude title compound (1.80 g, 2.99 mmol, 43%) as a white solid. LC-MS (ESI) m/z 602.6 [M+H]⁺. LC-MS RT=1.63 min; Method H.

Step 2: N-(1-((2R,3R,4S,5R)-4-(benzyloxy)-5-((benzyloxy)methyl)-3-hydroxy-5-methyltetrahydrofuran-2-yl)-5-fluoro-2-oxo-1,2-dihydropyrimidin-4-yl)benzamide. A solution of (2R,3R,4S,5R)-2-(4-benzamido-5-fluoro-2-oxopyrimidin-1(2H)-yl)-4-(benzyloxy)-5-((benzyloxy)methyl)-5-methyltetrahydrofuran-3-yl acetate (5.00 g, 8.31 mmol) in dioxane (243 mL) was treated with a solution of aqueous NaOH (1M, 25.4 mL, 25.4 mmol) at ambient temperature with stirring for ca. 3 h. The mixture was adjusted to pH 7 by addition of 1M aqueous HCl solution, then the mixture was diluted with water and the organics extracted with EtOAc. The organics were washed with saturated aqueous NaCl solution and dried (anhyd. Na₂SO₄), filtered and concentrated in vacuo. The residue was purified by flash chromatography on SiO₂ (eluent: 55% EtOAc in hexanes) to furnish the title compound (3.10 g, 5.54 mmol, 67%). LC-MS (ESI) m/z 560.4 [M+H]⁺. LC-MS RT=1.81 min; Method H.

Step 3: N-(1-((2R,3S,4S,5R)-4-(benzyloxy)-5-((benzyloxy)methyl)-3-hydroxy-5-methyltetrahydrofuran-2-yl)-5-fluoro-2-oxo-1,2-dihydropyrimidin-4-yl)benzamide. A solution of N-(1-((2R,3R,4S,5R)-4-(benzyloxy)-5-((benzyloxy)methyl)-3-hydroxy-5-methyltetrahydrofuran-2-yl)-5-fluoro-2-oxo-1,2-dihydropyrimidin-4-yl)benzamide (3.10 g, 5.54 mmol) in DCM (30.0 mL) was cooled to 0° C. with stirring, and Dess-Martin periodinane (2.91 g, 6.65 mmol) was added portion-wise to the reaction mixture. The mixture was then warmed to ambient temperature and stirred for ca. 16 h. The reaction mixture was then diluted with Et₂O, and the mixture washed successively with saturated aqueous NaHCO₃ solution and 10% aqueous Na₂S₂O₃ solution. The organics were dried (anhyd. Na₂SO₄), filtered and concentrated in vacuo. The residue was then dissolved in MeOH (30.0 mL), the resulting solution cooled to 0° C. with stirring, and sodium borohydride (218 mg, 5.54 mmol) added. The mixture was stirred at 0° C. for ca. 30 mins, then the reaction was quenched with water and the organics extracted with DCM. The combined organics were dried (anhyd. Na₂SO₄), filtered and concentrated in vacuo to afford the crude product (3.01 g, 5.38 mmol, 97) as a 1:1 mixture of 2′-epimers. To resolve the epimers, the entire crude product (3.01 g, 5.38 mmol) was dissolved in anhydrous DCM (43.4 mL) and treated with imidazole (1.47 g, 21.4 mmol) and TBSCl (2.45 g, 16.1 mmol) at 0° C. with stirring, leading to selective silylation of the 2′-@-epimer. The reaction was quenched with saturated aqueous NaHCO₃ solution, and the phases separated. The organics were dried (anhyd. Na₂SO₄), filtered and concentrated in vacuo. The residue was purified by flash chromatography on SiO₂ (eluent: 20-60% EtOAc in hexanes) to afford he title compound (1.20 g, 2.14 mmol, 80%) was recovered as a light yellow foam. ¹H NMR (400 MHz, CDCl₃) δ 8.25 (d, J=7.5 Hz, 2H), 8.06 (d, J=6.2 Hz, 1H,), 7.58-7.51 (m, 1H), 7.47-7.25 (m, 12H), 6.08 (dd, J=4.2, 1.4 Hz, 1H), 4.76 (d, J=12.0 Hz, 1H), 4.62-4.46 (m, 4H), 4.04 (d, J=3.2 Hz, 1H), 3.66 (d, J=10.2 Hz, 1H), 3.45 (d, J=10.1 Hz, 1H), 1.26 (s, 3H). LC-MS (ESI) m/z 560.6 [M+H]+. LC-MS RT=1.52 min; Method H.

Step 4: 4-amino-1-((2R,3S,4S,5R)-4-(benzyloxy)-5-((benzyloxy)methyl)-3-hydroxy-5-methyltetrahydrofuran-2-yl)-5-fluoropyrimidin-2(1H)-one. DBU (114 μL, 751 μmol) was added dropwise to a stirring solution of N-(1-((2R,3S,4S,5R)-4-(benzyloxy)-5-((benzyloxy)methyl)-3-hydroxy-5-methyltetrahydrofuran-2-yl)-5-fluoro-2-oxo-1,2-dihydropyrimidin-4-yl)benzamide (280 mg, 500 μmol) in MeOH (5.26 mL) at ambient temperature, and the mixture warmed to 50° C. with stirring for ca. 16 h. The reaction mixture was cooled to ambient temperature, adsorbed onto silica gel, and purified by flash chromatography on SiO₂ (eluent: 15% MeOH in DCM) to furnish the title compound (200 mg, 457 μmol, 57%). ¹H NMR (400 MHz, CDCl₃) δ 9.87 (br. S, 1H), 8.17 (d, 0.1=6.5 Hz, 1H), 7.32-7.17 (m, 10H), 6.64 (br. S, 1H), 6.18 (d, J=5.7 Hz, 1H), 6.05 (br. S, 1H), 4.85 (d, J=12.1 Hz, 1H), 4.84-4.79 (m, 1H), 4.60 (d, J=12.1 Hz, 1H), 4.47 (s, 2H), 4.07 (d, J=6.6 Hz, 1H), 3.55 (d, J=10.3 Hz, 1H), 3.40 (d, J=10.2 Hz, 1H), 1.24 (s, 3H). LC-MS (ESI) m/z 456.5 [M+H]+. LC-MS RT=1.19 min; Method H.

Step 5: 4-amino-1-((2R,3S,4S,5R)-3,4-dihydroxy-5-(hydroxymethyl)-5-methyltetrahydrofuran-2-yl)-5-fluoropyrimidin-2(1H)-one (Cpd. No. 212). Palladium(II) chloride (74.0 mg, 417 μmol) was added under nitrogen to a solution of 4-amino-1-((2R,3S,4S,5R)-4-(benzyloxy)-5-((benzyloxy)methyl)-3-hydroxy-5-methyltetrahydrofuran-2-yl)-5-fluoropyrimidin-2(1H)-one (190 mg, 417 μmol) in MeOH (16.8 mL) at ambient temperature. The mixture was sparged with hydrogen from a balloon for ca. 1 min, then the reaction mixture was stirred under hydrogen (1 atm) for ca. 1 h. Et₃N (ca. 5 mL) was added, and the mixture was filtered through a short pad of Celite®. The filtrate was concentrated in vacuo and the residue purified by flash chromatography on SiO₂ (eluent: 20% MeOH in DCM) to furnish Cpd. No. 193 (65 mg, 252 μmol, 73%) as an amorphous white solid. ¹H NMR (400 MHz, CD3OD) δ 8.10 (d, J=6.9 Hz, 1H), 6.11 (dd, J=5.0, 1.9 Hz, 1H), 4.31 (app. t, J=4.8 Hz, 1H), 4.10 (d, J=4.6 Hz, 1H), 3.65 (d, A of AB, J_{AB}=11.7 Hz, 1H), 3.62 (d, B of AB, J_{AB}=11.7 Hz, 1H) 1.18 (s, 3H). LC-MS (ESI) m/z 276.2 [M+H]+. LC-MS RT=0.21 min; Method A.

### Example 28

### Synthesis of 2-amino-9-((2R,4S,5R)-5-(fluoromethyl)-4-hydroxy-5-(hydroxymethyl)tetrahydrofuran-2-yl)-9H-purin-6-ol (Cpd. No. 104)

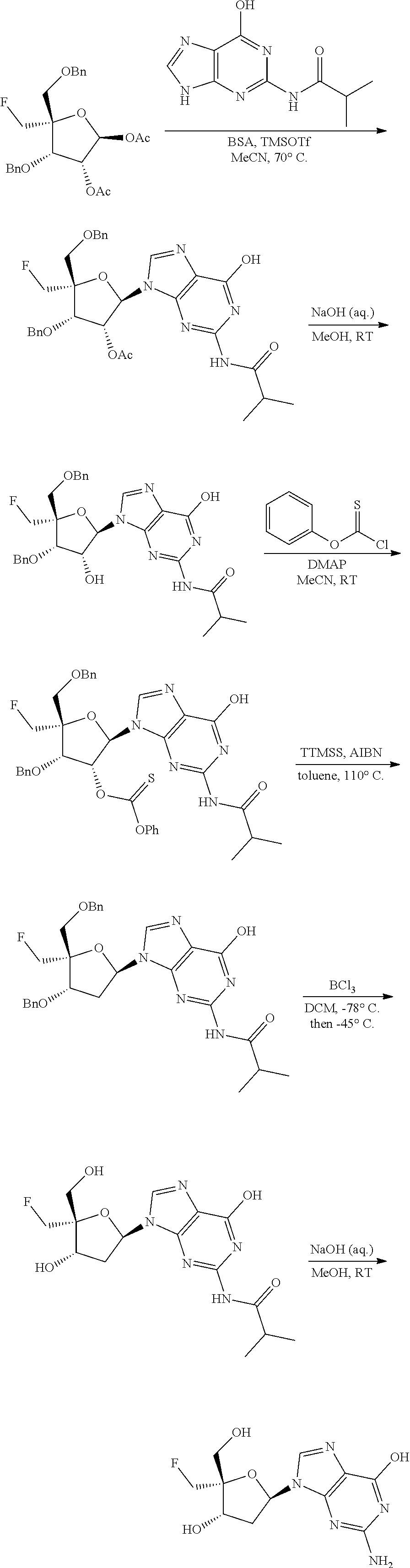

Step 1: (2R,3R,4S,5R)-4-(benzyloxy)-5-((benzyloxy)methyl)-5-(fluoromethyl)-2-(6-hydroxy-2-isobutyramido-9H-purin-9-yl)tetrahydrofuran-3-yl acetate. A mixture of N-(6-hydroxy-9H-purin-2-yl)-2-methyl-propanamide (1.20 g, 5.42 mmol) in acetonitrile (15.0 mL) was degassed with N2(3×). Then N,O-bis(trimethylsilyl)acetamide (2.80 g, 13.8 mmol) was added. After that, the reaction mixture was stirred at 70° C. for 1 h, then cooled to room temperature. Trimethylsilyl trifluoromethanesulfonate (2.00 g, 9.00 mmol) was added and then (2S,3R,4S,5R)-4-(benzyloxy)-5-((benzyloxy)methyl)-5-(fluoromethyl)tetrahydrofuran-2,3-diyl diacetate (2.0 g, 4.48 mmol) in acetonitrile (15.0 mL) was added. The reaction mixture was stirred at 75° C. for 16 h. After cooling to 25° C., the reaction mixture was diluted with water (30 mL), extracted with EtOAc (40 mL), washed with saturated aqueous NaCl solution (30 mL), dried (anhyd. Na₂SO₄), filtered, and concentrated under reduced pressure to give the crude product, which was purified by silica gel chromatography (eluent: 0-3% MeOH in DCM) to afford the title compound (1.20 g, 1.97 mmol, 44%) as a yellow oil. ¹H NMR (400 MHz, CDCl₃) δ 12.03 (br s, 1H), 8.38 (s, 1H), 7.91 (s, 1H), 7.37-7.28 (m, 10H), 6.10 (d, J=6.0 Hz, 1H), 5.77 (t, J=5.6 Hz, 1H), 4.66-4.51 (m, 7H), 3.75-3.64 (m, 2H), 2.60-2.51 (m, 1H), 2.05 (s, 3H), 1.29-1.22 (m, 6H).

Step 2: N-(9-((2R,3R,4S,5R)-4-(benzyloxy)-5-((benzyloxy)methyl)-5-(fluoromethyl)-3-hydroxytetrahydrofuran-2-yl)-6-hydroxy-9H-purin-2-yl)isobutyramide. To a solution of (2R,3R,4S,5R)-4-(benzyloxy)-5-((benzyloxy)methyl)-5-(fluoromethyl)-2-(6-hydroxy-2-isobutyramido-9H-purin-9-yl)tetrahydrofuran-3-yl acetate (1.00 g, 1.65 mmol) in MeOH (15.0 mL) was added aqueous NaOH (2 M, 2.00 mL) at 25° C. After stirring at 25° C. for 1 h, the reaction mixture was adjusted to pH=7 with aqueous HCl (1 M) and then concentrated in vacuo. The residue was purified by silica gel chromatography (eluent: 0-3% MeOH in DCM) to afford the title compound (0.35 g, 619 μmol, 38% yield) as a white solid. ¹H NMR (400 MHz, DMSO-d6) δ 12.06 (s, 1H), 11.65 (s, 1H), 8.16 (s, 1H), 7.38-7.29 (m, 10H), 5.91 (d, J=7.2 Hz, 1H), 5.87 (d, J=5.6 Hz, 1H), 5.03-4.96 (m, 1H), 4.89 (d, J=11.6 Hz, 1H), 4.67-4.52 (m, 5H), 4.28 (d, J=4.6 Hz, 1H), 3.73-3.61 (m, 2H), 2.78-2.70 (m, 1H), 1.08 (d, J=6.8 Hz, 6H).

Step 3: O-((2R,3R,4S,5R)-4-(benzyloxy)-5-((benzyloxy)methyl)-5-(fluoromethyl)-2-(6-hydroxy-2-isobutyramido-9H-purin-9-yl)tetrahydrofuran-3-yl)O-phenyl carbonothioate. To a solution of N-(9-((2R,3R,4S,5R)-4-(benzyloxy)-5-((benzyloxy)methyl)-5-(fluoromethyl)-3-hydroxytetrahydrofuran-2-yl)-6-hydroxy-9H-purin-2-yl)isobutyramide (770 mg, 1.36 mmol) in acetonitrile (15.0 mL) was added DMAP (220 mg, 1.80 mmol), then O-phenyl chlorothionoformate (352 mg, 2.04 mmol) was added dropwise at 0° C. The resulting mixture was stirred at 25° C. for 1 h. After that, the reaction mixture was diluted with water (10 mL), extracted with EtOAc (20 mL), washed with brine (20 mL, dried over Na₂SO₄, filtered and concentrated. The residue was purified by silica gel chromatography (eluent: 0-3% MeOH in DCM) to afford the title compound (820 mg, 1.17 mmol, 86% yield) as a white solid. LC-MS (ESI) m/z 702.1 [M+H]⁺. LC-MS RT=1.025 min; Method C.

Step 4: N-(9-((2R,4S,5R)-4-(benzyloxy)-5-((benzyloxy)methyl)-5-(fluoromethyl)tetrahydrofuran-2-yl)-6-hydroxy-9H-purin-2-yl)isobutyramide. A solution of O-((2R,3R,4S,5R)-4-(benzyloxy)-5-((benzyloxy)methyl)-5-(fluoromethyl)-2-(6-hydroxy-2-isobutyramido-9H-purin-9-yl)tetrahydrofuran-3-yl) 0-phenyl carbonothioate (820 mg, 1.17 mmol) in toluene (12.0 mL) was purged with N2 for ca. 5 min. Then AIBN (96.0 mg, 585 μmol) and TTMSS (872 mg, 3.51 mmol) were added. The resulting reaction mixture was stirred at 110° C. for 2 h. After cooling to room temperature, the reaction mixture was concentrated to residue which was purified by silica gel chromatography (eluent: 0-3% MeOH in DCM) to afford the title compound (540 mg, 983 μmol, 84%) as a white solid. ¹H NMR (400 MHz, DMSO-d6) δ 11.99 (s, 1H), 11.64 (s, 1H), 8.23 (s, 1H), 7.37-7.24 (m, 10H), 6.29 (t, J=6.8 Hz, 1H), 4.70-4.64 (m, 2H), 4.58-4.49 (m, 5H), 3.63-3.57 (m, 2H), 2.95-2.87 (m, 1H), 2.80-2.68 (m, 2H), 1.11 (d, J=6.4 Hz, 6H).

Step 5: N-(9-((2R,4S,5R)-5-(fluoromethyl)-4-hydroxy-5-(hydroxymethyl)tetrahydrofuran-2-yl)-6-hydroxy-9H-purin-2-yl)isobutyramide. To a solution of N-(9-((2R,4S,5R)-4-(benzyloxy)-5-((benzyloxy)methyl)-5-(fluoromethyl)tetrahydrofuran-2-yl)-6-hydroxy-9H-purin-2-yl)isobutyramide (100 mg, 182 umol) in DCM (5.00 mL) was degassed with N2 (3×) and cooled to −78° C. with stirring, then a solution of BCl₃ (1 M in DCM, 0.55 mL, 0.55 mmol) was added dropwise at −78° C. After that, the reaction mixture was stirred at 445° C. for 0.5 h. Then the reaction was quenched with MeOH (1 mL) at −45° C. The mixture was allowed to warm to 0° C., then the mixture was adjusted to pH >7 with 1 mL NH₃·H₂O (28% purity) and concentrated. The residue was purified by prep-HPLC (eluent: acetonitrile 0-30%/NH₃·H₂O+NH₄HCO₃ in water) to afford the title compound (21 mg, 56.9 μmol, 31% yield) as a white solid. ¹H NMR (400 MHz, MeOD-d₄) δ 8.23 (s, 1H), 6.43 (t, J=6.8 Hz, 1H), 4.73-4.66 (m, 2H), 4.60-4.52 (m, 1H), 3.75-3.68 (m, 2H), 2.88-2.81 (m, 1H), 2.76-2.66 (m, 1H), 2.57-2.46 (m, 1H), 1.24 (d, J=6.8 Hz, 6H).

Step 6: 2-amino-9-((2R,4S,5R)-5-(fluoromethyl)-4-hydroxy-5-(hydroxymethyl)tetrahydrofuran-2-yl)-9H-purin-6-ol (Cpd. No. 104). To a solution of N-(9-((2R,4S,5R)-5-(fluoromethyl)-4-hydroxy-5-(hydroxymethyl)tetrahydrofuran-2-yl)-6-hydroxy-9H-purin-2-yl)isobutyramide (20.0 mg, 54.1 μmol) in MeOH (3.00 mL) was added NaOH (2 M, 0.5 mL). The reaction mixture was stirred at 25° C. for 16 h. Then the reaction mixture was concentrated to residue and purified by prep-HPLC (acetonitrile 0-30%/NH₃·H₂O+NH₄HCO₃ in water) to afford Cpd. No. 104 (5.0 mg, 16.7 μmol, 31% yield) as a white solid. ¹H NMR (400 MHz, MeOD-d4) δ 7.93 (s, 1H), 6.48 (br s, 2H), 6.23-6.15 (m, 1H), 5.42 (d, J=4.4 Hz, 1H), 5.13 (t, J=5.6 Hz, 1H), 4.68-4.40 (m, 3H), 3.54-3.44 (m, 2H), 2.77-2.67 (m, 1H), 2.29-2.19 (m, 1H). LC-MS (ESI) m/z 300.1 [M+H]*. LC-MS RT=0.297 min; Method F

### Example 29

### Cancer Cell Proliferation Activity

Dose response relationships on cell proliferation was assessed for representative Compounds of the Disclosure on cancer cell lines. Briefly, compound treatment of cells started one day after seeding with a final DMSO concentration of 0.1%, and was performed by nanodrop-dispensing using a Tecan Dispenser. 0.1% DMSO (solvent) and Staurosporine (10 μM) served as high control (100% viability) and low control (0% viability), respectively. Compound 13 was tested at 10, 3, 1, 0.3, 0.1, 0.03, 0.01, and 0.003 μM.

Cells were cultured in the appropriate media. For the assays, cells were seeded in white cell culture-treated flat and clear bottom multiwell plates and incubated at 37° C. overnight before compound was added. After incubation for 72 h at 37° C. at 5% or 10% CO₂ dependent on the medium, cell plates were equilibrated to room temperature for one hour, CellTiterGlo reagent (Promega) was added and luminescence was measured approximately an hour later using a luminometer.

Raw data were converted into percent cell viability relative to the high and low control, which were set to 100% and 0%, respectively. IC₅₀ calculation was performed using GraphPad Prism software with a variable slope sigmoidal response fitting model using 0% viability as bottom constraint and 100% viability as top constraint.

The IC₅₀ values are summarized in Tables 9A-1, Table 10A, and Table 10B.

TABLE 9A

| | Cpd. No. | 1 | 2 | 3 | 4 | 6 |
|---|---|---|---|---|---|---|
| Cell Line | Cancer Type | IC₅₀ (μM) | | | | |
| 5637 | bladder | 0.88 | 8.67 | 5.02 | 0.03 | >50 |
| 22RV1 | prostate | 1.86 | >50 | >50 | 0.40 | >50 |
| 786O | kidney | 0.62 | >50 | >50 | >50 | >50 |
| A204 | muscle | 0.55 | 1.00 | 1.10 | 0.14 | 7.27 |
| A2780 | ovary | 0.91 | >50 | >50 | 0.68 | >50 |
| A375 | skin | 0.53 | >50 | 4.58 | 0.73 | >50 |
| A431 | skin | 0.53 | >50 | >50 | 0.04 | >50 |
| A549 | lung | >50 | >50 | 3.33 | 0.24 | >50 |
| A673 | bone | 2.56 | >50 | >50 | 0.51 | >50 |
| ACHN | kidney | >50 | 3.20 | >50 | 0.05 | >50 |
| ASPC1 | pancreas | 0.47 | >50 | >50 | 0.01 | >50 |
| BT20 | breast | 0.55 | >50 | >50 | >50 | >50 |
| BXPC3 | pancreas | | >50 | >50 | 0.01 | >50 |
| C33A | endometrial | 0.95 | 4.92 | 2.67 | 0.13 | >50 |
| CACO2 | colon | 1.04 | >50 | >50 | >50 | >50 |
| CAKI1 | kidney | 0.06 | 1.36 | 3.25 | 0.40 | >50 |
| CALU6 | lung | 0.65 | >50 | 6.06 | 0.25 | >50 |
| CASKI | endometrial | 0.31 | >50 | >50 | 6.17 | >50 |
| CLS439 | bladder | >50 | >50 | >50 | 17.60 | >50 |
| COLO205 | colon | 1.06 | >50 | 2.31 | 0.06 | >50 |
| COLO678 | colon | >50 | >50 | >50 | >50 | >50 |
| DLD1 | colon | 2.07 | >50 | >50 | 3.66 | >50 |
| DU145 | prostate | 0.19 | >50 | >50 | 1.34 | >50 |
| EFO21 | ovary | 0.52 | >50 | >50 | 3.62 | >50 |
| EJ28 | bladder | 0.49 | >50 | >50 | 0.19 | >50 |
| GRANTA- | hematological | >50 | 1.12 | 1.06 | 0.03 | 0.46 |
| 519 | | | | | | |
| HCT116 | colon | 0.82 | >50 | 3.65 | 1.18 | >50 |
| HCT15 | colon | 2.78 | >50 | >50 | 1.58 | >50 |
| HEK293 | kidney | 0.71 | >50 | 3.46 | >50 | >50 |
| HELA | endometrial | 1.07 | >50 | 5.34 | >50 | >50 |
| HEPG2 | liver | 0.67 | >50 | >50 | 0.54 | >50 |
| HL-60 | hematological | 0.83 | >50 | 3.42 | 0.81 | >50 |
| HS578T | breast | 0.85 | >50 | >50 | >50 | >50 |
| HS729 | muscle | 1.24 | >50 | 4.91 | 1.41 | >50 |
| HT1080 | connective | >50 | >50 | 3.43 | 0.19 | >50 |
| | tissue | | | | | |
| HT29 | colon | 0.53 | >50 | >50 | 0.16 | >50 |
| IGROV1 | ovary | 0.38 | >50 | >50 | 1.65 | >50 |
| IMR90 | lung | 0.64 | >50 | 5.39 | >50 | >50 |
| J82 | bladder | 0.54 | >50 | >50 | 1.08 | >50 |
| JAR | placenta | 0.44 | >50 | >50 | >50 | >50 |
| JEG3 | placenta | 0.67 | >50 | >50 | 0.10 | >50 |
| JIMT1 | breast | 0.16 | >50 | 11.84 | 0.89 | >50 |
| K-562 | hematological | >50 | >50 | 5.04 | >50 | >50 |
| KASUMI-1 | hematological | 0.15 | >50 | 0.50 | 0.40 | >50 |
| L-363 | hematological | 0.57 | >50 | >50 | >50 | >50 |
| LOVO | colon | 0.29 | >50 | >50 | 0.03 | >50 |
| MCF7 | breast | >50 | >50 | 2.60 | 1.76 | >50 |
| MDAMB231 | breast | >50 | >50 | 2.96 | 0.37 | >50 |
| MDAMB435 | skin | 1.72 | >50 | >50 | 2.52 | >50 |
| MDAMB436 | breast | 0.37 | >50 | 3.65 | 0.14 | >50 |
| MDAMB468 | breast | 0.69 | >50 | >50 | 0.77 | >50 |
| MG63 | bone | >50 | >50 | 3.93 | 0.68 | >50 |
| MHHES1 | bone | 0.60 | >50 | 4.87 | 1.93 | >50 |
| MIAPACA2 | pancreas | >50 | >50 | >50 | >50 | >50 |
| MINO | hematological | 0.25 | 0.25 | 1.51 | 0.01 | 0.68 |
| MT3 | breast | 0.51 | >50 | 3.68 | 0.06 | >50 |
| MV4-11 | hematological | 0.17 | 3.40 | 2.14 | 0.14 | >50 |
| NCI-H23 | lung | 0.83 | >50 | 1.81 | 0.98 | >50 |
| NCIH292 | lung | >50 | >50 | 5.41 | >50 | >50 |
| NCIH358M | lung | 0.56 | >50 | 2.69 | 0.07 | >50 |
| NCIH460 | lung | 0.97 | 5.15 | >50 | 0.20 | >50 |
| NCIH82 | lung | 0.61 | >50 | >50 | 0.39 | >50 |
| OVCAR3 | ovary | 0.26 | >50 | 3.08 | 3.09 | >50 |
| OVCAR4 | ovary | 0.96 | >50 | 3.18 | >50 | >50 |
| PANC1 | pancreas | >50 | >50 | >50 | 1.62 | >50 |
| PANC1005 | pancreas | 0.44 | >50 | >50 | >50 | >50 |
| PBMC | hematological | 0.31 | >50 | >50 | 0.70 | >50 |
| PC3 | prostate | 0.87 | >50 | 4.81 | 2.60 | >50 |
| PLCPRF5 | liver | 3.20 | >50 | >50 | >50 | >50 |
| RAMOS | hematological | 0.94 | 1.77 | 1.41 | 0.16 | 8.68 |
| RD | muscle | 1.46 | >50 | 2.67 | 0.42 | >50 |
| RDES | bone | 0.41 | >50 | >50 | 6.01 | >50 |
| SAOS2 | bone | 0.64 | >50 | 1.70 | 0.09 | 2.05 |
| SF268 | brain | >50 | >50 | >50 | 0.22 | >50 |
| SF295 | brain | 1.42 | >50 | >50 | 3.90 | >50 |
| SKBR3 | breast | 0.15 | 17.04 | 3.45 | 0.03 | >50 |
| SKHEP1 | liver | >50 | >50 | 3.02 | 0.77 | >50 |
| SKLMS1 | uterus | 0.47 | >50 | >50 | 1.15 | >50 |
| SKMEL28 | skin | 1.26 | >50 | 4.19 | 2.90 | >50 |
| SKMEL5 | skin | >50 | >50 | >50 | >50 | >50 |
| SKNAS | brain | 1.14 | >50 | 0.96 | >50 | >50 |
| SKNSH | brain | 0.75 | >50 | 0.91 | >50 | >50 |
| SKOV3 | ovary | 0.76 | >50 | 2.45 | 0.41 | >50 |
| SNB75 | brain | 2.37 | >50 | >50 | 2.82 | >50 |
| SU-DHL-10 | hematological | 0.12 | 1.97 | 5.56 | 0.33 | 2.55 |
| SU-DHL-6 | hematological | 0.43 | >50 | >50 | 0.45 | >50 |
| SW620 | colon | 0.73 | >50 | >50 | 0.24 | >50 |
| T24 | bladder | 1.46 | >50 | >50 | 0.40 | >50 |
| TE671 | muscle | 4.78 | >50 | 3.08 | 0.17 | >50 |
| THP-1 | hematological | >50 | >50 | 3.01 | 2.14 | >50 |
| U20S | bone | 0.84 | >50 | >50 | >50 | >50 |
| U87MG | brain | >50 | >50 | >50 | 1.87 | >50 |
| UMUC3 | bladder | >50 | >50 | >50 | 1.34 | >50 |
| UO31 | kidney | 0.63 | >50 | >50 | >50 | >50 |
| WSU-NHL | hematological | 0.72 | 2.50 | 2.34 | 0.38 | 2.32 |

TABLE 9B

| Cpd. No. | Gemcitabine | 105 | 112 | 104 | 126 | 133 |
|---|---|---|---|---|---|---|
| Cell Line | IC₅₀ (μM) | | | | | |
| 5637 | 0.0016 | 0.74 | 2.89 | 3.94 | 1.00 | 4.09 |
| 22RV1 | 0.013 | 0.31 | 1.50 | 5.15 | 1.57 | >>50 |
| 786O | 0.0041 | 2.51 | >50 | >50 | 0.52 | 2.59 |
| A204 | 0.0009 | 2.55 | 5.28 | 0.17 | 0.29 | 4.91 |
| A2780 | 0.0024 | 3.50 | >50 | >50 | 1.26 | 4.54 |
| A375 | 0.0023 | 3.51 | 6.30 | >50 | 0.26 | 3.67 |
| A431 | 0.0023 | 0.05 | 0.34 | 3.21 | 0.34 | 2.29 |
| A549 | 0.0031 | 0.53 | 2.52 | 18.48 | >50 | 4.96 |
| A673 | 0.0035 | 2.81 | 5.33 | >50 | 2.31 | >50 |
| ACHN | 0.0015 | 0.27 | 1.51 | 3.06 | 3.30 | >50 |
| ASPC1 | 0.0052 | 0.16 | 0.58 | 2.41 | 0.42 | 2.99 |
| BT20 | 0.007 | >50 | >50 | >50 | 0.52 | 4.14 |
| BXPC3 | 0.0024 | 0.02 | 0.08 | 5.32 | >50 | 7.13 |
| C33A | 0.0044 | 1.58 | 3.92 | 4.70 | 1.79 | 4.28 |
| CACO2 | 0.007 | >50 | >50 | >50 | 0.72 | 5.32 |
| CAKI1 | 0.0027 | >50 | >50 | 1.40 | 0.03 | 0.41 |
| CALU6 | 0.0061 | 1.71 | 4.68 | >50 | 0.61 | 3.07 |
| CASKI | 0.0059 | 2.47 | 7.06 | >50 | 0.26 | 2.33 |
| CLS439 | 0.0035 | >50 | >50 | >50 | >50 | >50 |
| COLO205 | 0.0202 | 0.31 | 1.83 | 3.78 | 1.30 | 5.86 |
| COLO678 | 0.0053 | >50 | >50 | 4.91 | 5.81 | 9.54 |
| DLD1 | 0.0053 | 3.74 | >50 | >50 | 1.98 | >50 |
| DU145 | 0.0021 | 1.00 | 3.15 | 3.56 | 0.12 | 3.30 |
| EFO21 | 0.0019 | 2.78 | 16.93 | >50 | 0.57 | 3.75 |
| EJ28 | 0.0021 | 1.72 | 5.41 | 5.74 | 0.56 | 5.11 |
| GRANTA- | 0.0005 | 0.65 | 3.30 | 1.01 | 1.03 | >50 |
| 519 | | | | | | |
| HCT116 | 0.002 | 5.53 | >50 | 4.11 | 0.93 | 3.96 |
| HCT15 | 0.0047 | 2.95 | >50 | >50 | 1.81 | >50 |
| HEK293 | 0.0209 | 4.35 | >50 | >50 | 0.74 | 5.30 |
| HELA | 0.006 | >50 | >50 | 2.39 | 0.55 | 2.73 |
| HEPG2 | 0.002 | 0.77 | 3.40 | 6.08 | 0.79 | 3.32 |
| HL-60 | 0.0022 | 3.87 | >50 | 3.84 | 0.52 | 5.42 |
| HSS78T | 0.0038 | >50 | >50 | >50 | 0.82 | 3.71 |
| HS729 | 0.0036 | 1.37 | >50 | 2.42 | 1.36 | 8.57 |
| HT1080 | 0.0017 | 2.69 | >50 | >50 | >50 | >50 |
| HT29 | 0.0035 | 0.53 | 2.80 | >50 | 0.42 | 2.16 |
| IGROV1 | 0.0048 | 2.99 | >50 | 4.72 | 0.43 | 2.72 |
| IMR.90 | 0.0026 | >50 | >50 | >50 | 0.91 | 2.76 |
| J82 | 0.0051 | 1.00 | 1.32 | 4.45 | 0.54 | 3.18 |
| JAR | 0.002 | 5.83 | >50 | >50 | 0.46 | 3.66 |
| JEG3 | 0.0053 | 1.17 | 4.20 | >50 | 0.37 | 3.36 |
| JIMT1 | 0.0009 | 7.28 | >50 | 2.21 | 0.07 | 1.43 |
| K-562 | 0.0142 | 5.83 | >50 | >50 | >50 | >50 |
| KASUMI-1 | 0.0035 | >50 | >50 | 0.23 | 0.06 | 1.54 |
| L-363 | 0.0034 | 2.80 | 5.63 | >50 | 0.69 | 3.16 |
| LOVO | 0.0023 | 0.51 | 1.95 | >50 | 0.26 | 3.61 |
| MCF7 | 0.0041 | 0.17 | 1.04 | >50 | >50 | >50 |
| MDAMB231 | 0.0009 | >50 | >50 | 2.39 | 2.69 | >50 |
| MDAMB435 | 0.0028 | 2.44 | >50 | 5.26 | 1.25 | >50 |
| MDAMB436 | 0.0018 | 0.42 | 1.43 | 3.02 | 0.25 | 3.36 |
| MDAMB468 | 0.0019 | 2.88 | >50 | >50 | 0.79 | 2.58 |
| MG63 | 0.0048 | 1.81 | 4.70 | >50 | >50 | >50 |
| MHHES1 | 0.002 | 2.29 | >50 | >50 | 0.86 | 3.07 |
| MIAPACA2 | 0.0068 | 2.43 | 6.39 | >50 | >50 | >50 |
| MINO | 0.0003 | 0.58 | 2.30 | 0.28 | 0.12 | 2.52 |
| MT3 | 0.0009 | 0.90 | 3.17 | 0.85 | 0.50 | 2.87 |
| MV4-11 | 0.0011 | 0.18 | 0.79 | 3.81 | 0.07 | 3.35 |
| NCI-H23 | 0.0011 | 1.41 | 4.86 | 5.22 | 0.48 | 3.33 |
| NCIH292 | 0.0044 | >50 | >50 | 2.81 | 5.31 | >50 |
| NCIH358M | 0.0035 | 1.34 | 9.06 | 0.21 | 0.30 | 2.68 |
| NCIH460 | 0.005 | 0.60 | 2.29 | 1.33 | 0.62 | 5.43 |
| NCIH82 | 0.0076 | 4.60 | >50 | 5.51 | 0.61 | 3.40 |
| OVCAR3 | 0.0031 | 5.89 | >50 | 2.39 | 0.14 | 1.92 |
| OVCAR4 | 0.0049 | 2.79 | >50 | >50 | 0.74 | 4.25 |
| PANC1 | 0.0097 | 4.99 | >50 | >50 | >50 | >50 |
| PANC1005 | 0.0079 | >50 | >50 | 2.97 | 0.24 | 2.98 |
| PBMC | 12.3 | 4.98 | >50 | >50 | 1.99 | >50 |
| PC3 | 0.0026 | 8.20 | >50 | 1.66 | 1.09 | 3.83 |
| PLCPRF5 | 0.0092 | 1.72 | 3.79 | 10.55 | 2.16 | >50 |
| RAMOS | 0.0006 | 3.14 | >50 | 3.63 | 0.56 | 3.68 |
| RD | 0.0023 | 3.49 | >50 | >50 | 2.32 | 4.61 |
| RDES | 0.0037 | 1.29 | 3.48 | 1.72 | 0.37 | 3.07 |
| SAOS2 | 0.0005 | 1.14 | 6.60 | 1.14 | 0.27 | 4.32 |
| SF268 | 0.0041 | 1.07 | 3.94 | >50 | >50 | >50 |
| SF295 | 0.0033 | 1.20 | 3.65 | >50 | 1.94 | >50 |
| SKBR3 | 0.0006 | 0.04 | 0.22 | 4.37 | 0.10 | 1.09 |
| SKHEP1 | 0.0047 | 1.36 | >50 | 4.61 | 4.78 | 5.13 |
| SKLMS1 | 0.0041 | 1.14 | 3.55 | 11.43 | 0.60 | 4.34 |
| SKMEL28 | 0.0043 | >50 | >50 | >50 | 1.18 | 3.69 |
| SKMELS | 0.0032 | 5.10 | >50 | >50 | >50 | 4.92 |
| SKNAS | 0.0036 | >50 | >50 | 2.50 | 0.78 | 2.78 |
| SKNSH | 0.0033 | >50 | >50 | 1.45 | 0.64 | 2.65 |
| SKOV3 | 0.0237 | 0.81 | 3.18 | 5.08 | 1.03 | 3.01 |
| SNB75 | 0.0053 | >50 | >50 | >50 | 2.60 | 9.06 |
| SU-DHL-10 | 0.0041 | 4.95 | >50 | 1.61 | 0.46 | 4.84 |
| SU-DHL-6 | 0.0068 | 5.49 | >50 | 3.73 | 0.36 | 3.29 |
| SW620 | 0.003 | 0.39 | 1.71 | 2.52 | 0.57 | 3.44 |
| T24 | 0.0014 | 2.84 | 5.25 | 2.40 | 0.92 | 3.90 |
| TE671 | 0.0083 | 5.92 | >50 | >50 | 3.14 | >50 |
| THP-1 | 0.0011 | 1.69 | 5.57 | 0.51 | 0.11 | >50 |
| U2OS | 0.0057 | >50 | >50 | >50 | 1.12 | 2.64 |
| U87MG | 0.0015 | >50 | >50 | 1.91 | 5.62 | >50 |
| UMUC3 | 0.0043 | 2.45 | 7.75 | >50 | >50 | >50 |
| UO31 | 0.0037 | >50 | >50 | 1.33 | 0.30 | 2.15 |
| WSU-NHL | 0.0045 | 0.61 | 5.66 | 2.78 | 0.56 | 3.41 |

TABLE 9C

| Cpd. No. | | 123 | 129 | 140 | 173 | 150 |
|---|---|---|---|---|---|---|
| Cell Line | Cancer Type | IC₅₀ (μM) | | | | |
| 5637 | bladder | >50 | 0.56 | 0.66 | 0.22 | 4.05 |
| 22RV1 | prostate | 1.06 | 1.08 | 1.24 | 0.22 | >50 |
| 786O | kidney | >50 | 0.30 | 0.40 | 0.11 | 2.06 |
| A204 | muscle | 2.55 | 0.22 | 0.27 | 0.07 | 3.33 |
| A2780 | ovary | >50 | 0.74 | 0.77 | 0.20 | 4.62 |
| A375 | skin | >50 | 0.20 | 0.21 | 0.14 | 2.32 |
| A431 | skin | 2.60 | 0.33 | 0.29 | 0.07 | 2.32 |
| AS49 | lung | >50 | 3.01 | 4.06 | 0.24 | >50 |
| A673 | bone | 5.25 | 1.44 | 1.95 | 0.57 | >50 |
| ACHN | kidney | 4.02 | 2.54 | 2.85 | 0.28 | >50 |
| ASPC1 | pancreas | >50 | 0.22 | 0.23 | 0.05 | 2.30 |
| BT20 | breast | >50 | 0.34 | 0.39 | 0.10 | 2.03 |
| BXPC3 | pancreas | 6.34 | 2.53 | 3.38 | 0.21 | >50 |
| C33A | endometrial | 1.75 | 0.63 | 0.65 | 0.19 | 4.21 |
| CACO2 | colon | >50 | 0.61 | 0.66 | 0.28 | 3.60 |
| CAKI1 | kidney | >50 | 0.02 | 0.03 | 0.01 | 0.32 |
| CALU6 | lung | >50 | 0.30 | 0.30 | 0.06 | 4.12 |
| CASKI | endometrial | >50 | 0.16 | 0.20 | 0.06 | 1.69 |
| CLS439 | bladder | >50 | 3.26 | 9.83 | 0.61 | >50 |
| COLO205 | colon | >50 | 0.76 | 0.81 | 0.26 | >50 |
| COLO678 | colon | >50 | 1.99 | 4.15 | 0.39 | >50 |
| DLD1 | colon | 4.59 | 2.03 | 2.49 | 0.26 | >50 |
| DU145 | prostate | 5.46 | 0.12 | 0.16 | 0.02 | 1.47 |
| EFO21 | ovary | >50 | 0.33 | 0.54 | 0.13 | 3.32 |
| EJ28 | bladder | >50 | 0.22 | 0.44 | 0.09 | 3.49 |
| GRANTA- | hema- | 3.15 | 2.60 | 3.46 | 0.56 | 4.01 |
| 519 | tological | | | | | |
| HCT116 | colon | >50 | 0.48 | 0.62 | 0.23 | 2.89 |
| HCT15 | colon | >50 | 1.46 | 2.07 | 0.30 | >50 |
| HEK293 | kidney | >50 | 0.38 | 0.46 | 0.14 | 2.46 |
| HELA | endometrial | >50 | 0.44 | 0.49 | 0.11 | 4.53 |
| HEPG2 | liver | 5.08 | 0.30 | 0.45 | 0.11 | 2.96 |
| HL-60 | hema- | >50 | 0.33 | 0.67 | 0.07 | 3.85 |
| | tological | | | | | |
| HS578T | breast | >50 | 0.55 | 0.47 | 0.09 | 6.12 |
| HS729 | muscle | >50 | 0.87 | 0.86 | 0.21 | 12.31 |
| HT1080 | connective | >50 | 3.63 | >50 | 0.72 | >50 |
| | tissue | | | | | |
| HT29 | colon | >50 | 0.22 | 0.30 | 0.16 | 1.46 |
| IGROV1 | ovary | >50 | 0.20 | 0.34 | 0.05 | 2.03 |
| IMR90 | lung | >50 | 0.41 | 0.45 | 0.15 | 4.24 |
| J82 | bladder | 4.88 | 0.47 | 0.38 | 0.15 | 4.64 |
| JAR | placenta | >50 | 0.32 | 0.33 | 0.05 | 1.74 |
| JEG3 | placenta | >50 | 0.29 | 0.38 | 0.04 | 2.82 |
| JIMT1 | breast | >50 | 0.08 | 0.09 | 0.02 | 1.23 |
| K-562 | hema- | >50 | 5.37 | 3.95 | 0.50 | >50 |
| | tological | | | | | |
| KASUMI-1 | hema- | >50 | 0.06 | 0.08 | 0.02 | 2.16 |
| | tological | | | | | |
| L-363 | hema- | >50 | 0.29 | 0.42 | 0.04 | 3.36 |
| | tological | | | | | |
| LOVO | colon | 3.55 | 0.14 | 0.19 | 0.04 | 1.61 |
| MCF7 | breast | >50 | 9.70 | >50 | 2.40 | >50 |
| MDAMB231 | breast | >50 | 2.85 | 7.32 | 0.57 | >50 |
| MDAMB435 | skin | >50 | 1.40 | 1.69 | 0.25 | >50 |
| MDAMB436 | breast | 5.24 | 0.19 | 0.25 | 0.07 | 3.22 |
| MDAMB468 | breast | >50 | 0.27 | 0.24 | 0.10 | 4.45 |
| MG63 | bone | >50 | >50 | >50 | 2.36 | >50 |
| MHHES1 | bone | 3.07 | 0.40 | 0.50 | 0.12 | 2.91 |
| MIAPACA2 | pancreas | >50 | >50 | >50 | 2.77 | >50 |
| MINO | hema- | 3.91 | 0.15 | 0.18 | 0.03 | 1.08 |
| | tological | | | | | |
| MT3 | breast | 3.77 | 0.28 | 0.36 | 0.10 | 3.90 |
| MV4-11 | hema- | 0.38 | 0.05 | 0.16 | 0.03 | 0.63 |
| | tological | | | | | |
| NCI-H23 | lung | 4.05 | 0.41 | 0.64 | 0.07 | 3.29 |
| NCIH292 | lung | >50 | 4.17 | 2.97 | 0.47 | >50 |
| NCIH358M | lung | >50 | 0.27 | 0.43 | 0.15 | 2.63 |
| NCIH460 | lung | 2.80 | 0.50 | 0.60 | 0.15 | 3.37 |
| NCIH82 | lung | 5.25 | 0.39 | 0.49 | 0.07 | 3.77 |
| OVCAR3 | ovary | >50 | 0.17 | 0.18 | 0.08 | 2.05 |
| OVCAR4 | ovary | >50 | 0.56 | 0.60 | 0.13 | 3.52 |
| PANC1 | pancreas | >50 | >50 | >50 | 2.82 | >50 |
| PANC1005 | pancreas | >50 | 0.23 | 0.37 | 0.13 | 1.41 |
| PBMC | hema- | >50 | >50 | >50 | >50 | >50 |
| | tological | | | | | |
| PC3 | prostate | >50 | 0.47 | 0.66 | 0.13 | 5.77 |
| PLCPRF5 | liver | 5.05 | 2.02 | 4.95 | 0.63 | >50 |
| RAMOS | hema- | >50 | 0.52 | 0.60 | 0.08 | 2.44 |
| | tological | | | | | |
| RD | muscle | >50 | 0.64 | 0.87 | 0.19 | 6.72 |
| RDES | bone | 3.65 | 0.25 | 0.27 | 0.06 | 4.48 |
| SAOS2 | bone | >50 | 0.30 | 0.36 | 0.10 | 3.56 |
| SF268 | brain | 14.59 | 5.03 | >50 | 0.67 | >50 |
| SF295 | brain | >50 | 1.65 | 1.73 | 0.44 | >50 |
| SKBR3 | breast | 3.44 | 0.05 | 0.07 | 0.01 | 1.33 |
| SKHEP1 | liver | 3.15 | 2.41 | 3.41 | 0.19 | >50 |
| SKLMS1 | uterus | 2.88 | 0.30 | 0.34 | 0.09 | 2.29 |
| SKMEL28 | skin | >50 | 0.68 | 0.67 | 0.15 | 9.02 |
| SKMEL5 | skin | >50 | 3.17 | 4.01 | 0.42 | >50 |
| SKNAS | brain | >50 | 0.37 | 0.63 | 0.16 | 6.83 |
| SKNSH | brain | >50 | 0.26 | 0.32 | 0.14 | 2.91 |
| SKOV3 | ovary | >50 | 0.46 | 0.62 | 0.10 | 3.62 |
| SNB75 | brain | 4.52 | 1.38 | 1.51 | 0.24 | 14.87 |
| SU-DHL-10 | hema- | >50 | 0.46 | 0.53 | 0.20 | 2.62 |
| | tological | | | | | |
| SU-DHL-6 | hema- | 3.92 | 0.24 | 0.36 | 0.05 | 2.10 |
| | tological | | | | | |
| SW620 | colon | >50 | 0.36 | 0.54 | 0.09 | 3.12 |
| T24 | bladder | 3.01 | 0.60 | 0.71 | 0.14 | 6.26 |
| TE671 | muscle | >50 | 2.44 | 1.92 | 0.62 | 12.60 |
| THP-1 | hema- | 2.49 | 2.27 | >50 | 0.98 | 5.50 |
| | tological | | | | | |
| U2OS | bone | >50 | 0.51 | 0.61 | 0.08 | >50 |
| U87MG | brain | 5.23 | 3.13 | 3.66 | 0.38 | >50 |
| UMUC3 | bladder | >50 | 3.04 | 3.40 | 0.46 | >50 |
| UO31 | kidney | >50 | 0.24 | 0.28 | 0.05 | 4.54 |
| WSU-NHL | hema- | 4.26 | 0.46 | 0.43 | 0.04 | 4.87 |
| | tological | | | | | |

TABLE 9D

| | Cpd. No. | 190 | 193 | 195 | 197 | 198 |
|---|---|---|---|---|---|---|
| Cell Line | Cancer Type | IC₅₀ (μM) | | | | |
| 5637 | bladder | 3.87 | >50 | 0.23 | 0.004 | 0.45 |
| 22RV1 | prostate | >50 | 5.77 | 0.54 | 0.084 | 1.33 |
| 786O | kidney | >50 | 9.53 | 0.11 | >50 | 1.07 |
| A204 | muscle | 0.17 | 0.06 | 0.14 | 0.716 | 0.02 |
| A2780 | ovary | 5.39 | 3.47 | 0.39 | 0.213 | 0.72 |
| A375 | skin | >50 | 3.61 | 0.11 | 0.183 | 0.71 |
| A431 | skin | 3.80 | 2.52 | 0.07 | 0.002 | 0.47 |
| A549 | lung | 2.90 | 2.32 | 0.06 | 0.075 | 0.44 |
| A673 | bone | >50 | 3.26 | 0.70 | 0.110 | 1.74 |
| ACHN | kidney | 2.85 | 2.82 | 0.09 | 0.012 | 0.39 |
| ASPC1 | pancreas | >50 | >50 | 0.13 | 0.011 | 0.58 |
| BT20 | breast | >50 | 3.06 | 0.11 | >50 | 0.58 |
| BXPC3 | pancreas | >50 | 4.27 | 0.08 | 0.0004 | 0.54 |
| C33A | endometrial | 2.08 | 1.46 | 0.38 | 0.047 | 0.24 |
| CACO2 | colon | >50 | >50 | 0.28 | >50 | >50 |
| CAKI1 | kidney | 1.07 | 1.58 | 0.02 | >50 | 0.11 |
| CALU6 | lung | 5.25 | 3.82 | 0.04 | 0.076 | 0.72 |
| CASKI | endometrial | >50 | 3.06 | 0.05 | >50 | 3.96 |
| CLS439 | bladder | >50 | >50 | 0.40 | >50 | 2.27 |
| COLO205 | colon | 3.03 | 1.57 | 0.33 | 0.018 | 0.21 |
| COLO678 | colon | >50 | >50 | 0.10 | >50 | 3.76 |
| DLD1 | colon | >50 | 4.02 | 0.51 | 8.968 | 2.65 |
| DU145 | prostate | 3.18 | 1.40 | 0.03 | 0.425 | 0.31 |
| EFO21 | ovary | >50 | 4.59 | 0.15 | >50 | 0.79 |
| EJ28 | bladder | >50 | 4.43 | 0.14 | 0.066 | 1.53 |
| GRANTA- | hematological | 1.24 | 0.89 | 0.16 | 0.010 | 0.07 |
| 519 | | | | | | |
| HCT116 | colon | 3.28 | 2.79 | 0.25 | 1.113 | 0.47 |
| HCT15 | colon | 3.10 | 2.14 | 0.29 | 1.378 | 0.46 |
| HEK293 | kidney | >50 | >50 | 0.20 | >50 | >50 |
| HELA | endometrial | >50 | >50 | 0.08 | >50 | 1.00 |
| HEPG2 | liver | >50 | 3.57 | 0.17 | 0.089 | 1.00 |
| HL-60 | hematological | >50 | 1.63 | 0.10 | 0.247 | 1.01 |
| HS578T | breast | >50 | >50 | 0.06 | >50 | 0.97 |
| HS729 | muscle | 4.74 | 5.86 | 0.15 | >50 | 0.58 |
| HT1080 | connective | 4.73 | 2.78 | 0.27 | 0.046 | 0.38 |
| | tissue | | | | | |
| HT29 | colon | >50 | >50 | 0.26 | 0.060 | 1.27 |
| IGROV1 | ovary | 4.31 | 2.14 | 0.08 | 1.586 | 0.51 |
| IMR90 | lung | >50 | 4.16 | 0.06 | >50 | 1.58 |
| J82 | bladder | >50 | 5.54 | 0.16 | 0.216 | 0.76 |
| JAR | placenta | >50 | >50 | 0.10 | >50 | >50 |
| JEG3 | placenta | >50 | >50 | 0.10 | 0.037 | 3.34 |
| JIMT1 | breast | 2.23 | 2.82 | 0.03 | >50 | 0.19 |
| K-562 | hematological | >50 | >50 | 0.53 | >50 | 4.45 |
| KASUMI-1 | hematological | 2.23 | 0.37 | 0.02 | 0.076 | 0.27 |
| L-363 | hematological | >50 | >50 | 0.09 | >50 | >50 |
| LOVO | colon | 2.74 | 2.33 | 0.03 | 0.006 | 0.29 |
| MCF7 | breast | >50 | >50 | 0.39 | 0.181 | 3.17 |
| MDAMB231 | breast | 2.23 | 1.45 | 0.24 | >50 | 0.10 |
| MDAMB435 | skin | 4.70 | 4.74 | 0.32 | 0.924 | 1.30 |
| MDAMB436 | breast | >50 | >50 | 0.08 | 0.041 | 0.95 |
| MDAMB468 | breast | >50 | >50 | 0.07 | >50 | 3.11 |
| MG63 | bone | >50 | >50 | 1.73 | 0.361 | 0.48 |
| MHHES1 | bone | >50 | 2.26 | 0.17 | 2.565 | 1.66 |
| MIAPACA2 | pancreas | >50 | >50 | 1.75 | 0.904 | 1.15 |
| MINO | hematological | 0.43 | 0.25 | 0.02 | 0.002 | 0.06 |
| MT3 | breast | 1.08 | 0.58 | 0.12 | 0.016 | 0.08 |
| MV4-11 | hematological | 3.80 | 4.77 | 0.02 | 0.044 | 0.84 |
| NCI-H23 | lung | >50 | 7.88 | 0.22 | 0.334 | 0.72 |
| NCIH292 | lung | >50 | 5.76 | 0.15 | 0.114 | 0.92 |
| NCIH358M | lung | 1.04 | 2.52 | 0.14 | 0.019 | 0.12 |
| NCIH460 | lung | 5.68 | 2.83 | 0.17 | 0.061 | 0.77 |
| NCIH82 | lung | >50 | 3.37 | 0.17 | 0.087 | 1.15 |
| OVCAR3 | ovary | 2.57 | 2.75 | 0.08 | >50 | 0.64 |
| OVCAR4 | ovary | >50 | >50 | 0.15 | >50 | 8.05 |
| PANCI | pancreas | >50 | 3.60 | 2.25 | >50 | 0.62 |
| PANC1005 | pancreas | >50 | >50 | 0.18 | >50 | >50 |
| PBMC | hematological | 2.61 | >50 | 0.02 | 3.020 | 0.71 |
| PC3 | prostate | 1.46 | 2.04 | 0.22 | >50 | 0.18 |
| PLCPRF5 | liver | >50 | >50 | 1.04 | >50 | >50 |
| RAMOS | hematological | 2.69 | 3.01 | 0.14 | 0.292 | 0.24 |
| RD | muscle | >50 | >50 | 0.21 | 0.066 | 1.03 |
| RDES | bone | >50 | >50 | 0.04 | 10.398 | 1.90 |
| SAOS2 | bone | 1.49 | 1.12 | 0.10 | 0.006 | 0.11 |
| SF268 | brain | 3.80 | 2.77 | 0.30 | 0.187 | 0.26 |
| SF295 | brain | >50 | >50 | 0.22 | >50 | 3.25 |
| SKBR3 | breast | 4.56 | 3.50 | 0.03 | 0.005 | 0.26 |
| SKHEP1 | liver | 2.54 | 2.90 | 0.06 | 1.321 | 0.32 |
| SKLMS1 | uterus | >50 | >50 | 0.14 | 0.298 | 0.86 |
| SKMEL28 | skin | >50 | >50 | 0.10 | >50 | 1.32 |
| SKMEL5 | skin | >50 | >50 | 0.09 | >50 | 6.18 |
| SKNAS | brain | >50 | 6.05 | 0.12 | >50 | 1.22 |
| SKNSH | brain | >50 | 4.78 | 0.08 | >50 | >50 |
| SKOV3 | ovary | >50 | >50 | 0.09 | 0.071 | 1.46 |
| SNB75 | brain | >50 | 4.46 | 0.22 | >50 | 0.55 |
| SU-DHL-10 | hematological | 2.73 | 3.24 | 0.21 | 1.155 | 0.56 |
| SU-DHL-6 | hematological | 2.87 | 2.94 | 0.06 | 0.143 | 0.31 |
| SW620 | colon | 4.60 | >50 | 0.08 | 0.082 | 1.14 |
| T24 | bladder | 3.21 | 2.32 | 0.30 | 0.098 | 0.29 |
| TE671 | muscle | >50 | 4.85 | 0.73 | 0.082 | 1.02 |
| THP-1 | hematological | 3.44 | 2.96 | 0.20 | 0.131 | 0.65 |
| U20S | bone | >50 | >50 | 0.07 | >50 | 2.45 |
| U87MG | brain | 2.91 | 1.06 | 0.15 | >50 | 0.40 |
| UMUC3 | bladder | >50 | 4.20 | 0.19 | 0.228 | 1.21 |
| UO31 | kidney | 8.13 | 3.89 | 0.05 | >50 | 0.55 |
| WSU-NHL | hematological | 1.67 | 0.49 | 0.04 | 0.448 | 0.15 |

TABLE 9E

| Cpd. No. | 178 | 169 | 170 | 171 | 172 | 184 |
|---|---|---|---|---|---|---|
| Cell Line | IC₅₀ (μM) | | | | | |
| 5637 | 0.16 | 0.001 | 2.40 | 0.95 | 0.06 | 0.19 |
| 22RV1 | 0.25 | 0.002 | 79.52 | 2.89 | 0.50 | 0.22 |
| 786O | 0.07 | 0.017 | 29.63 | 1.57 | 0.13 | 0.05 |
| A204 | 0.03 | 0.051 | 0.53 | 0.19 | 0.01 | 0.02 |
| A2780 | 0.15 | 0.008 | 7.16 | 1.67 | 0.02 | 0.15 |
| A375 | 0.04 | 0.007 | 9.75 | 0.57 | 0.05 | 0.04 |
| A431 | 0.05 | 0.0004 | 11.10 | 1.24 | 0.07 | 0.04 |
| A549 | 0.37 | 0.014 | 8.86 | 1.33 | 0.01 | 0.04 |
| A673 | 0.36 | 0.003 | 10.85 | 2.53 | 0.02 | 0.27 |
| ACHN | 0.46 | 0.003 | 12.67 | 1.05 | 0.05 | 0.02 |
| ASPC1 | 0.03 | 0.008 | 7.85 | 0.32 | 0.03 | 0.03 |
| BT20 | 0.07 | >50 | 38.19 | 2.12 | 0.21 | 0.03 |
| BXPC3 | 0.52 | 0.001 | 6.87 | 0.43 | 0.03 | 0.08 |
| C33A | 0.06 | 0.009 | 9.56 | 0.41 | 0.05 | 0.05 |
| CACO2 | 0.16 | >50 | 49.22 | 3.52 | 0.39 | 0.19 |
| CAKI1 | 0.01 | 0.045 | 2.46 | 0.13 | 0.05 | 0.01 |
| CALU6 | 0.03 | 0.029 | 17.30 | 0.40 | 0.10 | 0.03 |
| CASKI | 0.04 | 0.023 | 106.60 | 0.99 | 0.38 | 0.03 |
| CLS439 | 0.77 | 0.023 | 45.50 | 2.64 | 0.18 | 0.06 |
| COLO205 | 0.17 | 0.001 | 2.80 | 0.42 | 0.01 | 0.12 |
| COLO678 | 0.65 | >50 | 21.79 | 0.63 | 0.13 | 0.06 |
| DLD1 | 0.35 | 0.016 | 11.42 | 1.03 | 0.10 | 0.09 |
| DU145 | 0.02 | 0.010 | 5.44 | 0.34 | 0.06 | 0.02 |
| EFO21 | 0.08 | 0.476 | 7.34 | 0.46 | 0.12 | 0.07 |
| EJ28 | 0.06 | 0.004 | 10.41 | 1.06 | 0.27 | 0.03 |
| GRANTA-519 | 2.84 | 0.009 | 1.66 | 0.68 | 0.01 | 0.11 |
| HCT116 | 0.16 | 0.023 | 5.26 | 1.03 | 0.02 | 0.14 |
| HCT15 | 0.14 | 0.015 | 12.42 | 2.01 | 0.08 | 0.04 |
| HEK293 | 0.07 | 0.030 | 35.21 | 3.61 | 0.26 | 0.05 |
| HELA | 0.07 | 1.532 | 6.72 | 0.31 | 0.19 | 0.07 |
| HEPG2 | 0.06 | 0.002 | 15.33 | 2.75 | 0.08 | 0.04 |
| HL-60 | 0.04 | 0.008 | 2.05 | 0.23 | 0.03 | 0.04 |
| HS578T | 0.07 | >50 | 36.63 | 1.64 | 0.18 | 0.08 |
| HS729 | 0.12 | 0.015 | 3.52 | 0.44 | 0.17 | 0.10 |
| HT1080 | 2.09 | 0.008 | 4.09 | 2.79 | 0.03 | 0.07 |
| HT29 | 0.05 | 0.002 | 18.41 | 0.70 | 0.06 | 0.07 |
| IGROV1 | 0.03 | 0.022 | 13.60 | 1.28 | 0.08 | 0.05 |
| IMR90 | 0.05 | >50 | 11.75 | 0.54 | 0.14 | 0.03 |
| J82 | 0.08 | 0.005 | 19.36 | 1.81 | 0.46 | 0.10 |
| JAR | 0.03 | 0.018 | 45.90 | 1.60 | 0.08 | 0.04 |
| JEG3 | 0.04 | 0.002 | 56.79 | 2.57 | 0.06 | 0.04 |
| JIMT1 | 0.02 | 0.034 | 1.66 | 0.18 | 0.05 | 0.02 |
| K-562 | 1.17 | 0.094 | 41.82 | 5.68 | 0.17 | 0.12 |
| KASUMI-1 | 0.02 | 0.016 | 0.55 | 0.06 | 0.01 | 0.01 |
| L-363 | 0.02 | 0.003 | 33.05 | 0.92 | 0.22 | 0.02 |
| LOVO | 0.02 | 0.002 | 9.73 | 0.44 | 0.04 | 0.02 |
| MCF7 | 2.55 | 0.002 | 27.20 | 3.06 | 0.14 | 0.10 |
| MDAMB231 | 1.06 | 0.381 | 2.88 | 0.16 | 0.04 | 0.04 |
| MDAMB435 | 0.19 | 0.017 | 14.18 | 2.47 | 0.19 | 0.05 |
| MDAMB436 | 0.05 | 0.003 | 3.35 | 0.62 | 0.05 | 0.04 |
| MDAMB468 | 0.09 | 0.018 | 23.62 | 1.44 | 0.13 | 0.08 |
| MG63 | 3.61 | 0.010 | 15.52 | 1.63 | 0.26 | 0.39 |
| MHHES1 | 0.10 | 0.005 | 5.05 | 0.46 | 0.01 | 0.07 |
| MIAPACA2 | 7.25 | 0.016 | 35.39 | 2.95 | 0.18 | 0.13 |
| MINO | 0.02 | 0.001 | 0.61 | 0.04 | 0.01 | 0.01 |
| MT3 | 0.04 | 0.004 | 2.00 | 0.23 | 0.02 | 0.09 |
| MV4-11 | 0.02 | 0.001 | 5.77 | 0.27 | 0.19 | 0.02 |
| NCI-H23 | 0.06 | 0.008 | 8.19 | 2.04 | 0.06 | 0.04 |
| NCIH292 | 0.83 | 0.019 | 8.01 | 2.33 | 0.03 | 0.09 |
| NCIH358M | 0.07 | 0.006 | 0.29 | 0.06 | 0.02 | 0.07 |
| NCIH460 | 0.04 | 0.002 | 32.53 | 1.95 | 0.03 | 0.04 |
| NCIH82 | 0.07 | 0.006 | 21.26 | 1.60 | 0.06 | 0.06 |
| OVCAR3 | 0.03 | 0.015 | 1.13 | 0.35 | 0.02 | 0.04 |
| OVCAR4 | 0.08 | 0.538 | 16.32 | 1.86 | 0.29 | 0.05 |
| PANCI | 14.91 | 0.028 | 16.55 | 5.06 | 0.06 | 0.13 |
| PANC1005 | 0.06 | 0.553 | 5.70 | 0.66 | 0.09 | 0.05 |
| PBMC | 0.32 | >50 | 0.29 | 0.15 | 7.45 | 0.18 |
| PC3 | 0.07 | 0.014 | 2.54 | 0.28 | 0.09 | 0.04 |
| PLCPRF5 | 0.45 | 0.019 | 20.08 | 2.84 | 0.21 | 0.23 |
| RAMOS | 0.13 | 0.018 | 5.10 | 0.42 | 0.01 | 0.14 |
| RD | 0.07 | 0.010 | 11.94 | 0.96 | 0.07 | 0.06 |
| RDES | 0.03 | 0.007 | 35.34 | 0.82 | 0.15 | 0.02 |
| SAOS2 | 0.05 | 0.003 | 0.45 | 0.11 | 0.01 | 0.05 |
| SF268 | 1.88 | 0.007 | 16.33 | 3.02 | 0.16 | 0.16 |
| SF295 | 0.23 | 0.007 | 86.50 | 3.58 | 0.26 | 0.16 |
| SKBR3 | 0.01 | 0.000 | 1.58 | 0.14 | 0.01 | 0.01 |
| SKHEP1 | 0.45 | 0.016 | 5.36 | 0.28 | 0.08 | 0.02 |
| SKLMS1 | 0.04 | 0.011 | 44.29 | 3.02 | 0.64 | 0.04 |
| SKMEL28 | 0.10 | 0.112 | 11.14 | 0.98 | 0.05 | 0.10 |
| SKMELS | 0.58 | 0.029 | 15.93 | 1.97 | 0.38 | 0.05 |
| SKNAS | 0.07 | >50 | 29.49 | 2.55 | 0.08 | 0.03 |
| SKNSH | 0.03 | 0.151 | 22.19 | 2.26 | 0.02 | 0.03 |
| SKOV3 | 0.06 | 0.028 | 4.22 | 0.51 | 0.13 | 0.05 |
| SNB75 | 0.16 | >50 | 50.59 | 1.47 | 0.87 | 0.10 |
| SU-DHL-10 | 0.44 | 0.226 | 5.61 | 0.73 | 0.18 | 0.18 |
| SU-DHL-6 | 0.03 | 0.007 | 38.63 | 0.98 | 0.04 | 0.01 |
| SW620 | 0.05 | 0.002 | 17.39 | 0.68 | 0.10 | 0.03 |
| T24 | 0.09 | 0.005 | 5.89 | 0.56 | 0.04 | 0.06 |
| TE671 | 0.37 | 0.008 | 11.12 | 0.78 | 0.09 | 0.15 |
| THP-1 | 1.77 | 0.002 | 5.86 | 0.13 | 0.22 | 0.02 |
| U2OS | 0.06 | >50 | 13.79 | 0.96 | 0.13 | 0.04 |
| U87MG | 2.01 | >50 | 5.59 | 0.62 | 0.03 | 0.03 |
| UMUC3 | 0.98 | 0.007 | 21.58 | 0.97 | 0.09 | 0.03 |
| UO31 | 0.04 | 0.031 | 7.43 | 0.42 | 0.04 | 0.05 |
| WSU-NHL | 0.03 | 0.001 | 1.91 | 0.34 | 0.02 | 0.03 |

TABLE 10A

| | Cpd. No. | 5 | 185 | 130 | 95 | 107 |
|---|---|---|---|---|---|---|
| Cell Line | Cancer Type | IC₅₀ (μM) | | | | |
| 5637 | bladder | 4.93 | 0.001 | 2.06 | 3.90 | 2.22 |
| A204 | muscle | 2.32 | 0.077 | 1.71 | 1.35 | >50 |
| ACHN | kidney | 3.67 | 0.004 | 3.83 | >50 | 3.84 |
| ASPC1 | pancreas | 2.62 | 0.005 | 0.62 | 4.62 | 1.58 |
| BXPC3 | pancreas | >50 | 0.002 | 3.28 | >50 | 1.00 |
| CACO2 | colon | 7.85 | 0.116 | 1.50 | >50 | >50 |
| GRANTA-519 | hematological | >50 | 0.002 | 5.22 | 4.44 | 1.86 |
| KASUMI-1 | hematological | 1.40 | 0.024 | 0.21 | 1.63 | >50 |
| LOVO | colon | 2.75 | 0.007 | 0.49 | >50 | >50 |
| MDAMB231 | breast | >50 | 0.076 | >50 | 1.69 | >50 |
| MDAMB436 | breast | 2.90 | 0.006 | 0.53 | 3.85 | >50 |
| MINO | hematological | 1.75 | 0.004 | 0.30 | 2.54 | 2.34 |
| NCIH358M | lung | 1.98 | 0.012 | 0.93 | 1.59 | >50 |
| SKBR3 | breast | 1.06 | 0.001 | 0.12 | >50 | 0.97 |

TABLE 10B

| | Cpd. No. | 109 | 115 | 223 |
|---|---|---|---|---|
| Cell Line | Cancer Type | IC₅₀ (μM) | | |
| 5637 | bladder | 0.01 | 7.01 | >50 |
| A204 | muscle | 4.02 | 0.63 | 1.83 |
| ACHN | kidney | 1.26 | 2.81 | >50 |
| ASPC1 | pancreas | 2.93 | >50 | >50 |
| BXPC3 | pancreas | 0.50 | >50 | >50 |
| CACO2 | colon | 3.28 | >50 | >50 |
| GRANTA-519 | hematological | 0.92 | 2.22 | 1.40 |
| KASUMI-1 | hematological | 1.69 | 2.13 | 1.46 |
| LOVO | colon | 2.54 | >50 | >50 |
| MDAMB231 | breast | 0.40 | 3.40 | 6.28 |
| MDAMB436 | breast | 3.49 | 4.09 | >50 |
| MINO | hematological | 0.37 | 1.72 | 2.70 |
| NCIH358M | lung | 0.01 | 1.07 | 2.96 |
| SKBR3 | breast | 0.98 | 3.45 | >50 |

### Example 30

### Metabolism by Human Cytidine Deaminase

A human cytidine deaminase (CDA) assay based on substrate absorbance was used to determine deamination, e.g., the deamination of cytidine to uridine, see, e.g., Frances et al., Molecular Therapy 28:P357-366 (2020). rates for representative Compounds of the Disclosure according to the following protocol.

### Step 1. Enzyme Prep:

In assay buffer (100 mM Tris-HCl, pH 7.5, 100 mM KCl) prepare 20 nM CDA (from Sigma, #SRP6372) from 27.3 μM stock

Prepare 0.39 μM CDA from 27.3 μM stock (this for the generation product for compounds that do not produce a corresponding deaminated product)

### Step 2. Compound Dilution:

Substrate, e.g. a Compound of the Disclosure, serial dilution in DMSO: In a shallow well ProxiPlate conduct 4-fold serial dilution of substrate to make 25 mM and 6.25 mM concentrations.

### Step 3. 2.5× Substrate Intermediate in Assay Buffer:

Transfer 7.2 μl of each of the substrate DMSO stock in step 2 into a 500 μl 96-Well V-bottom Microplate, clear SBS footprint, cat #P-96-450V-C

Add 352.8 μl assay buffer to the wells

For deaminated product compounds mix 4 μl product with 196 μl assay buffer

### Step 4. Assay—UV-Transparent 96-Well Assay Plate:

Enzymatic product generation (for substrates without synthetic product): 2.4 μl of the 2.5× intermediate substrate stocks (from step 3) was mixed with 117.6 μl assay buffer followed by 180 μl of 0.39 μM CDA in a 1.5 ml Eppendorf tube. This was incubated at 37° C. for 1 h. 200 μl of this mixture was transferred into the assay plate to serve as the product control. Substrate+CDA: transfer 80 ul of the 2.5× intermediate substrate stocks (from step 3) into assay plate

Substrate+buffer: mix 80 ul of the 2.5× intermediate substrate stocks (from step 3) with 120 ul assay buffer into substrate control (no enzyme) wells

Product+buffer: mix 80 ul of the 2.5× intermediate product stocks (from step 3) with 120 ul assay buffer into product control (no enzyme) wells

Incubate the plate alongside the 20 nM CDA enzyme intermediate stock at 37° C. to temperature equilibrate reagents

Add 120 μl of the 20 nM enzyme mix into the Substrate+CDA wells and start reading at 290 nm every 30 s for about 60 min

### Final Reaction Conditions:

12 nM CDA

50 and 200 uM substrate and product

In 100 mM Tris, pH 7.5, 100 mM KCl, 0.8% carryover DMSO

Reaction volume: 200 ul

5 min incubation at 37° C.

Absorbance at 290 nm every 30 s at 37° C. for 1 h using FlexStation 3

Step 5: DATA processing:

Convert A290 to concentration of substrate remaining at time t after enzyme addition:

$${A}_{290} ( {S}_{t} - {P}_{o} \} / {A}_{290} ( {S}_{o} - {P}_{o} ) \times [ {S}_{I} ]$$

where:
Sₜ=absorbance reading of: substrate+CDA wells at time t
Sₒ=Average absorbance reading of: substrate+buffer wells
Pₒ=Average absorbance reading of: product+buffer wells
[S]ₜ=initial substrate concentration
Use GraphPad to plot [substrate] against time to calculate initial velocity or slope (μMs⁻¹) i.e. the linear range of each curve
Express rate in s⁻¹ by dividing initial velocity by the total enzyme concentration.

Plot rate against [substrate] and fit data to standard Michaelis Menten equation to obtain k_{cat} and K_{M} parameters.

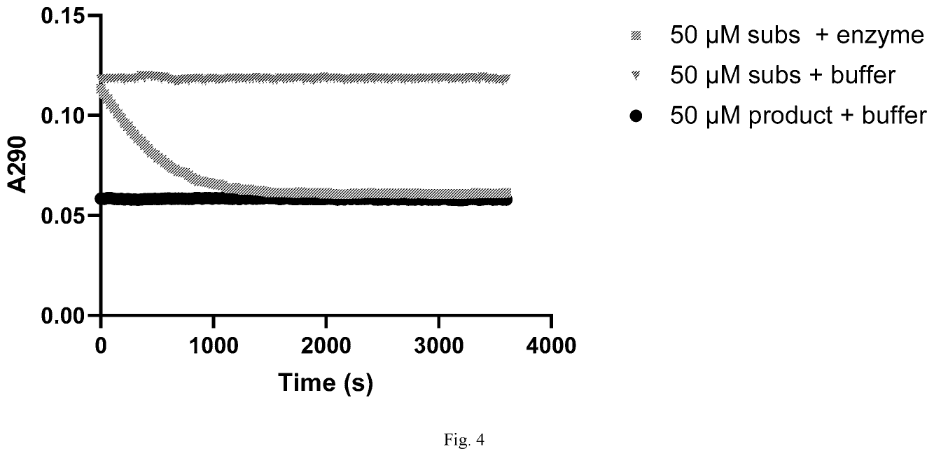

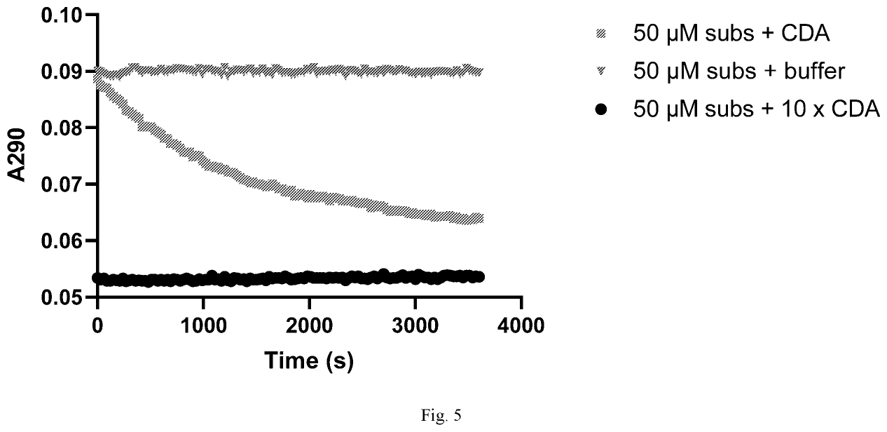

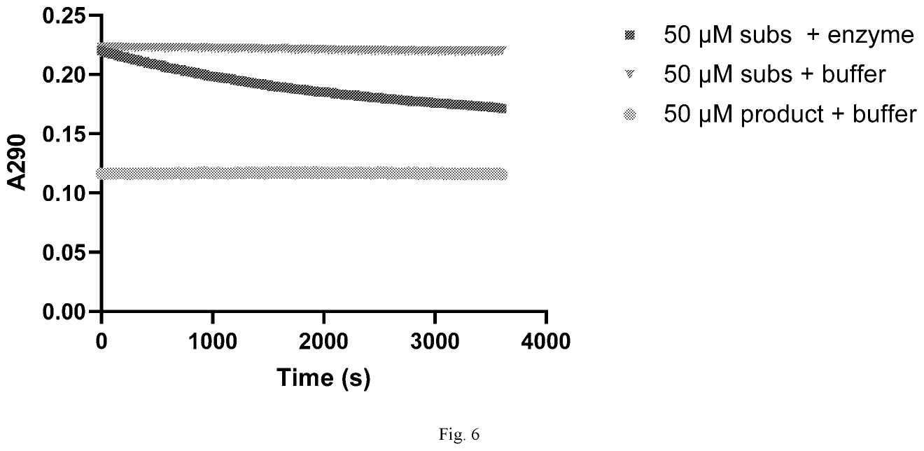

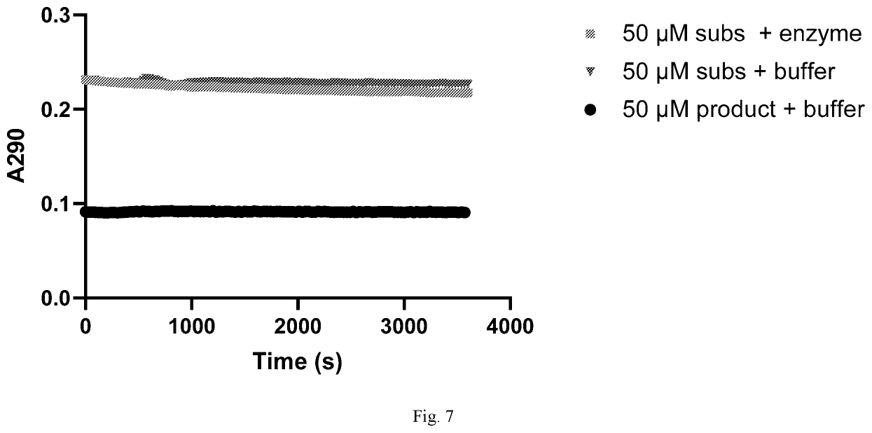

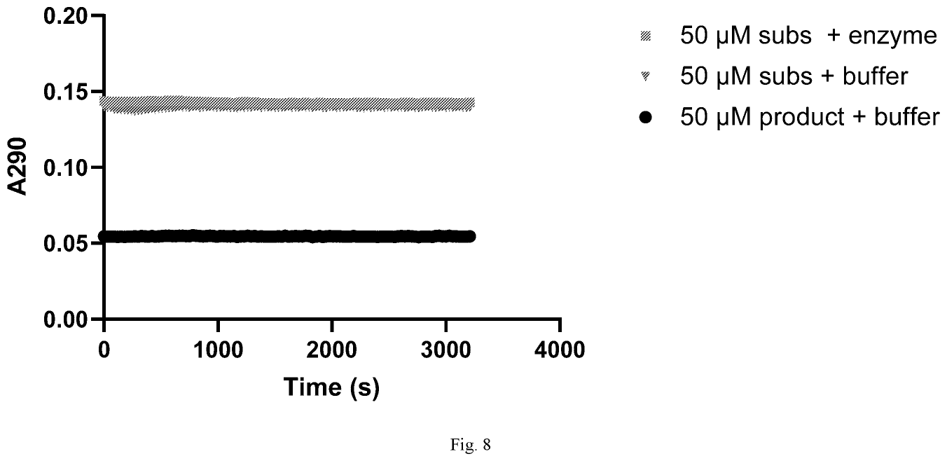

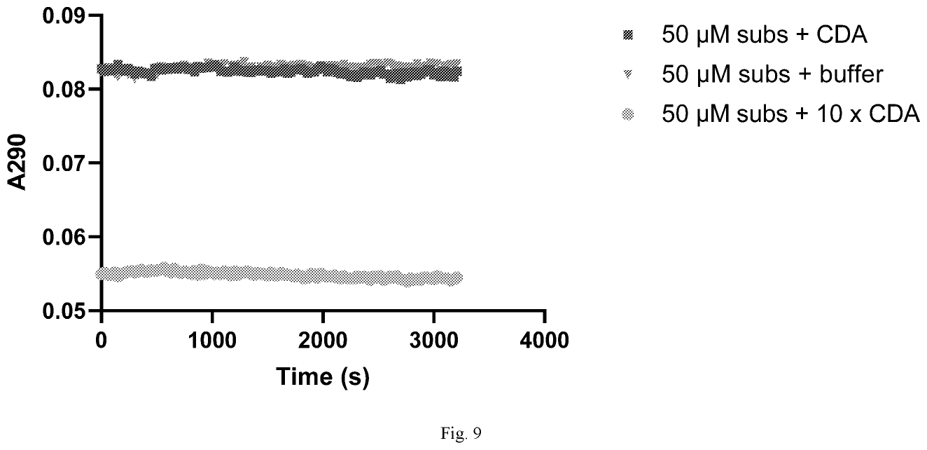

The results of the CDA assay are provided in FIGS. 4-9 and Table 11. These data show that certain Compounds of the Disclosure are surprising more stable than, e.g., cytidine and gemcitabine, to

TABLE 11

| | | | | | | K_{cat}/K_{M} | |
|---|---|---|---|---|---|---|---|
| | | K_{cat} (s⁻¹) | | K_{M} (μM) | | (M⁻¹s⁻¹) × 10⁵ | |
| Substrate | n | Average | STDEV | Average | STDEV | Average | STDEV |
| cytidine | 4 | 15.6 | 0.4 | 69 | 8.4 | 2.3 | 0.29 |
| Gemcitabine | 2 | 26.8 | 8.8 | 520.8 | 232.6 | 0.53 | 0.07 |
| Cpd. No. 4 | 2 | 13.3 | | 1003 | 684 | 0.23 | 0.02 |
| Cpd. No. 6 | 2 | Inactive | | | | | |
| Cpd. No. 190 | 2 | Inactive | | | | | |
| Cpd. No. 193 | 2 | 2.8 | 1.8 | 196 | 152.7 | 0.16 | 0.03 |
| Cpd. No. 197 | 2 | | | Inactive | | | |
| Cpd. No. 198 | 2 | Inactive | | | | | |
| Cpd. No. 212 | 2 | 6.1 | 5.5 | 1057.8 | 614.1 | 0.05 | 0.02 |

Inactive = Rates of substrate conversion were less than 0.003 μMs⁻¹ or 1 s⁻¹ at 200 μM and thus could not be fitted

All patents, patent application, and publications cited herein are fully incorporated by reference herein.

The breadth and scope of the present disclosure should not be limited by any of the above-described exemplary embodiments, but should be defined only in accordance with the following claims and their equivalents.

1. A compound selected from the group consisting of: 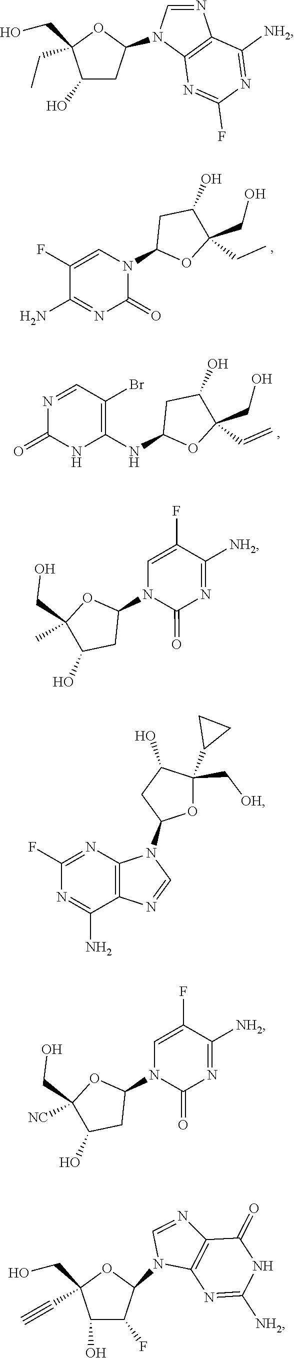 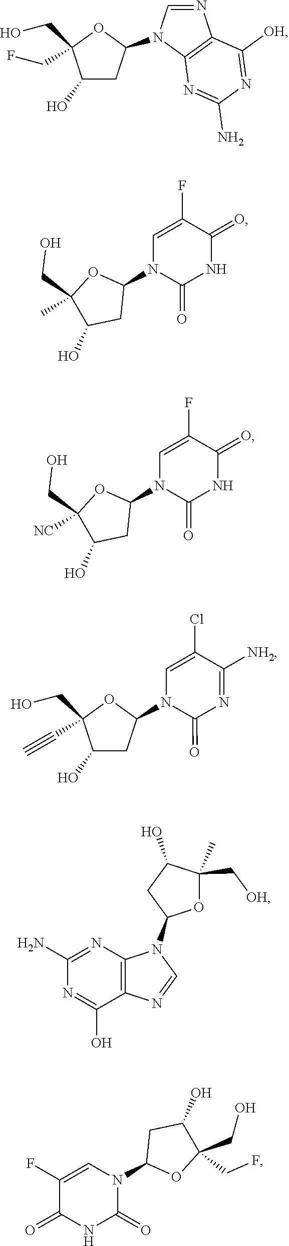 [SMILES: Nc1nc(O)c2ncn([C@H]3C[C@H](O)[C@](CO)(CF)O3)c2n1 ; C[C@]1(CO)O[C@@H](n2cc(F)c(=O)[nH]c2=O)C[C@@H]1O ; N#C[C@]1(CO)O[C@@H](n2cc(F)c(=O)[nH]c2=O)C[C@@H]1O ; C#C[C@]1(CO)O[C@@H](n2cc(Cl)c(N)nc2=O)C[C@@H]1O ; C[C@]1(CO)O[C@@H](n2cnc3c(O)nc(N)nc32)C[C@@H]1O ; O=c1[nH]c(=O)n([C@H]2C[C@H](O)[C@](CO)(CF)O2)cc1F] 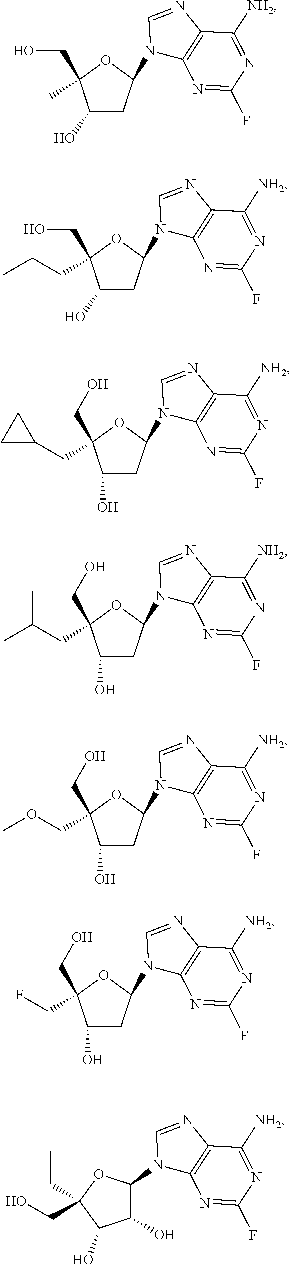 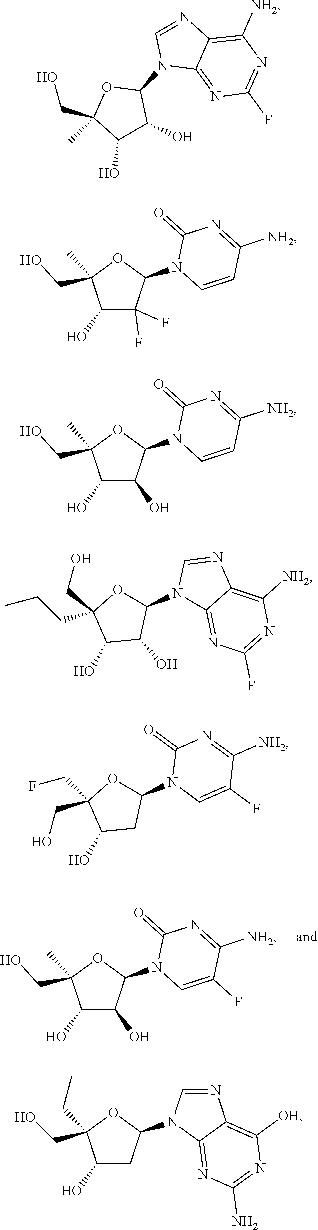
or a pharmaceutically acceptable salt, solvate, and/or tautomer thereof, and/or mono-, di-, or trisphosphates thereof.

2. A pharmaceutical composition comprising the compound of claim 1, or a pharmaceutically acceptable salt, solvate, and/or tautomer thereof, and one or more pharmaceutically acceptable excipients.

3. A method of treating cancer in a subject in need thereof, the method comprising administering a therapeutically effective amount of the compound of claim 1 to the subject, wherein the cancer is adrenal cancer, acinic cell carcinoma, acoustic neuroma, acral lentigious melanoma, acrospiroma, acute eosinophilic leukemia, acute erythroid leukemia, acute lymphoblastic leukemia, acute megakaryoblastic leukemia, acute monocytic leukemia, acute promyelocytic leukemia, adenocarcinoma, adenoid cystic carcinoma, adenoma, adenomatoid odontogenic tumor, adenosquamous carcinoma, adipose tissue neoplasm, adrenocortical carcinoma, adult T-cell leukemia/lymphoma, aggressive NK-cell leukemia, AIDS-related lymphoma, alveolar rhabdomyosarcoma, alveolar soft part sarcoma, ameloblastic fibroma, anaplastic large cell lymphoma, anaplastic thyroid cancer, angioimmunoblastic T-cell lymphoma, angiomyolipoma, angiosarcoma, astrocytoma, atypical teratoid rhabdoid tumor, B-cell chronic lymphocytic leukemia, B-cell prolymphocytic leukemia, B-cell lymphoma, basal cell carcinoma, biliary tract cancer, bladder cancer, blastoma, bone cancer, Brenner tumor, Brown tumor, Burkitt's lymphoma, breast cancer, brain cancer, carcinoma, carcinoma in situ, carcinosarcoma, cartilage tumor, cementoma, myeloid sarcoma, chondroma, chordoma, choriocarcinoma, choroid plexus papilloma, clear-cell sarcoma of the kidney, colon cancer, craniopharyngioma, cutaneous T-cell lymphoma, cervical cancer, colorectal cancer, Degos disease, desmoplastic small round cell tumor, diffuse large B-cell lymphoma, dysembryoplastic neuroepithelial tumor, dysgerminoma, embryonal carcinoma, endocrine gland neoplasm, endodermal sinus tumor, enteropathy-associated T-cell lymphoma, esophageal cancer, fetus in fetu, fibroma, fibrosarcoma, follicular lymphoma, follicular thyroid cancer, ganglioneuroma, gastrointestinal cancer, germ cell tumor, gestational choriocarcinoma, giant cell fibroblastoma, giant cell tumor of the bone, glial tumor, glioblastoma, glioma, gliomatosis cerebri, glucagonoma, gonadoblastoma, granulosa cell tumor, gynandroblastoma, gallbladder cancer, gastric cancer, hairy cell leukemia, hemangioblastoma, head and neck cancer, hemangiopericytoma, hematological malignancy, hepatoblastoma, hepatocellular carcinoma, hepatosplenic T-cell lymphoma, Hodgkin's lymphoma, non-Hodgkin's lymphoma, invasive lobular carcinoma, intestinal cancer, kidney cancer, laryngeal cancer, lentigo maligna, lethal midline carcinoma, leukemia, leydig cell tumor, liposarcoma, lung cancer, lymphangioma, lymphangiosarcoma, lymphoepithelioma, lymphoma, acute lymphocytic leukemia, acute myelogeous leukemia, chronic lymphocytic leukemia, liver cancer, small cell lung cancer, non-small cell lung cancer, MALT lymphoma, malignant fibrous histiocytoma, malignant peripheral nerve sheath tumor, malignant triton tumor, mantle cell lymphoma, marginal zone B-cell lymphoma, mast cell leukemia, mediastinal germ cell tumor, medullary carcinoma of the breast, medullary thyroid cancer, medulloblastoma, melanoma, meningioma, merkel cell cancer, mesothelioma, metastatic urothelial carcinoma, mixed Mullerian tumor, mucinous tumor, multiple myeloma, muscle tissue neoplasm, mycosis fungoides, myxoid liposarcoma, myxoma, myxosarcoma, nasopharyngeal carcinoma, neurinoma, neuroblastoma, neurofibroma, neuroma, nodular melanoma, ocular cancer, oligoastrocytoma, oligodendroglioma, oncocytoma, optic nerve sheath meningioma, optic nerve tumor, oral cancer, osteosarcoma, ovarian cancer, Pancoast tumor, papillary thyroid cancer, paraganglioma, pinealoblastoma, pineocytoma, pituicytoma, pituitary adenoma, pituitary tumor, plasmacytoma, polyembryoma, precursor T-lymphoblastic lymphoma, primary central nervous system lymphoma, primary effusion lymphoma, preimary peritoneal cancer, prostate cancer, pancreatic cancer, pancreatic ductal cancer, pharyngeal cancer, pseudomyxoma periotonei, renal cell carcinoma, renal medullary carcinoma, retinoblastoma, rhabdomyoma, rhabdomyosarcoma, Richter's transformation, rectal cancer, sarcoma, Schwannomatosis, seminoma, Sertoli cell tumor, sex cord-gonadal stromal tumor, signet ring cell carcinoma, skin cancer, small blue round cell tumors, small cell carcinoma, soft tissue sarcoma, somatostatinoma, soot wart, spinal tumor, splenic marginal zone lymphoma, squamous cell carcinoma, synovial sarcoma, Sezary's disease, small intestine cancer, squamous carcinoma, stomach cancer, T-cell lymphoma, testicular cancer, thecoma, thyroid cancer, transitional cell carcinoma, throat cancer, urachal cancer, urogenital cancer, urothelial carcinoma, uveal melanoma, uterine cancer, verrucous carcinoma, visual pathway glioma, vulvar cancer, vaginal cancer, Waldenstrom's macroglobulinemia, Warthin's tumor, or Wilms' tumor.

4. (canceled)

5. The method of claim 3, wherein the cancer is breast cancer, colon cancer, lung cancer, pancreatic ductal cancer, prostate cancer, ovarian cancer, or head and neck cancer.

6-15. (canceled)

16. The method of claim 3, wherein the cells of the cancer exhibit a deficiency or loss of function of BRCA1 and/or BRCA2 genes.

17. The method of claim 3, wherein the cancer overexpresses a DNA damage repair enzyme.

18. (canceled)

19. The method of claim 3 further comprising administering a therapeutically effective amount of an optional therapeutic agent to the subject.

20. The method of claim 19, wherein the optional therapeutic agent comprises a poly ADP ribose polymerase (PARP) inhibitor, an ATM inhibitor, a wee1 inhibitor, and/or an ATR inhibitor.

21. The method of claim 20, wherein the optional therapeutic agent is a PARP inhibitor.

22. The method of claim 21, wherein the PARP inhibitor is olaparib, rucaparib, niraparib, or talazoparib.

23. The method of claim 19, wherein the cancer is prostate cancer, and the optional therapeutic agent comprises buserelin, degarelix, goserelin, histrelin, leuprolide, relugolix, triptorelin, bicalutamide, enzalutamide, bicalutamide, apalutamide, darolutamide, nilutamide, flutamide, abiraterone acetate, topilutamide, cyproterone acetate, megestrol acetate, chlormadinone acetate, spironolactone, oxendolone, or osaterone acetate.

24. The method of claim 19, wherein the cancer is breast cancer and the optional therapeutic agent comprises Soltamox® (tamoxifen), Arimidex® (anastrozole), Femara® (letrozole), Aromasin® (exemestane), Herceptin® (trastuzumab), Abraxane® (paclitaxel), Cytoxan® (cyclophosphamide), Taxol® (paclitaxel), Afinitor® (everolimus), Taxotere® (docetaxel), Xeloda® (capecitabine), Trexall® (methotrexate), Faslodex (fulvestrant), Adriamycin® (doxorubicin), Perjeta® (pertuzumab), Gemzar (gemcitabine), Tykerb® (lapatinib), Adrucil® (fluorouracil), Ibrance® (palbociclib), Verzenio® (abemaciclib), Fareston® (toremifene), Halaven® (eribulin), Menest, Kadcyla® (ado-trastuzumab emtransine), Androxy® (fluoxymesterone), Avastin® (bevacizumab), esterified estrogens, Herzuma® (trastuzumab), Ixempra® (ixabepilone), Kanjinti® (trastuzumab), Kisqali® (ribociclib), Ogivri® (trastuzumab), Ontruzant® (trastuzumab), Tepadina® (thiotepa), Trazimera® (trastuzumab), Velban® (vinblastine), Piqray® (alpelisib), Tecentriq® (atezolizumab), Enhertu® (fam-trastuzumab deruxtecan), Herceptin, Hylecta™ (hyaluronidase/trastuzumab), Infugem® (gemcitabine), Kisqali® Femara® Co-Pack (ribociclib and letrozole), Talzenna® (talazoparib), Trodelvy® (sacituzumab) or Tukysa™ (tukatinib).

25. The method of claim 19, wherein the cancer is colon cancer and the optional therapeutic agent comprises Xeloda® (capecitabine), Eloxatin® (oxaliplatin), fluorouracil, Avastin® (bevacizumab), leucovorin, Camptosar® (irinotecan), Stivarga® (regorafenib), Erbitux® (cetuximab), Vectibix® (panitumumab), Lonsurf® (tipiracil/trifluridine), Zaltrap® (ziv-aflibercept), Betaseron® (interferon beta-1b), Fusilev® (levoleucovorin), Wellcocorin® (methotrexate), Keytruda® (pembrolizumab), Mvasi® (bevacizumab-awwb), Cyramza® (ramucirumab), Yervoy® (ipilmumab), Opdivo® (nivolumab), Braftovi® (encorafenib), Khapzory® (levoleucovorin) or Zirabev® (bevacizumab-bvzr).

26. The method of claim 19, wherein the cancer is lung cancer and the optional therapeutic agent comprises Etopophos® (etoposide), Hycamtin® (topotecan), VePesid® (etoposide), Toposar® (etoposide), Opdivo® (nivolumab), Keytruda® (pembrolizumab), Tecentriq® (atezolizumab), Imfinizi® (durvalumab), methotrexate, cyclophosphamide, Carboplatin, Cisplatin, docetaxel, Gemcitabine, Irinotecan, Paclitaxel, Pemetrexed, Vinblastine, or Vinorelbine.

27. The method of claim 19, wherein the cancer is pancreatic ductal cancer and optional therapeutic agent comprises Gemzar® (Gemcitabine), fluorouracil, Afinitor® (everolimus), Tarceva® (erlotinib), Abraxane® (paclitaxel), capecitabine, Sutent® (sunitinib), pancreatin, methotrexate, Zanosar® (streptozocin), Mutamycin® (mitomycin), Onivyde® (irinotecan), bevacizumab, cetuximab, Infugem® (gemcitabine) or Lynparza® (olaparib).

28. The method of claim 19, wherein the cancer is head and neck cancer and optional therapeutic agent comprises Erbituz® (cetuximab), Taxotere® (docetaxel), Trexall® (methotrexate), Keytruda® (pembrolizumab) or Opdivo® (nivolumab).

29. The method of claim 19, wherein the cancer is prostate cancer and optional therapeutic agent comprises Suprefact® (buserelin), Firmagon® (degarelix), Zoladex® (goserelin), Vantas® (histrelin), Eligard® (leuprolide), Orgovyx® (relugolix), Trelstar® (triptorelin), Casodex® (bicalutamide), Eulexin® (flutamide), Nilandron® (nilutamide), Zytiga® (biraterone acetate), Erleada® (apalutamide), or Xtandi® (enzalutamide).

30. A kit comprising the compound of claim 1, or a pharmaceutically acceptable salt, solvate, and/or tautomer thereof, and instructions for administering the compound, or a pharmaceutically acceptable salt, solvate, and/or tautomer thereof to a subject having cancer.

31. (canceled)

32. (canceled)

33. A method, comprising administering a therapeutically effective amount of a compound of claim 1 to a subject in need thereof, wherein:
(a) the subject has cancer; and
(b) the cancer is characterized as having an overexpression of one or more DNA damage repair enzymes.

34. A method of treating a subject having cancer, the method comprising:
(a) determining whether an overexpression of one or more DNA damage repair enzymes is present or absent in a biological sample taken from the subject; and
(b) administering a therapeutically effective amount a compound of claim 1 to the subject if an overexpression of one or more DNA damage repair enzymes is present in the biological sample.

35-37. (canceled)
